(19) United States
(12) Patent Application Publication
(10) Pub. No.: US 2010/0311741 A1
(43) Pub. Date: Dec. 9, 2010

(54) Bicyclosulfonyl Acid (BCSA) Compounds and Their Use as Therapeutic Agents

(75) Inventors: Aigars Jirgensons, Riga (LV); Gundars Leitis, Riga (LV); Ivars Kalvinsh, Riga (LV); Daniel Robinson, Abingdon (GB); Paul Finn, Oxford (GB); Nagma Khan, Didcot (GB)

Correspondence Address: SWANSON & BRATSCHUN, L.L.C., 8210 SOUTHPARK TERRACE, LITTLETON, CO, 80120, US

(73) Assignee: INHIBOX LTD., Oxford Oxfordshire (GB)

(21) Appl. No.: 12/599,855

(22) PCT Filed: May 16, 2008
(86) PCT No.: PCT/GB2008/001683

§ 371 Date: Nov. 12, 2009

Related U.S. Application Data
(60) Provisional application No. 60/924,518, filed on May 18, 2007.

(51) Int. Cl. A61K 31/5377; C07D 275/06; C07D 417/10; C07D 417/04; C07D 513/04; C07D 333/52; C12N 5/07; A61K 31/428; A61K 31/426; A61K 31/4439; A61K 31/454; A61K 31/4709; A61K 31/381; A61K 31/437; A61P 19/02; A61P 29/00; A61P 17/06; A61P 31/18; A61P 35/00; A61P 11/06; A61P 11/00; A61P 1/00; A61P 9/04; A61P 9/10; A61P 3/04; A61P 31/00; A61P 37/00; A61P 25/00; A61P 5/48

(52) U.S. Cl. 514/233.8; 548/207; 548/181; 544/135; 546/271.1; 546/198; 546/167; 549/53; 546/114; 435/375; 514/373; 514/365; 514/338; 514/321; 514/314; 514/443; 514/301

(57) ABSTRACT

This invention pertains generally to the field of therapeutic compounds, and more particularly, to certain bicyclosulfonyl acid (BCSA) compounds which act as inhibitors of Tumour Necrosis Factor-α Converting Enzyme (TACE). The compounds are useful in the treatment of conditions mediated by TNF-α, such as rheumatoid arthritis; inflammation; psoriasis; septic shock; graft rejection; cachexia; anorexia; congestive heart failure; post ischaemic reperfusion injury; inflammatory disease of the central nervous system; inflammatory bowel disease; insulin resistance; HIV infection; cancer; chronic obstructive pulmonary disease (COPD); and asthma. The present invention also pertains to pharmaceutical compositions comprising such compounds, and the use of such compounds and compositions, both in vitro and in vivo, in the inhibition of TACE, and in the treatment of conditions that are ameliorated by the inhibition of TACE.

### RELATED APPLICATION

This application is related to U.S. patent application No. 60/924,518 filed 18 May 2007, the contents of which are incorporated herein by reference in their entirety.

### TECHNICAL FIELD

This invention pertains generally to the field of therapeutic compounds, and more particularly, to certain bicyclosulfonyl acid (BCSA) compounds which act as inhibitors of Tumour Necrosis Factor-α Converting Enzyme (TACE). The compounds are useful in the treatment of conditions mediated by TNF-α, such as such as rheumatoid arthritis; inflammation; psoriasis; septic shock; graft rejection; cachexia; anorexia; congestive heart failure; post-ischaemic reperfusion injury; inflammatory disease of the central nervous system; inflammatory bowel disease; insulin resistance; HIV infection; cancer; chronic obstructive pulmonary disease (COPD); and asthma. The present invention also pertains to pharmaceutical compositions comprising such compounds, and the use of such compounds and compositions, both in vitro and in vivo, in the inhibition of TACE, and in the treatment of conditions that are ameliorated by the inhibition of TACE.

### BACKGROUND

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### TACE

TACE (TNF-α Converting Enzyme) catalyses the formation of TNF-α from the membrane bound TNF-α precursor protein. TNF-α is a pro-inflammatory cytokine that is believed to have a role in numerous diseases, including the following:

Rheumatoid arthritis (see, e.g., Shire et al., 1998; Isomaki et al., 1997; Camussi et al., 1998).

Inflammation (see, e.g., Ksontini et al., 1988).

Psoriasis (see e.g. Le et al., 2005; Palladino et al., 2003).

Septic shock (see, e.g., Mathison et al., 1988, Miethke et al., 1992).

Graft rejection (see, e.g., Piguet et al., 1987).

Cachexia (see, e.g., Beutler et al., 1988).

Anorexia (see, e.g., Schattner et al., 1990).

Congestive heart failure (see, e.g., Packer et al., 1995; Ferrari et al., 1995).

Post-ischaemic reperfusion injury (see, e.g., Gu et al, 2006).

Inflammatory disease of the central nervous system (see, e.g., Grau et al., 1987).

Inflammatory bowel disease (see, e.g., McDonald et al., 1990).

Insulin resistance (see, e.g., Hotamisligil et al., 1993).

HIV infection (see, e.g., Peterson et al., 1992; Pallares-Trujillo et al., 1995).

Cancer (see, e.g., Old, 1985).

Chronic obstructive pulmonary disease (COPD) or asthma (see e.g. Trifilieff et al., 2002).

Additional examples of such diseases include: osteoarthritis, ulcerative colitis, Crohn's disease, multiple sclerosis, and degenerative cartilage loss.

A number of research groups have synthesized hydroxamic acid compounds comprising a sulfonamide group as potential anti-proliferative or anti-inflammatory agents (see, e.g., Levin et al, 1999; Ohtani et al, 1993; Owen et al, 2000, Yu et al, 2006).

Although a number of TACE inhibitors are known, many of these compounds are peptidic or peptide-like which suffer from problems in bioavailability and pharmacokinetic profile. Additionally, many of these compounds display non-selectivity, being potent inhibitors of matrix metalloproteases, and in particular MMP-1 (collagenase 1). MMP-1 inhibition has been postulated to cause joint pain in clinical trials of metalloproteases inhibitors (see, e.g., Scrip, 1988).

Long acting, selective, orally bioavailable, non-peptide inhibitors of TACE would thus be highly desirable for the treatment of the conditions described above.

### SUMMARY OF THE INVENTION

One aspect of the invention pertains to certain “bicyclosulfonyl acid” (BCSA) compounds, as described herein.

Another aspect of the present invention pertains to a pharmaceutical composition comprising a BCSA compound, as described herein, and a pharmaceutically acceptable carrier, diluent, or excipient.

Another aspect of the present invention pertains to a method of preparing a pharmaceutical composition comprising admixing a BCSA compound, as described herein, and a pharmaceutically acceptable carrier, diluent, or excipient.

Another aspect of the present invention pertains to a BCSA compound, as described herein, for use in a method of treatment (e.g., of a disease or disorder) of the human or animal body by therapy.

Another aspect of the present invention pertains to use of a BCSA compound, as described herein, in the manufacture of a medicament for the treatment (e.g., of a disease or disorder) of the human or animal body.

Another aspect of the present invention pertains to a method of treatment (e.g., of a disease or disorder) comprising administering to a patient in need of treatment a therapeutically effective amount of a BCSA compound, as described herein, preferably in the form of a pharmaceutical composition.

In one embodiment, the treatment is treatment of a disease or disorder that is mediated by TACE, for example, a disease or disorder that is known to be mediated by TACE.

In one embodiment, the treatment is treatment of a disease or disorder that is ameliorated by the inhibition of TACE, for example, a disease or disorder that is known to be ameliorated by the inhibition of TACE.

In one embodiment, the treatment is treatment of a disease or disorder that is treated by a TACE inhibitor, for example, a disease or disorder that is known to be treated by a TACE inhibitor.

In one embodiment, the treatment is treatment of rheumatoid arthritis; inflammation; psoriasis; septic shock; graft rejection; cachexia; anorexia; congestive heart failure; post-ischaemic reperfusion injury; inflammatory disease of the central nervous system; inflammatory bowel disease; insulin resistance; HIV infection; cancer; chronic obstructive pulmonary disease (COPD); or asthma.

In one embodiment, the treatment is treatment of: osteoarthritis, ulcerative colitis, Crohn's disease, multiple sclerosis, or degenerative cartilage loss.

In one embodiment, the treatment is treatment of inflammation.

In one embodiment, the treatment is treatment of rheumatoid arthritis.

In one embodiment, the treatment is treatment of psoriasis.

Another aspect of the present invention pertains to a method of inhibiting TACE in a cell, in vitro or in vivo, comprising contacting said cell with an effective amount of a BCSA compound, as described herein.

Another aspect of the present invention pertains to a method of regulating (e.g., inhibiting) cytokine release (e.g., TNF-α release) in a cell, in vitro or in vivo, comprising contacting said cell with an effective amount of a BCSA compound, as described herein.

Another aspect of the present invention pertains to a kit comprising (a) a BCSA compound, as described herein, preferably provided as a pharmaceutical composition and in a suitable container and/or with suitable packaging; and (b) instructions for use, for example, written instructions on how to administer the compound/composition.

Another aspect of the present invention pertains to compounds obtainable by a method of synthesis as described herein, or a method comprising a method of synthesis as described herein.

Another aspect of the present invention pertains to compounds obtained by a method of synthesis as described herein, or a method comprising a method of synthesis as described herein.

Another aspect of the present invention pertains to novel intermediates, as described herein, which are suitable for use in the methods of synthesis described herein.

Another aspect of the present invention pertains to the use of such novel intermediates, as described herein, in the methods of synthesis described herein.

As will be appreciated by one of skill in the art, features and preferred embodiments of one aspect of the invention will also pertain to other aspects of the invention.

### DETAILED DESCRIPTION OF THE INVENTION

### Compounds

One aspect of the present invention pertains to compounds of the following formula, and pharmaceutically acceptable salts, hydrates, and solvates thereof (collectively referred to herein as “bicyclosulfonyl acid” (BCSA) compounds):

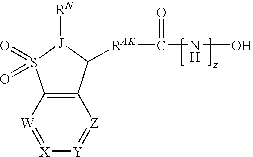

wherein:
W is independently —N═ or —CR^{PW}═;
X is independently —N═ or —CR^{PZ}═;
Y is independently —N═ or —CR^{PY}═;
Z is independently —N═ or —CR^{PZ}═;
each of —R^{PW}, —R^{PX}, —R^{PY}, and —R^{PZ}, if present, is independently —H or —R^{RS1};
wherein each —R^{RS1}, if present, is independently a ring substituent;
and wherein z is 0 or 1;
and wherein -J< is independently —N< or —CH<;
and wherein:
-R^{AK}- is independently:
a covalent bond,

### -R^{AK1}-, R^{AK2}-, -R^{AK3}-,

### -R^{AK4}-, -R^{AK1}-R^{AK4}, -R^{AK4}-R^{AK1}-, -R^{AK1}-R^{AK4}-R^{AK1}-,

### -R^{AK5}-, -R^{AK1}-R^{AK5}, -R^{AK5}-R^{AK1}-, or -R^{AK1}-R^{AK5}-R^{AK1}-;

wherein:
each -R^{AK1}- is independently saturated aliphatic C₁₋₆ alkylene, and is optionally substituted;
-R^{AK2}- is independently aliphatic C₂₋₆alkenylene, and is optionally substituted;
-R^{AK3}- is independently aliphatic C₂₋₆alkynylene, and is optionally substituted;
each -R^{AK4}- is independently saturated C₃₋₆cycloalkylene, and is optionally substituted; and
each -R^{AK5}- is independently C₃₋₆cycloalkenylene, and is optionally substituted;
and wherein:
-R^{N} is independently —H, —R^{NN}, -R^{NNN} or -L^{N}-R^{NNN};
wherein:
-L^{N}- is independently saturated aliphatic C₁₋₆alkylene, and is optionally substituted;
-R^{NN} is independently C₁₋₆alkyl, and is optionally substituted; and
-R^{NNN} is independently C₃₋₆cycloalkyl, C₃₋₇heterocyclyl, C₆₋₁₀carboaryl, or C₅₋₁₀heteroaryl, and is optionally substituted.

### Stereochemistry

Many of the chemical structures shown herein indicate one or more specific stereoisomeric configurations. Similarly, many of the chemical structures shown herein are silent in this respect, and do not indicate any stereoisomeric configuration. Similarly, many of the chemical structures shown herein indicate the specific stereoisomeric configurations at one or more positions, but are silent with respect to one or more other positions. Where a chemical structure herein is silent with respect to the stereoisomeric configuration at a position, that structure is intended to depict all possible stereoisomeric configurations at that position, both individually, as if each possible stereoisomeric configuration was individually recited, and also as a mixture (e.g., a racemic mixture) of stereoisomers.

Note, in particular, that the ring carbon atom adjacent to the group J (i.e., the atom marked with an asterisk (*) in the following formula) is necessarily a chiral centre.

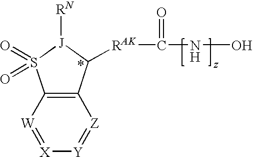

In one embodiment, the ring carbon atom adjacent to the group J (i.e., the atom marked with an asterisk (*)) has a configuration as shown in the following formula:

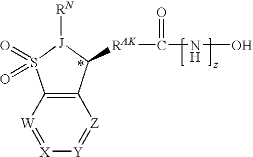

In one embodiment, the ring carbon atom adjacent to the group J (i.e., the atom marked with an asterisk (*)) has a configuration as shown in the following formula:

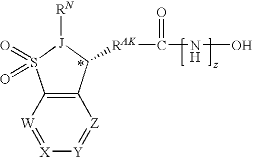

In one embodiment, the ring carbon atom adjacent to the group J (i.e., the atom marked with an asterisk (*)) is in the (R) configuration.

In one embodiment, the ring carbon atom adjacent to the group J (i.e., the atom marked with an asterisk (*)) is in the (S) configuration.

### The Groups W, X, Y, and Z

In one embodiment:

W is independently —N═ or —CR^{PW}═,
X is independently —N═ or —CR^{PX}═,
Y is independently —N═ or —CR^{PY}═, and
Z is independently —N═ or —CR^{PZ}═;
wherein exactly one or exactly two of W, X, Y, and Z is —N═.

In one embodiment:

W is independently —N═ or —CR^{PW}═,
X is independently —N═ or —CR^{PX}═,
Y is independently —N═ or —CR^{PY}═, and
Z is independently —N═ or —CR^{PZ}═;
wherein exactly one of W, X, Y, and Z is —N═.

In one embodiment:

W is independently —CR^{PW}═,
X is independently —CR^{PX}═,
Y is independently —CR^{PY}═, and
Z is independently —CR^{PZ}═.

In one embodiment:

W is independently —N═,
X is independently —CR^{PX}═,
Y is independently —CR^{PY}═, and
Z is independently —CR^{PZ}═.

In one embodiment:

W is independently —CR^{PW}═,
X is independently —N═,
Y is independently —CR^{PY}═, and
Z is independently —CR^{PZ}═.

In one embodiment:

W is independently —CR^{PW}═,
X is independently —CR^{PX}═,
Y is independently —N═, and
Z is independently —CR^{PZ}═.

In one embodiment:

W is independently —CR^{PW}═,
X is independently —CR^{PX}═,
Y is independently —CR^{PY}═, and
Z is independently —N═.

In one embodiment, each of —R^{PW}, —R^{PX}, —R^{PY}, and —R^{PZ}, if present, is independently —H.

The Group —[NH]_{z}—

In one embodiment, z is independently 1.

In one embodiment, z is independently 0.

### The Group J

In one embodiment, -J< is independently —N<.

In one embodiment, -J< is independently —CH<.

### The Group -R^{AK}

In one embodiment, -R^{AK}- is independently:

a covalent bond,

### -R^{AK1}-, -R^{AK2}-, -R^{AK3}-,

### -R^{AK4}-, -R^{AK1}-R^{AK4}-, -R^{AK4}-R^{AK1}-, -R^{AK1}-R^{AK4}-R^{AK1},

### -R^{AK5}-, -R^{AK1}-R^{AK5}-, -R^{AK5}-R^{AK1}-, or -R^{AK1}-R^{AK5}-R^{AK1}-.

In one embodiment, -R^{AK}- is independently:

### -R^{AK1}-, -R^{AK2}-, -R^{AK3}-,

### -R^{AK4}, -R^{AK1}-R^{AK4}-, -R^{AK4}-R^{AK1}-, -R^{AK1}-R^{AK4}-R^{AK1}-,

### -R^{AK5}-, -R^{AK1}-R^{AK5}-, -R^{AK5}-R^{AK1}-, or -R^{AK1}-R^{AK5}-R^{AK1}-.

In one embodiment, -R^{AK}- is independently:

### -R^{AK1}-, -R^{AK2}-, -R^{AK3}-,

### -R^{AK4}-, -R^{AK1}-R^{AK4}-, -R^{AK4}-R^{AK1}-, or -R^{AK1}-R^{AK4}-R^{AK1}-.

In one embodiment, -R^{AK}- is independently -R^{AK1}-, -R^{AK2}-, or -R^{AK3}-.

In one embodiment, -R^{AK}- is independently -R^{AK1}- or -R^{AK2}-.

In one embodiment, -R^{AK}- is independently -R^{AK1}-.

In one embodiment, -R^{AK}- is independently -R^{AK2}-.

In one embodiment, -R^{AK}- is independently -R^{AK3}-.

In one embodiment, -R^{AK}- is independently -R^{AK1}- or a covalent bond.

In one embodiment, -R^{AK}- is independently a covalent bond.

In one embodiment, -R^{AK}- is independently:

### -R^{AK4}-, -R^{AK1}-R^{AK4}-, -R^{AK4}-R^{AK1}-, or -R^{AK1}-R^{AK4}-R^{AK1}-.

In one embodiment, -R^{AK}- is independently -R^{AK4}-.

In one embodiment, -R^{AK}- is independently -R^{AK1}-R^{AK4}.

In one embodiment, -R^{AK}- is independently -R^{AK4}-R^{AK1}-.

In one embodiment, -R^{AK}- is independently -R^{AK1}-R^{AK4}-R^{AK1}-.

### The Group —R^{AK1}-

In one embodiment, each -R^{AK1}-, if present, is independently saturated aliphatic C₁₋₆alkylene; and is optionally substituted.

In one embodiment, each -R^{AK1}-, if present, is independently saturated aliphatic C₁₋₄alkylene; and is optionally substituted.

In one embodiment, each -R^{AK1}-, if present, is independently unsubstituted or substituted, for example, with one or more substitutents, for example, with one or more (e.g., 1, 2, 3) substituents -R^{G1}.

In one embodiment, each -R^{AK1}-, if present, is independently unsubstituted.

In one embodiment, each -R^{AK1}-, if present, is independently —(CH₂)_{q}—, wherein q is independently 1, 2, 3, 4, 5, or 6.

In one embodiment, each -R^{AK1}-, if present, is independently —(CH₂)—, —(CH₂)₂—, —(CH₂)₃—, or —(CH₂)₄—.

In one embodiment, each -R^{AK1}-, if present, is independently —(CH₂)—, —(CH₂)₂—, or —(CH₂)₃—.

In one embodiment, each -R^{AK1}-, if present, is independently —(CH₂)— or —(CH₂)₂—.

In one embodiment, each —R^{AK1}—, if present, is independently —(CH₂)—.

### The Group —R^{AK2}-

In one embodiment, -R^{AK2}-, if present, is independently aliphatic C₂₋₆alkenylene; and is optionally substituted.

The term “C₂₋₆alkenylene”, as used herein, pertains to a divalent bidentate aliphatic hydrocarbyl group having from 2 to 6 carbon atoms and having at least one carbon-carbon double bond, but no carbon-carbon triple bonds.

In one embodiment, -R^{AK2}-, if present, is independently aliphatic C₂₋₄alkenylene; and is optionally substituted.

In one embodiment, -R^{AK2}-, if present, is independently unsubstituted or substituted, for example, with one or more substitutents, for example, with one or more (e.g., 1, 2, 3) substituents -R^{G1}.

In one embodiment, —R^{AK2}—, if present, is independently unsubstituted.

In one embodiment, —R^{AK2}—, if present, is independently:

### —CH═CH—,

### —C(CH₃)═CH—, —CH═C(CH₃)—,

### —CH═CH—CH₂—,

### —C(CH₃)═CH—CH₂—, —CH═C(CH₃)—CH₂—, —CH═CH—CH(CH₃)—,

### —CH₂—CH═CH—,

### —CH(CH₃)—CH═CH—, —CH₂—C(CH₃)═CH—, —CH₂—CH═C(CH₃)—,

### —CH═CH—CH₂—CH₂—, —CH₂—CH═CH—CH₂—, or —CH₂—CH₂—CH═CH—.

### The Group —R^{AK3}-

In one embodiment, -R^{AK3}-, if present, is independently aliphatic C₂₋₆alkynylene; and is optionally substituted.

The term “C₂₋₆alkynylene”, as used herein, pertains to a divalent bidentate aliphatic hydrocarbyl group having at least one carbon-carbon triple bond, and, optionally also one or more carbon-carbon double bonds.

In one embodiment, -R^{AK3}-, if present, is independently aliphatic C₂₋₄alkynylene; and is optionally substituted.

In one embodiment, -R^{AK3}-, if present, is independently unsubstituted or substituted, for example, with one or more substitutents, for example, with one or more (e.g., 1, 2, 3) substituents —R^{G1}.

In one embodiment, -R^{AK3}-, if present, is independently unsubstituted.

In one embodiment, -R^{AK3}-, if present, is independently:

### —C≡C—,

### —C≡C—CH₂—, —C≡C—CH(CH₃)—,

### —CH₂—C≡C—, —CH(CH₃)—C≡C—,

### —C≡C—CH₂—CH₂—, —C≡C—CH(CH₃)—CH₂—, —C≡C—CH₂—CH(CH₃)—,

### —CH₂—C≡C—CH₂—, —CH(CH₃)—C≡C—CH₂—, —CH₂—C═C—CH(CH₃)—,

### —CH₂—CH₂—C≡C—, —CH(CH₃)—CH₂—C≡C—, —CH₂—CH(CH₃)—C≡C—,

### —C≡C—CH═CH—, —C≡C—C(CH₃)═CH—, —C≡C—CH═C(CH₃)—,

### —CH═CH—C≡C—, —C(CH₃)═CH—C≡C—, or —CH═C(CH₃)—C≡C—.

### The Groups -R^{AK4}-, -R^{AK1}-R^{AK4}-, -R^{AK4}-R^{AK1}-, and -R^{AK1}-R^{AK4}-R^{AK1}-

In one embodiment, each -R^{AK4}-, if present, is independently saturated C₃₋₆cycloalkylene; and is optionally substituted.

The term “saturated C₃₋₆cycloalkylene”, as used herein, pertains to a divalent bidentate saturated carbocyclic group having from 3 to 6 ring atoms, wherein said ring atoms are carbon atoms, and wherein one or two of said ring atoms are points of attachment.

In one embodiment, each -R^{AK4}-, if present, is independently saturated C₃₋₅cycloalkylene; and is optionally substituted.

In one embodiment, each -R^{AK4}-, if present, is independently saturated C₃₋₄cycloalkylene; and is optionally substituted.

In one embodiment, each -R^{AK4}-, if present, is independently saturated C₄₋₆cycloalkylene; and is optionally substituted.

In one embodiment, each -R^{AK4}-, if present, is independently saturated C₅₋₆cycloalkylene; and is optionally substituted.

In one embodiment, each -R^{AK4}-, if present, is independently unsubstituted or substituted, for example, with one or more substitutents, for example, with one or more (e.g., 1, 2, 3) substituents —R^{G1}.

In one embodiment, each -R^{AK4}-, if present, is independently unsubstituted.

In one embodiment, each -R^{AK4}-, if present, is independently: cyclopropyl-di-yl, cyclobutyl-di-yl, cyclopentyl-di-yl, or cyclohexyl-di-yl.

In one embodiment, each -R^{AK4}-, if present, is independently cyclopropyl-di-yl.

In one embodiment, each -R^{AK4}-, if present, is independently cyclopropyl-1,1-di-yl.

In one embodiment, each -R^{AK1}-R^{AK4}-, if present, is independently: methylene-cyclopropyl-di-yl, methylene-cyclobutyl-di-yl, methylene-cyclopentyl-di-yl, or methylene-cyclohexyl-di-yl.

In one embodiment, each -R^{AK4}-R^{AK1}-, if present, is independently: cyclopropyl-di-yl-methylene, cyclobutyl-di-yl-methylene, cyclopentyl-di-yl-methylene, or cyclohexyl-di-yl-methylene.

In one embodiment, -R^{AK1}-R^{AK4}-R^{AK1}-, if present, is independently: methylene-cyclopropyl-di-yl-methylene, methylene-cyclobutyl-di-yl-methylene, methylene-cyclopentyl-di-yl-methylene, or methylene-cyclohexyl-di-yl-methylene.

### The Group -R^{AK5}-

In one embodiment, each -R^{AK5}-, if present, is independently C₃₋₆cycloalkenylene; and is optionally substituted.

The term “C₃₋₆cycloalkenylene”, as used herein, pertains to a divalent bidentate carbocyclic group having from 3 to 6 ring atoms and having at least one carbon-carbon double bond in the ring, but no carbon-carbon triple bonds in the ring, wherein said ring atoms are carbon atoms, and wherein one or two of said ring atoms are points of attachment.

In one embodiment, each -R^{AK5}-, if present, is independently C₃₋₆cycloalkenylene; and is optionally substituted.

In one embodiment, each -R^{AK5}-, if present, is independently C₃₋₄cycloalkenylene; and is optionally substituted.

In one embodiment, each -R^{AK5}-, if present, is independently C₄₋₆cycloalkenylene; and is optionally substituted.

In one embodiment, each -R^{AK5}-, if present, is independently C₅₋₆cycloalkenylene; and is optionally substituted.

In one embodiment, each -R^{AK5}-, if present, is independently unsubstituted or substituted, for example, with one or more substitutents, for example, with one or more (e.g., 1, 2, 3) substituents -R^{G1}.

In one embodiment, each -R^{AK5}-, if present, is independently unsubstituted.

In one embodiment, each -R^{AK5}-, if present, is independently: cyclopropenyl-di-yl, cyclobutenyl-di-yl, cyclopentenyl-di-yl, or cyclohexenyl-di-yl.

In one embodiment, each -R^{AK1}-R^{AK5}-, if present, is independently: methylene-cyclopropenyl-di-yl, methylene-cyclobutenyl-di-yl, methylene-cyclopentenyl-di-yl, or methylene-cyclohexenyl-di-yl.

In one embodiment, each -R^{AK5}-R^{AK1}-, if present, is independently: cyclopropenyl-di-yl-methylene, cyclobutenyl-di-yl-methylene, cyclopentenyl-di-yl-methylene, or cyclohexenyl-di-yl-methylene.

In one embodiment, -R^{AK1}-R^{AK5}-RAK¹-, if present, is independently: methylene-cyclopropenyl-di-yl-methylene, methylene-cyclobutenyl-di-yl-methylene, methylene-cyclopentenyl-di-yl-methylene, or methylene-cyclohexenyl-di-yl-methylene.

### Substituents —R^{G1}

In one embodiment, each -R^{G1}, if present, is independently —F, —Cl, —Br, —I, —OH, —OR^{A1}, —OCF₃, —C(═O)OH, —C(═O)OR^{A1}, —NH₂, —NHR^{A1}, —NR^{Al}₂, —NR^{A2}R^{A3}, —C(═O)—NH₂, —C(═O)—NHR^{A1}, —C(═O)—NR^{Al}₂, —C(═O)—NR^{A2}R^{A3}, phenyl, or benzyl; wherein each R^{A1} is independently C₁₋₄alkyl, phenyl, or benzyl; and each —NR^{A2}R^{A3} is independently pyrrolidino, piperidino, piperizino, or morpholino, and is independently unsubstituted or substituted with one or more groups selected from C₁₋₃alkyl and —CF₃.

In one embodiment, each —R^{G1}, if present, is independently —F, —Cl, —Br, —I, —OH, —OMe, —OEt, or —OCF₃.

### The Group —R^{N}

In one embodiment, -R^{N} is independently —H, -R^{NN}, -R^{NNN}, or -L^{N}-R^{NNN}.

In one embodiment, -R^{N} is independently —H, -R^{NNN}, or -L^{N}-R^{NNN}.

In one embodiment, -R^{N} is independently —H or -R^{NN}.

In one embodiment, -R^{N} is independently -R^{NNN} or -L^{N}-R^{NNN}.

In one embodiment, -R^{N} is independently —H.

In one embodiment, -R^{N} is independently -R^{NN}.

In one embodiment, -R^{N} is independently -R^{NNN}.

In one embodiment, —R^{N} is independently -L^{N}-R^{NNN}.

### The Group -L^{N}-

In one embodiment, -L^{N}-, if present, is independently saturated aliphatic C₁₋₆alkylene, and is optionally substituted.

In one embodiment, -L^{N}-, if present, is independently saturated aliphatic C₁₋₃alkylene, and is optionally substituted.

In one embodiment, -L^{N}-, if present, is independently unsubstituted or substituted, for example, with one or more substitutents, for example, with one or more (e.g., 1, 2, 3) substituents -R^{G2}.

In one embodiment, -L^{N}-, if present, is independently unsubstituted.

In one embodiment, -L^{N}-, if present, is independently —CH₂—, —CH₂CH₂—, or —CH₂CH₂CH₂—.

In one embodiment, -L^{N}-, if present, is independently —CH₂— or —CH₂CH₂—.

In one embodiment, -L^{N}-, if present, is independently —CH₂—.

### Substituents —R^{G2}

In one embodiment, each -R^{G2}, if present, is independently —F, —Cl, —Br, —I, —OH, —OR^{A1}, —OCF₃, —C(═O)OH, —C(═O)OR^{A1}, —NH₂, —NHR^{A1}, —NR^{Al}₂, —NR^{A2}R^{A3}, —C(═O)—NH₂, —C(═O)—NHR^{A1}, —C(═O)—NR^{A1}₂, —C(═O)—NR^{A2}R^{A3}, phenyl, or benzyl; wherein each R^{A1} is independently C₁₋₄alkyl, phenyl, or benzyl; and each —NR^{A2}R^{A3} is independently pyrrolidino, piperidino, piperizino, or morpholino, and is independently unsubstituted or substituted with one or more groups selected from C₁₋₃alkyl and —CF₃.

In one embodiment, each -R^{G2}, if present, is independently —F, —Cl, —Br, —I, —OH, —OMe, —OEt, or —OCF₃.

### The Group -R^{NN}

In one embodiment, -R^{NN}, if present, is independently C₁₋₆alkyl, and is optionally substituted.

In one embodiment, -R^{NN}, if present, is independently C₁₋₄alkyl, and is optionally substituted.

In one embodiment, -R^{NN}, if present, is independently unsubstituted or substituted, for example, with one or more substitutents, for example, with one or more (e.g., 1, 2, 3) substituents -R^{G3}.

In one embodiment, -R^{NN}, if present, is independently unsubstituted.

In one embodiment, -R^{NN}, if present, is independently -Me, -Et, -nPr, or -iPr.

### Substituents -R^{G3}

In one embodiment, each -R^{G3}, if present, is independently —F, —Cl, —Br, —I, —OH, —OR^{A1}, —OCF₃, —C(═O)OH, —C(═O)OR^{A1}, —NH₂, —NHR^{A1}, —NR^{Al}₂, —NR^{A2}R^{A3}, —C(═O)—NH₂, —C(═O)—NHR^{A1}, —C(═O)—NR^{A1}₂, —C(═O)—NR^{A2}R^{A3}; wherein each R^{A1} is independently C₁₋₄alkyl, phenyl, or benzyl; and each —NR^{A2}R^{A3} is independently pyrrolidino, piperidino, piperizino, or morpholino, and is independently unsubstituted or substituted with one or more groups selected from C₁₋₃alkyl and —CF₃.

In one embodiment, each —R^{G3}, if present, is independently —F, —Cl, —Br, —I, —OH, —OMe, —OEt, or —OCF₃.

### The Group -R^{NNN}

In one embodiment, -R^{NNN}, if present, is independently C₃₋₆cycloalkyl, C₃₋₇heterocyclyl, C₆₋₁₀carboaryl, or C₆₋₁₀heteroaryl; and is optionally substituted.

In one embodiment, -R^{NNN}, if present, is independently cyclopropyl, cyclobutyl, cyclopentyl, cyclohexyl, pyrrolidinyl, imidazolidinyl, pyrazolidinyl, piperidinyl, piperizinyl, morpholinyl, thiomorpholinyl, azepinyl, diazepinyl, phenyl, naphthyl, furanyl, thienyl, pyrrolyl, imidazolyl, pyrazolyl, triazolyl, oxazolyl, isoxazoyl, thiazolyl, isothiazolyl, pyridyl, pyrazinyl, pyrimidinyl, pyridazinyl, benzofuranyl, isobenzofuranyl, indazolyl, purinyl, quinolinyl, isoquinolinyl, naphthyridinyl, quinoxalinyl, quinazolinyl, cinnolinyl, indoyl, isoindolyl, carbazolyl, carbolinyl, acridinyl, phenoxazinyl, or phenothiazinyl; and is optionally substituted.

In one embodiment, -R^{NNN}, if present, is independently C₆₋₁₀carboaryl or C₆₋₁₀heteroaryl, and is optionally substituted.

In one embodiment, -R^{NNN}, if present, is independently phenyl, naphthyl, furanyl, thienyl, pyrrolyl, imidazolyl, pyrazolyl, triazolyl, oxazolyl, isoxazoyl, thiazolyl, isothiazolyl, pyridyl, pyrazinyl, pyrimidinyl, pyridazinyl, benzofuranyl, isobenzofuranyl, indazolyl, purinyl, quinolinyl, isoquinolinyl, naphthyridinyl, quinoxalinyl, quinazolinyl, cinnolinyl, indoyl, isoindolyl, carbazolyl, carbolinyl, acridinyl, phenoxazinyl, or phenothiazinyl; and is optionally substituted.

In one embodiment, -R^{NNN}, if present, is independently phenyl, naphthyl, furanyl, thienyl, pyrrolyl, imidazolyl, pyrazolyl, triazolyl, oxazolyl, isoxazoyl, thiazolyl, isothiazolyl, pyridyl, pyrazinyl, pyrimidinyl, or pyridazinyl; and is optionally substituted.

In one embodiment, -R^{NNN}, if present, is independently phenyl, naphthyl, pyridyl, pyrazinyl, pyrimidinyl, pyridazinyl, or pyrazolyl; and is optionally substituted.

In one embodiment, -R^{NNN}, if present, is independently phenyl, naphthyl, pyridyl, or pyrazolyl; and is optionally substituted.

In one embodiment, -R^{NNN}, if present, is independently phenyl or naphthyl; and is optionally substituted.

In one embodiment, -R^{NNN}, if present, is independently phenyl; and is optionally substituted.

In one embodiment, -R^{NNN}, if present, is independently unsubstituted or substituted, for example, unsubstituted or substituted with one or more (e.g., 1, 2, 3) substituents.

In one embodiment, -R^{NNN}, if present, is independently phenyl; and is optionally substituted at the para position; and is unsubstituted at all other positions.

In one embodiment, each substituent on -R^{NNN}, if present, is independently -R^{S}.

In one embodiment, -R^{NNN}, if present, is independently unsubstituted.

### Substituents —R^{RS1}

In one embodiment, each -R^{RS1}, if present, is independently as defined for -R^{S}.

In one embodiment, each —R^{RS1}, if present, is independently —F, —Cl, —Br, —I, —R^{A1}, —CF₃, —OH, —OR^{A1}, —OCF₃, —C(═O)OH, —C(═O)OR^{A1}, —NH₂, —NHR^{A1}, —NR^{A1}₂, —NR^{A2}R^{A3}, —C(═O)—NH₂, —C(═O)—NHR^{A1}, —C(═O)—NR^{A1}₂, —C(═O)—NR^{A2}R^{A3}, phenyl, or benzyl; wherein each R^{A1} is independently C₁₋₄alkyl, phenyl, or benzyl; and each —NR^{A2}R^{A3} is independently pyrrolidino, piperidino, piperazino, or morpholino, and is independently unsubstituted or substituted with one or more groups selected from C₁₋₃alkyl and —CF₃; and additionally, two adjacent groups -R^{RS1}, if present, may form —OCH₂O—, —OCH₂CH₂O—, or —OCH₂CH₂CH₂O—.

In one embodiment, each -R^{RS1}, if present, is independently —F, —Cl, —Br, —I, -Me, -Et, —CF₃, —OH, —OMe, —OEt, —OCF₃, or phenyl; and additionally, two adjacent groups -R^{RS1}, if present, may form —OCH₂CH₂O—.

In one embodiment, each -R^{RS1}, if present, is independently —F, —Cl, —Br, -Me, —CF₃, —OMe, —OEt, or phenyl; and additionally, two adjacent groups —R^{RS1}, if present, may form —OCH₂CH₂O—.

### Substituents —R^{S}

In one embodiment, each -R^{S}, if present, is independently:

—F, —Cl, —Br, —I,

—R^{D1},

—CF₃, —CH₂CF₃, —CF₂CF₂H,

—OH,

-L¹-OH,

—O-L¹-OH,

—OR^{D1},

-L¹-OR^{D1},

—O-L¹-OR^{D1},

—OCF₃, —OCH₂CF₃, —OCF₂CF₂H,

—SH,

—SR^{D1}, —SCF₃,

—CN,

—NO₂,

—NH₂, —NHR^{D1}, —NR^{D1}₂, —NR^{N1}R^{N2},

-L¹-NH₂, -L¹-NHR^{D1}, -L¹-NR^{D1}₂, -L¹-NR^{N1}R^{N2},

—O-L¹-NH₂, —O-L¹-NHR^{D1}, —O-L¹-NR^{D1}₂, —O-L¹-NR^{N1}R^{N2}, —NH-L¹-NH₂, —NH-L¹-NHR^{D1}, —NH-L¹-NR^{D1}₂, —NH-L¹-NR^{N1}R^{N2}, —NR^{D1}-L¹-NH₂, —NR^{D1}-L¹-NHR^{D1}, —NR^{D1}-L¹-NR^{D1}₂, —NR^{D1}-L¹-NR^{N1}R^{N2},

—C(═O)OH,

—C(═O)OR^{D1},

—C(═O)NH₂, —C(═O)NHR^{D1}, —C(═O)NR^{D1}₂, —C(═O)NR^{N1}R^{N2},

—NHC(═O)R^{D1}, —NR^{D1}C(═O)R^{D1},

—NHC(═O)OR^{D1}, —NR^{D1}C(═O)OR^{D1},

—OC(═O)NH₂, —OC(═O)NHR^{D1}, —OC(═O)NR^{D1}₂, —OC(═O)NR^{N1}R^{N2},

—OC(═O)R^{D1},

—C(═O)R^{D1},

—NHC(═O)NH₂, —NHC(═O)NHR^{D1}, —NHC(═O)NR^{D1}₂, —NHC(═O)NR^{N1}R^{N2},

—NR^{D1}C(═O)NH₂, —NR^{D1}C(═O)NHR^{D1}, —NR^{D1}C(═O)NR^{D1}₂, —NR^{D1}C(═O)NR^{N1}R^{N2},

—NHS(═O)₂R^{D1}, —NR^{D1}S(═O)₂R^{D1},

—S(═O)₂NH₂, —S(═O)₂NHR^{D1}, —S(═O)₂NR^{D1}₂, —S(═O)₂NR^{N1}R^{N2},

—S(═O)R^{D1},

—S(═O)₂R^{D1},

—OS(═O)₂R^{D1},

—S(═O)₂OR^{D1},

═O,

═NR^{D1},

═NOH, or

═NOR^{D1};

and additionally, two ring adjacent groups -R^{S}, if present, may together form a group —O-L²-O—;

wherein:
each -L¹- is independently saturated aliphatic C₁₋₆alkylene, aliphatic C₂₋₅alkenylene, or aliphatic C₂₋₅alkynylene;
each -L²- is independently saturated aliphatic C₁₋₃alkylene;

in each group —NR^{N1}R^{N2}, -R^{N1} and -R^{N2}, taken together with the nitrogen atom to which they are attached, form a 5-, 6-, or 7-membered non-aromatic ring having exactly 1 ring heteroatom or exactly 2 ring heteroatoms, wherein one of said exactly 2 ring heteroatoms is N, and the other of said exactly 2 ring heteroatoms is independently N, O, or S;

each -R^{D1} is independently:
-R^{E1}, -R^{E2}, -R^{E3}, -R^{E4}, -R^{E5}, -R^{E6}, -R^{E7}, -R^{E8},
-L³-R^{E4}, -L³-R^{E6}, -L³-R^{E6}, -L³-R^{E7}, or -L³-R^{E8};
   wherein:

each -R^{E1} is independently saturated aliphatic C₁₋₆alkyl;

each -R^{E2} is independently aliphatic C₂₋₆alkenyl;

each -R^{E3} is independently aliphatic C₂₋₆alkynyl;

each -R^{E4} is independently saturated C₃₋₆cycloalkyl;

each -R^{E6} is independently C₃₋₆cycloalkenyl;

each -R^{E6} is independently non-aromatic C₃₋₇heterocyclyl;

each -R^{E7} is independently C₆₋₁₄carboaryl;

each -R^{E8} is independently C₅₋₁₄heteroaryl;

each -L³- is independently saturated aliphatic C₁₋₃alkylene;

and wherein:

each C₁₋₆alkyl, C₂₋₆alkenyl, C₂₋₆alkynyl, C₃₋₆cycloalkyl, C₃₋₆cycloalkenyl, non-aromatic C₃₋₇heterocyclyl, C₆₋₁₄-carboaryl, C₅₋₁₄heteroaryl, and C₁₋₃alkylene is optionally substituted, for example, with one or more (e.g., 1, 2, 3) substituents -R^{G4}, wherein each -R^{G4} is independently:

—F, —Cl, —Br, —I,

-R^{F1},

—CF₃, —CH₂CF₃, —CF₂CF₂H,

—OH,

-L⁴-OH,

—O-L⁴- OH,

—OR^{F1},

-L⁴-OR^{F1},

—O-L⁴-OR^{F1},

—OCF₃, —OCH₂CF₃, —OCF₂CF₂H,

—SH,

—SR^{F1}, —SCF₃,

—CN,

—NO₂,

—NH₂, —NHR^{F1}, —NR^{F1}₂, —NR^{N3}R^{N4},

-L⁴-NH₂, -L⁴-NHR^{F1}, -L⁴-NR^{F1}₂, or -L⁴-NR^{N3}R^{N4},

—O-L⁴-NH₂, —O-L⁴-NHR^{F1}, —O-L⁴-NR^{F1}₂, —O-L⁴-NR^{N3}R^{N4},

—NH-L⁴-NH₂, —NH-L⁴-NHR^{F1}, —NH-L⁴-NR^{F1}₂, —NH-L⁴-NR^{N3}R^{N4},

—NR^{F1}-L⁴-NH₂, —NR^{F1}-L⁴-NHR^{F1}, —NR^{F1}-L⁴- NR^{F1}₂, —NR^{F1}-L⁴-NR^{N3}R^{N4},

—C(═O)OH,

—C(═O)OR^{F1},

—C(═O)NH₂, —C(═O)NHR^{F1}, —C(═O)NR^{F1}₂, or —C(═O)NR^{N3}R^{N4};

wherein:

each —R^{F1} is independently saturated aliphatic C₁₋₄alkyl, phenyl, or benzyl;

each -L⁴- is independently saturated aliphatic C₁₋₅alkylene; and

in each group —NR^{N3}R^{N4}, -R^{N3} and -R^{N4}, taken together with the nitrogen atom to which they are attached, form a 5-, 6-, or 7-membered non-aromatic ring having exactly 1 ring heteroatom or exactly 2 ring heteroatoms, wherein one of said exactly 2 ring heteroatoms is N, and the other of said exactly 2 ring heteroatoms is independently N, O, or S.

In one embodiment, each -R^{S}, if present, is independently:

—F, —Cl, —Br, —I,

-R^{D1},

—CF₃, —CH₂CF₃, —CF₂CF₂H,

—OH,

-L¹-OH,

—O-L¹-OH,

—OR^{D1},

-L¹-OR^{D1},

—O-L¹-OR^{D1},

—OCF₃, —OCH₂CF₃, —OCF₂CF₂H,

—SH,

—SR^{D1}, —SCF₃,

—CN,

—NO₂,

—NH₂, —NHR^{D1}, —NR^{D1}₂, —NR^{N1}R^{N2},

-L¹-NH₂, -L¹-NHR^{D1}, -L¹-NR^{D1}₂, -L¹-NR^{N1}R^{N2},

—O-L¹-NH₂, —O-L¹-NR^{D1}₂, —O-L¹-NR^{N1}R^{N2},

—NH-L¹-NH₂, —NH—C—NHR^{D1}, —NH-L¹-NR^{D1}₂, —NH-L¹-NR^{N1}R^{N2},

—NR^{D1}-L¹-NH₂, —NR^{D1}-L¹-NHR^{D1}, —NR^{D1}-L¹-NR^{D1}₂, —NR^{D1}-L¹-NR^{N1}R^{N2},

—C(═O)OH,

—C(═O)OR^{D1},

—C(═O)NH₂, —C(═O)NHR^{D1}, —C(═O)NR^{D1}₂, —C(═O)NR^{N1}R^{N2},

—NHC(═O)R^{D1}, —NR^{D1}C(═O)R^{D1},

—OC(═O)R^{D1},

—C(═O)R^{D1},

—NHS(═O)₂R^{D1}, —NR^{D1}S(═O)₂R^{D1},

—S(═O)₂NH₂, —S(═O)₂NHR^{D1}, —S(═O)₂NR^{D1}₂, or —S(═O)₂NR^{N1}R^{N2};

and additionally, two ring adjacent groups -R^{S}, if present, may together form a group —O-L²-O—.

In one embodiment, each -R^{S}, if present, is independently —OR^{D1}.

In one embodiment, each group —NR^{N1}R_{N2}, if present, is independently pyrrolidino, imidazolidino, pyrazolidino, piperidino, piperizino, morpholino, thiomorpholino, azepino, or diazepino, and is independently unsubstituted or substituted, for example, with one or more (e.g., 1, 2, 3) groups selected from C₁₋₃alkyl and —CF₃.

In one embodiment, each group —NR^{N1}R^{N2}, if present, is independently pyrrolidino, piperidino, piperizino, or morpholino, and is independently unsubstituted or substituted, for example, with one or more (e.g., 1, 2, 3) groups selected from C₁₋₃alkyl and —CF₃.

In one embodiment, each -R^{D1}, if present, is independently:

-R^{E1}, -R^{E3}, -R^{E4}, -R^{E7}, -R^{E8},

-L³-R^{E4}, -L³-R^{E7}, or -L³-R^{E8}.

In one embodiment, each -R^{D1}, if present, is independently:

-R^{E1}, -R^{E3}, -R^{E7}, -R^{E8}, -L³-R^{E7}, or -L³R^{E8}.

In one embodiment, each -R^{D1}, if present, is independently -L³-R^{E7} or -L³-R^{E8}.

In one embodiment, each -R^{D1}, if present, is independently -R^{E3}.

In one embodiment, each -R^{E1}, if present, is independently methyl, ethyl, n-propyl, i-propyl, n-butyl, i-butyl, s-butyl, or t-butyl, and is optionally substituted.

In one embodiment, each -R^{E2}, if present, is independently aliphatic C₂₋₄alkenyl, and is optionally substituted.

In one embodiment, each -R^{E2}, if present, is independently —CH₂—CH═CH₂, and is optionally substituted.

In one embodiment, each -R^{E3}, if present, is independently aliphatic C₃₋₅alkynyl, and is optionally substituted.

In one embodiment, each -R^{E3}, if present, is independently —CH₂—C≡CH, —CH(CH₃)—C≡CH, —CH₂—C≡C—CH₃, —CH(CH₃)—C≡C—CH₃, —CH₂—C≡C—CH₂—CH₃, or —CH₂—CH₂—C≡CH, and is optionally substituted.

In one embodiment, each -R^{E4}, if present, is independently cyclopropyl, cyclobutyl, cyclopentyl, or cyclohexyl, and is optionally substituted.

In one embodiment, each -R^{E6}, if present, is independently azetidinyl, pyrrolidinyl, imidazolidinyl, pyrazolidinyl, piperidinyl, piperazinyl, morpholinyl, azepinyl, diazepinyl, tetrahydrofuranyl, tetrahydropyranyl, dioxanyl, and is optionally substituted.

In one embodiment, each -R^{E6}, if present, is independently pyrrolidinyl, piperidinyl, piperazinyl, morpholinyl, tetrahydrofuranyl, or tetrahydropyranyl, and is optionally substituted.

In one embodiment, each -R^{E7}, if present, is independently phenyl or naphthyl; and is optionally substituted.

In one embodiment, each -R^{E7}, if present, is independently phenyl; and is optionally substituted.

In one embodiment, each -R^{E8}, if present, is independently furanyl, thienyl, pyrrolyl, imidazolyl, pyrazolyl, triazolyl, oxazolyl, isoxazoyl, thiazolyl, isothiazolyl, pyridyl, pyrazinyl, pyrimidinyl, pyridazinyl, benzofuranyl, isobenzofuranyl, indazolyl, purinyl, quinolinyl, isoquinolinyl, naphthyridinyl, quinoxalinyl, quinazolinyl, cinnolinyl, indolyl, isoindolyl, carbazolyl, carbolinyl, acridinyl, phenoxazinyl, or phenothiazinyl; and is optionally substituted.

In one embodiment, each -R^{E8}, if present, is independently furanyl, thienyl, pyrrolyl, imidazolyl, pyrazolyl, triazolyl, oxazolyl, isoxazolyl, thiazolyl, isothiazolyl, pyridyl, pyrazinyl, pyrimidinyl, pyridazinyl, quinolinyl, or isoquinolinyl; and is optionally substituted.

In one embodiment, each -R^{E8}, if present, is independently furanyl, pyrrolyl, pyrazolyl, triazolyl, oxazolyl, isoxazolyl, thiazolyl, isothiazolyl, pyridyl, quinolinyl, or isoquinolinyl; and is optionally substituted.

In one embodiment, each -L¹-, if present, is independently saturated aliphatic C₁₋₅alkylene or aliphatic C₂₋₅alkynylene.

In one embodiment, each -L¹-, if present, is independently saturated aliphatic C₁₋₅alkylene.

In one embodiment, each -L¹-, if present, is independently saturated aliphatic C₂₋₅alkylene.

In one embodiment, each -L²-, if present, is independently —CH₂— or —CH₂CH₂—.

In one embodiment, each -L²-, if present, is independently —CH₂CH₂—.

In one embodiment, each -L³-, if present, is independently —CH₂—.

In one embodiment, each —R^{G4}, if present, is independently selected from:

—F, —Cl, —Br, —I,

—R^{F1},

—CF₃, —CH₂CF₃, —CF₂CF₂H,

—OH,

-L⁴-OH,

—O-L⁴-OH,

-L⁴-OR^{F1},

—O-L⁴-OR^{F1},

—OCF₃, —OCH₂CF₃, —OCF₂CF₂H,

—SR^{F1},

—NH₂, —NHR^{F1}, —NR^{F1}₂, —NR^{N3}R^{N4},

-L⁴-NH₂, -L⁴-NHR^{F1}, -L⁴-NR^{F1}₂, or -L⁴-NR^{N3}R^{N4},

—O-L⁴-NH₂, —O-L⁴-NHR^{F1}, —O-L⁴-NR^{F1}₂, —O-L⁴-NR^{N3}R^{N4},

—NH-L⁴-NH₂, —NH-L⁴-NHR^{F1}, —NH-L⁴-NR^{F1}₂, —NH-L⁴-NR^{N3}R^{N4},

—NR^{F1}-L⁴-NH₂, —NR^{F1}-L⁴-NHR^{F1}, —NR^{F1}-L⁴NR^{F1}₂, or —NR^{F1}-L⁴-NR^{N3}R^{N4}.

In one embodiment, each —R^{G4}, if present, is independently selected from:

—F, —Cl, —Br, —I,

—OH,

—OR^{FI},

—NH₂, —NHR^{F1}, —NR^{F1}₂, and —NR^{N3}R^{N4}.

In one embodiment, each group —NR^{N3}R^{N4}, if present, is independently pyrrolidino, imidazolidino, pyrazolidino, piperidino, piperizino, morpholino, thiomorpholino, azepino, or diazepino, and is independently unsubstituted or substituted, for example, with one or more (e.g., 1, 2, 3) groups selected from C₁₋₃alkyl and —CF₃.

In one embodiment, each group —NR^{N3}R^{N4}, if present, is independently pyrrolidino, piperidino, piperizino, or morpholino, and is independently unsubstituted or substituted, for example, with one or more (e.g., 1, 2, 3) groups selected from C₁₋₃alkyl and —CF₃.

In one embodiment, each —R^{F1}, if present, is independently saturated aliphatic C₁₋₄alkyl.

In one embodiment, each -L⁴-, if present, is independently saturated aliphatic C₂₋₅alkylene.

### Some Preferred Combinations

In one preferred embodiment:

W is independently —CR^{PW}═;

X is independently —CR^{PX}═;

Y is independently —CR^{PY}═;

Z is independently —CR^{PZ}═;

each of -R^{PW}, -R^{PX}, -R^{PY}, and -R^{PZ}, if present, is independently —H or -R^{RS1};

z is 1;

-J< is independently —N<;

-R^{AK}- is independently -R^{AK1}-;

-R^{AK1}- is independently —CH₂—; and

—R^{N} is independently -R^{NNN}.

In one preferred embodiment, additionally, each -R^{RS1}, if present, is independently —F, —Cl, —Br, —I, -Me, -Et, —CF₃, —OH, —OMe, —OEt, —OCF₃, or phenyl; and additionally, two adjacent groups —R^{RS1}, if present, may form —OCH₂CH₂O—

In one preferred embodiment, additionally, -R^{NNN} is independently phenyl; and is optionally substituted, for example, with one or more (e.g., 1, 2, 3) substituents —R^{S}.

In one preferred embodiment, additionally, -R^{NNN} is independently phenyl; and is optionally substituted at the para position, for example, with a substituent -R^{S}; and is unsubstituted at all other positions.

In one preferred embodiment, additionally, -R^{NNN} is independently phenyl; and is optionally substituted with a substituent -R^{S}, wherein -R^{S} is independently —OR^{D1}.

In one preferred embodiment, additionally, -R^{NNN} is independently phenyl; and is optionally substituted at the para position with a substituent -R^{S}, and is unsubstituted at all other positions, wherein -R^{S} is independently —OR^{D1}.

In one preferred embodiment, additionally, -R^{NNN} is independently phenyl; and is optionally substituted with a substituent -R^{S}, wherein -R^{S} is independently —OR^{D1}, wherein -R^{D1} is independently -L³-R^{E7} or -L³-R^{E8}, wherein -L³- is independently —CH₂—.

In one preferred embodiment, additionally, -R^{NNN} is independently phenyl; and is optionally substituted at the para position with a substituent -R^{S}, and is unsubstituted at all other positions, wherein -R^{S} is independently —OR^{D1}, wherein —R^{D1} is independently -L³-R^{E7} or -L³-R^{E8}, wherein -L³- is independently —CH₂—.

In one preferred embodiment, additionally, -R^{NNN} is independently phenyl; and is optionally substituted with a substituent -R^{S}, wherein -R^{S} is independently —OR^{D1}, wherein -R^{D1} is independently -R^{E3}.

In one preferred embodiment, additionally, -R^{NNN} is independently phenyl; and is optionally substituted at the para position with a substituent -R^{S}, and is unsubstituted at all other positions, wherein -R^{S} is independently —OR^{D1}, wherein -R^{D1} is independently -R^{E3}.

### Molecular Weight

In one embodiment, the BCSA compound has a molecular weight of from 227 to 1200.

In one embodiment, the bottom of range is from 240, 250, 275, 300, or 350.

In one embodiment, the top of range is 1100, 1000, 900, 800, 700, or 600.

In one embodiment, the range is 240 to 600.

### Combinations

Each and every compatible combination of the embodiments described above is explicitly disclosed herein, as if each and every combination was individually and explicitly recited.

### Examples of Specific Embodiments

In one embodiment, the compounds are selected from compounds of the following formulae and pharmaceutically acceptable salts, hydrates, and solvates thereof:

| ID No. | Cmpd No. | Structure |
|---|---|---|
| IX-001 | 5.1 | 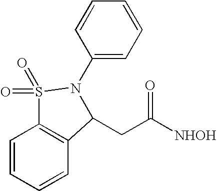 |
| IX-002 | (+)-(S)-5.1 | 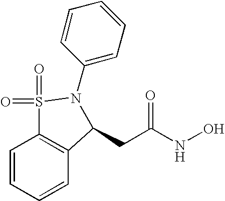 |
| IX-003 | (−)-(R)-5.1 | 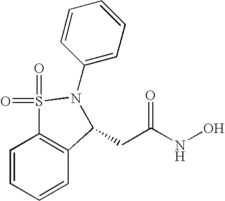 |
| IX-004 | 5.2 | 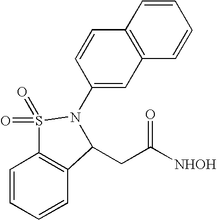 |
| IX-005 | 5.3 | 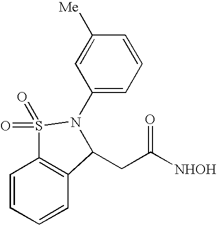 |
| IX-006 | 5.4 | 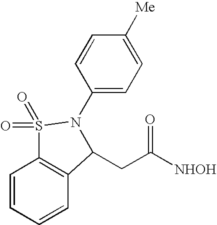 |
| IX-007 | 5.5 | 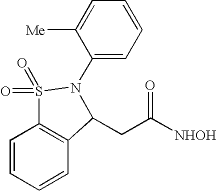 |
| IX-008 | 5.6 | 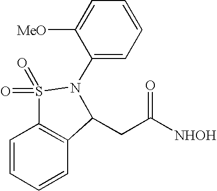 |
| IX-009 | 5.7 | 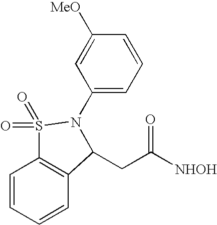 |
| IX-010 | 5.8 | 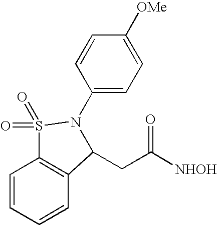 |
| IX-011 | 5.9 | 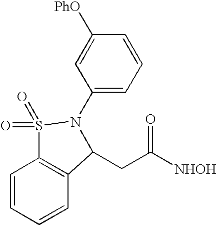 |
| IX-012 | 5.10 | 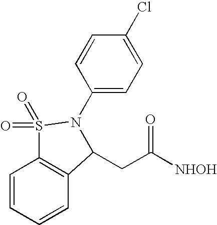 |
| IX-013 | 5.11 | 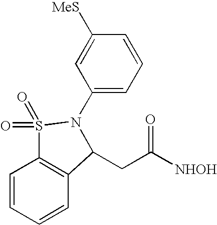 |
| IX-014 | 5.12 | 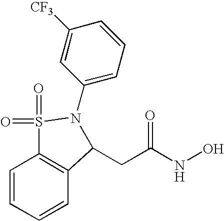 |
| IX-015 | 5.13 | 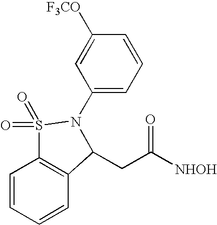 |
| IX-016 | 5.14 | 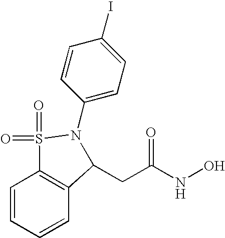 |
| IX-017 | 5.15 | 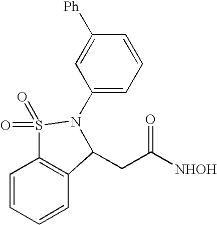 |
| IX-018 | 5.16 | 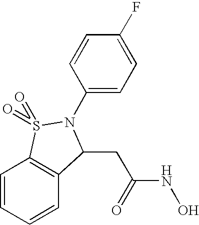 |
| IX-019 | 5.17 | 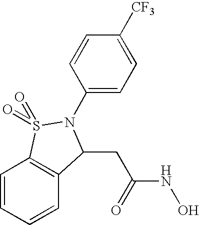 |
| IX-020 | 5.18 | 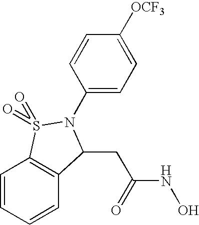 |
| IX-021 | 5.19 | 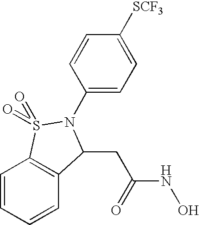 |
| IX-022 | 5.20 | 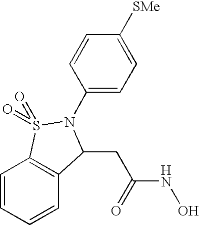 |
| IX-023 | 5.21 | 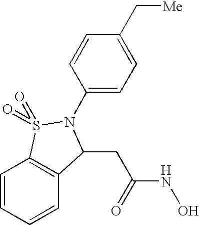 |
| IX-024 | 5.22 | 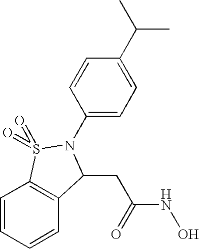 |
| IX-025 | 5.23 | 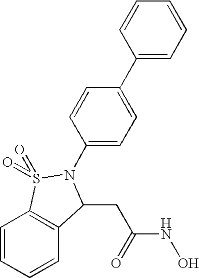 |
| IX-026 | 5.24 | 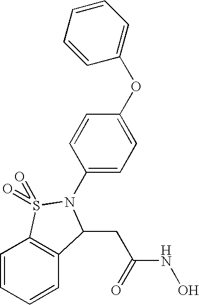 |
| IX-027 | 5.25 | 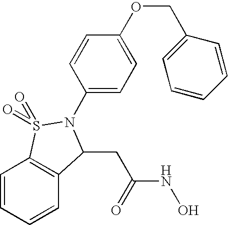 |
| IX-028 | 5.26 | 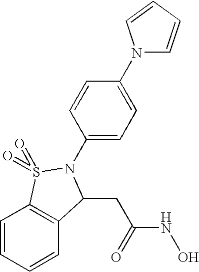 |
| IX-029 | 5.27 | 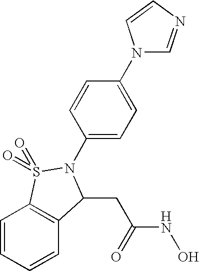 |
| IX-030 | 5.28 | 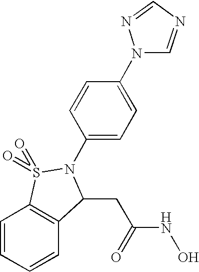 |
| IX-031 | 5.29 | 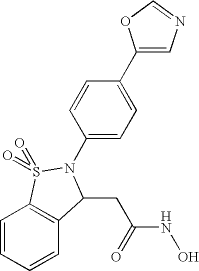 |
| IX-032 | 5.30 | 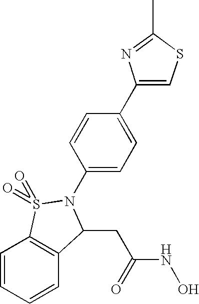 |
| IX-033 | 5.31 | 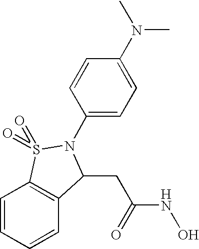 |
| IX-034 | 5.32 | 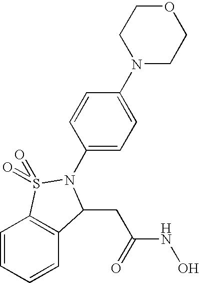 |
| IX-035 | 5.33 | 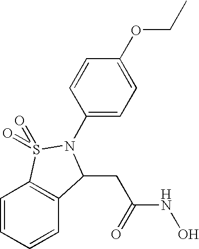 |
| IX-036 | 5.34 | 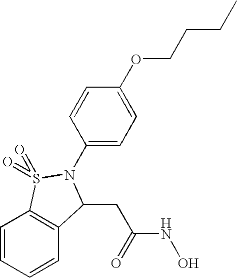 |
| IX-037 | 5.35 | 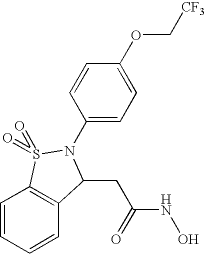 |
| IX-038 | 5.36 | 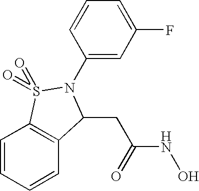 |
| IX-039 | 5.37 | 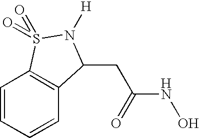 |
| IX-040 | 5.38 | 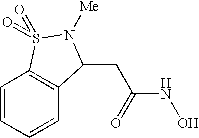 |
| IX-041 | 5.39 | 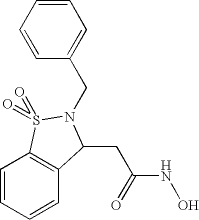 |
| IX-042 | 5.40 | 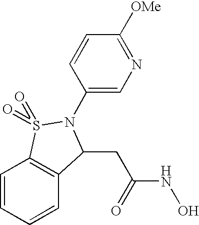 |
| IX-043 | 5.41 | 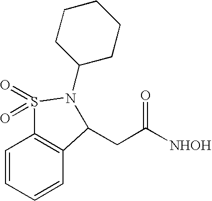 |
| IX-044 | 5.42 | 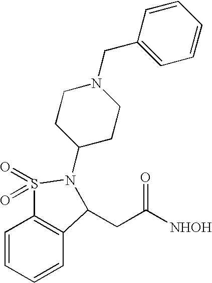 |
| IX-045 | 5.43 | 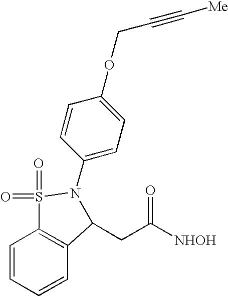 |
| IX-046 | (+)-5.43 | 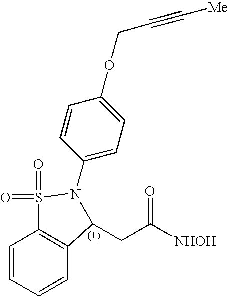 |
| IX-047 | (−)-5.43 | 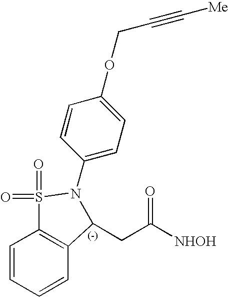 |
| IX-048 | 5.44 | 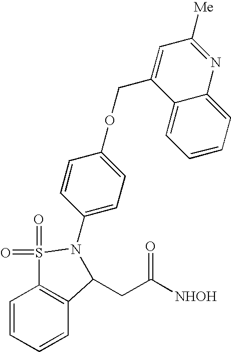 |
| IX-049 | (+)-5.44 | 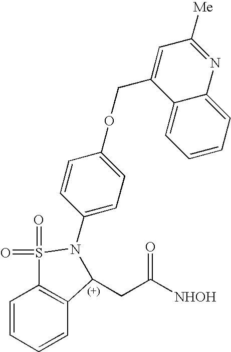 |
| IX-050 | (−)-5.44 | 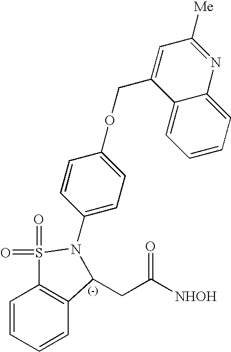 |
| IX-051 | 5.45 | 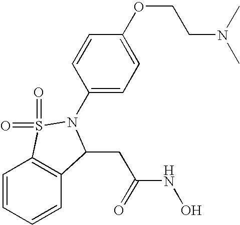 |
| IX-052 | 5.46 | 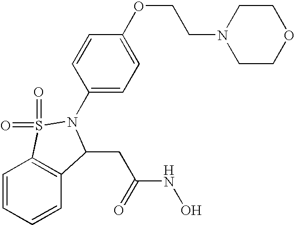 |
| IX-053 | 5.47 | 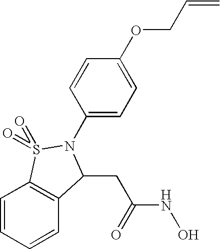 |
| IX-054 | 5.48 | 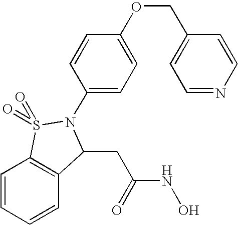 |
| IX-055 | 5.49 | 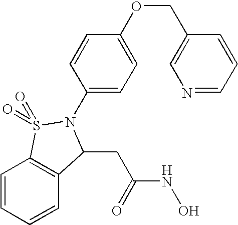 |
| IX-056 | 5.50 | 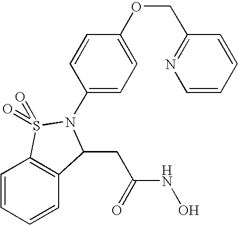 |
| IX-057 | 5.51 | 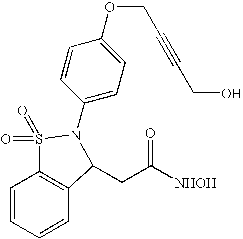 |
| IX-058 | 5.52 | 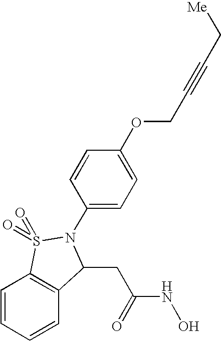 |
| IX-059 | 5.53 | 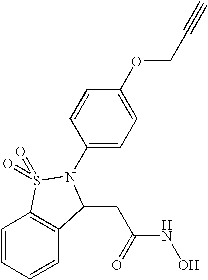 |
| IX-060 | 5.54 | 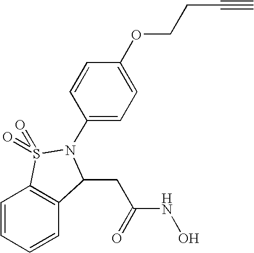 |
| IX-061 | 5.55 | 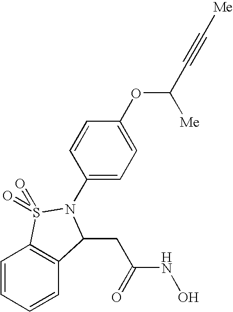 |
| IX-062 | 5.56 | 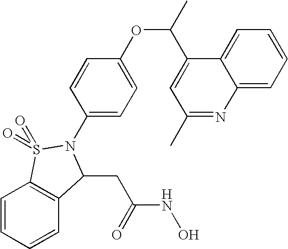 |
| IX-063 | 5.57 | 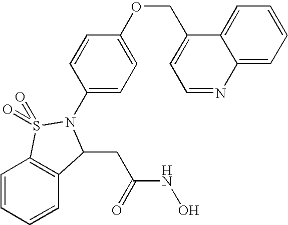 |
| IX-064 | 5.58 | 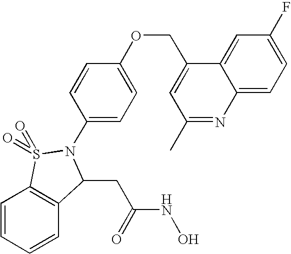 |
| IX-065 | 5.59 | 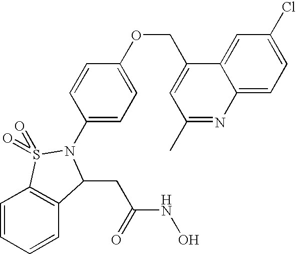 |
| IX-066 | 5.60 | 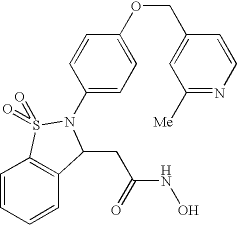 |
| IX-067 | 5.61 | 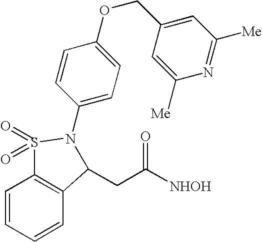 |
| IX-068 | 5.62 | 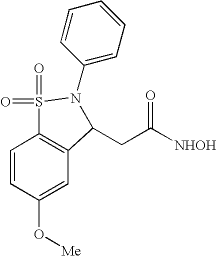 |
| IX-069 | 5.63 | 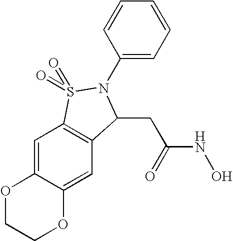 |
| IX-070 | 5.64 | 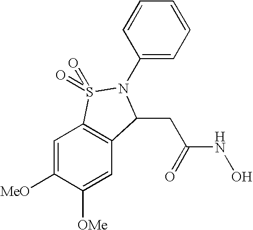 |
| IX-071 | 5.65 | 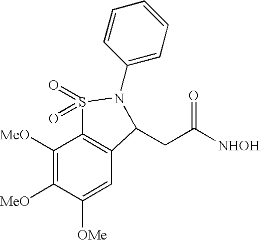 |
| IX-072 | 5.66 | 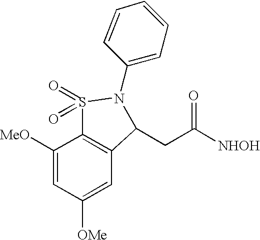 |
| IX-073 | 5.67 | 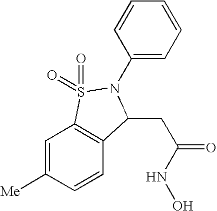 |
| IX-074 | 5.68 | 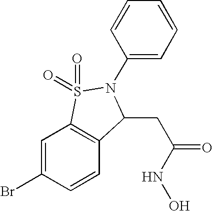 |
| IX-075 | 24 | 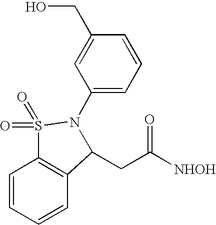 |
| IX-076 | 29 | 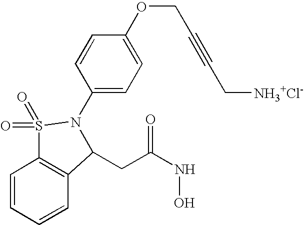 |
| IX-077 | 36 | 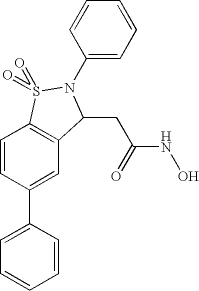 |
| IX-078 | 39 | 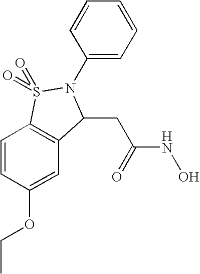 |
| IX-079 | 43 | 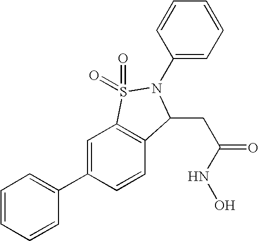 |
| IX-080 | 48.1 | 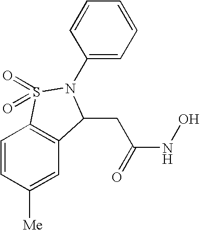 |
| IX-081 | 48.2 | 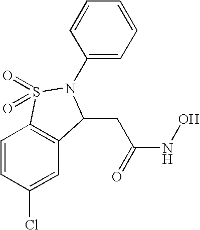 |
| IX-082 | 54.1 | 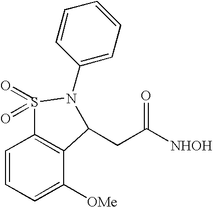 |
| IX-083 | 54.2 | 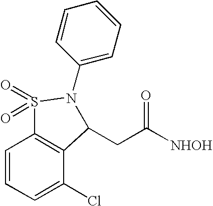 |
| IX-084 | 54.3 | 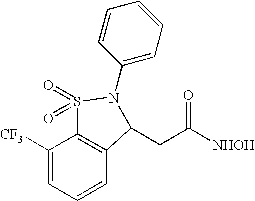 |
| IX-085 | 54.4 | 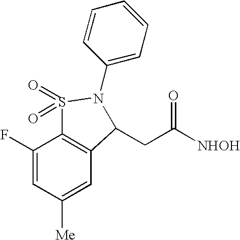 |
| IX-086 | 54.5 | 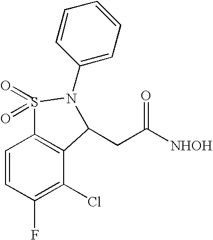 |
| IX-087 | 54.6 | 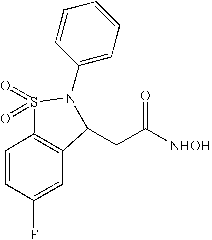 |
| IX-088 | 54.7 | 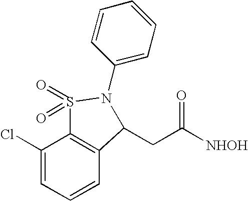 |
| IX-089 | 54.8 | 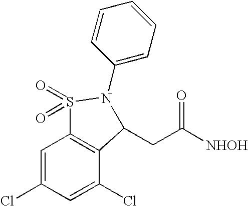 |
| IX-090 | 54.9 | 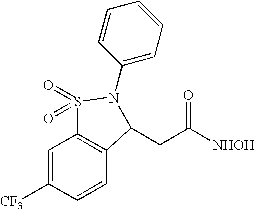 |
| IX-091 | 54.10 | 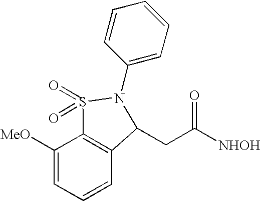 |
| IX-092 | 54.11 | 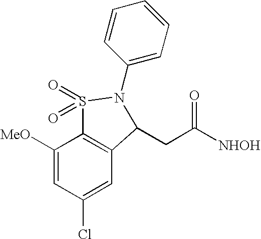 |
| IX-093 | 54.12 | 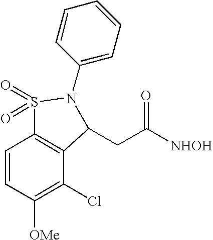 |
| IX-094 | 57 | 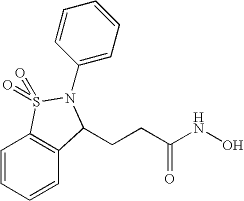 |
| IX-095 | 62.1 | 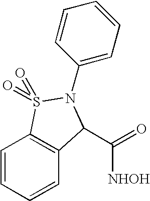 |
| IX-096 | 62.2 | 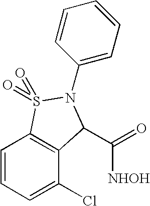 |

In one embodiment, the compounds are selected from compounds of the following formulae and pharmaceutically acceptable salts, hydrates, and solvates thereof:

| ID No. | Cmpd No. | Structure |
|---|---|---|
| IX-097 | 72 | 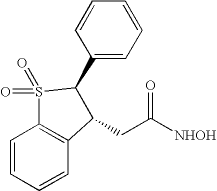 |

In one embodiment, the compounds are selected from compounds of the following formulae and pharmaceutically acceptable salts, hydrates, and solvates thereof:

| ID No. | Cmpd No. | Structure |
|---|---|---|
| IX-098 | 77 | 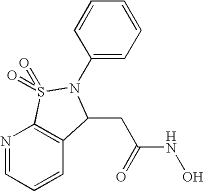 |

In one embodiment, the compounds are selected from compounds of the following formulae and pharmaceutically acceptable salts, hydrates, and solvates thereof:

| ID No. | Cmpd. No. | Structure |
|---|---|---|
| IX-099 | 4.1 | 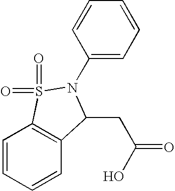 |
| IX-100 | 4.43 | 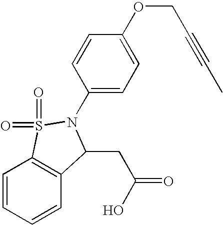 |
| IX-101 | 4.44 | 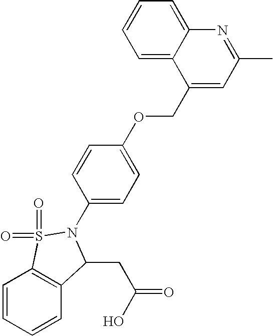 |

### Additional Examples

In one embodiment, the compounds are selected from compounds of the following formulae and pharmaceutically acceptable salts, hydrates, and solvates thereof:

| | |
|---|---|
| 1 | 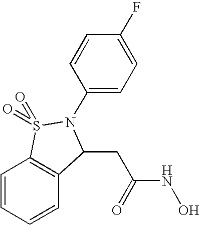 |
| 2 | 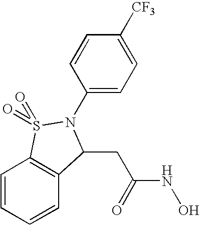 |
| 3 | 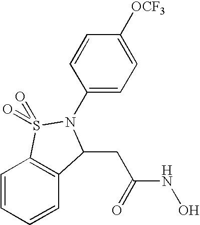 |
| 4 | 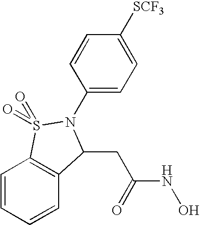 |
| 5 | 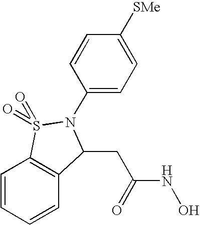 |
| 6 | 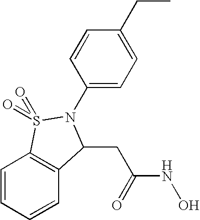 |
| 7 | 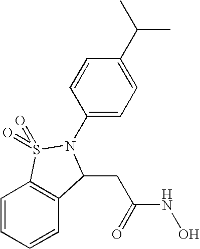 |
| 8 | 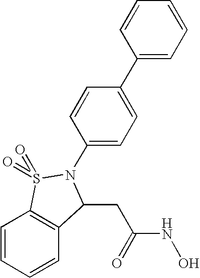 |
| 9 | 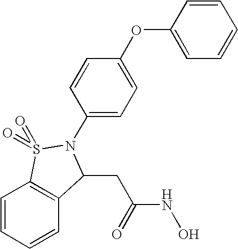 |
| 10 | 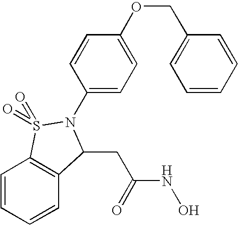 |
| 11 | 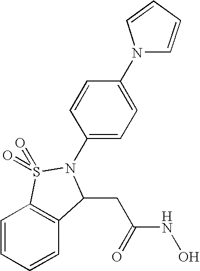 |
| 12 | 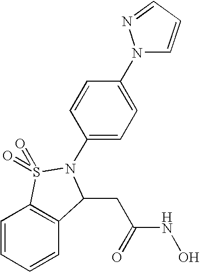 |
| 13 | 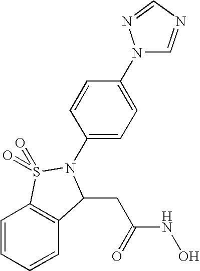 |
| 14 | 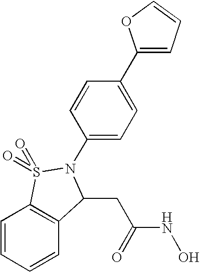 |
| 15 | 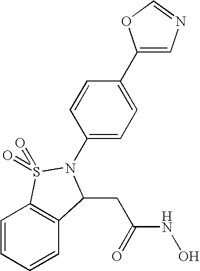 |
| 16 | 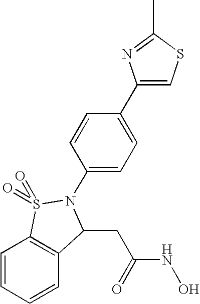 |
| 17 | 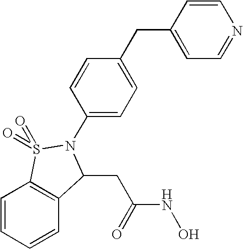 |
| 18 | 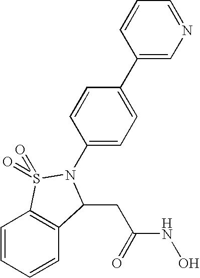 |
| 19 | 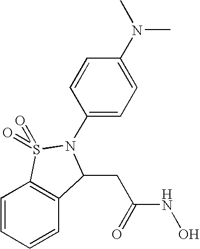 |
| 20 | 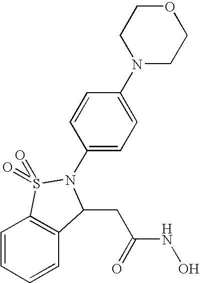 |
| 21 | 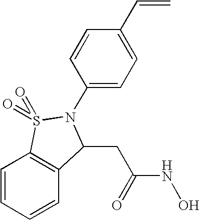 |
| 22 | 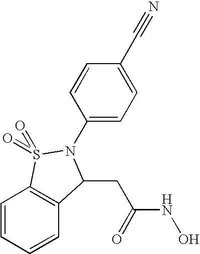 |
| 23 | 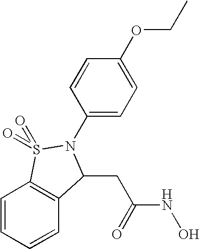 |
| 24 | 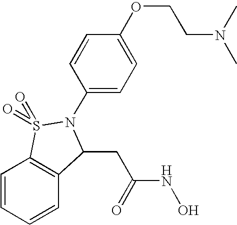 |
| 25 | 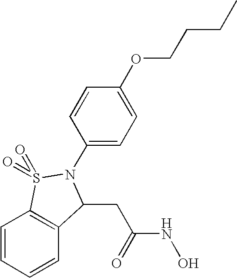 |
| 26 | 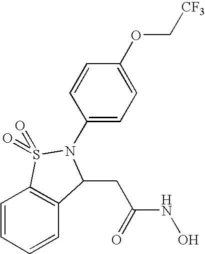 |
| 27 | 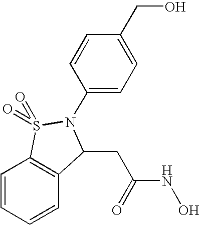 |
| 28 | 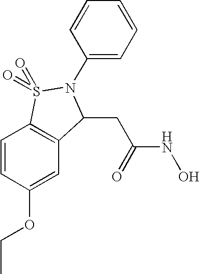 |
| 29 | 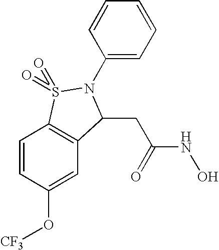 |
| 30 | 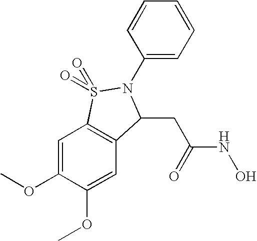 |
| 31 | 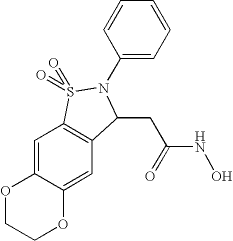 |
| 32 | 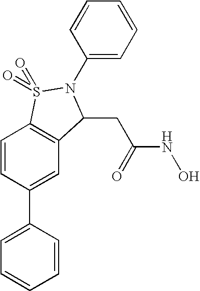 |
| 33 | 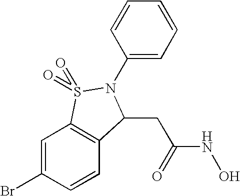 |
| 34 | 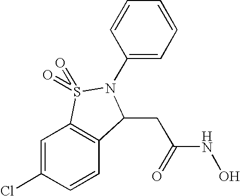 |
| 35 | 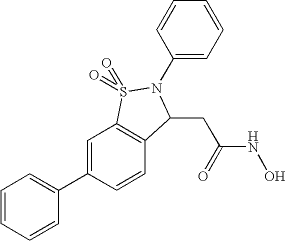 |
| 36 | 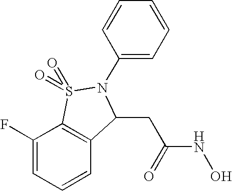 |

In one embodiment, the compounds are selected from compounds of the following formulae and pharmaceutically acceptable salts, hydrates, and solvates thereof:

| | |
|---|---|
| 37 | 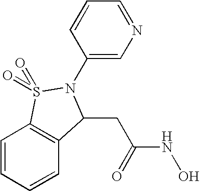 |
| 38 | 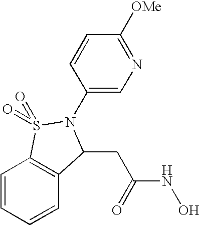 |
| 39 | 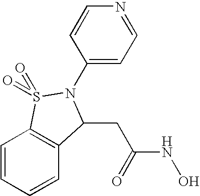 |
| 40 | 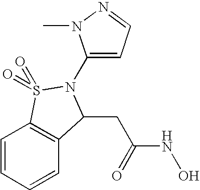 |
| 41 | 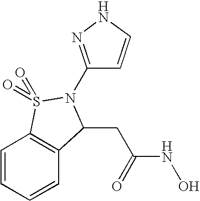 |

In one embodiment, the compounds are selected from compounds of the following formulae and pharmaceutically acceptable salts, hydrates, and solvates thereof:

| | |
|---|---|
| 42 | 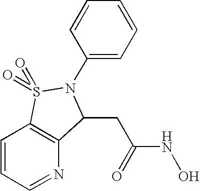 |
| 43 | 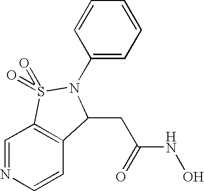 |
| 44 | 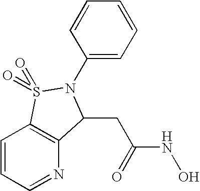 |

In one embodiment, the compounds are selected from compounds of the following formulae and pharmaceutically acceptable salts, hydrates, and solvates thereof:

| | |
|---|---|
| 45 | 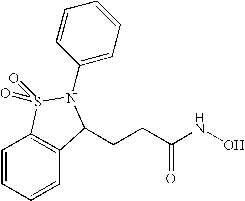 |
| 46 | 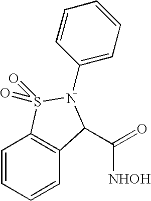 |

In one embodiment, the compounds are selected from compounds of the following formulae and pharmaceutically acceptable salts, hydrates, and solvates thereof:

| | |
|---|---|
| 47 | 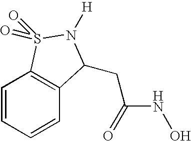 |
| 48 | 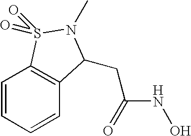 |
| 49 | 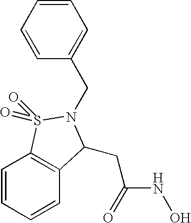 |
| 50 | 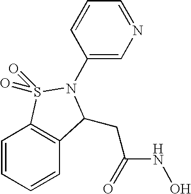 |
| 51 | 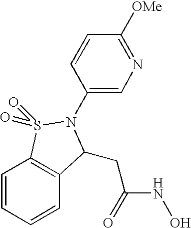 |
| 52 | 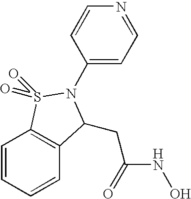 |
| 53 | 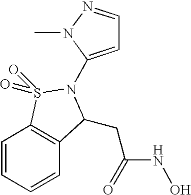 |
| 54 | 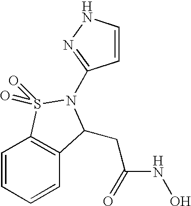 |

### Substantially Purified Forms

One aspect of the present invention pertains to BCSA compounds, as described herein, in substantially purified form and/or in a form substantially free from contaminants.

In one embodiment, the substantially purified form is at least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless specified, the substantially purified form refers to the compound in any stereoisomeric or enantiomeric form. For example, in one embodiment, the substantially purified form refers to a mixture of stereoisomers, i.e., purified with respect to other compounds. In one embodiment, the substantially purified form refers to one stereoisomer, e.g., optically pure stereoisomer. In one embodiment, the substantially purified form refers to a mixture of enantiomers. In one embodiment, the substantially purified form refers to a equimolar mixture of enantiomers (i.e., a racemic mixture, a racemate). In one embodiment, the substantially purified form refers to one enantiomer, e.g., optically pure enantiomer.

In one embodiment, the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

Unless specified, the contaminants refer to other compounds, that is, other than stereoisomers or enantiomers. In one embodiment, the contaminants refer to other compounds and other stereoisomers. In one embodiment, the contaminants refer to other compounds and the other enantiomer.

In one embodiment, the substantially purified form is at least 60% optically pure (i.e., 60% of the compound, on a molar basis, is the desired stereoisomer or enantiomer, and 40% is the undesired stereoisomer or enantiomer), e.g., at least 70% optically pure, e.g., at least 80% optically pure, e.g., at least 90% optically pure, e.g., at least 95% optically pure, e.g., at least 97% optically pure, e.g., at least 98% optically pure, e.g., at least 99% optically pure.

### Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

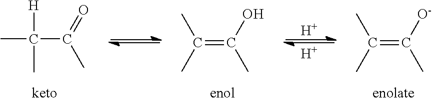

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NFIR₃⁺, NR₄⁺). Examples of some suitable substituted ammonium ions are those derived from ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Solvates and Hydrates

It may be convenient or desirable to prepare, purify, and/or handle a corresponding solvate of the compound. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate and hydrate forms thereof.

### Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

Unless otherwise specified, a reference to a particular compound also includes chemically protected forms thereof.

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulfonyl)ethyloxy amide (—NH-Psec); or, in suitable cases (e.g., cyclic amines), as a nitroxide radical (>N—O.).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀aryl-C₁₋₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

### Prodrugs

It may be convenient or desirable to prepare, purify, and/or handle the compound in the form of a prodrug. The term “prodrug,” as used herein, pertains to a compound which, when metabolised (e.g., in vivo), yields the desired active compound. Typically, the prodrug is inactive, or less active than the desired active compound, but may provide advantageous handling, administration, or metabolic properties.

Unless otherwise specified, a reference to a particular compound also includes prodrugs thereof.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Chemical Synthesis

Several methods for the chemical synthesis of BCSA compounds of the present invention are described herein. These and/or other well known methods may be modified and/or adapted in known ways in order to facilitate the synthesis of additional compounds within the scope of the present invention.

### Uses

The BCSA compounds described herein are useful, for example, in the treatment of diseases and conditions that are ameliorated by the inhibition of TACE.

### Use in Methods of Inhibiting TACE and Methods of Regulating Cytokine Release

One aspect of the present invention pertains to a method of inhibiting TACE in a cell, in vitro or in vivo, comprising contacting said cell with an effective amount of a BCSA compound, as described herein.

Suitable assays for determining TACE inhibition are known in the art and/or are described herein.

Another aspect of the present invention pertains to a method of regulating (e.g., inhibiting) cytokine release (e.g., TNF-α release) in a cell, in vitro or in vivo, comprising contacting said cell with an effective amount of a BCSA compound, as described herein.

Suitable assays for determining regulation (e.g., inhibition) of cytokine release are known in the art and/or are described herein.

In one embodiment, the method is performed in vitro.

In one embodiment, the method is performed in vivo.

In one embodiment, the BCSA compound is provided in the form of a pharmaceutically acceptable composition.

Any type of cell may be treated, including but not limited to, lung, gastrointestinal (including, e.g., bowel, colon), breast (mammary), ovarian, prostate, liver (hepatic), kidney (renal), bladder, pancreas, brain, and skin.

### Use in Methods of Therapy

Another aspect of the present invention pertains to a BCSA compound as described herein for use in a method of treatment (e.g., of a disease or disorder) of the human or animal body by therapy.

### Use in the Manufacture of Medicaments

Another aspect of the present invention pertains to use of a BCSA compound, as described herein, in the manufacture of a medicament for use in treatment (e.g., of a disease or disorder).

In one embodiment, the medicament comprises the BCSA compound.

### Methods of Treatment

Another aspect of the present invention pertains to a method of treatment (e.g., of a disease or disorder) comprising administering to a patient in need of treatment a therapeutically effective amount of a BCSA compound, as described herein, preferably in the form of a pharmaceutical composition.

### Conditions Treated—Conditions Mediated by TACE

In one embodiment (e.g., of use in methods of therapy, of use in the manufacture of medicaments, of methods of treatment), the treatment is treatment of a disease or disorder that is mediated by TACE, for example, a disease or disorder that is known to be mediated by TACE.

A disease or disorder that is mediated by TACE is, for example, a disease or disorder in which TACE and/or the action of TACE is important or necessary, e.g., for the onset, progress, expression, etc. of that disease or disorder.

### Conditions Treated—Conditions Ameliorated by the Inhibition of TACE

In one embodiment (e.g., of use in methods of therapy, of use in the manufacture of medicaments, of methods of treatment), the treatment is treatment of a disease or disorder that is ameliorated by the inhibition of TACE, for example, a disease or disorder that is known to be ameliorated by the inhibition of TACE.

### Conditions Treated—Conditions Treated by TACE Inhibitors

In one embodiment (e.g., of use in methods of therapy, of use in the manufacture of medicaments, of methods of treatment), the treatment is treatment of a disease or disorder that is treated by a TACE inhibitor, for example, a disease or disorder that is known to be treated by a TACE inhibitor.

### Conditions Treated—Particular Conditions

In one embodiment (e.g., of use in methods of therapy, of use in the manufacture of medicaments, of methods of treatment), the treatment is treatment of: rheumatoid arthritis; inflammation; psoriasis; septic shock; graft rejection; cachexia; anorexia; congestive heart failure; post-ischaemic reperfusion injury; inflammatory disease of the central nervous system; inflammatory bowel disease; insulin resistance; HIV infection; cancer; chronic obstructive pulmonary disease (COPD); or asthma.

In one embodiment (e.g., of use in methods of therapy, of use in the manufacture of medicaments, of methods of treatment), the treatment is treatment of: osteoarthritis, ulcerative colitis, Crohn's disease, multiple sclerosis, or degenerative cartilage loss.

In one embodiment, the treatment is treatment of inflammation.

In one embodiment, the treatment is treatment of rheumatoid arthritis.

In one embodiment, the treatment is treatment of psoriasis.

### Conditions Treated—Cancer etc.

In one embodiment, the treatment is treatment of: cancer.

In one embodiment, the treatment is treatment of: lung cancer, small cell lung cancer, non-small cell lung cancer, gastrointestinal cancer, stomach cancer, bowel cancer, colon cancer, rectal cancer, colorectal cancer, thyroid cancer, breast cancer, ovarian cancer, endometrial cancer, prostate cancer, testicular cancer, liver cancer, kidney cancer, renal cell carcinoma, bladder cancer, pancreatic cancer, brain cancer, glioma, sarcoma, osteosarcoma, bone cancer, skin cancer, squamous cancer, Kaposi's sarcoma, melanoma, malignant melanoma, lymphoma, or leukemia.

In one embodiment, the treatment is treatment of:

a carcinoma, for example a carcinoma of the bladder, breast, colon (e.g., colorectal carcinomas such as colon adenocarcinoma and colon adenoma), kidney, epidermal, liver, lung (e.g., adenocarcinoma, small cell lung cancer and non-small cell lung carcinomas), oesophagus, gall bladder, ovary, pancreas (e.g., exocrine pancreatic carcinoma), stomach, cervix, thyroid, prostate, skin (e.g., squamous cell carcinoma);

a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma;

a hematopoietic tumor of myeloid lineage, for example acute and chronic myelogenous leukemias, myelodysplastic syndrome, or promyelocytic leukemia;

a tumour of mesenchymal origin, for example fibrosarcoma or habdomyosarcoma;

a tumor of the central or peripheral nervous system, for example astrocytoma, neuroblastoma, glioma or schwannoma;

melanoma; seminoma; teratocarcinoma; osteosarcoma; xenoderoma pigmentoum; keratoctanthoma; thyroid follicular cancer; or Kaposi's sarcoma.

In one embodiment, the treatment is treatment of solid tumour cancer.

In one embodiment (e.g., of use in methods of therapy, of use in the manufacture of medicaments, of methods of treatment), the treatment is treatment of: a hyperproliferative skin disorder.

In one embodiment, the treatment is treatment of: psoriasis, actinic keratosis, and/or non-melanoma skin cancer.

### Conditions Treated—Inflammation etc.

In one embodiment (e.g., of use in methods of therapy, of use in the manufacture of medicaments, of methods of treatment), the treatment is treatment of: an inflammatory disease.

In one embodiment, the treatment is treatment of: an inflammatory disease involving pathological activation of T- and B-cell lymphocytes, neutrophils, and/or Mast cells.

In one embodiment, the treatment is treatment of: an inflammatory disease, such as rheumatoid arthritis, osteoarthritis, rheumatoid spondylitis, gouty arthritis, traumatic arthritis, rubella arthritis, psoriatic arthritis, and other arthritic conditions; Alzheimer's disease; toxic shock syndrome, the inflammatory reaction induced by endotoxin or inflammatory bowel disease; tuberculosis; atherosclerosis; muscle degeneration; Reiter's syndrome; gout; acute synovitis; sepsis; septic shock; endotoxic shock; gram negative sepsis; adult respiratory distress syndrome; cerebral malaria; chronic pulmonary inflammatory disease; silicosis; pulmonary sarcoisosis; bone resorption diseases; reperfusion injury; graft versus host reaction; allograft rejections; fever and myalgias due to infection, such as influenza, cachexia, in particular cachexia secondary to infection or malignancy, cachexia secondary to acquired immune deficiency syndrome (AIDS); AIDS; ARC (AIDS related complex); keloid formation; scar tissue formation; Crohn's disease; ulcerative colitis; pyresis; chronic obstructive pulmonary disease (COPD); acute respiratory distress syndrome (ARDS); asthma; pulmonary fibrosis; bacterial pneumonia.

In one preferred embodiment, the treatment is treatment of: an arthritic condition, including rheumatoid arthritis and rheumatoid spondylitis; inflammatory bowel disease, including Crohn's disease and ulcerative colitis; and chronic obstructive pulmonary disease (COPD).

In one preferred embodiment, the treatment is treatment of: an inflammatory disorder characterized by T-cell proliferation (T-cell activation and growth), for example, tissue graft rejection, endotoxin shock, and glomerular nephritis.

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, alleviation of symptoms of the condition, amelioration of the condition, and cure of the condition. Treatment as a prophylactic measure (i.e., prophylaxis) is also included. For example, use with patients who have not yet developed the condition, but who are at risk of developing the condition, is encompassed by the term “treatment.”

For example, treatment of cancer includes the prophylaxis of cancer, reducing the incidence of cancer, alleviating the symptoms of cancer, etc.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

### Combination Therapies

The term “treatment” includes combination treatments and therapies, in which two or more treatments or therapies are combined, for example, sequentially or simultaneously. For example, the BCSA compounds described herein may also be used in combination therapies, e.g., in conjunction with other agents, for example, other TACE inhibitors, other cytotoxic agents, other anticancer agents, etc. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets.

For example, it may be beneficial to combine treatment with a BCSA compound as described herein with one or more other (e.g., 1, 2, 3, 4) agents or therapies that regulates cell growth or survival or differentiation via a different mechanism, thus treating several characteristic features of cancer development.

One aspect of the present invention pertains to a BCSA compound as described herein, in combination with one or more additional therapeutic agents, as described below.

The particular combination would be at the discretion of the physician who would select dosages using his common general knowledge and dosing regimens known to a skilled practitioner.

The agents (i.e., the BCSA compound described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., the BCSA compound described here, plus one or more other agents) may be formulated together in a single dosage form, or alternatively, the individual agents may be formulated separately and presented together in the form of a kit, optionally with instructions for their use.

### Other Uses

The BCSA compounds described herein may also be used as cell culture additives to inhibit TACE, to inhibit cytokine release (e.g., TNF-α release), etc.

The BCSA compounds described herein may also be used as part of an in vitro assay, for example, in order to determine whether a candidate host is likely to benefit from treatment with the compound in question.

The BCSA compounds described herein may also be used as a standard, for example, in an assay, in order to identify other compounds, other TACE inhibitors, etc.

### Kits

One aspect of the invention pertains to a kit comprising (a) a BCSA compound as described herein, or a composition comprising a compound as described herein, e.g., preferably provided in a suitable container and/or with suitable packaging; and

(b) instructions for use, e.g., written instructions on how to administer the compound or composition.

The written instructions may also include a list of indications for which the active ingredient is a suitable treatment.

### Routes of Administration

The BCSA compound or pharmaceutical composition comprising the BCSA compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject/Patient

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

### Formulations

While it is possible for the BCSA compound to be administered alone, it is preferable to present it as a pharmaceutical formulation (e.g., composition, preparation, medicament) comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one BCSA compound, as described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The compound may be dissolved in, suspended in, or admixed with one or more other pharmaceutically acceptable ingredients. The compound may be presented in a liposome or other microparticulate which is designed to target the compound, for example, to blood components or one or more organs.

Formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, losenges, pastilles, as well as patches, adhesive plasters, depots, and reservoirs. Losenges typically comprise the compound in a flavored basis, usually sucrose and acacia or tragacanth. Pastilles typically comprise the compound in an inert matrix, such as gelatin and glycerin, or sucrose and acacia. Mouthwashes typically comprise the compound in a suitable liquid carrier.

Formulations suitable for sublingual administration include tablets, losenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, losenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent. The tablets may optionally be coated or scored and may be formulated so as to provide slow or controlled release of the compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the compound and a paraffinic or a water-miscible ointment base.

Creams are typically prepared from the compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the compound through the skin or other affected areas. Examples of such dermal penetration enhancers include dimethylsulfoxide and related analogues.

Emulsions are typically prepared from the compound and an oily phase, which may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for ocular administration include eye drops wherein the compound is dissolved or suspended in a suitable carrier, especially an aqueous solvent for the compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the BCSA compounds, and compositions comprising the BCSA compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the BCSA compound is in the range of about 100 μg to about 250 mg (more typically about 100 μg to about 25 mg) per kilogram body weight of the subject per day. Where the compound is a salt, an ester, an amide, a prodrug, or the like, the amount administered is calculated on the basis of the parent compound and so the actual weight to be used is increased proportionately.

### EXAMPLES

The following examples are provided solely to illustrate the present invention and are not intended to limit the scope of the invention, as described herein.

### General Synthesis

Cyclic sulphonamide derivatives (5.1)-(5.68) were prepared as follows (Scheme 1). Sulphonylation of amines (2.1)-(2.61) with sulphonylchlorides (1.1)-(1.8) was followed by heating to enable the cyclization. Some esters (3) were isolated and hydrolyzed under acidic conditions to provide the corresponding carboxylic acids (4). Some intermediate esters (3) were transformed to carboxylic acids (4) without isolation by prolonged heating in the same reaction pot that led to hydrolysis of ester functionality. Carboxylic acids (4.1)-(4.68) were converted to the corresponding hydroxamic acids (5.1)-(5.68) by using one of the three methods (Conditions A-C, Scheme 1).

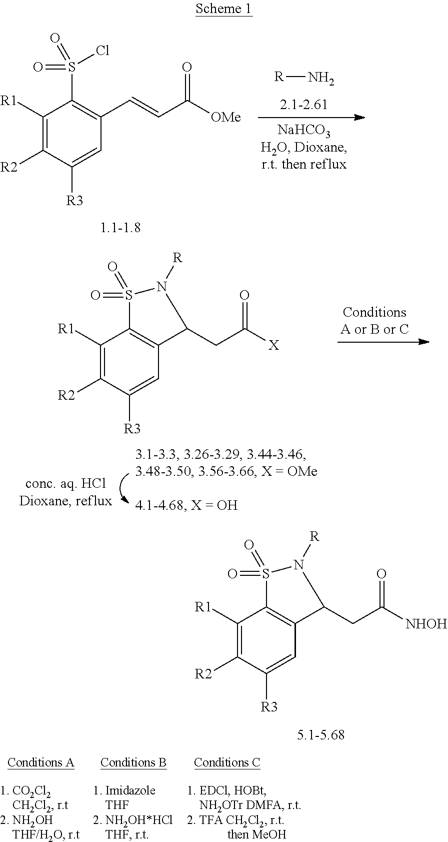

Sulphonylchloride (1.1) (where R1=R2=R3=H) used for the synthesis of sulphonamides (5.1)-(5.61) was prepared according to the known procedure (see, e.g., Finn et al., 2005). Sulphonylchlorides (1.2)-(1.6) needed for the synthesis of sulphonamides (5.62)-(5.66) were prepared by regioselective chlorosulphonylation of the known unsaturated esters (7.1)-(7.5) (see e.g., Imashiro, 2004; Westman et al., 2001; E1-Batta et al., 2007; Mahajan et al., 2005; Skretas et al., 2007).

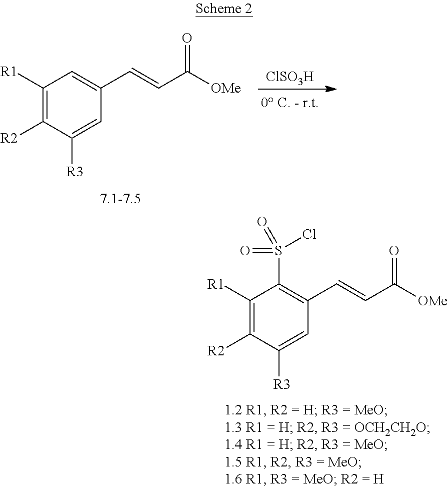

Sulphonylchlorides (1.7)-(1.8) needed for the synthesis of sulphonamides (5.67)-(5.68) were prepared starting from aminobenzenesulphonic acids (8.1)-(8.2) (Scheme 3). These were transformed to diazonium salts (9.1)-(9.2) that were subsequently used for the Heck reaction to give unsaturated esters (10.1)-(10.2). The intermediates (10.1)-(10.2) were transformed to sulphonylchlorides (1.7)-(1.8) by the reaction with thionylchloride.

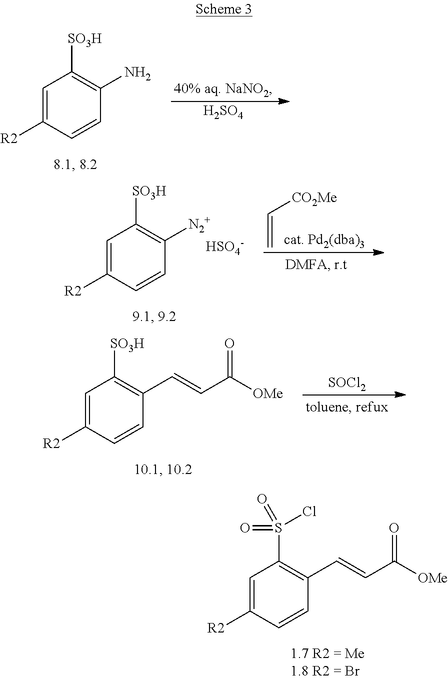

Amines (2.1)-(2.42) used for the synthesis of compounds (5.1)-(5.42) were commercially available. Amines (2.43)-(2.44) needed for the synthesis of sulphonamides (5.43)-(5.44) were obtained by O-alkylation of para-hydroxyaniline (11) with but-2-yn-1-yl methanesulphonate (12) (see, e.g., Brummond et al., 2004) and 4-chloromethyl-2-methylquinoline (13) (see, e.g., Duan et al., 2002) to give anilines (2.43) and (2.44), respectively (Scheme 4).

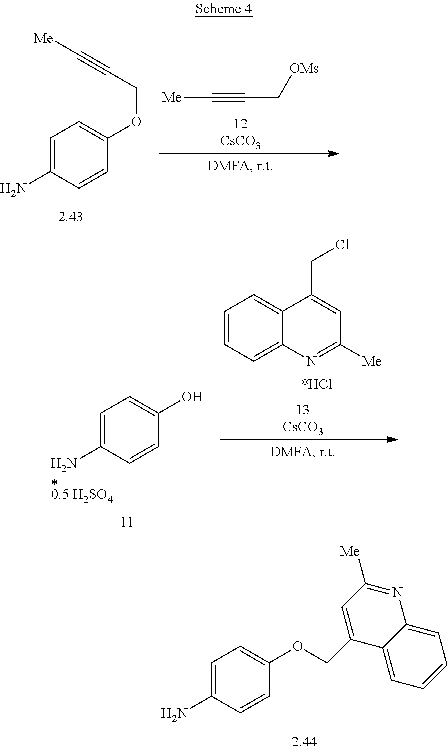

Amines (2.45)-(2.61) needed for the synthesis of sulphonamides (5.45)-(5.61) were obtained by O-alkylation of para-hydroxynitrobenzene (14) with alkylating agents (15.1)-(15.17) and subsequent reduction of the nitro group in the resulting intermediates (16.1)-(16.17) by using one of the three conditions for the reduction (Scheme 5, Conditions A-C).

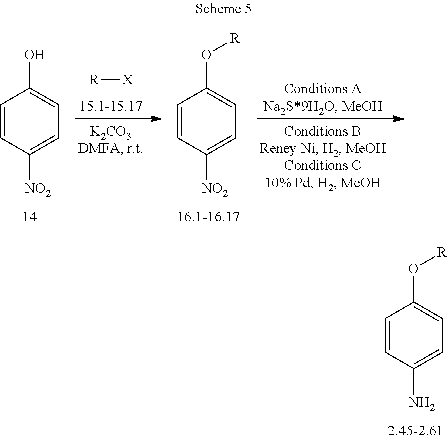

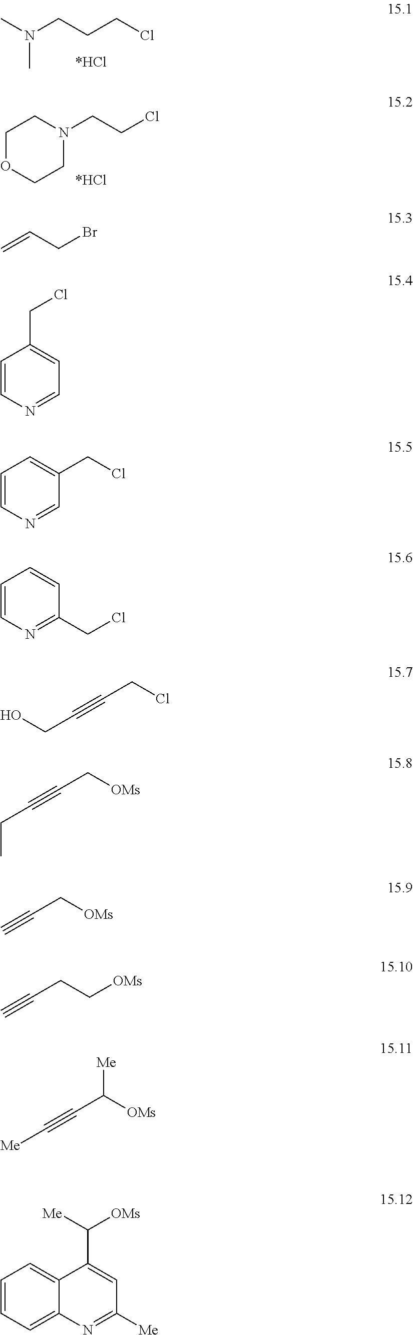

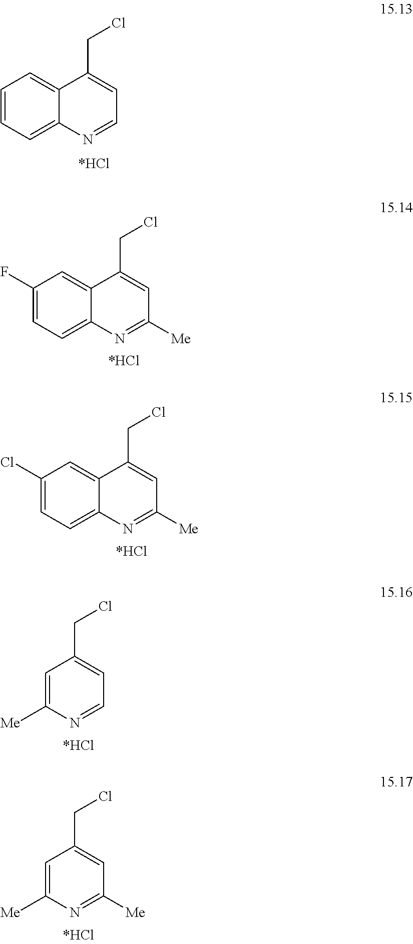

Alkylating agents (15.1)-(15.7) needed for the synthesis of anilines (2.45)-(2.51) were commercially available. Alkylating agents (15.8)-(15.11) needed for the synthesis of anilines (2.52)-(2.55) were prepared according to the literature procedures (see e.g., White et al., 1982; Jackson et al., 1988; Thibault et al., 2006; Marshall et. al., 2000).

Alkylating agent (15.12) needed for the synthesis of aniline (2.56) was prepared according to the method shown in Scheme 6. 2-Methyl-4-hydroxymethylquinoline (17) was oxidized with Dess-Martin periodinane to give aldehyde. Methylmagnesium bromide addition to intermediate aldehyde provided a secondary alcohol that was treated with methanesulphonylchloride to give alkylating agent (15.12).

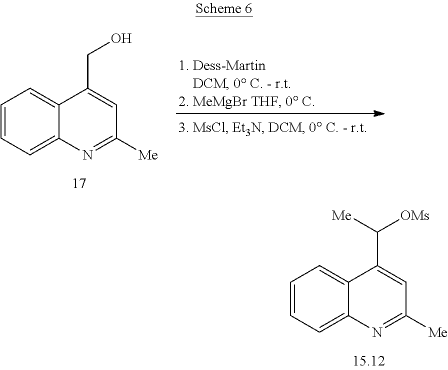

4-Chloromethylquinoline (15.13) needed for the synthesis of aniline (2.57) was prepared from known 4-hydroxymethylquinoline (18) (see, e.g., Boutros et. al., 2000) (Scheme 7).

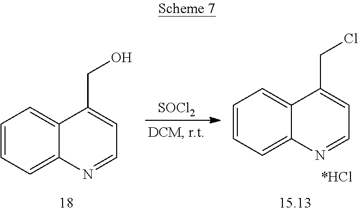

The synthesis of alkylating agents (15.14) and (15.15) needed for the preparation of anilines (2.58) and (2.59) were started from carboxylic acids (19.1) and (19.2) that were prepared according to the literature procedures (see, e.g., Yen et. al., 1958; Buchman et al., 1946) (Scheme 8). Carboxylic acids (19.1) and (19.2) were transformed to their esters that were subsequently reduced to alcohols. These intermediates were transformed to the required chloromethylquinolines (15.14) and (15.15) by the reaction with thionylchloride.

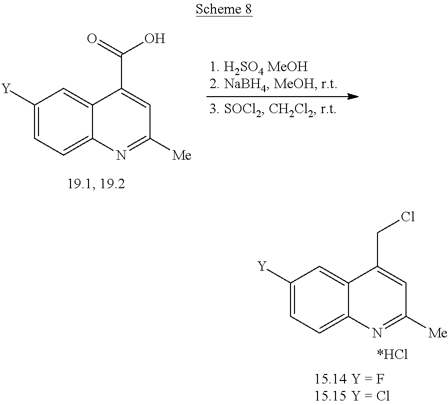

The synthesis of 4-chloromethylpyridines (15.16) and (15.17) needed for the preparation of anilines (2.60) and (2.61) were prepared starting from 4-methylpyridine derivatives (20.1) and (20.2) (Scheme 9). Alcohols (21.1) and (21.2) were prepared according to the known route (see, e.g. Ragan et al., 2002) and were transformed to 4-chloromethylpyridines (15.16) and (15.17).

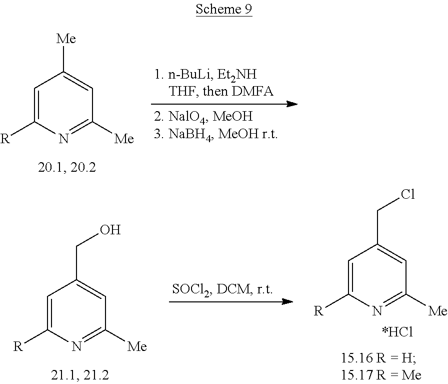

To prepare hydroxamic acid (24), sulphonylchloride (1.1) was first transformed to unsaturated ester (23) in the reaction of with substituted aniline (22) (Scheme 10). The reaction of ester (23) with hydroxylamine under basic conditions led to intramolecular cyclization and formation of hydroxamic acid (24).

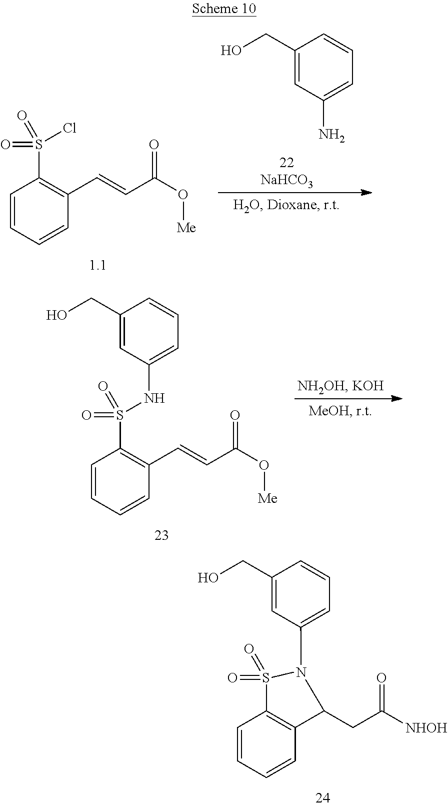

The synthesis of hydroxamic acid (29) is shown in Scheme 11. Free hydroxyl group in intermediate (16.7) (Scheme 5) was mezylated. Methanesulphonate group was replaced with azido group, azide reduced and the resulting amine was protected with tert-butoxycarbonyl group to give an intermediate (25). The reduction of the nitro group gave aniline (26) the reaction of which with sulphonylchloride (1.1) provided cyclic carboxylic acid (27). Carboxylic acid was transformed to hydroxamic acid (28) the N-tert-butoxycarbonyl protecting group in which was cleaved to give the final product (29) as hydrochloride salt.

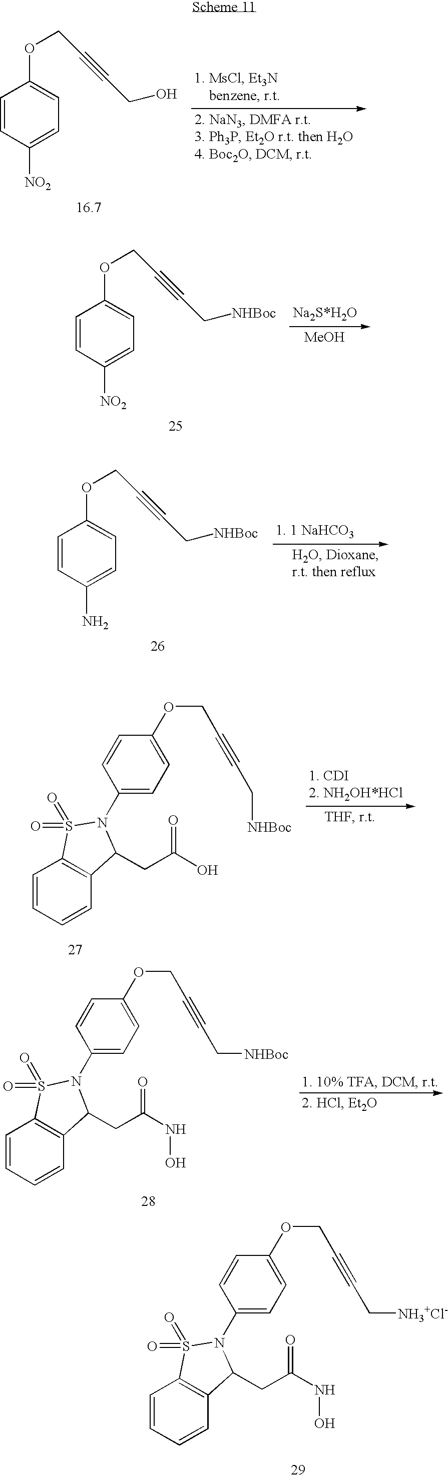

Hydroxamic acid (36) was prepared according to the Scheme 12. Known unsaturated ester (30) (see, e.g., Eberbach et al., 1986) was regioselectively chlorosulphonylated and the product (31) used for the reaction with aniline (2.1) to give the cyclic ester (32). Phenolic hydroxy group was sulphonylated with triflic anhydride and the resulting product (33) used for the Suzuki-Miyaura coupling with phenylboronic acid. The ester functionality in the intermediate (34) was hydrolyzed and carboxylic acid (35) transformed to hydroxamic acid (36).

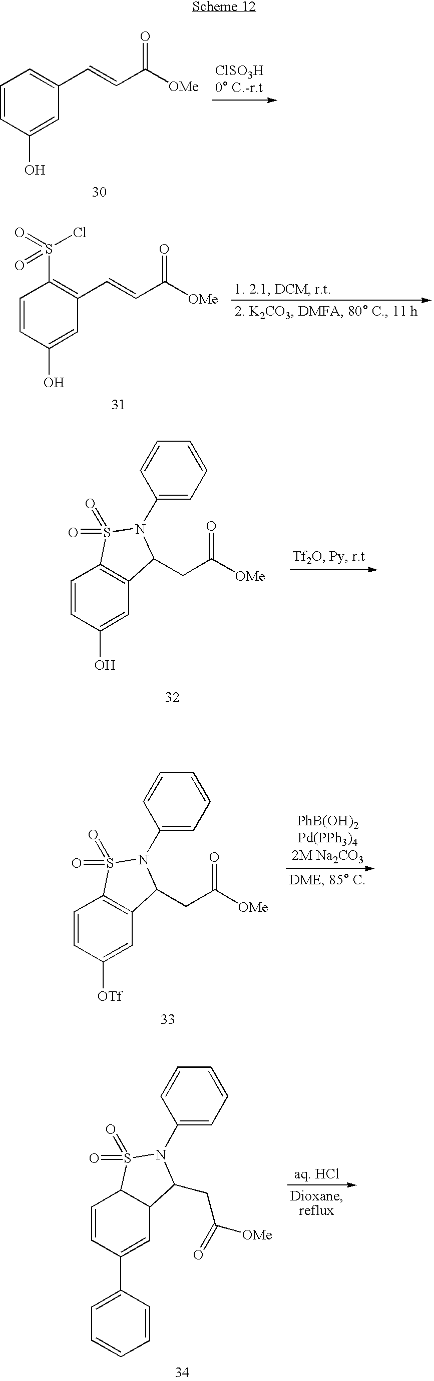

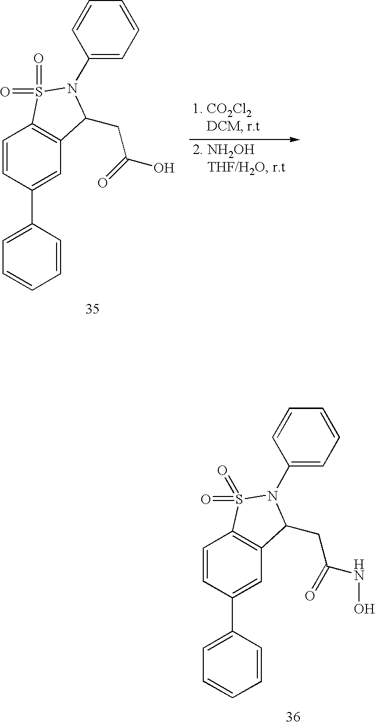

Hydroxamic acid (39) was prepared from cyclic ester (32). This was O-alkylated and the product (37) was hydrolyzed to give carboxylic acid (38) that in turn was transformed to hydroxamic acid (39).

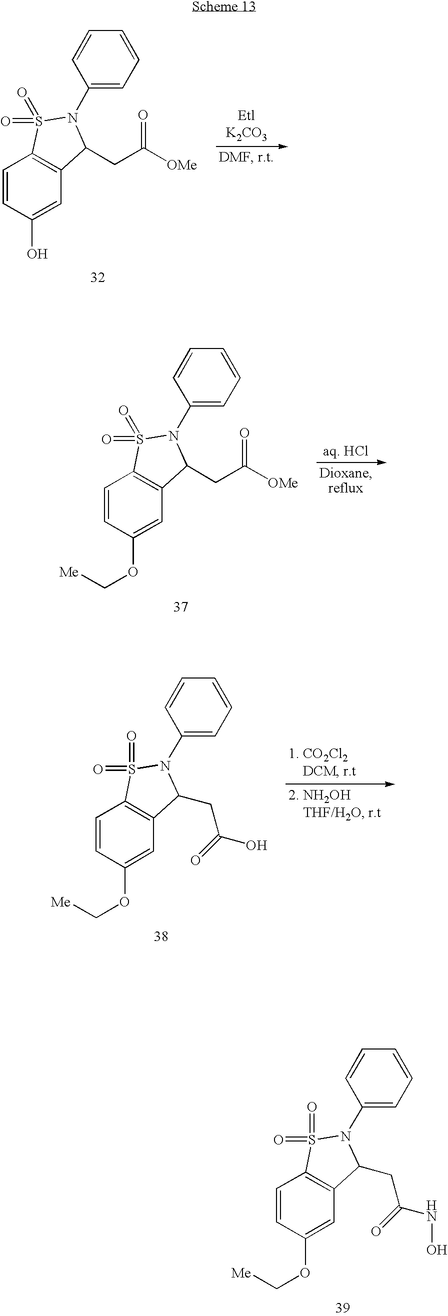

The synthesis of hydroxamic acid (43) is outlined in the Scheme 14. The reaction of sulphonylchloride (1.8) with aniline (2.1) gave unsaturated ester (40). This was used for the Suzuki-Miyaura coupling with phenylboronic acid to give an intermediate (41) that underwent cyclization and subsequent hydrolysis to yield carboxylic acid (42) that in turn was transformed to hydroxamic acid (43).

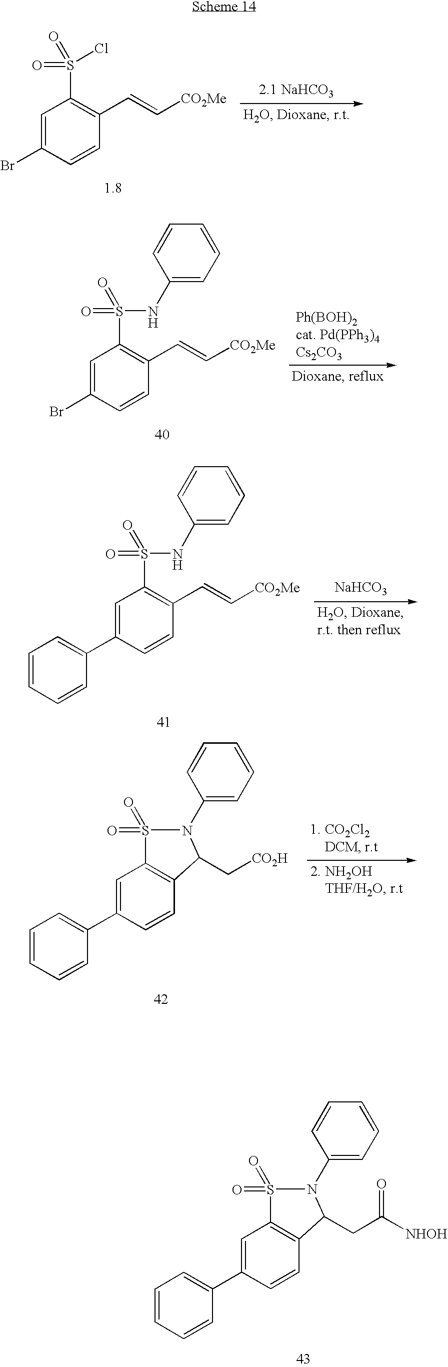

Hydroxamic acids (48.1) and (48.2) were prepared starting from commercially available sulphonamides (44.1) and (44.2) (Scheme 15). These were lithiated at the ortho-position to sulfonamide functionality (see, e.g., MacNeil et al., 2001) followed by iodination that led to intermediates (45.1) and (45.2). Heck reaction of aryliodides (45.1) and (45.2) with methyl acrylate provided cyclic esters (46.1) and (46.2). These were hydrolyzed to carboxylic acids (47.1) and (47.2) that were further transformed to hydroxamic acids (48.1) and (48.2).

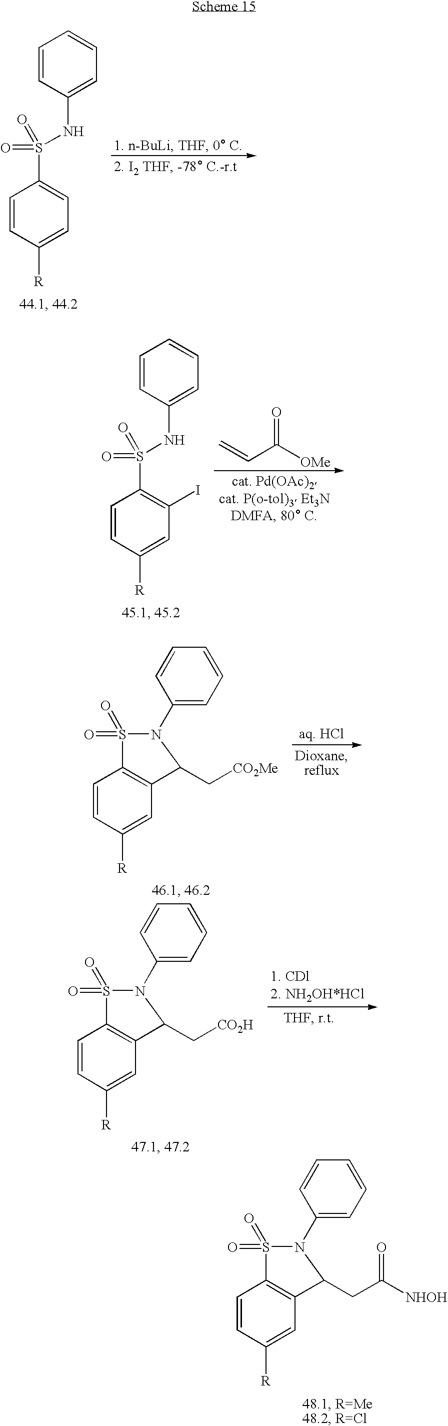

Hydroxamic acids (54.1)-(54.9) were prepared by different approach (Scheme 16).

Sulphonamides (50.1)-(50.9) were obtained from commercially available sulphonylchlorides (49.1)-(49.9) and used for directed ortho-lithiation, formylation reaction sequence to provide intermediates (51.1)-(51.9). Olefination reaction of these intermediates gave cyclic esters (52.1)-(52.9) that were hydrolyzed to acids (53.1)-(53.9) and these were further transformed to hydroxamic acids (54.1)-(54.9).

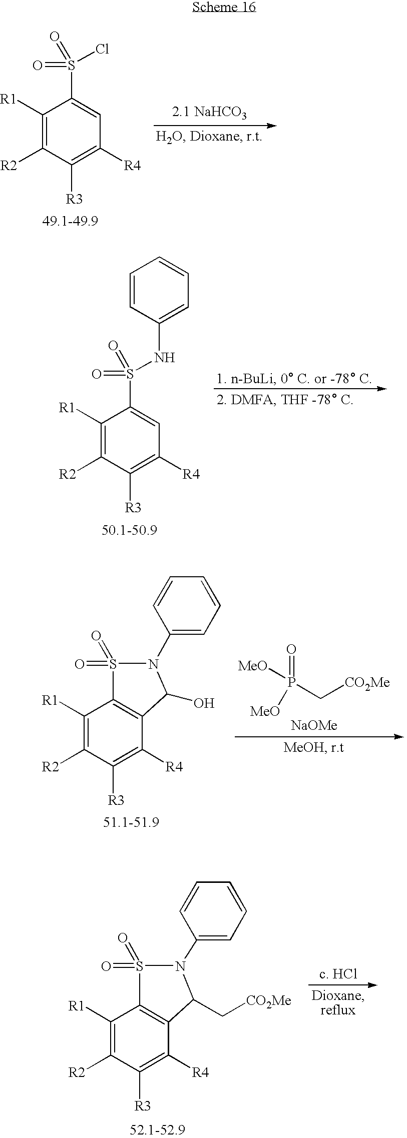

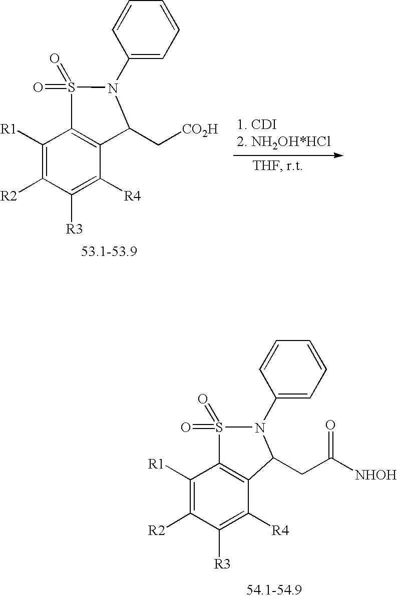

Sulphonamide (50.10) was prepared from sulphonylchloride (49.10) and was used for ortho-lithiation, formylation reaction sequence. This gave dehalogenated product (51.10) that was further transformed to hydroxamic acid (54.10) by using already established synthetic route (Scheme 17).

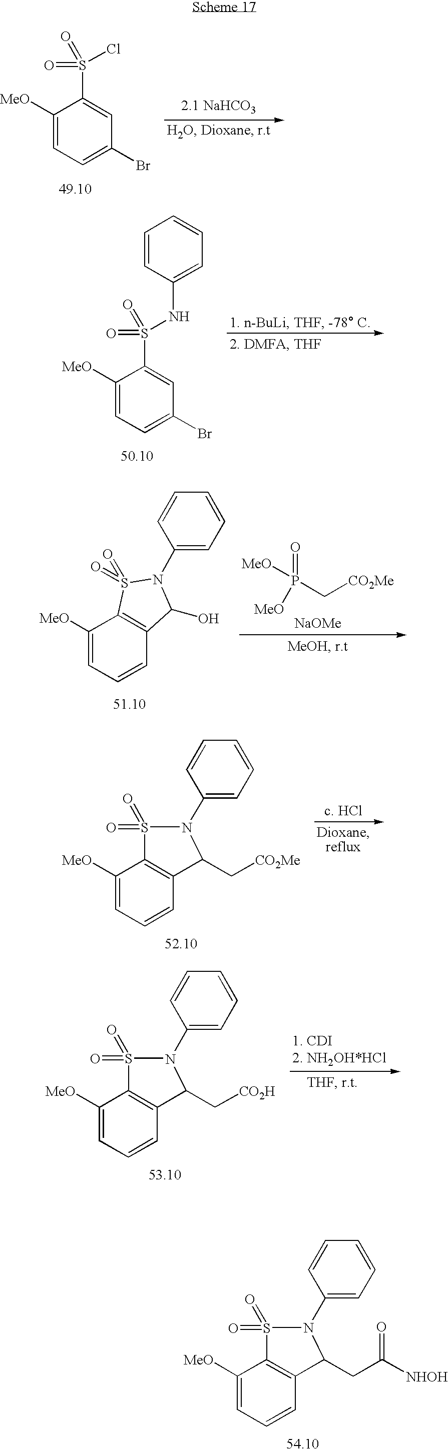

Hydroxamic acid (54.11) was obtained according to the Scheme 18. Sulphonamide (50.11) was prepared from sulphonylchloride (49.11) and subjected to ortho-lithiation, formylation reaction sequence to give intermediate (51.11). The latter was used for olefination reaction giving product 52.11 with fluoro group replaced to methoxy group. This was further transformed to hydroxamic acid (54.11) using established procedures.

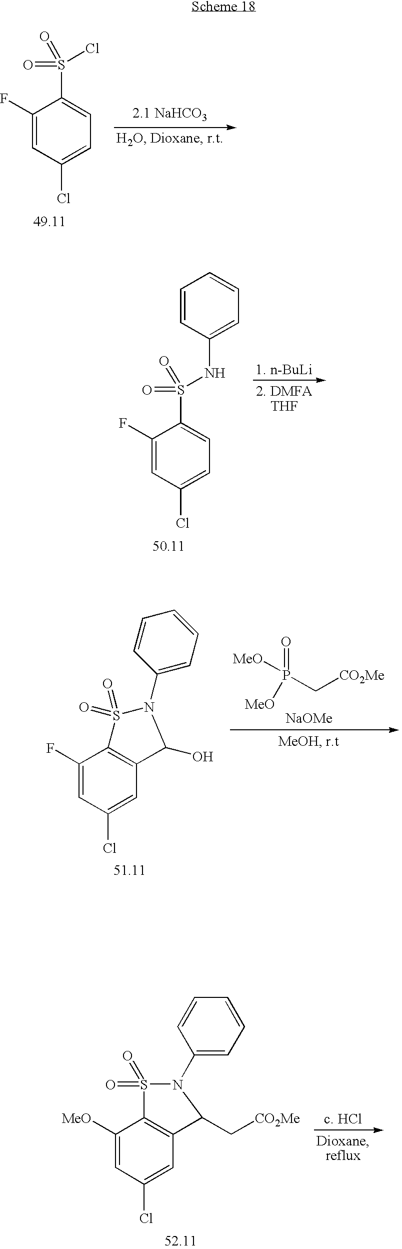

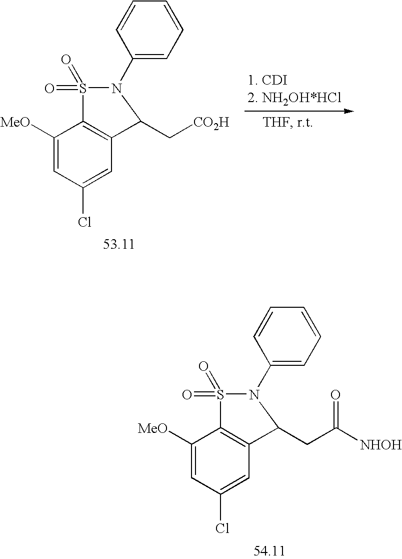

Cyclic intermediate (51.5) gave product (52.12) having fluoro group replaced with metoxygroup besides the product (52.5) in the olefination reaction (Scheme 19). Cyclic ester (52.12) was transformed to hydroxamic acid (54.12) using established procedures.

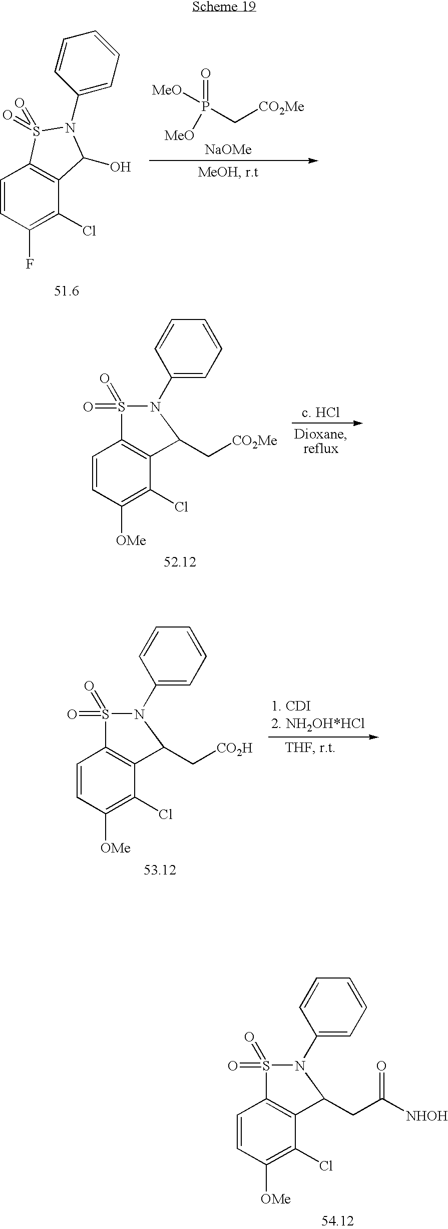

Hydroxamic acid (57) was prepared starting from ester (3.1) (Scheme 20). This was reduced and the resulting primary alcohol transformed to chloride. Chloride was replaced with cyanide to give intermediate (55) that was hydrolyzed and the resulting carboxylic acid (56) further transformed to hydroxamic acid (57).

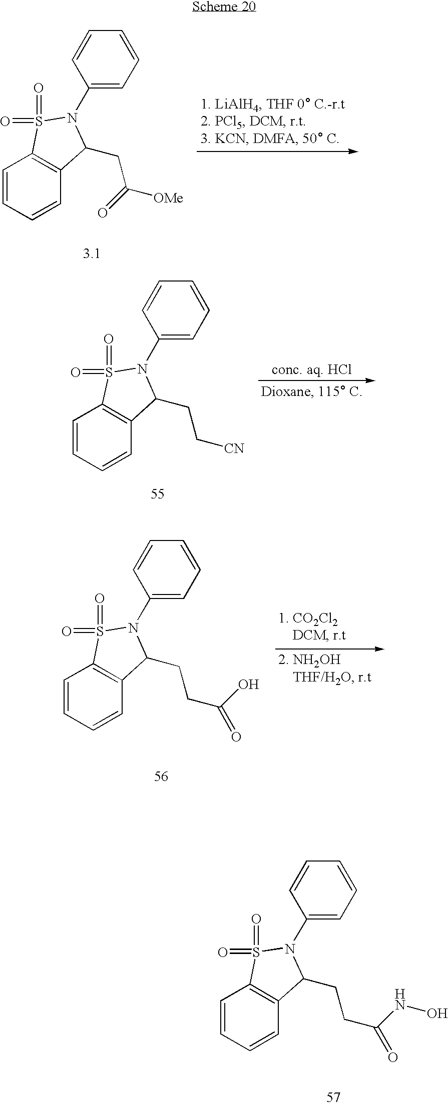

The synthesis of hydroxamic acids (62.1)-(62.2) was performed according to the Scheme 21. Sulphonamides (59.1)-(59.2) prepared from sulphonylchlorides (58.1)-(58.2) were transformed to carboxylic acid esters (60.1)-(60.2) according to the published route (see, e.g., Takahashi et al., 2003). Esters (60.1)-(60.2) were hydrolyzed and the resulting carboxylic acids (61.1)-(61.2) were transformed to hydroxamic acids (62.1)-(62.2).

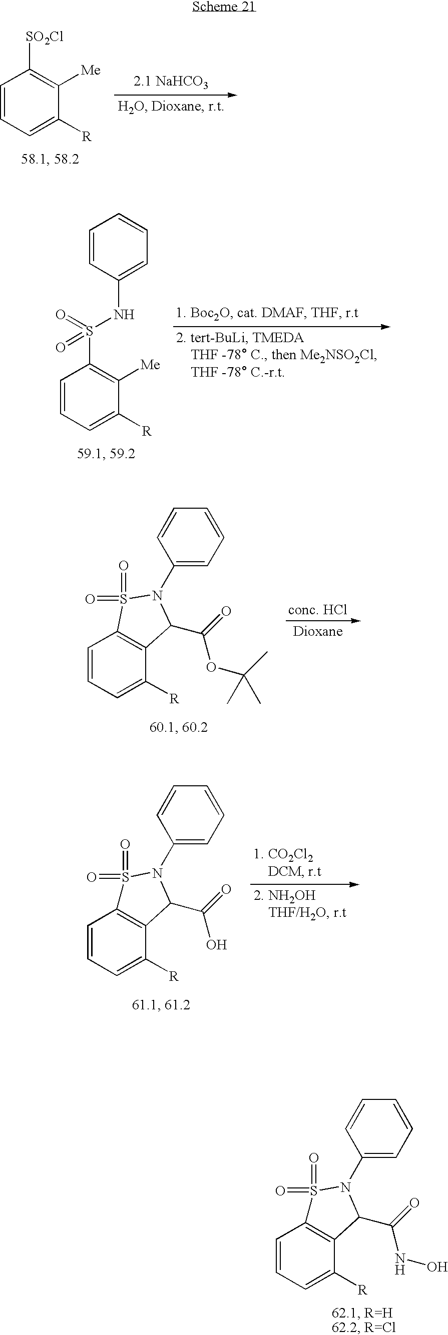

Stereoisomers of cyclic sulphonamides (5.1), (5.43) and (5A4) were prepared in enantiomerically pure form (Scheme 22). For this purpose, (R)-phenylglycinol was acylated with racemic acids (4.1), (4.43) and (4.44) to give the corresponding amides as a mixture of diastereomers (S,R)-(63.1),(63.2),(63.3) and (R,R)-(63.1),(62.3),(63.3) that were separated by means of chromatography. Separated amides (S,R)-(63.1),(63.2),(63.3) and (R,R)-(63.1),(63.2),(63.3) were hydrolyzed to enantiomerically pure acid isomers (S)-(4.1),(4.43),(4.44) and (R)-(4.1),(4.43),(4.44) that were further transformed to enantiomerically pure hydroxamic acids (+)-(5.1), (+)-(5.43), (+)-(5.44) and (−)-(5.1), (−)-(5.43), and (−)-(5.44).

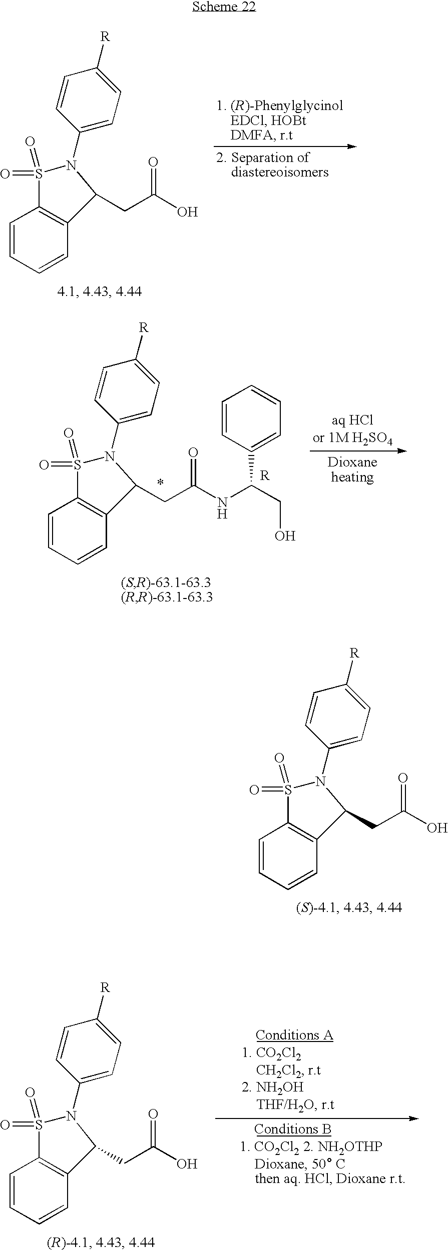

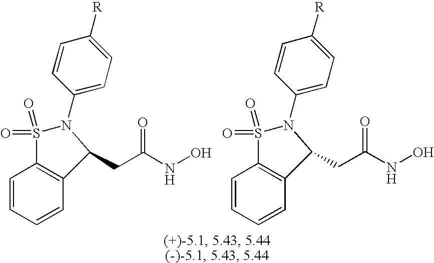

Hydroxamic acid (72) was prepared as follows (Scheme 23). Salicylaldehyde (64) was treated with N,N-dimethylthiocarbamoylchloride to give thiocarbamate (65). This was subjected to the Newman-Kwart rearrangement providing S-carbamoyl thiosalicylaldehyde (66). Carbamoyl group in (66) was cleaved with MeONa and the resulting thiolate in situ alkylated with benzyl bromide to give S-benzylthiosalicylaldehyde (67). Subsequent Wittig reaction of aldehyde (67) gave unsaturated ester (68). Sulphide group in ester (68) was oxidised to give sulphone (69) that was transformed to cyclic product (70) as a result of NaHCO₃ promoted intramolecular Michael reaction. Hydrolysis of the ester (70) under acidic conditions gave acid (71) that was transformed to hydroxamic acid (72).

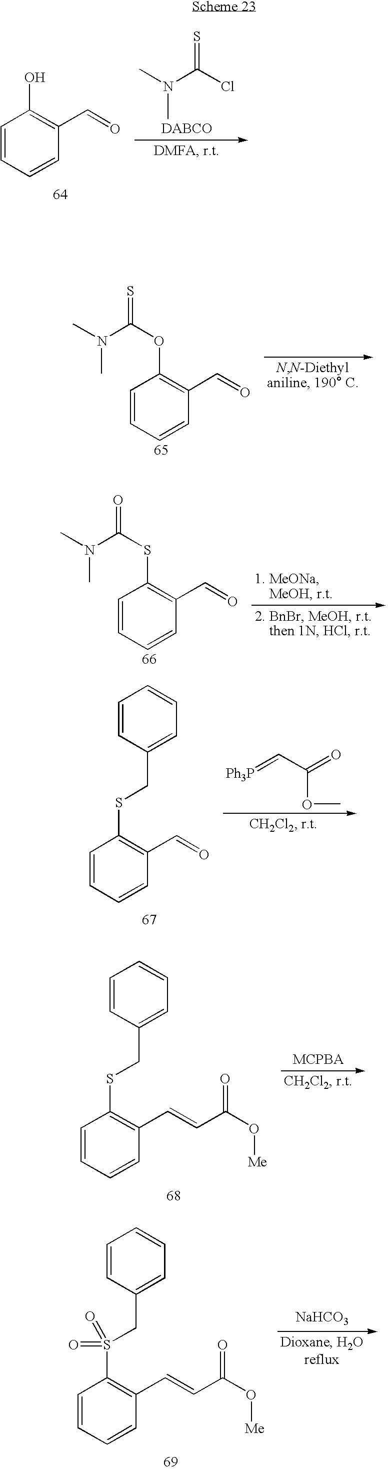

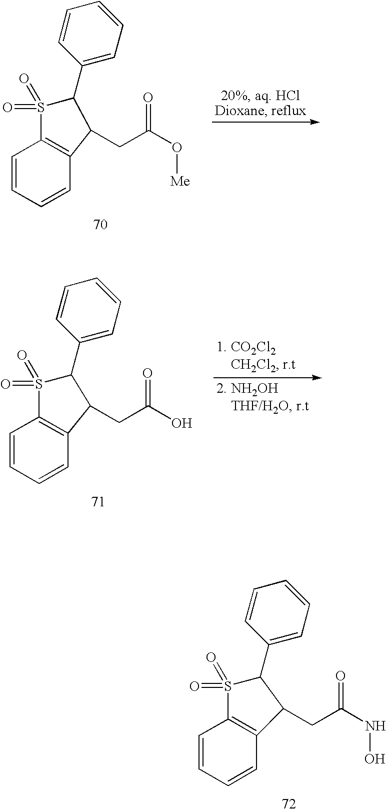

Hydroxamic acid (77) was prepared starting from known sulphonamide (73) (see, e.g., Goulaouic-Dubois et al., 1995). Orhto-lithiation, iodination reaction sequence provided iodide (74) that was used for the Heck reaction with methyl acrylate giving cyclic ester (75). This was hydrolyzed to carboxylic acid (76) that was further transformed to hydroxamic acid (77).

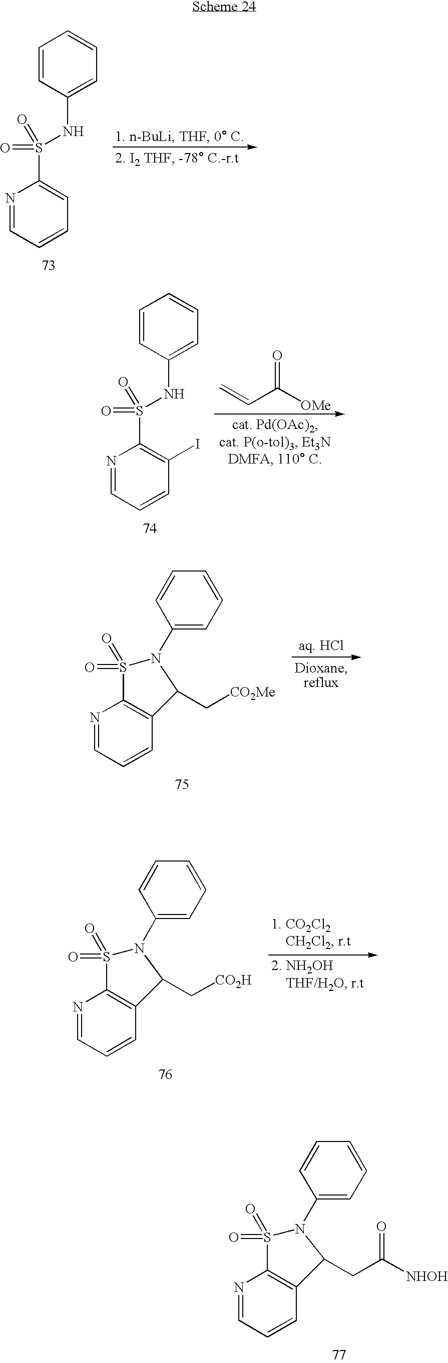

### General procedure for the preparation of (E)-3-(2-chlorosulfonylphenynacrylic acid methyl esters (1.2)-(1.6)

Method A: Chlorosulphonic acid (3.5 mL, 52 mmol) was cooled in an ice bath and to this added was unsaturated ester (7) (1.0 g, 5.2 mmol). The mixture was stirred while cooling starting material disappeared (TLC control, 30 minutes to 6 hours) and thoroughly poured into ice water. In the case the precipitate has formed, it was collected on a filter, washed with water and dried in vacuo to give the products (1). In the case no precipitate has formed, the aqueous phase was extracted with CHCl₃, combined organic phase was dried over Na₂SO₄ and the solvent removed in vacuo to give crude product (1) that was used for the next step without additional purification.

Following a method analogous to Method A, the following compounds were obtained as crude products.

| Synthesis | Method | Name | Stucture |
|---|---|---|---|
| 1 | A | (E)-3-(2-Chlorosulfonyl-5- methoxyphenyl)acrylic acid methyl ester (1.2) | 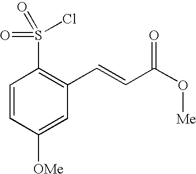 |
| 2 | A | (E)-3-(7-Chlorosulfonyl-2,3- dihydro-benzo[1,4]dioxin-6- yl)acrylic acid methyl ester (1.3) | 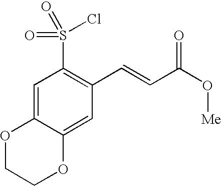 |
| 3 | A | (E)-3-(2-Chlorosulfonyl-4,5- dimethoxyphenyl)acrylic acid methyl ester (1.4) | 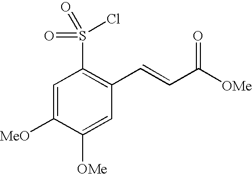 |
| 4 | A | (E)-3-(2-Chlorosulfonyl-3,4,5- trimethoxyphenyl)acrylic acid methyl ester (1.5) | 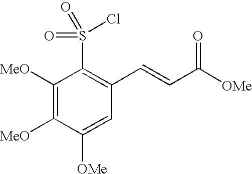 |
| 5 | A | (E)-3-(2-Chlorosulfonyl-3,5- dimethoxyphenyl)acrylic acid methyl ester (1.6) | 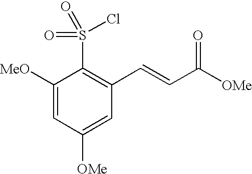 |

Compound (1.2): Slightly grey powder (0.88 g, 59%). ¹H-NMR (CDCl₃, TMS) δ: 3.85 (3H, s); 3.94 (3H, s); 6.41 (1H, d, 15 Hz); 7.01 (1H, dd, 2 Hz and 9 Hz); 7.16 (1H, d, 2 Hz); 8.07 (1H, d, 9 Hz) and 8.46 ppm (1H, d, 15 Hz).

### General procedure for the synthesis of (E)-3-(2-chlorosulfonylphenyl)acrylic acid methyl esters (1.7) and (1.8)

Method B: 2-Aminobenzenesulphonic acid (8) (10 mmol) was suspended in sulphuric acid (5 mL) and the reaction mixture cooled in an ice bath. To this added was 40% aqueous NaNO₂ (2 mL) and the mixture was stirred for 1 h. Et₂O was added and the precipitate was collected on a filter. The crude product (9) (2.35 g) obtained was suspended in DMFA (7 mL) under inert atmosphere and Pd₂(dba)₃ (30 mg) was added followed by methyl acrylate (2.7 mmol, 30 mmol). The reaction mixture was stirred at room temperature for 10 h. Solvent was removed in vacuo to obtain crude sulphonic acid (10). To this added was toluene (7 mL) and thionylchloride (5.5 mL, 80 mmol). The resulting mixture was refluxed for 4 h, cooled to room temperature and filtered. The solution was concentrated in vacuo to give sulphonylchloride (1.7) or (1.8) as a crude product.

Following a method analogous to Method B, the following compounds were obtained as crude products.

| Synthesis | Method | Name | Stucture |
|---|---|---|---|
| 6 | B | (E)-3-(2-Chlorosulfonyl-4- methylphenyl)acrylic acid methyl ester (1.7) | 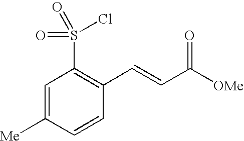 |
| 7 | B | (E)-3-(2-Chlorosulfonyl-4- bromophenyl)acrylic acid methyl ester (1.8) | 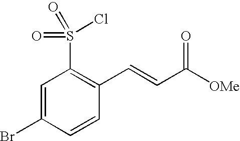 |

### Synthesis 8

### 4-(But-2-yn-1-yloxy)phenylamine (2.43)

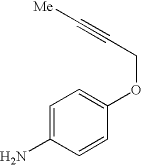

A mixture of para-hydroxyaniline sulphate (11) (395 mg, 2.5 mmol), but-2-yn-1-yl methanesulphonate (12) (370 mg, 2.5 mmol) and Cs₂CO₃ (2.44 g, 7.5 mmol) in DMFA (10 mL) was heated at 60° C. for 6 h. The mixture was poured into water (50 mL) and extracted with EtOAc (20 mL). The organic phase was separated and dried over Na₂SO₄. The solution was filtered and evaporated to give crude product (2.43) (140 mg, 35%) as a dark oil that was used for the next step without purification. ¹H-NMR (CDCl₃, TMS) δ: 1.84 (3H, t, 2 Hz); 4.53 (2H, m); 6.62 (2H, d, 8 Hz) and 6.78 ppm (2H, d, 8 Hz).

### Synthesis 9

### 4-(2-Methylquinolin-4-methyloxy)aniline (2.44)

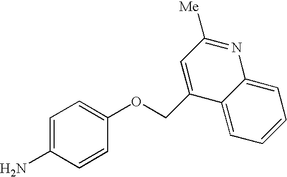

A mixture of para-hydroxyaniline sulphate (11) (207 mg, 1 mmol), 4-chloromethyl-2-methyl-quinoline hydrochloride (13) (228 mg, 1 mmol) and Cs₂CO₃ (1.63 g, 5 mmol) in DMFA (5 mL) was stirred at room temperature for 3 h. The mixture was poured into water (50 mL) and extracted with EtOAc (20 mL). The organic phase was separated and dried over Na₂SO₄. The solution was filtered and evaporated. The residue was purified by flash chromatography on silica gel eluting with EtOAc to give product (2.44) (165 mg, 63%). ¹H-NMR (CDCl₃, TMS) δ: 2.73 (3H, s); 3.9 (2H, br s); 5.40 (2H, s); 6.65 (2H, d, 9 Hz); 6.85 (2H, d, 9 Hz); 7.44 (1H, s); 7.50 (1H, t, 8 Hz); 7.68 (1H, t, 8 Hz); 7.90 (1H, d) and 8.07 ppm (1H, d, 8 Hz).

### General Procedure for the Preparation of Anilines (2.45)-(2.61)

Method C: 4-Nitrophenol (14) (3.1 g, 2.2 mmol), alkylating agent (15) and K₂CO₃ (920 mg, 6.7 mmol) was suspended in DMF (7 mL). The resulting suspension was stirred at room temperature for 48 h and poured into water (70 mL). The product was taken into EtOAc (70 mL). The organic phase was separated and washed with brine (70 mL). The extract was dried over Na₂SO₄, filtered and the solvent removed in vacuo to give practically pure intermediate (16).

For the synthesis of anilines (2.45) and (2.46), intermediates (16.1) and (16.2) (6.5 mmol) were dissolved in EtOH (15 mL) and 10% Pd/C (95 mg) was added to the solution. The mixture was stirred under H₂ atmosphere until full conversion of the starting material (ca 4 h). The mixture was passed trough celite column and the solvent removed in vacuo to give anilines (2.45) and (2.46) as crude products. For the synthesis of aniline (2.48), Raney Nickel was used as a hydrogenation catalyst.

For the synthesis of anilines (2.47), (2.49)-(2.61) the corresponding intermediates (16) (0.3 mmol) were dissolved in methanol (2 mL) and Na₂S×9H₂O (1 mmol) was added to the solution and the mixture was set to reflux until full conversion of the starting material (ca 2 h). The solvent was removed in vacuo and the residue partitioned between the water and EtOAc. The organic phase was dried over Na₂SO₄, filtered and evaporated. The residue was purified by means of the column chromatography on silica gel or used for the next step without purification.

Following a method analogous to Method C, the following compounds were obtained as crude products.

| Synthesis | Method | Name | Stucture |
|---|---|---|---|
| 10 | C | 4-(2- Dimethylaminoethoxy)aniline (2.45) | 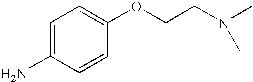 |
| 11 | C | 4-(2-Morpholin-4-yl- ethoxy)aniline (2.46) | 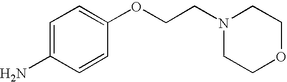 |
| 12 | C | 4-Allyloxyaniline (2.47) | 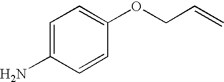 |
| 13 | C | 4-(Pyridin-4-ylmethoxy)aniline (2.48) | 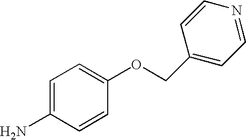 |
| 14 | C | 4-(Pyridin-3-ylmethoxy)aniline (2.49) | 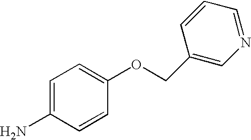 |
| 15 | C | 4-(Pyridin-2-ylmethoxy)aniline (2.50) | 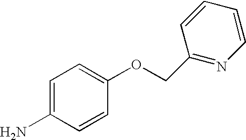 |
| 16 | C | 4-(4-Aminophenoxy)but-2-yn-1- ol (2.51) | 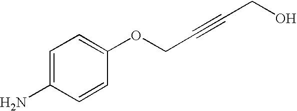 |
| 17 | C | 4-Pent-2-ynyloxyaniline (2.52) | 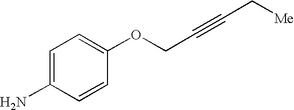 |
| 18 | C | 4-Prop-2-ynyloxyaniline (2.53) | 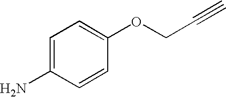 |
| 19 | C | 4-Prop-2-ynyloxyaniline (2.54) | 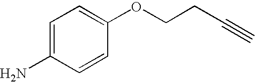 |
| 20 | C | 4-(1-Methyl-but-2- ynyloxy)aniline (2.55) | 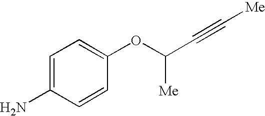 |
| 21 | C | 4-[1-(2-Methyl-quinolin-4- yl)ethoxy]aniline (2.56) | 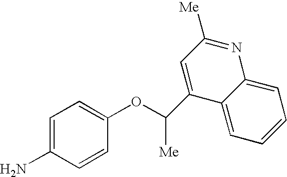 |
| 22 | C | 4-(Quinolin-4-ylmethoxy)aniline (2.57) | 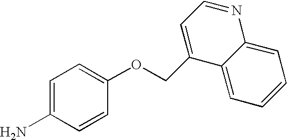 |
| 23 | C | 4-(6-Fluoro-2-methyl-quinolin-4- ylmethoxy)aniline (2.58) | 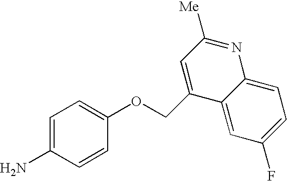 |
| 24 | C | 4-(6-Chloro-2-methyl-quinolin-4- ylmethoxy)aniline (2.59) | 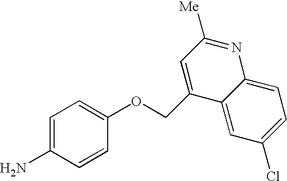 |
| 25 | | 4-(2-Methylpyridin-4- ylmethoxy)aniline (2.60) | 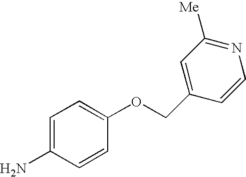 |
| 26 | | 4-(2,6-Dimethylpyridin-4- ylmethoxy)aniline (2.61) | 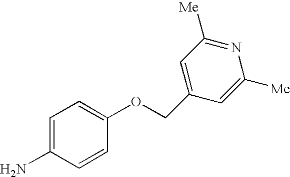 |

### General procedure for the preparation of (1,1-dioxo-2-phenyl-2,3-dihydro-1H-benzo[d]isothiazol-3-yl)acetic acid methyl esters (3.1)-(3.3), (3.26)-(3.29), (3.44)-(3.46), (3.48)-(3.50), (3.56)-(3.66)

Method D: To a solution of suphonylchloride (1) (1 mmol) and amine (2) (1 mmol) in dioxane (5 mL) added was 1M aqueous solution of NaHCO₃ (3 mL). The resulting mixture was stirred at room temperature for 2 hours and then refluxed for 2 hours. After cooling to room temperature, water (20 mL) and EtOAc (20 mL) was added. The organic phase was separated and washed with brine (20 mL) and dried over Na₂SO₄. The solution was filtered and evaporated and the residue was purified by flash chromatography on silica gel eluting with a mixture of light petroleum ether and EtOAc.

Following a method analogous to Method D, the following compounds were obtained as crude products.

| Synthesis | Method | Name | Stucture |
|---|---|---|---|
| 27 | D | 2-(1,1-Dioxo-2-phenyl-2,3- dihydro-6-benzo[d]isothiazol-3- yl)acetic acid methyl ester (3.1) | 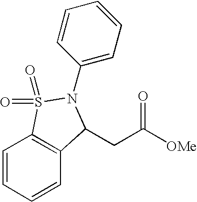 |
| 28 | D | 2-(2-naphthalen-2-yl-1,1-dioxo- 2,3-dihydro-1H- benzo[d]isothiazol-3-yl)acetic acid methyl ester (3.2) | 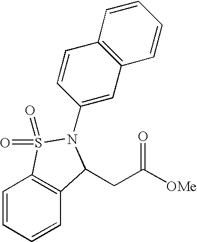 |
| 29 | D | 2-(1,1-Dioxo-2-m-tolyl-2,3- dihydro-1H-benzo[d]isothiazol- 3-yl)acetic acid methyl ester (3.3) | 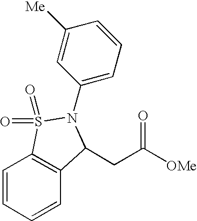 |
| 30 | D | 2-[1,1-Dioxo-2-(4-pyrrol-1-yl- phenyl)-2,3-dihydro-1H- benzo[d]isothiazol-3-yl]-acetic acid methyl ester (3.26) | 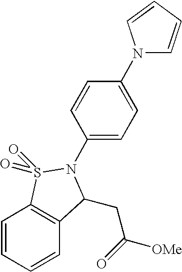 |
| 31 | D | 2-[2-(4-Imidazol-1-yl-phenyl)- 1,1-dioxo-2,3-dihydro-1H- benzo[d]isothiazol-3-yl]acetic acid methyl ester (3.27) | 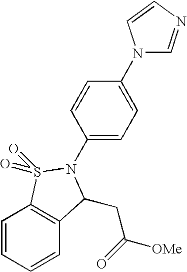 |
| 32 | D | 2-[1,1-Dioxo-2-(4-[1,2,4]triazol- 1-yl-phenyl)-2,3-dihydro-1H- benzo[d]isothiazol-3-yl]acetic acid methyl ester (3.28) | 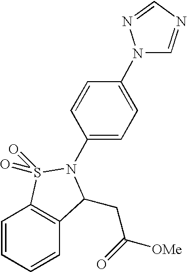 |
| 33 | D | 2-[2-(4-Oxazol-5-yl-phenyl)-1,1- dioxo-2,3-dihydro-1H- benzo[d]isothiazol-3-yl]acetic acid methyl ester (3.29) | 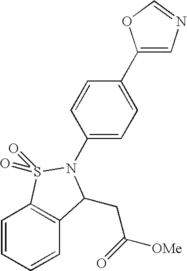 |
| 34 | D | {2-[4-(2-Methyl-quinolin-4- ylmethoxy)-phenyl]-1,1-dioxo- 2,3-dihydro-1H- benzo[d]isothiazol-3-yl}acetic acid methyl ester (3.44) | 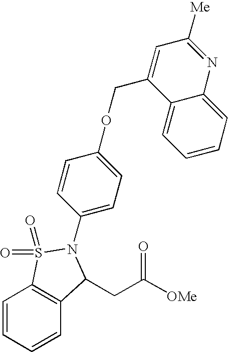 |
| 35 | D | 2-{2-[4-(2-Dimethylamino- ethoxy)phenyl]-1,1-dioxo-2,3- dihydro-1H-benzo[d]isothiazol- 3-yl}acetic acid (3.45) | 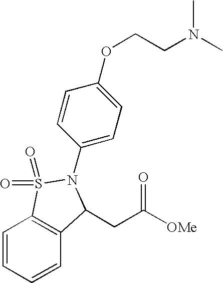 |
| 36 | D | 2-{2-[4-(2-Morpholin-4-yl- ethoxy)-phenyl]-1,1-dioxo-2,3- dihydro-1H-benzo[d]isothiazol- 3-yl}acetic acid methyl ester (3.46) | 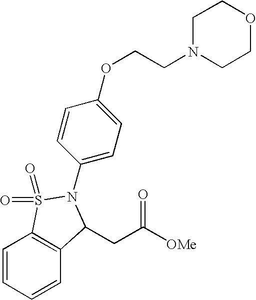 |
| 37 | D | 2-{1,1-Dioxo-2-[4-(pyridin-4- ylmethoxy)phenyl]-2,3-dihydro- 1H-benzo[d]isothiazol-3-yl}- acetic acid methyl ester (3.48) | 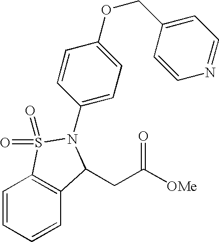 |
| 38 | D | 2-{1,1-Dioxo-2-[4-(pyridin-3- ylmethoxy)phenyl]-2,3-dihydro- 1H-benzo[d]isothiazol-3-yl}- acetic acid methyl ester (3.49) | 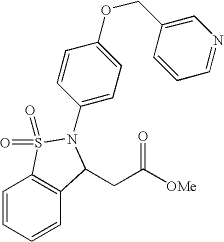 |
| 39 | D | 2-{1,1-Dioxo-2-[4-(pyridin-2- ylmethoxy)phenyl]-2,3-dihydro- 1H-benzo[d]isothiazol-3-yl}- acetic acid methyl ester (3.50) | 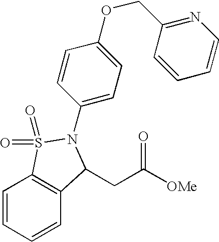 |
| 40 | D | 2-(2-{4-[1-(2-Methylquinolin-4- yl)ethoxy]-phenyl}-1,1-dioxo- 2,3-dihydro-1H- benzo[d]isothiazol-3-yl)-acetic acid methyl ester (3.56) | 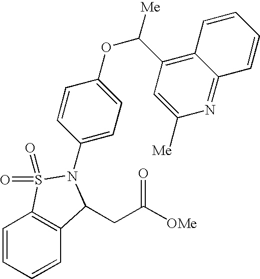 |
| 41 | D | 2-{1,1-Dioxo-2-[4-(quinolin-4- ylmethoxy)phenyl]-2,3-dihydro- 1H-benzo[d]isothiazol-3-yl}- acetic acid methyl ester (3.57) | 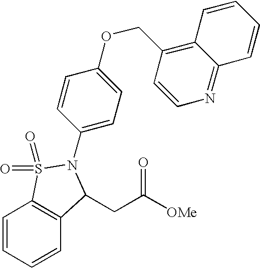 |
| 42 | D | 2-{2-[4-(6-Fluoro-2-methyl- quinolin-4-ylmethoxy)-phenyl]- 1,1-dioxo-2,3-dihydro-1H- benzo[d]isothiazol-3-yl}-acetic acid methyl ester (3.58) | 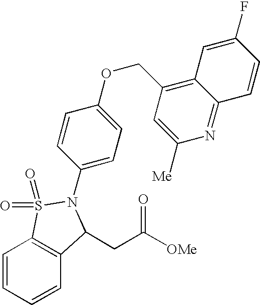 |
| 43 | D | 2-{2-[4-(6-Chloro-2-methyl- quinolin-4-ylmethoxy)-phenyl]- 1,1-dioxo-2,3-dihydro-1H- benzo[d]isothiazol-3-yl}-acetic acid methyl ester (3.59) | 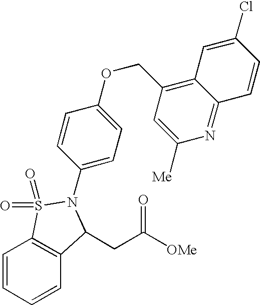 |
| 44 | D | 2-{2-[4-(2-Methylpyridin-4- ylmethoxy)phenyl]-1,1-dioxo- 2,3-dihydro-1H- benzo[d]isothiazol-3-yl}acetic acid methyl ester (3.60) | 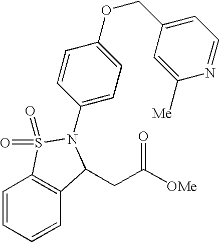 |
| 45 | D | 2-[4-(2,6-Dimethyl-pyridin-4- ylmethoxy)-phenyl]-1,1-dioxo- 2,3-dihydro-1H- benzo[d]isothiazol-3-yl}acetic acid methyl ester (3.61) | 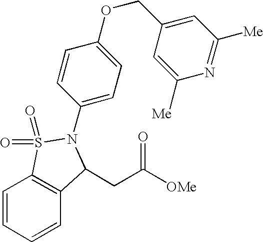 |
| 46 | D | 2-(5-Methoxy-1,1-dioxo-2- phenyl-2,3-dihydro-1H- benzo[d]isothiazol-3-yl)acetic acid methyl ester (3.62) | 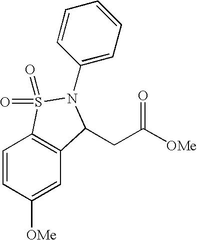 |
| 47 | D | 2-(1,1-Dioxo-2-phenyl-2,3,6,7- tetrahydro-1H-5,8-dioxa-thia-2- aza-cyclopenta[b]naphthalen-3- yl)acetic acid methyl ester (3.63) | 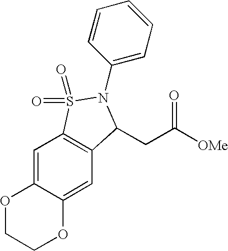 |
| 48 | D | 2-(5,6-Dimethoxy-1,1-dioxo-2- phenyl-2,3-dihydro-1H- benzo[d]isothiazol-3-yl)acetic acid methyl ester (3.64) | 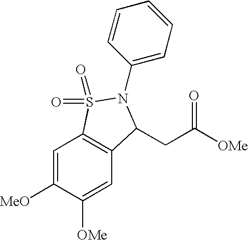 |
| 49 | D | 2-(5,6,7-Trimethoxy-1,1-dioxo- 2-phenyl-2,3-dihydro-1H- benzo[d]isothiazol-3-yl)acetic acid methyl ester (3.65) | 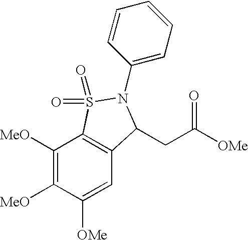 |
| 50 | D | 2-(5,7-Dimethoxy-1,1-dioxo-2- phenyl-2,3-dihydro-1H- benzo[d]isothiazol-3-yl)acetic acid methyl ester (3.66) | 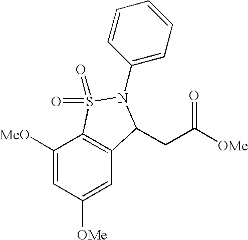 |

Compound (3.1): Yield 61%, ¹H-NMR (CDCl₃, TMS) δ: 2.74 (1H, dd, 8 Hz and 16 Hz); 2.97 (1H, dd, 4 Hz band 16 Hz); 3.59 (3H, s); 5.58 (1H, dd, 4 Hz and 8 Hz); 7.3-7.7 (8H, m) and 7.89 ppm (1H, d, 8 Hz).

Compound (3.2): Yield 71%, ¹H-NMR (DMSO-d₆, TMS) δ: 3.01 (2H, d, 5 Hz); 3.29 (3H, s); 5.92 (1H, t, 5 Hz) and 7.5-8.0 ppm (11H, m).

Compound (3.3): Yield 23%, ¹H-NMR (CDCl₃, TMS) δ: 2.40 (3H, s); 2.77 (1H, dd, 8 Hz and 16 Hz); 2.97 (1H, dd, 4 Hz and 16 Hz); 3.61 (3H, s); 5.56 (1H, dd, 4 Hz and 8 Hz); 7.1-7.7 (7H, m) and 7.89 ppm (1H, d, 8 Hz).

Compound (3.62): Yield 66%, ¹H-NMR (CDCl₃, TMS) δ: 2.76 (1H, dd, 8 Hz and 16 Hz); 2.96 (1H, dd, 4 Hz and 16 Hz); 3.60 (3H, s); 3.70 (3H, s); 3.89 (3H, s); 5.53 (1H, dd, 4 Hz and 8 Hz); 6.9-7.5 (7H, m) and 7.77 ppm (1H, d, 8 Hz).

### General procedures for preparation of 2-(1,1-dioxo-2-phenyl-2,3-dihydro-1H-benzo[d]isothiazol-3-yl)acetic acids (4)

Method E: From esters (3). A solution of ester (3) (1 mmol) in a mixture of dioxane (20 mL) and concentrated aqueous HCl (5 mL) was stirred in room temperature for 2 days. Solvents were evaporated and replaced with fresh dioxane (20 mL) and concentrated aqueous HCl (5 mL). Stirring was continued for additional 2 days, until complete disappearance of starting material (TLC control; if necessary solvent system was replaced once more). Solvents were evaporated to give product (4).

Method F: From sulphonylchlorides (1) and amines (2). To a solution of sulphonylchloride (1) (1 mmol) and amine (2) (1 mmol) in dioxane (5 mL) added was 1 M aqueous solution of NaHCO₃ (3 mL). The resulting mixture was stirred at room temperature for 2 hours and then refluxed for 8 hours. After cooling to room temperature, water (20 mL) and EtOAc (20 mL) were added. The aqueous phase was separated and acidified to pH˜2 with concentrated aqueous HCl and extracted with EtOAc (20 mL). The organic phase was washed with brine (20 mL) and dried over Na₂SO₄. The solution was filtered and evaporated to give the residue with product (4) content ˜30-80%. In most cases it was used for further transformation without purification.

Following a method analogous to Method E or Method F, the following compounds were obtained as crude products.

| Synthesis | Method | Name | Stucture |
|---|---|---|---|
| 51 | E | 2-(1,1-Dioxo-2-phenyl-2,3-dihydro- 6-benzo[d]isothiazol-3-yl)acetic acid (4.1) | 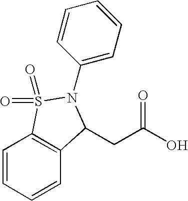 |
| 52 | E | 2-(2-Naphthalen-2-yl-1,1-dioxo- 2,3-dihydro-1H-benzo[d]isothiazol- 3-yl)acetic acid (4.2) | 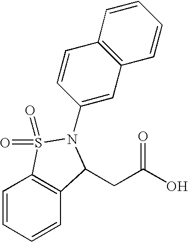 |
| 53 | E | 2-(1,1-Dioxo-2-m-tolyl-2,3-dihydro- 1H-benzo[d]isothiazol-3-yl)acetic acid (4.3) | 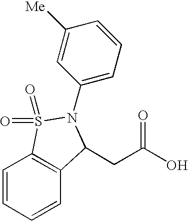 |
| 54 | F | 2-(1,1-Dioxo-2-p-tolyl-2,3-dihydro- 1H-benzo[d]isothiazol-3-yl)acetic acid (4.4) | 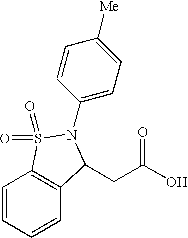 |
| 55 | F | 2-(1,1-Dioxo-2-o-tolyl-2,3-dihydro- 1H-benzo[d]isothiazol-3-yl)acetic acid (4.5) | 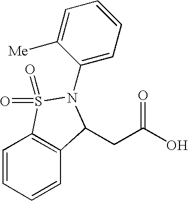 |
| 56 | F | 2-[2-(2-Methoxyphenyl)-1,1-dioxo- 2,3-dihydro-1H-benzo[d]isothiazol- 3-yl]acetic acid (4.6) | 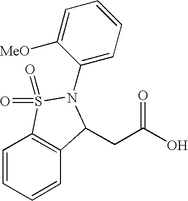 |
| 57 | F | 2-[2-(3-Methoxyphenyl)-1,1-dioxo- 2,3-dihydro-1H-benzo[d]isothiazol- 3-yl]acetic acid (4.7) | 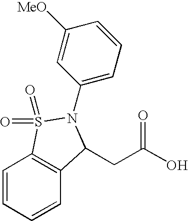 |
| 58 | F | 2-[2-(4-Methoxyphenyl)-1,1-dioxo- 2,3-dihydro-1H-benzo[d]isothiazol- 3-yl]acetic acid (4.8) | 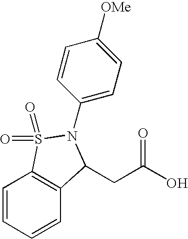 |
| 59 | F | 2-[2-(3-Phenoxyphenyl)-1,1-dioxo- 2,3-dihydro-1H-benzo[d]isothiazol- 3-yl]acetic acid (4.9) | 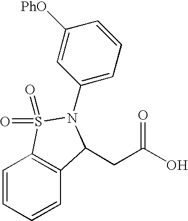 |
| 60 | F | 2-[2-(4-Chlorophenyl)-1,1-dioxo- 2,3-dihydro-1H-benzo[d]isothiazol- 3-yl]acetic acid (4.10) | 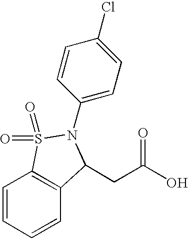 |
| 61 | F | 2-[2-(3-Methylsulfanylphenyl)-1,1- dioxo-2,3-dihydro-1H- benzo[d]isothiazol-3-yl]acetic acid (4.11) | 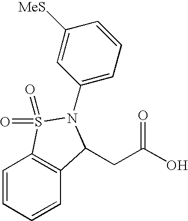 |
| 62 | F | 2-[2-(3-Trifluoromethylphenyl)-1,1- dioxo-2,3-dihydro-1H- benzo[d]isothiazol-3-yl]acetic acid (4.12) | 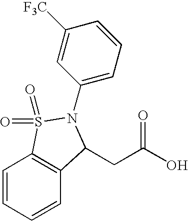 |
| 63 | F | 2-[2-(3-Trifluoromethyloxyphenyl)- 1,1-dioxo-2,3-dihydro-1H- benzo[d]isothiazol-3-yl]acetic acid (4.13) | 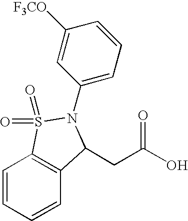 |
| 64 | F | 2-[2-(4-Iodophenyl)-1,1-dioxo-2,3- dihydro-1H-benzo[d]isothiazol-3- yl]acetic acid (4.14) | 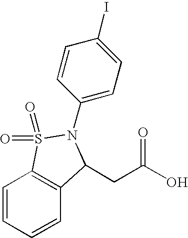 |
| 65 | F | 2-(2-Biphenyl-3-yl-1,1-dioxo-2,3- dihydro-1H-benzo[d]isothiazol-3- yl)acetic acid (4.15) | 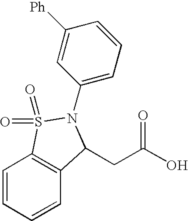 |
| 66 | F | 2-[2-(4-Fluoro-phenyl)-1,1-dioxo- 2,3-dihydro-1H-benzo[d]isothiazol- 3-yl]acetic acid (4.16) | 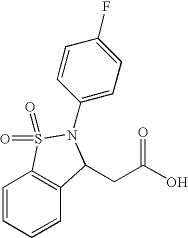 |
| 67 | F | 2-[2-(4-Trifluormethylphenyl)-1,1- dioxo-2,3-dihydro-1H- benzo[d]isothiazol-3-yl]acetic acid (4.17) | 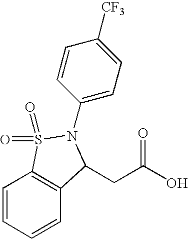 |
| 68 | F | 2-[2-(4-Trifluormethyloxyphenyl)- 1,1-dioxo-2,3-dihydro-1H- benzo[d]isothiazol-3-yl]acetic acid (4.18) | 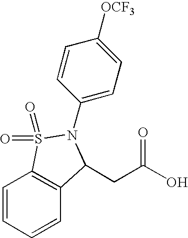 |
| 69 | F | 2-[1,1-Dioxo-2-(4- trifluoromethylsulfanylphenyl)-2,3- dihydro-1H-benzo[d]isothiazol-3- yl]-acetic acid (4.19) | 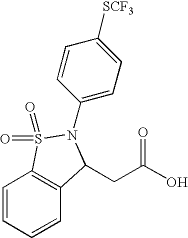 |
| 70 | F | 2-[1,1-Dioxo-2-(4- methylsulfanylphenyl)-2,3-dihydro- 1H-benzo[d]isothiazol-3-yl]acetic acid (4.20) | 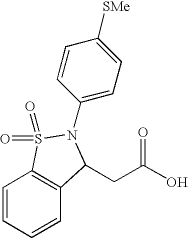 |
| 71 | F | 2-[1,1-Dioxo-2-(4-ethylphenyl)-2,3- dihydro-1H-benzo[d]isothiazol-3- yl]-acetic acid (4.21) | 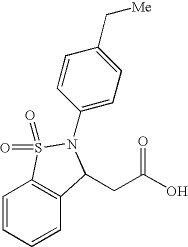 |
| 72 | F | 2-[1,1-Dioxo-2-(4- isopropylphenyl)-2,3-dihydro-1H- benzo[d]isothiazol-3-yl]acetic acid (4.22) | 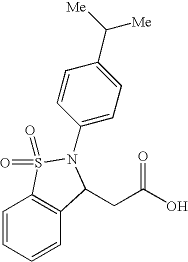 |
| 73 | F | 2-(2-Biphenyl-4-yl-1,1-dioxo-2,3- dihydro-1H-benzo[d]isothiazol-3- yl)acetic acid (4.23) | 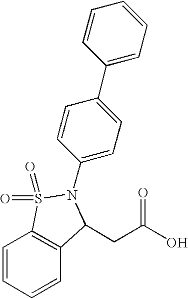 |
| 74 | F | 2-[1,1-Dioxo-2-(4-phenoxy- phenyl)-2,3-dihydro-1H- benzo[d]isothiazol-3-yl]acetic acid (4.24) | 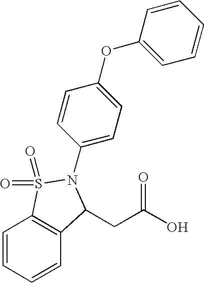 |
| 75 | F | 2-[2-(4-Benzyloxy-phenyl)-1,1- dioxo-2,3-dihydro-1H- benzo[d]isothiazol-3-yl]acetic acid (4.25) | 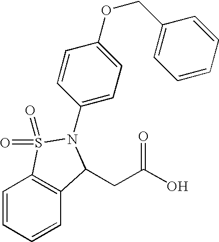 |
| 76 | E | 2-[1,1-Dioxo-2-(4-pyrrol-1-yl- phenyl)-2,3-dihydro-1H- benzo[d]isothiazol-3-yl]acetic acid (4.26) | 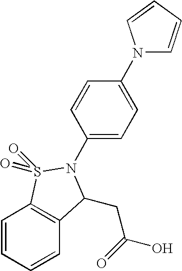 |
| 77 | E | 2-[2-(4-Imidazol-1-yl-phenyl)-1,1- dioxo-2,3-dihydro-1H- benzo[d]isothiazol-3-yl]acetic acid (4.27) | 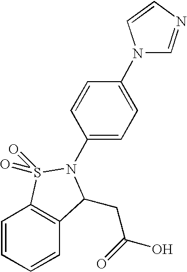 |
| 78 | E | 2-[1,1-Dioxo-2-(4-[1,2,4]triazol-1- yl-phenyl)-2,3-dihydro-1H- benzo[d]isothiazol-3-yl]acetic acid (4.28) | 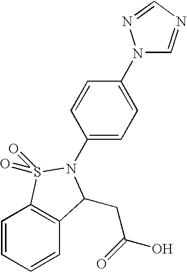 |
| 79 | E | 2-[2-(4-Oxazol-5-yl-phenyl)-1,1- dioxo-2,3-dihydro-1H- benzo[d]isothiazol-3-yl]acetic acid (4.29) | 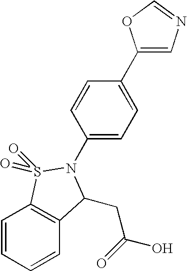 |
| 80 | F | 2-{2-[4-(2-Methylthiazol-4- yl)phenyl]-1,1-dioxo-2,3-dihydro- 1H-benzo[d]isothiazol-3-yl}-acetic acid (4.30) | 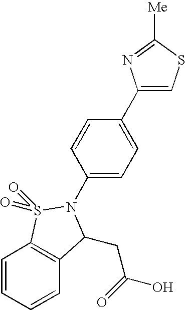 |
| 81 | F | 2-[2-(4-Dimethylaminophenyl)-1,1- dioxo-2,3-dihydro-1H- benzo[d]isothiazol-3-yl]acetic acid (4.31) | 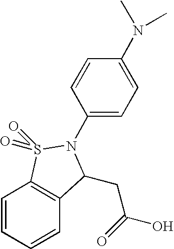 |
| 82 | F | 2-[2-(4-Morpholin-4-yl-phenyl)-1,1- dioxo-2,3-dihydro-1H- benzo[d]isothiazol-3-yl]-acetic acid (4.32) | 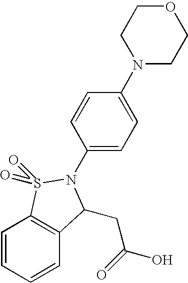 |
| 83 | F | 2-[2-(4-Ethoxyphenyl)-1,1-dioxo- 2,3-dihydro-1H-benzo[d]isothiazol- 3-yl]acetic acid (4.33) | 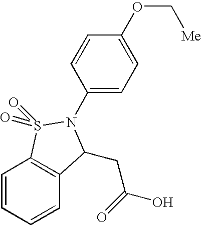 |
| 84 | F | 2-[2-(4-Butoxyphenyl)-1,1-dioxo- 2,3-dihydro-1H-benzo[d]isothiazol- 3-yl]acetic acid (4.34) | 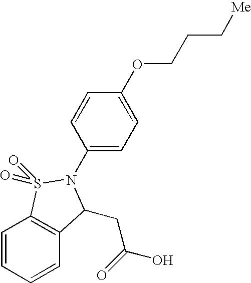 |
| 85 | F | 2-{1,1-Dioxo-2-[4-(2,2,2- trifluoroethoxy)phenyl]-2,3- dihydro-1H-benzo[d]isothiazol-3- yl}acetic acid (4.35) | 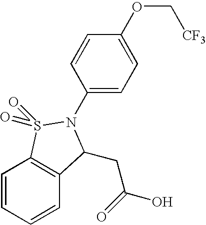 |
| 86 | F | 2-[2-(3-Fluoro-phenyl)-1,1-dioxo- 2,3-dihydro-1H-benzo[d]isothiazol- 3-yl]acetic acid (4.36) | 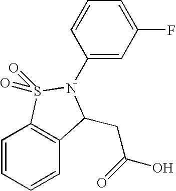 |
| 87 | F | (1,1-Dioxo-2,3-dihydro-1H- benzo[d]isothiazol-3-yl)acetic acid (4.37) | 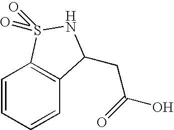 |
| 88 | F | 2-(2-Methyl-1,1-dioxo-2,3-dihydro- 1H-benzo[d]isothiazol-3-yl)acetic acid (4.38) | 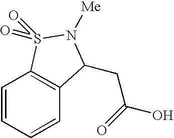 |
| 89 | F | 2-(2-Benzyl-1,1-dioxo-2,3-dihydro- 1H-benzo[d]isothiazol-3-yl)acetic acid (4.39) | 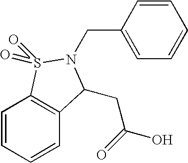 |
| 90 | F | 2-[2-(6-Methoxy-pyridin-3-yl)-1,1- dioxo-2,3-dihydro- 1Hbenzo[d]isothiazol-3-yl]acetic acid (4.40) | 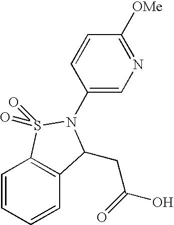 |
| 91 | F | 2-(2-Cyclohexyl-1,1-dioxo-2,3- dihydro-1H-benzo[d]isothiazol-3- yl)acetic acid (4.41) | 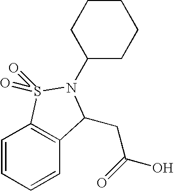 |
| 92 | F | 2-(1-Benzyl-piperidin-4-yl)-1,1- dioxo-2,3-dihydro-1H- benzo[d]isothiazol-3-yl]acetic acid (4.42) | 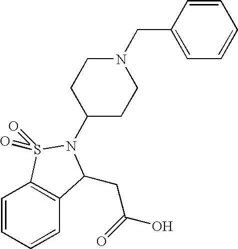 |
| 93 | F | 2-[2-(4-But-2-ynyloxyphenyl)-1,1- dioxo-2,3-dihydro-1H- benzo[d]isothiazol-3-yl]acetic acid (4.43) | 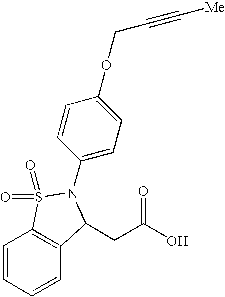 |
| 94 | E | 2-{2-[4-(2-Methylquinolin-4- ylmethoxy)phenyl]-1,1-dioxo-2,3- dihydro-1H-benzo[d]isothiazol-3- yl}acetic acid (4.44) | 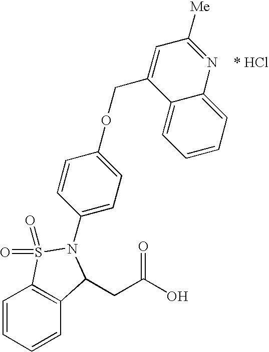 |
| 95 | E | {2-[4-(2-Dimethylamino-ethoxy)- phenyl]-1,1-dioxo-2,3-dihydro-1H- benzo[d]isothiazol-3-l}-acetic acid hydrochloride (4.45) | 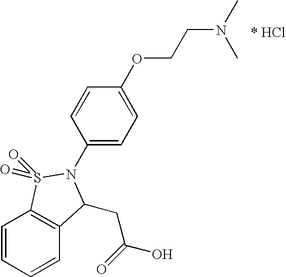 |
| 96 | E | 2-{2-[4-(2-Dimethylamino-ethoxy)- phenyl]-1,1-dioxo-2,3-dihydro-1H- benzo[d]isothiazol-3-l}-acetic acid hydrochloride (4.46) | 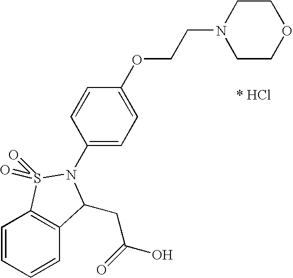 |
| 97 | F | 2-[2-(4-Allyloxy-phenyl)-1,1-dioxo- 2,3-dihydro-1H-benzo[d]isothiazol- 3-yl]acetic acid (4.47) | 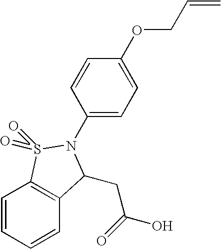 |
| 98 | E | 2-{1,1-Dioxo-2-[4-(pyridin-4- ylmethoxy)phenyl]-2,3-dihydro-1H- benzo[d]isothiazol-3-yl}-acetic acid (4.48) | 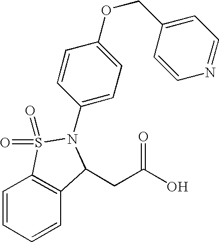 |
| 99 | E | 2-{1,1-Dioxo-2-[4-(pyridin-3- ylmethoxy)phenyl]-2,3-dihydro-1H- benzo[d]isothiazol-3-yl}-acetic acid (4.49) | 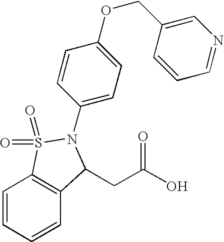 |
| 100 | E | 2-{1,1-Dioxo-2-[4-(pyridin-2- ylmethoxy)phenyl]-2,3-dihydro-1H- benzo[d]isothiazol-3-yl}-acetic acid (4.50) | 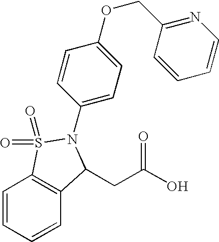 |
| 101 | F | 2-{2-[4-(4-Hydroxy-but-2-ynyloxy)- phenyl]-1,1-dioxo-2,3-dihydro-1H- benzo[d]isothiazol-3-yl}acetic acid (4.51) | 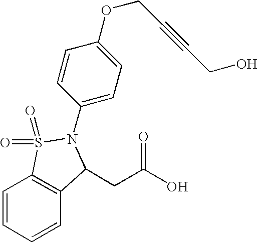 |
| 102 | F | 2-[1,1-Dioxo-2-(4-pent-2-ynyloxy- phenyl)-2,3-dihydro-1H- benzo[d]isothiazol-3-yl]acetic acid (4.52) | 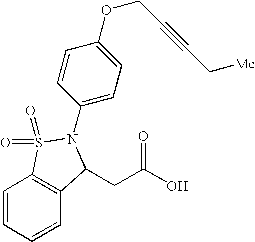 |
| 103 | F | 2-[1,1-Dioxo-2-(4-prop-2-ynyloxy- phenyl)-2,3-dihydro-1H- benzo[d]isothiazol-3-yl]acetic acid (4.53) | 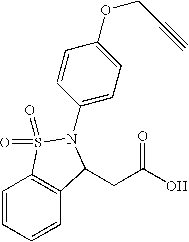 |
| 104 | F | 2-[2-(4-But-3-ynyloxy-phenyl)-1,1- dioxo-2,3-dihydro-1H- benzo[d]isothiazol-3-yl]acetic acid (4.54) | 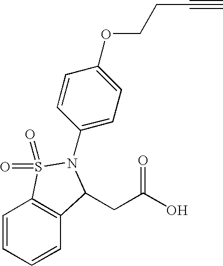 |
| 105 | F | 2-[2-(4-But-3-ynyloxy-phenyl)-1,1- dioxo-2,3-dihydro-1H- benzo[d]isothiazol-3-yl]acetic acid (4.55) | 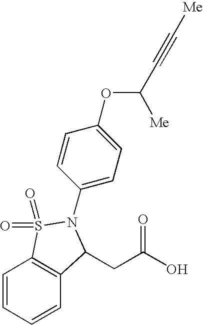 |
| 106 | E | 2-(2-{4-[1-(2-Methylquinolin-4- yl)ethoxy]-phenyl}-1,1-dioxo-2,3- dihydro-1H-benzo[d]isothiazol-3- yl)acetic acid (4.56) | 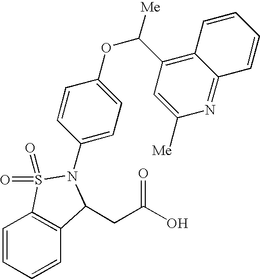 |
| 107 | E | 2-{1,1-Dioxo-2-[4-(quinolin-4- ylmethoxy)phenyl]-2,3-dihydro-1H- benzo[d]isothiazol-3-yl}-acetic acid (4.57) | 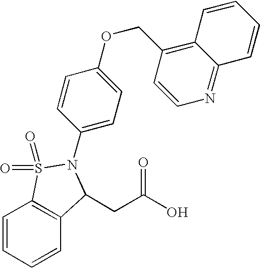 |
| 108 | E | 2-{2-[4-(6-Fluoro-2-methyl- quinolin-4-ylmethoxy)-phenyl]-1,1- dioxo-2,3-dihydro-1H- benzo[d]isothiazol-3-yl}acetic acid (4.58) | 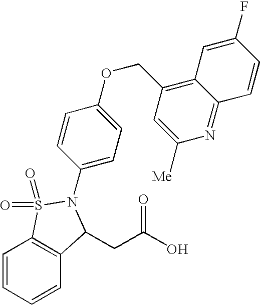 |
| 109 | E | 2-{2-[4-(6-Chloro-2- methylquinolin-4-ylmethoxy)- phenyl]-1,1-dioxo-2,3-dihydro-1H- benzo[d]isothiazol-3-yl}-acetic acid (4.59) | 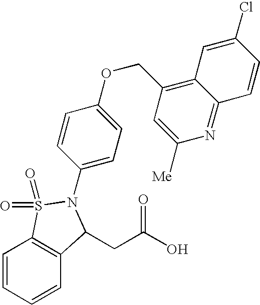 |
| 110 | E | 2-[4-(2-Methyl-pyridin-4- ylmethoxy)-phenyl]-1,1-dioxo-2,3- dihydro-1H-benzo[d]isothiazol-3- yl}acetic acid (4.60) | 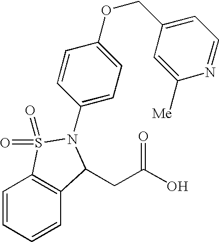 |
| 111 | E | 2-[4-(2,6-Dimethyl-pyridin-4- ylmethoxy)phenyl]-1,1-dioxo-2,3- dihydro-1H-benzo[d]isothiazol-3- yl}acetic acid (4.61) | 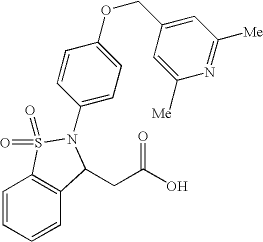 |
| 112 | E | 2-(5-Methoxy-1,1-dioxo-2-phenyl- 2,3-dihydro-1H-benzo[d]isothiazol- 3-yl)acetic acid (4.62) | 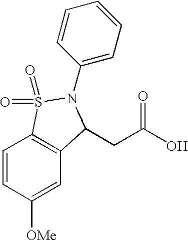 |
| 113 | E | 2-(1,1-Dioxo-2-phenyl-2,3,6,7- tetrahydro-1H-5,8-dioxa-thia-2- aza-cyclopenta[b]naphthalen-3- yl)acetic acid (4.63) | 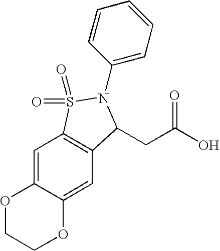 |
| 114 | E | 2-(5,6-Dimethoxy-1,1-dioxo-2- phenyl-2,3-dihydro-1H- benzo[d]isothiazol-3-yl)acetic acid (4.64) | 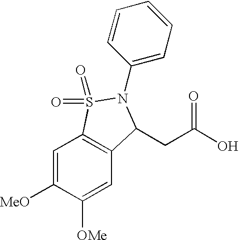 |
| 115 | E | 2-(5,6,7-Trimethoxy-1,1-dioxo-2- phenyl-2,3-dihydro-1H- benzo[d]isothiazol-3-yl)acetic acid (4.65) | 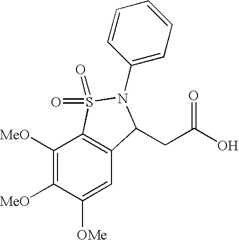 |
| 116 | E | 2-(5,7-Dimethoxy-1,1-dioxo-2- phenyl-2,3-dihydro-1H- benzo[d]isothiazol-3-yl)acetic acid (4.66) | 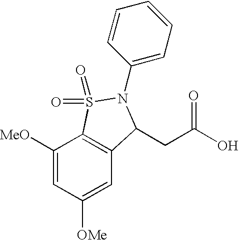 |
| 117 | F | 2-(6-Methyl-1,1-dioxo-2-phenyl- 2,3-dihydro-1H-benzo[d]isothiazol- 3-yl)acetic acid (4.67) | 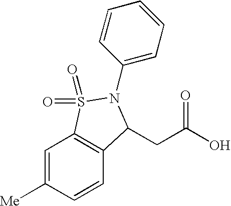 |
| 118 | F | (6-Bromo-1,1-dioxo-2-phenyl-2,3- dihydro-1H-benzo[d]isothiazol-3- yl)acetic acid (4.68) | 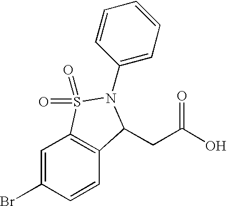 |

Compound (4.1): Yield 65%, melting point 178-179° C. (dec.), ¹H-NMR (DMSO-d₆, TMS) δ: 2.73 (1H, dd, 7 Hz and 16 Hz); 2.91 (1H, dd, 4 Hz and 16 Hz); 5.70 (1H, t, 5 Hz); 7.3-7.6 (5H, m) and 7.6-8.1 ppm (4H, m).

Compound (4.2): Yield 64%, ¹H-NMR (DMSO-d₆, TMS) δ: 2.83 (1H, dd, 5 Hz and 16 Hz); 2.94 (1H, dd, 5 Hz and 16 Hz); 5.86 (1H, t, 5 Hz) and 7.5-8.1 ppm (11H, m).

Compound (4.3): Yield 23%, ¹H-NMR (DMSO-d₆, TMS) δ: 2.35 (3H, s); 2.72 (1H, dd, 4 Hz and 16 Hz); 2.90 (1H, dd, 7 Hz and 16 Hz); 5.67 (1H, t, 5 Hz); 7.16 (1H, d, 7 Hz); 7.2-7.4 (3H, m); 7.6-7.9 (3H, m) and 7.95 ppm (1H, d, 7 Hz)).

Compound (4.43): Yield 61%, melting point 160-161° C., ¹H-NMR (DMSO-d₆, TMS) δ: 1.86 (3H, t, 2 Hz); 2.73 (1H, dd, 16 Hz and 6 Hz); 2.85 (1H, dd, 16 Hz and 4 Hz); 4.79 (2H, d, 2 Hz); 5.50 (1H, t, 5 Hz); 7.06 (2H, d, 9 Hz); 7.42 (2H, d, 9 Hz); 7.6-7.9 (3H, m); 7.94 (1H, d, 8 Hz) and 12.42 ppm (<1H, br s).

Compound (4.44): Yield 32%, ¹H-NMR (DMSO-d₆, TMS) δ: 2.67 (3H, s); 2.79 (2H, m); 5.51 (1H, br t, ˜5 Hz); 5.63 (2H, s); 7.26 (2H, d, 9 Hz); 7.45 (2H, d, 9 Hz); 7.5-7.8 (6H, m); 7.95 (2H, t, 7 Hz) and 8.11 ppm (1H, d, 8 Hz).

Compound (4.62): Yield 37%, ¹H-NMR (DMSO-d₆, TMS) δ: 2.78 (1H, dd, 6 Hz and 16 Hz); 2.91 (1H, dd, 4 Hz and 16 Hz); 3.88 (3H, s); 5.63 (1H, t, 5 Hz); 7.2-7.6 (7H, m) and 7.95 ppm (1H, d, 7 Hz).

### General procedures for preparation of (1,1-dioxo-2-phenyl-2,3-dihydro-1H-benzo[d]isothiazol-3-yl)-N-hydroxyacetamides (5)

Method G: To a solution of carboxylic acid (4) (1 mmol) in CH₂Cl₂ (10 mL) added was oxalylchloride (0.43 mL, 5 mmol) and a drop of DMFA. The resulting mixture was stirred at room temperature and evaporated. To the residue, added was a mixture prepared by dissolving hydroxylamine hydrochloride (347 mg, 5 mmol) in a mixture of THF (5 mL) and 1M aqueous NaHCO₃ (5 mL). The resulting suspension was stirred for 15 minutes and partitioned between EtOAc (50 mL) and water (30 mL). The organic phase was separated and washed with saturated NaHCO₃ (20 mL) and brine (20 mL). The solution was dried over Na₂SO₄, filtered and evaporated. The product was purified by preparative reverse phase chromatography and/or by crystallization.

Method H: CDl (4.5 mmol, 1.5 eq) was added to a solution of carboxylic acid (4) (3.0 mmol) in dry THF (5 mL). The reaction mixture was stirred for 1 h. To this added was finely powdered hydroxylamine hydrochloride (417 mg, 6 mmol). The resulting heterogeneous mixture was stirred overnight (ca 16 h). The mixture was diluted with 5% aq. KHSO₄ (30 mL) and extracted with EtOAC (2×30 mL). The combined organic phase was washed with brine (30 mL) and dried over Na₂SO₄. The extract was filtered and concentrated in vacuo to give the crude product. The product (5) was purified by preparative reverse phase chromatography and/or by crystallization.

Method I: A mixture of carboxylic acid (4) (0.24 mmol), O-tritylhydroxylamine (66 mg, 0.24 eq), EDCl (33 mg, 0.24 mg) and HOBt (46 mg, 0.24 mmol) in DMFA (2.4 mL) was stirred overnight and then diluted with saturated aqueous NaHCO₃ (25 mL). The resulting mixture was extracted with EtOAc (3×20 mL) and the combined organic phase washed with brine (20 mL). The extract was dried over Na₂SO₄ filtered and evaporated. The residue was purified by flash chromatography on silica gel eluting with a mixture of light petroleum ether and EtOAc. The resulting O-trityl protected hydroxamic acid (0.17 mmol) was dissolved in 10% (v/v) trifluoromethanesulphonic acid in DCM. The mixture was stirred at room temperature for 1 h and MeOH (1 mL) was added. Solvents were removed in vacuo and the residue was purified by preparative reverse phase chromatography to give the product (5).

Following a method analogous to Method G, Method H, or Method I, the following compounds were obtained as crude products.

| Synthesis | Method | Name | Stucture |
|---|---|---|---|
| 119 | G | 2-(1,1-Dioxo-2-phenyl-2,3- dihydro-6-benzo[d]isothiazol-3- yl)-N-hydroxyacetamide (5.1) | 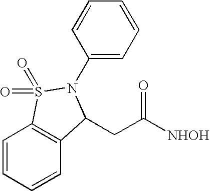 |
| 120 | G | 2-(2-Naphthalen-2-yl-1,1-dioxo- 2,3-dihydro-1H- benzo[d]isothiazol-3-yl)-N- hydroxyacetamide (5.2) | 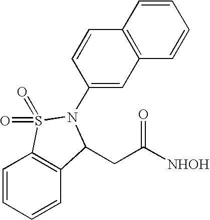 |
| 121 | G | 2-(1,1-Dioxo-2-m-tolyl-2,3- dihydro-1H-benzo[d]isothiazol- 3-yl)-N-hydroxyacetamide (5.3) | 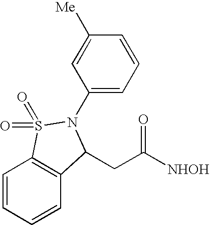 |
| 122 | G | 2-(1,1-Dioxo-2-p-tolyl-2,3- dihydro-1H-benzo[d]isothiazol- 3-yl)-N-hydroxyacetamide (5.4) | 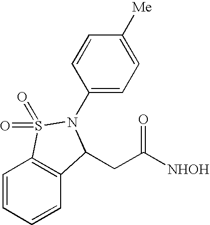 |
| 123 | G | 2-(1,1-Dioxo-2-o-tolyl-2,3- dihydro-1H-benzo[d]isothiazol- 3-yl)-N-hydroxyacetamide (5.5) | 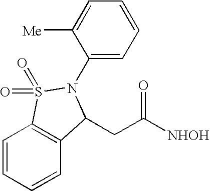 |
| 124 | G | 2-[2-(2-Methoxyphenyl)-1,1- dioxo-2,3-dihydro-1H- benzo[d]isothiazol-3-yl]-N- hydroxyacetamide (5.6) | 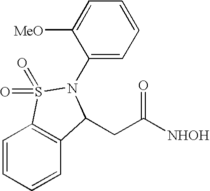 |
| 125 | G | 2-[2-(3-Methoxyphenyl)-1,1- dioxo-2,3-dihydro-1H- benzo[d]isothiazol-3-yl]-N- hydroxyacetamide (5.7) | 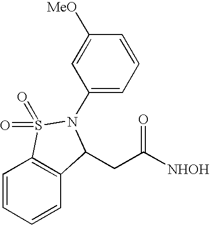 |
| 126 | G | 2-[2-(4-Methoxyphenyl)-1,1- dioxo-2,3-dihydro-1H- benzo[d]isothiazol-3-yl]-N- hydroxyacetamide (5.8) | 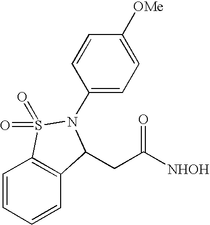 |
| 127 | G | 2-[2-(3-Phenoxyphenyl)-1,1- dioxo-2,3-dihydro-1H- benzo[d]isothiazol-3-yl]- N-hydroxyacetamide (5.9) | 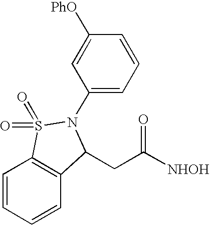 |
| 128 | G | 2-[2-(4-Chlorophenyl)-1,1-dioxo- 2,3-dihydro-1H- benzo[d]isothiazol-3-yl]- N-hydroxyacetamide (5.10) | 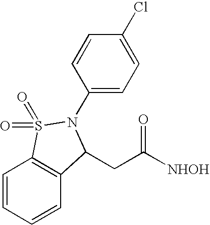 |
| 129 | G | 2-[2-(3-Methylsulfanylphenyl)- 1,1-dioxo-2,3-dihydro-1H- benzo[d]isothiazol-3-yl]-N- hydroxyacetamide (5.11) | 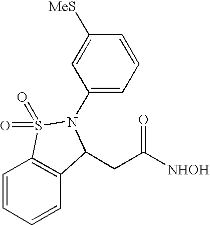 |
| 130 | G | 2-[2-(3-Trifluoromethylphenyl)- 1,1-dioxo-2,3-dihydro-1H- benzo[d]isothiazol-3-yl]-N- hydroxyacetamide (5.12) | 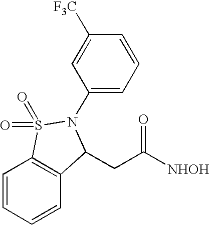 |
| 131 | G | 2-[2-(3- Trifluoromethyloxyphenyl)-1,1- dioxo-2,3-dihydro-1H- benzo[d]isothiazol-3-yl]- N-hydroxyacetamide (5.13) | 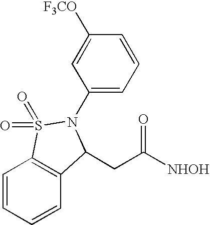 |
| 132 | G | 2-[2-(4-iodophenyl)-1,1-dioxo- 2,3-dihydro-1H- benzo[d]isothiazol-3-yl]- N-hydroxyacetamide (5.14) | 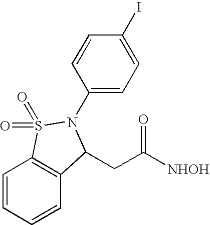 |
| 133 | G | 2-(2-Biphenyl-3-yl-1,1-dioxo- 2,3-dihydro-1H- benzo[d]isothiazol-3-yl)-N- hydroxyacetamide (5.15) | 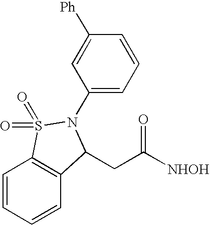 |
| 134 | G | 2-[2-(4-Fluoro-phenyl)-1,1- dioxo-2,3-dihydro-1H- benzo[d]isothiazol-3-yl]-N- hydroxyacetamide (5.16) | 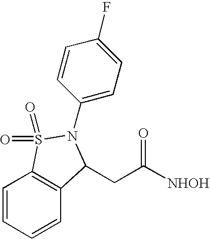 |
| 135 | G | 2-[2-(4-Trifluormethylphenyl)- 1,1-dioxo-2,3-dihydro-1H- benzo[d]isothiazol-3-yl]-N- hydroxyacetamide (5.17) | 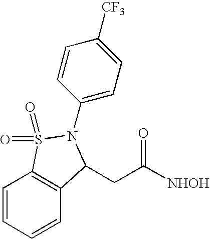 |
| 136 | G | 2-[2-(4- Trifluormethyloxyphenyl)-1,1- dioxo-2,3-dihydro-1H- benzo[d]isothiazol-3-yl]-N- hydroxyacetamide (5.18) | 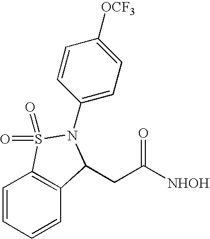 |
| 137 | H | 2-[1,1-Dioxo-2-(4- trifluoromethylsulfanylphenyl)- 2,3-dihydro-1H- benzo[d]isothiazol-3-yl]- -N- hydroxyacetamide (5.19) | 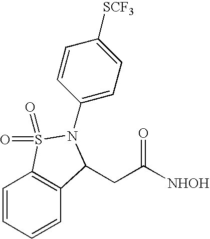 |
| 138 | H | 2-[1,1-Dioxo-2-(4- methylsulfanylphenyl)-2,3- dihydro-1H-benzo[d]isothiazol- 3-yl]-N-hydroxyacetamide (5.20) | 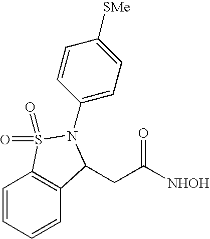 |
| 139 | H | 2-[1,1-Dioxo-2-(4-ethylphenyl)- 2,3-dihydro-1H- benzo[d]isothiazol-3-yl]-N- hydroxyacetamide (5.21) | 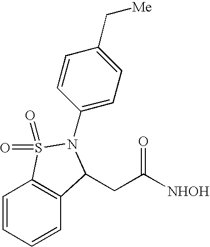 |
| 140 | G | 2-[1,1-Dioxo-2-(4- isopropylphenyl)-2,3-dihydro- 1H-benzo[d]isothiazol-3-yl]-N- hydroxyacetamide (5.22) | 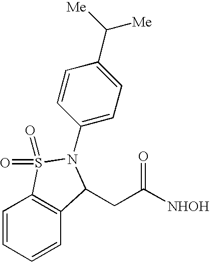 |
| 141 | H | 2-(2-Biphenyl-4-yl-1,1-dioxo- 2,3-dihydro-1H- benzo[d]isothiazol-3-yl)-N- hydroxyacetamide (5.23) | 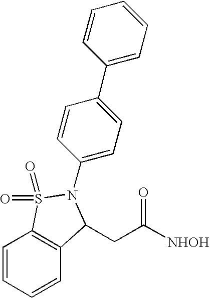 |
| 142 | G | 2-[1,1-Dioxo-2-(4-phenoxy- phenyl)-2,3-dihydro-1H- benzo[d]isothiazol-3-yl]-N- hydroxyacetamide (5.24) | 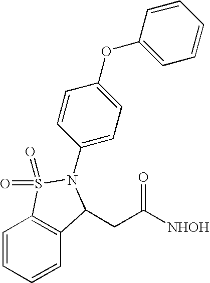 |
| 143 | G | 2-[2-(4-Benzyloxy-phenyl)-1,1- dioxo-2,3-dihydro-1H- benzo[d]isothiazol-3-yl]-N- hydroxyacetamide (5.25) | 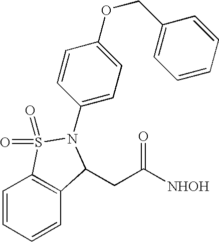 |
| 144 | H | 2-[1,1-Dioxo-2-(4-pyrrol-1-yl- phenyl)-2,3-dihydro-1H- benzo[d]isothiazol-3-yl]-N- hydroxyacetamide (5.26) | 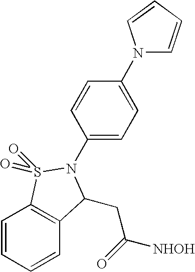 |
| 145 | H | 2-[2-(4-Imidazol-1-yl-phenyl)- 1,1-dioxo-2,3-dihydro-1H- benzo[d]isothiazol-3-yl]-N- hydroxyacetamide (5.27) | 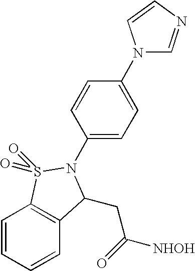 |
| 146 | H | 2-[1,1-Dioxo-2-(4-[1,2,4]triazol- 1-yl-phenyl)-2,3-dihydro-1H- benzo[d]isothiazol-3-yl]-N- hydroxyacetamide (5.28) | 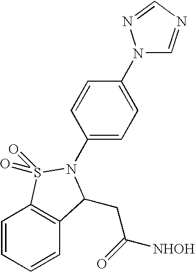 |
| 147 | H | 2-[2-(4-Oxazol-5-yl-phenyl)-1,1- dioxo-2,3-dihydro-1H- benzo[d]isothiazol-3-yl]-N- hydroxyacetamide (5.29) | 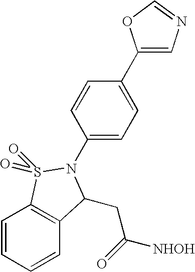 |
| 148 | H | 2-{2-[4-(2-Methylthiazol-4- yl)phenyl]-1,1-dioxo-2,3- dihydro-1H-benzo[d]isothiazol- 3-yl}-N-hydroxyacetamide (5.30) | 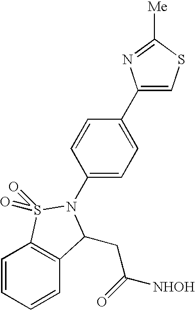 |
| 149 | G | 2-[2-(4-Dimethylaminophenyl)- 1,1-dioxo-2,3-dihydro-1H- benzo[d]isothiazol-3-yl]-N- hydroxyacetamide (5.31) | 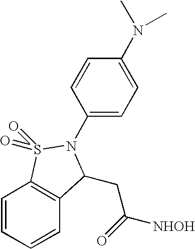 |
| 150 | G | 2-[2-(4-Morpholin-4-ylphenyl)- 1,1-dioxo-2,3-dihydro-1H- benzo[d]isothiazol-3-yl]-N- hydroxyacetamide (5.32) | 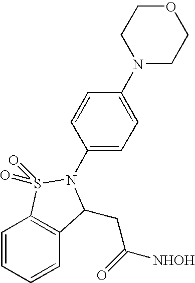 |
| 151 | G | 2-[2-(4-Ethoxyphenyl)-1,1- dioxo-2,3-dihydro-1H- benzo[d]isothiazol-3-yl]-N- hydroxyacetamide (5.33) | 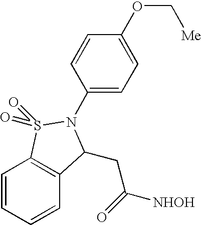 |
| 152 | G | 2-[2-(4-Butoxyphenyl)-1,1- dioxo-2,3-dihydro-1H- benzo[d]isothiazol-3-yl]-N- hydroxyacetamide (5.34) | 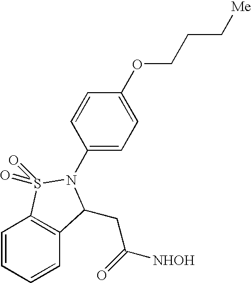 |
| 153 | G | 2-{1,1-Dioxo-2-[4-(2,2,2- trifluoroethoxy)phenyl]-2,3- dihydro-1H-benzo[d]isothiazol- 3-yl}-N-hydroxyacetamide (5.35) | 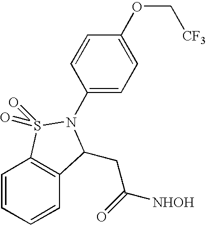 |
| 154 | G | 2-[2-(3-Fluoro-phenyl)-1,1- dioxo-2,3-dihydro-1H- benzo[d]isothiazol-3-yl]-N- hydroxyacetamide (5.36) | 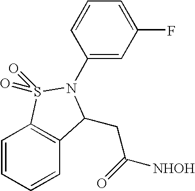 |
| 155 | G | 2-(1,1-Dioxo-2,3-dihydro-1H- benzo[d]isothiazol-3-yl)-N- hydroxyacetamide (5.37) | 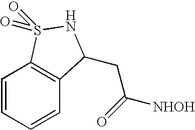 |
| 156 | G | 2-(2-Methyl-1,1-dioxo-2,3- dihydro-1H-benzo[d]isothiazol- 3-yl)-N-hydroxyacetamide (5.38) | 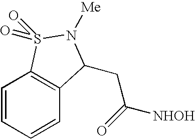 |
| 157 | G | 2-(2-Benzyl-1,1-dioxo-2,3- dihydro-1H-benzo[d]isothiazol- 3-yl)-N-hydroxyacetamide (5.39) | 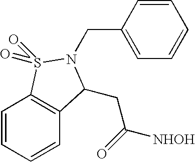 |
| 158 | G | 2-[2-(6-Methoxy-pyridin-3-yl)- 1,1-dioxo-2,3-dihydro- 1Hbenzo[d]isothiazol-3-yl]-N- hydroxyacetamide (5.40) | 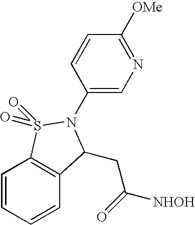 |
| 159 | H | 2-(2-Cyclohexyl-1,1-dioxo-2,3- dihydro-1H-benzo[d]isothiazol- 3-yl)-N-hydroxyacetamide (5.41) | 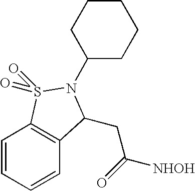 |
| 160 | H | 2-[2-(1-Benzylpiperidin-4-yl)- 1,1-dioxo-2,3-dihydro-1H- benzo[d]isothiazol-3-yl]-N- hydroxyacetamide (5.42) | 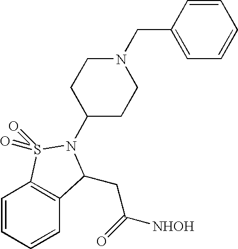 |
| 161 | G | 2-[2-(4-But-2-ynyloxyphenyl)- 1,1-dioxo-2,3-dihydro-1H- benzo[d]isothiazol-3-yl]- N-hydroxyacetamide (5.43) | 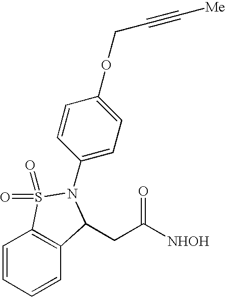 |
| 162 | G | 2-{2-[4-(2-Methylquinolin-4- ylmethoxy)phenyl]-1,1-dioxo- 2,3-dihydro- 1H-benzo[d]isothiazol-3-yl}-N- hydroxyacetamide (5.44) | 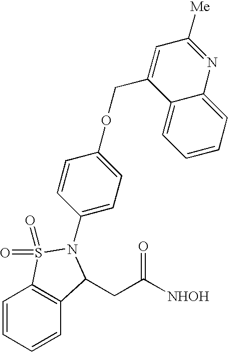 |
| 163 | G | 2-{2-[4-(2-Dimethylamino- ethoxy)-phenyl]-1,1-dioxo-2,3- dihydro-1H-benzo[d]isothiazol- 3-yl}--N-hydroxyacetamide (5.45) | 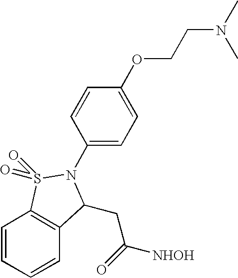 |
| 164 | H | 2-{2-[4-(2- Dimethylaminoethoxy)-phenyl]- 1,1-dioxo-2,3-dihydro-1H- benzo[d]isothiazol-3-l}-N- hydroxyacetamide (5.46) | 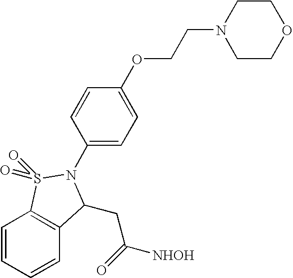 |
| 165 | H | 2-[2-(4-Allyloxy-phenyl)-1,1- dioxo-2,3-dihydro-1H- benzo[d]isothiazol-3-yl]-N- hydroxyacetamide (5.47) | 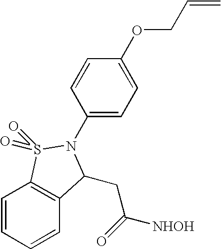 |
| 166 | H | 2-{1,1-Dioxo-2-[4-(pyridin-4- ylmethoxy)phenyl]-2,3-dihydro- 1H-benzo[d]isothiazol-3-yl}-N- hydroxyacetamide (5.48) | 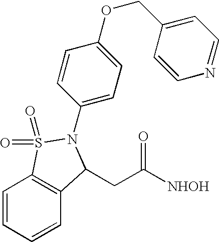 |
| 167 | H | 2-{1,1-Dioxo-2-[4-(pyridin-3- ylmethoxy)phenyl]-2,3-dihydro- 1H-benzo[d]isothiazol-3-yl}-N- hydroxyacetamide (5.49) | 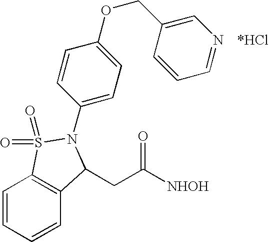 |
| 168 | H | 2-{1,1-Dioxo-2-[4-(pyridin-2- ylmethoxy)phenyl]-2,3-dihydro- 1H-benzo[d]isothiazol-3-yl}-N- hydroxyacetamide (5.50) | 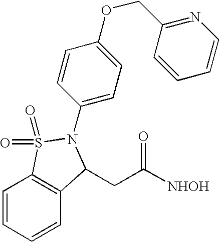 |
| 169 | I | 2-{2-[4-(4-Hydroxy-but-2- ynyloxy)-phenyl]-1,1-dioxo-2,3- dihydro-1H-benzo[d]isothiazol- 3-yl}-N-hydroxyacetamide id (5.51) | 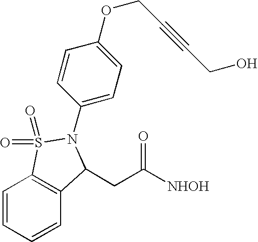 |
| 170 | G | 2-[1,1-Dioxo-2-(4-pent-2- ynyloxy-phenyl)-2,3-dihydro-1H- benzo[d]isothiazol-3-yl]-N- hydroxyacetamide (5.52) | 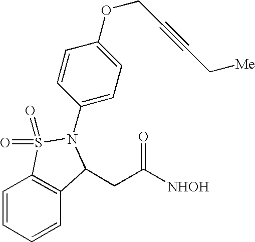 |
| 171 | G | 2-[1,1-Dioxo-2-(4-prop-2- ynyloxy-phenyl)-2,3-dihydro-1H- benzo[d]isothiazol-3-yl]-N- hydroxyacetamide (5.53) | 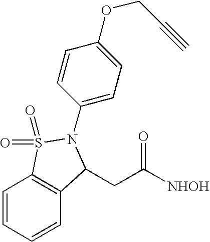 |
| 172 | H | 2-[2-(4-But-3-ynyloxy-phenyl)- 1,1-dioxo-2,3-dihydro-1H- benzo[d]isothiazol-3-yl]-N- hydroxyacetamide (5.54) | 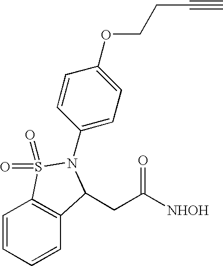 |
| 173 | G | 2-[2-(4-But-3-ynyloxy-phenyl)- 1,1-dioxo-2,3-dihydro-1H- benzo[d]isothiazol-3-yl]-N- hydroxyacetamide (5.55) | 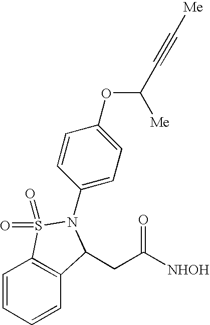 |
| 174 | H | 2-(2-{4-[1-(2-Methylquinolin-4- yl)ethoxy]-phenyl}-1,1-dioxo- 2,3-dihydro-1H- benzo[d]isothiazol-3-yl)-N- hydroxyacetamide (5.56) | 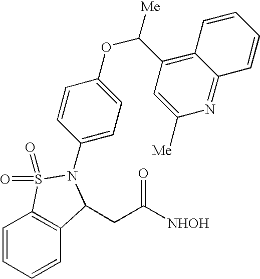 |
| 175 | H | 2-{1,1-Dioxo-2-[4-(quinolin-4- ylmethoxy)phenyl]-2,3-dihydro- 1H-benzo[d]isothiazol-3-yl}-N- hydroxyacetamide (5.57) | 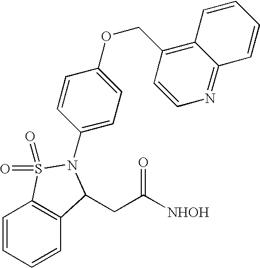 |
| 176 | H | 2-{2-[4-(6-Fluoro-2-methyl- quinolin-4-ylmethoxy)-phenyl]- 1,1-dioxo-2,3-dihydro-1H- benzo[d]isothiazol-3-yl}-N- hydroxyacetamide (5.58) | 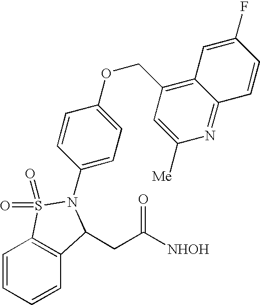 |
| 177 | H | 2-{2-[4-(6-Chloro-2- methylquinolin-4-ylmethoxy)- phenyl]-1,1-dioxo-2,3-dihydro- 1H-benzo[d]isothiazol-3-yl}-N- hydroxyacetamide (5.59) | 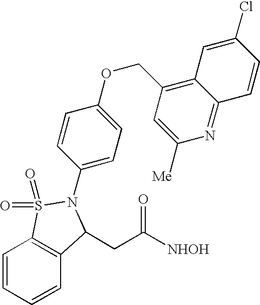 |
| 178 | H | 2-{2-[4-(2-Methyl-pyridin-4- ylmethoxy)-phenyl]-1,1-dioxo- 2,3-dihydro-1H- benzo[d]isothiazol-3-yl}-N- hydroxyacetamide (5.60) | 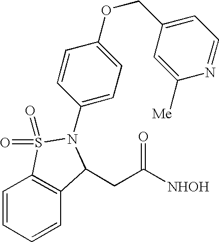 |
| 179 | H | 2-{2-[4-(2,6-Dimethyl-pyridin-4- ylmethoxy)phenyl]-1,1-dioxo- 2,3-dihydro-1H- benzo[d]isothiazol-3-yl}-N- hydroxyacetamide (5.61) | 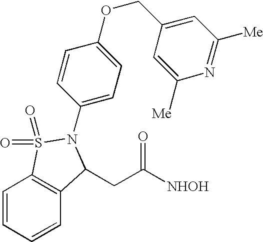 |
| 180 | G | 2-(5-Methoxy-1,1-dioxo-2- phenyl-2,3-dihydro-1H- benzo[d]isothiazol-3-yl)- N-hydroxyacetamide (5.62) | 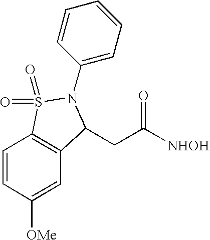 |
| 181 | G | 2-(1,1-Dioxo-2-phenyl-2,3,6,7- tetrahydro-1H-5,8-dioxa-thia-2- aza-cyclopenta[b]naphthalen-3- yl)-N-hydroxyacetamide (5.63) | 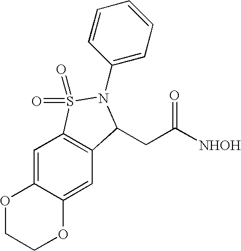 |
| 182 | G | 2-(5,6-Dimethoxy-1,1-dioxo-2- phenyl-2,3-dihydro-1H- benzo[d]isothiazol-3-yl)-N- hydroxyacetamide (5.64) | 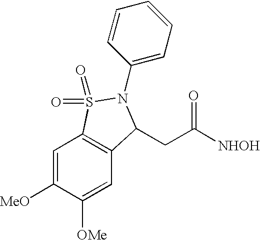 |
| 183 | H | 2(5,6,7-Trimethoxy-1,1-dioxo-2- phenyl-2,3-dihydro-1H- benzo[d]isothiazol-3-yl)-N- hydroxyacetamide (5.65) | 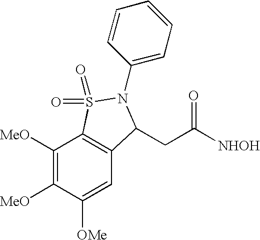 |
| 184 | H | 2-(5,7-Dimethoxy-1,1-dioxo-2- phenyl-2,3-dihydro-1H- benzo[d]isothiazol-3-yl)-N- hydroxyacetamide (5.66) | 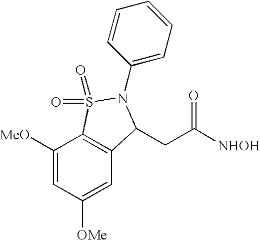 |
| 185 | H | 2-(6-Methyl-1,1-dioxo-2-phenyl- 2,3-dihydro-1H- benzo[d]isothiazol-3-yl)-N- hydroxyacetamide (5.67) | 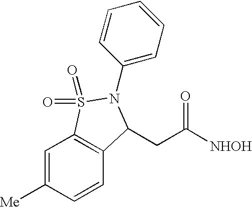 |
| 186 | H | 2-(6-Bromo-1,1-dioxo-2-phenyl- 2,3-dihydro-1H- benzo[d]isothiazol-3-yl)-N- hydroxyacetamide (5.68) | 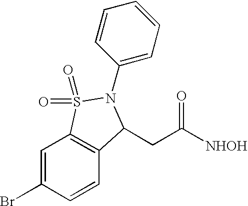 |

Compound (5.1): Yield 85%, melting point 141-143° C., ¹H-NMR (DMSO-d₆, TMS) δ: 2.32 (1H, dd, 9 Hz and 15 Hz); 2.64 (1H, dd, 4 Hz and 15 Hz); 5.73 (1H, dd, 4 Hz and 9 Hz); 7.2-7.4 (1H, m); 7.4-7.6 (4H, m); 7.6-7.9 (3H, m); 7.98 (1H, d, 8 Hz); 8.9 (1H, br s) and 10.5 ppm (1H, br s).

Compound (5.2): Yield 12%, amorphous, ¹H-NMR (DMSO-d₆, TMS) δ: 2.39 (1H, dd, 15 Hz and 9 Hz); 2.69 (1H, dd, 15 Hz and 4 Hz); 5.84 (1H, dd, 8 Hz and 4 Hz); 7.5-8.1 (11H, m); 8.89 (1H, s) and 10.49 ppm (1H, s).

Compound (5.3): Yield 45%, amorphous, ¹H-NMR (DMSO-d₆, TMS) δ: 2.29 (1H, dd, 9 Hz and 15 Hz); 2.35 (3H, s); 2.63 (1H, dd, 4 Hz and 15 Hz); 5.67 (1H, dd, 4 Hz and 9 Hz); 7.15 (1H, d, 7 Hz); 7.2-7.9 (6H, m); 7.97 (1H, d, 8 Hz); 8.94 (1H, br s) and 10.52 ppm (1H, br s).

Compound (5.4): Yield 12%, amorphous, ¹H-NMR (DMSO-d₆, TMS) δ: 2.30 (1H, dd, 8 Hz and 15 Hz); 2.58 (1H, dd, 4 Hz and 15 Hz); 5.60 (1H, dd, 4 Hz and 8 Hz); 7.29 (2H, d, 8 Hz); 7.37 (2H, d, 8 Hz); 7.6-7.9 (3H, m); 7.96 (1H, d, 8 Hz); 8.92 (1H, br s) and 10.51 ppm (1H, br s).

Compound (5.5): Yield 86%, amorphous, ¹H-NMR (DMSO-d₆, TMS) δ: 2.1-2.6 (5H, m); 5.34 (1H, t, 7 Hz); 7.2-7.9 (7H, m); 7.97 (1H, d, 8 Hz); 8.90 (1H, br s) and 10.55 ppm (1H, br s).

Compound (5.6): Yield 62%, amorphous, ¹H-NMR (DMSO-d₆, TMS) δ: 2.34 (1H, dd, 15 Hz and 8 Hz); 2.4-2.6 (1H, overlapped with DMSO); 3.76 (3H, s); 5.49 (1H, dd, 8 Hz and 5 Hz); 7.05 (1H, t, 7 Hz); 7.19 (1H, d, 8 Hz); 7.4-7.9 (5H, m); 7.95 (1H, d, 7 Hz); 8.86 (1H, s) and 10.51 ppm (1H, s).

Compound (5.7): Yield 62%, melting point 147-148° C., ¹H-NMR (DMSO-d₆, TMS) δ: 2.31 (1H, dd, 15 Hz and 9 Hz); 2.66 (1H, dd, 15 Hz and 4 Hz); 3.79 (3H, s); 5.71 (1H, dd, 9 Hz and 4 Hz); 6.91 (1H, d, 7 Hz); 7.05 (1H, s); 7.07 (1H, d, 7 Hz); 7.45 (1H, t, 8 Hz); 7.6-7.8 (3H, m); 7.98 (1H, d, 7 Hz); 8.93 (1H, s) and 10.52 ppm (1H, s).

Compound (5.8): Yield 51%, melting point 185-186° C., ¹H-NMR (DMSO-d₆, TMS) δ: 2.33 (1H, dd, 15 Hz and 8 Hz); 2.5-2.6 (1H, overlapped with DMSO); 3.80 (1H, s); 5.47 (1H, dd, 8 Hz and 4 Hz); 7.05 (2H, d, 9 Hz); 7.43 (2H, d, 9 Hz); 7.6-7.9 (3H, m); 7.94 (1H, d, 8 Hz); 8.89 (1H, s) and 10.50 ppm (1H, s).

Compound (5.9): Yield 54%, melting point 181-182° C., ¹H-NMR (DMSO-d₆, TMS) δ: 2.34 (1H, dd, 15 Hz and 8 Hz); 2.68 (1H, dd, 15 Hz and 4 Hz); 5.73 (1H, dd, 9 Hz and 4 Hz); 6.88 (1H, d, 8 Hz); 7.0-7.9 (11H, m); 7.97 (1H, d, 8 Hz); 8.93 (1H, s) and 10.51 ppm (1H, s).

Compound (5.10): Yield 10%, melting point 184-185° C., ¹H-NMR (DMSO-d₆, TMS) δ: 2.36 (1H, dd, 14 Hz and 8 Hz); 2.62 (1H, dd, 14 Hz and 8 Hz); 5.72 (1H, dd, 8 Hz and 4 Hz); 7.50 (2H, d, 8 Hz); 7.57 (2H, d, 8 Hz); 7.6-7.9 (3H, m); 7.99 (1H, d, 7 Hz); 8.89 (1H, s) and 10.49 ppm (1H, s).

Compound (5.11): Yield 53%, amorphous, ¹H-NMR (DMSO-d₆, TMS) δ: 2.33 (1H, dd, 15 Hz and 8 Hz); ˜2.5 (3H, overlapped with DMSO); 2.65 (1H, dd, 15 Hz and 4 Hz); 5.75 (1H, dd, 8 Hz and 4 Hz); 7.2-7.9 (7H, m); 7.98 (1H, d, 8 Hz); 8.93 (1H, s) and 10.51 ppm (1H, s).

Compound (5.12): Yield 12%, amorphous, ¹H-NMR (DMSO-d₆, TMS) δ: 2.40 (1H, dd, 15 Hz and 8 Hz); 2.66 (1H, dd, 15 Hz and 4 Hz); 5.90 (1H, dd, 8 Hz and 4 Hz); 7.6-7.9 (7H, m); 8.01 (1H, d, 7 Hz); 8.89 (1H, s) and 10.51 ppm (1H, s).

Compound (5.13): Yield 19%, amorphous, ¹H-NMR (DMSO-d₆, TMS) δ: 2.36 (1H, dd, 15

Hz and 8 Hz); 2.67 (1H, dd, 15 Hz and 4 Hz); 5.84 (1H, dd, 8 Hz and 4 Hz); 7.31 (1H, d, 8 Hz); 7.4-7.9 (1H, m); 8.00 (1H, d, 8 Hz); 8.92 (1H, s) and 10.51 ppm (1H, s).

Compound (5.14): Yield 27%, amorphous, ¹H-NMR (DMSO-d₆, TMS) δ: 2.36 (1H, dd, 8 Hz and 15 Hz); 2.62 (1H, dd, 4 Hz and 15 Hz); 5.70 (1H, dd, 4 Hz and 8 Hz); 7.27 (2H, d, 8 Hz); 7.6-7.9 (5H, m); 7.97 (1H, d, 8 Hz); 8.89 (1H, br s) and 10.48 ppm (1H, br s).

Compound (5.15): Yield 37%, amorphous, ¹H-NMR (DMSO-d₆, TMS) δ: 2.37 (1H, dd, 15 Hz and 8 Hz); 2.70 (1H, dd, 15 Hz and 4 Hz); 5.83 (1H, dd, 8 Hz and 4 Hz); 7.5-7.8 (12H, m); 7.98 (1H, d, 7 Hz); 8.91 (1H, s) and 10.52 ppm (1H, s).

Compound (5.16): Yield 52%, amorphous, ¹H-NMR (DMSO-d₆, TMS) δ: 2.36 (1H, dd, 15 Hz and 8 Hz); 2.56 (1H, dd, 15 Hz and 4 Hz); 5.60 (1H, dd, 8 Hz and 4 Hz); 7.33 (2H, t, 9 Hz); 7.52 (2H, dd, 9 Hz and 5 Hz); 7.61-7.83 (3H, m); 7.96 (1H, d, 7 Hz); 8.86 (1H, s) and 10.48 ppm (1H, s).

Compound (5.17): Yield 22%, amorphous, ¹H-NMR (DMSO-d₆, TMS) δ: 2.36 (1H, dd, 15 Hz and 9 Hz); 2.73 (1H, dd, 15 Hz and 4 Hz); 5.93 (1H, dd, 9 Hz and 4 Hz); 7.54-7.90 (7H, m); 8.03 (1H, d, 8 Hz); 8.94 (1H, s) and 10.52 ppm (1H, s).

Compound (5.18): melting point 178-179° C., ¹H-NMR (DMSO-d₆, TMS) δ: 2.37 (1H, dd, 15 Hz and 8 Hz); 2.64 (1H, dd, 15 Hz and 4 Hz); 5.75 (1H, dd, 8 Hz, and 4 Hz); 7.50-7.83 (7H, m); 8.00 (1H, d, 8 Hz); 8.91 (1H, s) and 10.51 ppm (1H, s).

Compound (5.19): Yield 9%, amorphous, ¹H-NMR (DMSO-d₆, TMS) δ: 2.32 (1H, dd, 14.7 Hz and 8.8 Hz); 2.70 (1H, dd, 14.7 Hz and 3.9 Hz); 5.86 (1H, dd, 8.8 Hz and 3.9 Hz); 7.5-7.9 (7H, m); 8.00 (1H, d, 7.8 Hz), 8.96 (1H, s) and 10.54 ppm (1H, s).

Compound (5.20): Yield 85% melting point, 197-198° C. (dec.) ¹H-NMR (DMSO-d₆, TMS) δ: 2.32 (1H, dd, 8.8 and 14.7 Hz); 2.49 (3H, s); 2.59 (1H, dd, 4.4 and 14.7); 5.61 (1H, dd, 4.4 and 8.1 Hz); 7.39 (2H, d, 8.8 Hz); 7.42 (2H, d, 8.8 Hz); 7.5-7.9 (3H, m); 7.95 (1H, d, 8.1 Hz); 8.89 (1H, s) and 10.49 ppm (1H, s).

Compound (5.21): Yield 48% amorphous, ¹H-NMR (DMSO-d₆, TMS) δ: 1.19 (3H, t, 7.3 Hz); 2.28 (1H, dd, 14.7 and 8.8 Hz); 2.5-2.7 (1H, m overlapped with DMSO); 2.62 (2H, q, 8.1 Hz); 5.59 (1H, dd, 8.8 and 3.7 Hz); 7.31 (2H, d, 8.8 Hz); 7.39 (2H, d, 8.8 Hz); 7.5-7.9 (3H, m); 7.95 (1H, d, 7.3 Hz); 8.91 (1H, s) and 10.50 ppm (1H, s).

Compound (522): Yield 42%, melting point 160-161° C., ¹H-NMR (DMSO-d₆, TMS) δ: 1.23 (6H, d, 7 Hz); 2.29 (1H, dd, 15 Hz and 9 Hz); 2.62 (1H, dd, 15 Hz and 4 Hz); 2.93 (1H, m); 5.61 (1H, dd, 9 Hz, and 4 Hz); 7.34-7.44 (4H, m); 7.59-7.84 (3H, m); 7.97 (1H, d, 7 Hz); 8.95 (1H, s) and 10.53 ppm (1H, s).

Compound (5.23): Yield 27%, melting point 207-208° C. (dec.), ¹H-NMR (DMSO-d₆, TMS) δ: 2.2-2.9 (2H, m, overlapped with DMSO); 5.7-5.8 (1H, m); 7.3-7.9 (12H, m); 7.99 (1H, d, 6.8 Hz); 8.93 (1H, s) and 10.53 ppm (1H, s).

Compound (5.24): Yield 47%, melting point, 174-175° C., ¹H-NMR (DMSO-d₆, TMS) δ: 2.35 (1H, dd, 15 Hz and 8 Hz); 2.60 (1H, dd, 15 Hz and 4 Hz); 5.57 (1H, dd, 8 Hz and 4 Hz); 7.06-7.21 (5H, m); 7.38-7.52 (4H, m); 7.73-7.84 (3H, m); 7.97 (1H, d, 8 Hz); 8.91 (1H, s) and 10.51 ppm (1H, s).

Compound (5.25): Yield 86%, melting point, 186-187° C. ¹H-NMR (DMSO-d₆, TMS) δ: 2.32 (1H, dd, 15 Hz and 8 Hz); ˜2.5 (1H, overlapped with DMSO); 5.14 (2H, s); 5.48 (1H, dd, 8 Hz, and 4 Hz); 7.13 (2H, d, 9 Hz); 7.37-7.82 (10H, m); 7.95 (1H, d, 8 Hz); 8.92 (1H, s) and 10.52 ppm (1H, s).

Compound (5.26): Yield 47%, melting point, 192-194° C. (dec.), ¹H-NMR (DMSO-d₆, TMS) δ: 2.32-2.67 (2H, m, 1H, overlapped with DMSO); 5.70 (1H, m); 6.29 (2H, s); 7.41 (2H, s); 7.40-7.95 (7H, m); 7.98 (1H, d, 7 Hz); 8.91 (1H, s) and 10.52 ppm (1H, s).

Compound (5.27): Yield 22%, melting point, 199-201° C. (dec.), ¹H-NMR (DMSO-d₆, TMS) δ: 2.34-2.63 (1H, m, overlapped with DMSO); 5.76 (1H, m); 7.13 (1H, s); 7.60-7.78 (8H, m); 7.99 (1H, d, 7.3 Hz); 8.29 (1H, s); 8.91 (1H, s) and 10.55 ppm (1H, s).

Compound (5.28): Yield 60%, melting point, 205-207° C. (dec.), ¹H-NMR (DMSO-d₆, TMS) δ: 2.41 (1H, dd, 14 Hz and 8 Hz); 2.67 (1H, dd, 14 Hz and 5 Hz); 5.80 (1H, dd, 8 Hz, and 5 Hz); 7.65-7.87 (5H, m); 7.99 (2H, d, 9 Hz); 8.00 (1H, d, 7 Hz); 8.27 (1H, s); 8.90 (1H, s); 9.33 (1H, s) and 10.51 ppm (1H, s).

Compound (5.29): Yield 27%, melting point, 138-140° C. (dec.), ¹H-NMR (DMSO-d₆, TMS) δ: 2.37 (1H, dd, 15 Hz and 8 Hz); 2.68 (1H, dd, 15 Hz and 4 Hz); 5.81 (1H, dd, 8 Hz, and 4 Hz); 7.56-7.87 (8H, m); 7.99 (1H, d, 7 Hz); 8.48 (1H, s); 8.92 (1H, s) and 10.52 ppm (1H, s).

Compound (5.30): Yield 38%, melting point, 179-181° C. (dec.), ¹H-NMR (DMSO-d₆, TMS) δ: 2.37 (1H, dd, overlapped with DMSO); 2.71 (3H, s); 5.74 (1H, dd, 8.1 Hz and 3.7 Hz); 7.51 (2H, d, 8.8 Hz); 7.61-7.84 (3H, m); 7.95-8.05 (4H, m); 8.91 (1H, s) and 10.53 ppm (1H, s).

Compound (5.31): Yield 39%. Melting point: 185-186° C., ¹H-NMR (DMSO-d₆, TMS) δ: 2.27 (1H, dd, 15 Hz and 9 Hz); ˜2.5 (1H, overlapped with DMSO); 2.94 (6H, s); 5.33 (1H, dd, 8 Hz and 4 Hz); 6.77 (2H, d, 9 Hz); 7.27 (2H, d, 9 Hz); 7.59-7.82 (3H, m); 7.92 (1H, d, 7 Hz); 8.92 (1H, s) and 10.52 ppm (1H, s).

Compound (5.32): Yield 61%, melting point, 204-206° C., ¹H-NMR (DMSO-d₆, TMS) δ: 2.29 (1H, dd, 15 Hz and 9 Hz); ˜2.5 (1H, overlapped with DMSO); 3.16 (4H, m); 3.75 (4H, m); 5.43 (1H, dd, 9 Hz and 4 Hz); 7.04 (2H, d, 9 Hz); 7.33 (2H, d, 9 Hz); 7.58-7.82 (3H, m); 7.94 (1H, d, 7 Hz); 8.92 (1H, s) and 10.52 ppm (1H, s).

Compound (5.33): Yield 60%, melting point, 177-178° C., ¹H-NMR (DMSO-d₆, TMS) δ: 1.35 (3H, t, 7 Hz); 2.31 (1H, dd, 15 Hz and 8 Hz); ˜2.5 (1H, overlapped with DMSO); 4.06 (2H, q, 7 Hz); 5.46 (1H, dd, 8 Hz, and 4 Hz); 7.03 (2H, d, 9 Hz); 7.40 (2H, d, 9 Hz); 7.59-7.82 (3H, m); 7.97 (1H, d, 7 Hz); 8.89 (1H, s) and 10.51 ppm (1H, s).

Compound (5.34): Yield 63%, melting point 165-166° C., ¹H-NMR (DMSO-d₆, TMS) δ: 0.94 (3H, t, 7 Hz); 1.39-1.50 (2H, m); 1.65-1.75 (2H, m); 2.28 (1H, dd, 15 Hz and 8 Hz); 2.55 (1H, dd, 15 Hz and 4 Hz); 4.00 (2H, t, 7 Hz); 5.47 (1H, dd, 8 Hz and 4 Hz); 7.04 (2H, d, 9 Hz); 7.39 (2H, d, 9 Hz); 7.60-7.82 (3H, m); 7.95 (1H, d, 7 Hz); 8.91 (1H, s) and 10.51 ppm (1H, s).

Compound (5.35): Yield 63%, amorphous, ¹H-NMR (DMSO-d₆, TMS) δ: 2.33 (1H, dd, 15 Hz and 8 Hz); 2.56 (1H, dd, 15 Hz and 4 Hz); 4.82 (2H, q, 9 Hz); 5.54 (1H, dd, 8 Hz, and 4 Hz); 7.18 (2H, d, 9 Hz); 7.46 (2H, d, 9 Hz); 7.61-7.84 (3H, m); 7.96 (1H, d, 7 Hz); 8.90 (1H, s) and 10.51 ppm (1H, s).

Compound (5.36): Yield 49% melting point 145-147° C. (dec.), ¹H-NMR (DMSO-d₆, TMS) δ: 2.34 (1H, dd, 8 Hz and 15 Hz); 2.66 (1H, dd, 4 Hz and 15 Hz); 5.77 (1H, dd, 4 Hz and 8 Hz); 7.1-7.3 (1H, m); 7.3-7.5 (2H, m); 7.5-7.9 (4H, m); 7.99 (1H, d, 7 Hz); 8.91 (1H, s) and 10.55 ppm (1H, s).

Compound (5.37): Yield 7%, melting point, 152-154° C., ¹H-NMR (DMSO-d₆, TMS) δ: 2.37 (1H, dd, 15 Hz and 9 Hz); 2.60 (1H, dd, 15 Hz and 6 Hz); 4.98 (1H, t, 7 Hz); 7.54-7.82 (3H, m); 7.80 (1H, d, 9 Hz); 8.80 (1H, br s); 8.92 (1H, br s) and 10.54 ppm (1H, s).

Compound (5.38): amorphous, ¹H-NMR (DMSO-d₆, TMS) δ: ˜2.5 (1H, overlapped with DMSO); 2.67 (1H, dd, 15 Hz and 6 Hz); 2.79 (3H, s); 4.76 (1H, t, 6 Hz); 7.60-7.77 (3H, m); 7.87 (1H, d, 7 Hz); 8.90 (1H, s) and 10.65 ppm (1H, s).

Compound (5.39): Yield 55%, amorphous, ¹H-NMR (DMSO-d₆, TMS) δ: 2.32 (1H, dd, 15 Hz and 8 Hz); 2.71 (1H, dd, 15 Hz and 6 Hz); 4.37 (1H, d, 15 Hz); 4.63 (1H, d, 15 Hz); 4.81 (1H, dd, 8 Hz and 6 Hz); 7.23-7.51 (6H, m); 7.51-7.75 (2H, m); 7.91 (1H, d, 8 Hz); 8.98 (1H, s) and 10.61 ppm (1H, s).

Compound (5.40): Yield 39% melting point 189-190° C., ¹H-NMR (DMSO-d₆, TMS) δ: 2.40-2.62 (2H, m, overlapped with DMSO); 3.89 (3H, s); 5.51 (1H, t, 6 Hz); 6.96 (1H, d, 8 Hz); 7.67-7.88 (4H, m); 7.98 (1H, d, 8 Hz); 8.24 (1H, d, 2 Hz); 8.85 (1H, s) and 10.50 ppm (1H, s).

Compound (5.41): Yield 58%, amorphous, ¹H-NMR (DMSO-d6, TMS) δ: 0.94-2.36 (10H, m); 2.31 (1H, dd, 14.7 Hz and 8.1 Hz); 2.72 (1H, dd, 14.7 Hz, 5.9 Hz); 3.42-3.54 (1H, m); 5.06 (1H, dd, 8.1 Hz, 5.1 Hz); 7.47-7.71 (3H, m); 7.79 (1H, d, 8.1 Hz); 8.95 (1H, s) and 10.57 ppm (1H, s).

Compound (5.42): Yield 58%, melting point 141-143° C., ¹H-NMR (DMSO-d₆, TMS) δ: 1.7-2.1 (6H, m); 2.30 (1H, dd, 8.1 Hz and 14.7 Hz); 2.69 (1H, dd, 5.1 Hz and 14.7 Hz); 2.8-3.0 (2H, m); 3.44 (2H, s); 3.3.2-3.6 (1H, m); 5.05 (1H, dd, 5.9 and 7.3 Hz); 7.2-7.4 (5H, m); 7.4-7.7 (3H, m); 7.78 (1H, d, 8.1 Hz); 8.96 (1H, s) and 10.57 ppm (1H, s).

Compound (5.43): Yield 25%, amorphous, ¹H-NMR (DMSO-d₆, TMS) δ: 1.84 (3H, s); 2.30 (1H, dd, 14 Hz and 7 Hz); 2.5-2.6 (1H, overlapped with DMSO); 4.77 (2H, d, 2 Hz); 5.47 (1H, dd, 8 Hz and 4 Hz); 7.06 (2H, d, 9 Hz); 7.40 (2H, d, 9 Hz); 7.5-7.8 (3H, m); 7.94 (1H, d, 7 Hz); 8.89 (1H, s) and 10.50 ppm (1H, s).

Compound (5.44): Yield 31%, m.p. 195-197° C., ¹H-NMR (DMSO-d₆, TMS) δ: 2.1-2.6 (2H, m); 2.68 (3H, s); 5.4-5.6 (1H, m); 5.65 (2H, s); 7.28 (2H, d, 9 Hz); 7.47 (2H, d, 9 Hz); 7.5-7.9 (6H, m); 7.9-8.1 (2H, m); 8.13 (1H, d, 7 Hz); 8.90 (1H, s) and 10.52 ppm (1H, s).

Compound (5.45): Yield 14%, amorphous, ¹H-NMR (DMSO-d₆, TMS) δ: 2.1-2.6 (2H, m); 2.21 (6H, s); 2.62 (2H, t, 6 Hz); 4.07 (2H, t, 6 Hz); 5.45 (2H, dd, 4 and 8 Hz); 7.04 (2H, d, 9 Hz); 7.39 (2H, d, 9 Hz); 7.5-7.9 (3H, m); 7.94 (1H, d, 7 Hz); 8.91 (1H, br. s) and 10.50 ppm (1H, br. s).

Compound (5.46): Yield 30%, amorphous, ¹H-NMR (DMSO-d₆, TMS) δ: 2.1-2.6 (2H, m); 2.21 (6H, s); 2.62 (2H, t, 6 Hz); 4.07 (2H, t, 6 Hz); 5.45 (2H, dd, 4 and 8 Hz); 7.04 (2H, d, 9 Hz); 7.39 (2H, d, 9 Hz); 7.5-7.9 (3H, m); 7.94 (1H, d, 7 Hz); 8.91 (1H, br. s) and 10.50 ppm (1H, br. s).

Compound (5.47): Yield 55%, melting point 166-168° C. (dec.), ¹H-NMR (DMSO-d₆, TMS) δ: 2.34-2.63 (2H, m, overlapped with DMSO); 4.59 (2H, d, 5.1 Hz); 2.29 (2H, dt, 13.9 Hz, 1.5 Hz); 5.45 (1H, dd, 7.8 Hz, 2.9 Hz); 5.95-6.14 (1H, m); 7.04 (2H, dd, 8.8 Hz, 2.0 Hz); 7.38 (2H, dd, 8.8 Hz, 2.0 Hz); 7.61-7.96 (4H, m); 8.89 (1H, s); 10.50 ppm (1H, s).

Compound (5.48): Melting point 212-214° C. (dec.), ¹H-NMR (DMSO-d₆, TMS) δ: 2.2-2.7 (2H, m, overlapped with DMSO); 5.23 (2H, s); 5.4-5.6 (1H, m); 7.14 (2H, d, 8.8 Hz); 7.35-7.55 (4H, m); 7.55-7.90 (3H, m); 7.95 (1H, d, 7.3 Hz); 8.59 (2H, d, 5.1 Hz); 8.91 (1H, br s) and 10.52 ppm (1H, s).

Compound (5.49): Amorphous powder, ¹H-NMR (DMSO-d₆, TMS) δ: 2.2-2.6 (2H, m, overlapped with DMSO); 5.33 (2H, s); 5.4-5.6 (1H, m); 7.17 (2H, d, 8.8 Hz); 7.45 (2H, d, 8.8 Hz); 7.5-7.9 (3H, m); 7.9-8.1 (2H, m); 8.58 (1H, d, 8.1 Hz); 8.8-8.9 (1H, m); 8.9-9.1 (1H, m) and 10.55 ppm (1H, s).

Compound (5.50): Melting point: >170° C. (dec.), ¹H-NMR (DMSO-d₆, TMS) δ: 2.2-2.6 (2H, m, overlapped with DMSO); 5.21 (2H, s); 5.4-5.6 (1H, m); 7.14 (2H, d, 8.8 Hz); 7.41 (2H, d, 8.8 Hz); 7.3-8.0 (7H, m); 8.58 (1H, d, 4.4 Hz); 8.89 (1H, s) and 10.49 ppm (1H, s).

Compound (5.51): Yield 23%, amorphous, ¹H-NMR (DMSO-d₆, TMS) δ: 2.50-2.56 (2H, m, overlapped with DMSO); 4.11 (2H, d, 5.8 Hz); 4.86 (2H, s); 5.25 (1H, t, 5.8 Hz); 5.48 (1H, dd, 8.4 Hz and 4.4 Hz); 7.09 (2H, dd, 8.7 Hz and 1.8 Hz); 7.43 (2H, dd, 9.1 Hz and 2.2 Hz); 7.62 (1H, d, 8.0 Hz); 7.69 (1H, t, 7.3 Hz); 7.79 (1H, t, 7.3 Hz); 7.95 (1H, d, 8.0 Hz); 8.89 (1H, s) and 10.50 ppm (1H, s).

Compound (5.52): Yield 7%, melting point, 135-137° C. (dec.), ¹H-NMR (DMSO-d₆, TMS) δ: 2.22-2.26 (2H, m); 2.36-2.58 (2H, m, overlapped with DMSO); 4.78 (2H, d, 2.0

Hz); 5.48 (1H, dd, 7.8 Hz, 2.9 Hz); 7.08 (2H, dd, 8.8 Hz, 2.0 Hz); 7.42 (2H, dd, 8.8 Hz, 2.0 Hz); 7.59-7.97 (4H, m); 8.90 (1H, s); 10.51 ppm (1H, s).

Compound (5.53): Yield 2%, melting point, 155-157° C. (dec.), ¹H-NMR (DMSO-d₆, TMS) δ: 2.37-2.59 (3H, m, overlapped with DMSO); 4.85 (2H, d, 2.2 Hz); 5.50 (1H, dd, 7.8 Hz, 2.9 Hz); 7.14 (2H, dd, 8.8 Hz, 2.0 Hz); 7.44 (2H, dd, 8.8 Hz, 2.0 Hz); 7.61-7.99 (4H, m); 8.91 (1H, s); 10.52 ppm (1H, s).

Compound (5.54): Yield 64%, melting point, 148-150° C. (dec.), ¹H-NMR (DMSO-d₆, TMS) δ: 2.30-2.50 (1H, m, overlapped with DMSO); 2.50-2.68 (3H, m, overlapped with DMSO); 4.10 (2H, t, 5.9 Hz); 5.50 (1H, dd, 7.8 Hz and 2.9 Hz); 7.06 (2H, dd, 8.8 Hz and 1.95 Hz); 7.40 (2H, dd, 8.8 Hz and 1.95 Hz); 7.59-7.97 (4H, m); 8.89 (1H, s) and 10.50 ppm (1H, s).

Compound (5.55): Yield 75%, melting point 152-154° C. (dec), ¹H-NMR (DMSO-dₛ, TMS) δ: 1.55 (3H, d, 5.9 Hz); 1.82 (3H, s); 2.30-2.50 (2H, m, overlapped with DMSO); 5.00-5.20 (1H, m); 5.50 (1H, dd, 7.8 Hz and 2.9 Hz); 7.08 (2H, dd, 8.8 Hz and 1.95 Hz); 7.41 (2H, dd, 8.8 Hz and 1.95 Hz); 7.60-7.98 (4H, m); 8.91 (1H, s) and 10.52 ppm (1H, s).

Compound (5.56): Yield 10%, melting point 200-204° C. (dec.), ¹H-NMR (DMSO-d₆, TMS) δ: 1.70 (3H, d, 5.8 Hz); 2.1-2.6 (2H, m, overlapped with DMSO); 2.63, (3H, s); 5.3-5.5 (1H, m); 6.2-6.4 (1H, s); 7.05 (2H, d, 8.8 Hz); 7.34 (2H, d, 8.8 Hz); 7.4-7.9 (6H, m); 7.9-8.1 (2H, m); 8.33 (1H, d, 8.0 Hz); 8.90 (1H, s) and 10.49 ppm (1H, s).

Compound (5.57): Yield 36%, melting point 218-220° C. (dec.), ¹H-NMR (DMSO-d₆, TMS) δ: 2.2-2.7 (2H, m); 5.4-5.6 (1H, m); 5.71 (2H, s); 7.27 (2H, d, 8.8 Hz); 7.46 (2H, d, 8.8 Hz); 7.5-7.9 (6H, m); 7.96 (1H, d, 7.3 Hz); 8.09 (1H, d, 8.8 Hz); 8.20 (1H, d, 8.0 Hz); 8.89 (1H, s); 8.93 (1H, d, 4.4 Hz) and 10.51 ppm (1H, s).

Compound (5.58): Yield 18%, melting point: >140° C. (dec.), ¹H-NMR (DMSO-d₆, TMS) δ: 2.32 (1H, dd, 8.7 and 14.6 Hz, partly overlapped with DMSO); 2.66 (3H, s); 5.51 (1H, dd, 3.7 and 8.1 Hz); 5.60 (2H, s); 7.28 (2H, d, 8.8 Hz); 7.46 (2H, d, 8.8 Hz); 7.5-7.8 (5H, m); 7.8-8.1 (3H, m); 8.89 (1H, s) and 10.49 ppm (1H, s).

Compound (5.59): Melting point>215° C. (dec.), ¹H-NMR (DMSO-d₆, TMS) δ: 2.2-2.7 (2H, m, overlapped with DMSO); 2.67 (3H, s); 4.9-5.6 (1H, m); 5.63 (2H, s); 7.20 (2H, d, 8.8 Hz); 7.46 (2H, d, 8.8 Hz); 7.5-7.9 (5H, m); 7.9-8.1 (2H, m); 8.22 (1H, d, 1.9 Hz); 8.88 (1H, s) and 10.50 ppm (1H, s).

Compound (5.60): Yield 76%, melting point>204° C. (dec.), ¹H-NMR (DMSO-d₆, TMS) δ: 2.30 (1H, dd, 8.1 and 14.7 Hz), 2.4-2.6 (1H, partly overlapped with DMSO); 3.29 (3H, s, overlapped with DMSO H2O); 5.16 (2H, s); 5.47 (1H, dd, 4.4 and 8.8 Hz); 7.12 (2H, d, 8.8 Hz); 7.23 (1H, d, 4.4 Hz); 7.31 (1H, s); 7.41 (2H, d, 8.8 Hz); 7.5-7.8 (3H, m); 7.93 (1H, d, 7.3 Hz); 8.43 (1H, d, 5.1 Hz); 8.88 (1H, d, 1.5 Hz) and 10.48 ppm (1H, s).

Compound (5.61): Yield 78%, melting point>210° C. (dec.) ¹H-NMR (DMSO-d₆, TMS) δ: 2.32 (1H, dd, 8.8 and 14.7 Hz), 2.43 (6H, s, overlapped with DMSO); 2.4-2.7 (1H, partly overlapped with DMSO); 5.12 (2H, s); 5.49 (1H, dd, 3.7 and 8.1 Hz); 7.0-7.2 (4H, m); 7.43 (2H, d, 8.8 Hz); 7.5-7.9 (3H, m); 7.95 (1H, d, 7.3 Hz); 8.89 (1H, s) and 10.50 ppm (1H, s).

Compound (5.62): Yield 68%, melting point 180-182° C., ¹H-NMR (DMSO-d₆, TMS) δ: 2.34 (1H, dd, 9 Hz and 15 Hz); 2.62 (1H, dd, 4 Hz and 15 Hz); 5.62 (1H, dd, 4 Hz and 9 Hz); 7.11 (1H, s); 7.2-7.6 (6H, m); 7.89 (1H, d, 9 Hz); 8.96 (1H, s) and 10.53 ppm (1H, s).

Compound (5.63): Melting point 202-204° C. (dec.), ¹H-NMR (DMSO-d₆, TMS) δ: 2.23 (1H, dd, 9 Hz and 15 Hz); 2.4-2.7 (1H, m, overlapped with DMSO); 4.33 (4H, m); 5.52 (1H, dd, 4 Hz and 9 Hz); 7.04 (1H, s); 7.2-7.6 (6H, m); 8.93 (1H, s) and 10.50 ppm (1H, s).

Compound (5.64): Melting point 197-199° C. (dec.), ¹H-NMR (DMSO-d₆, TMS) δ: 2.2-2.7 (2H, m overlapped with DMSO); 3.84 (3H, s); 3.86 (3H, s); 5.55 (1H, dd, 4 Hz and 9 Hz); 7.07 (1H, s); 7.2-7.4 (1H, m); 7.4-7.6 (5H, m); 8.94 (1H, s) and 10.52 ppm (1H, s).

Compound (5.65): Yield 68%, amorphous, ¹H-NMR (DMSO-d₆, TMS) δ: 2.2-2.7 (2H, m overlapped with DMSO); 3.82 (3H, s); 3.89 (3H, s); 4.00 (3H, s); 5.51 (1H, dd, 3.7 Hz and 8.1 Hz); 6.90 (1H, s); 7.2-7.6 (6H, m); 8.93 (1H, s) and 10.51 ppm (1H, s).

Compound (5.66): Yield 75%, amorphous, ¹H-NMR (DMSO-d₆, TMS) δ: 2.2-2.7 (2H, m overlapped with DMSO); 3.86 (3H, s); 3.93 (3H, s); 5.52 (1H, dd, 3.7 Hz and 8.1 Hz); 6.63 (1H, s); 6.77 (1H, s); 7.3-7.6 (5H, m); 8.92 (1H, d, 1.5 Hz) and 10.50 ppm (1H, d, 1.5 Hz).

Compound (5.67): Yield 46%, amorphous, ¹H-NMR (DMSO-d₆, TMS) δ: 2.27 (1H, dd, 14.7 Hz, 9.5 Hz); 2.45 (3H, s); 2.61 (1H, dd, 14.7 Hz, 3.7 Hz); 5.64 (1H, dd, 9.5 Hz, 3.7 Hz); 7.27-7.38 (1H, m); 7.44-7.54 (5H, m); 7.61 (1H, d, 8.1 Hz); 7.79 (1H, s); 8.93 (1H, s) and 10.53 ppm (1H, s).

Compound (5.68): Yield 38%, melting point, 182-183° C., ¹H-NMR (DMSO-d₆, TMS) δ: 2.33 (1H, dd, 8.8 Hz and 14.6 Hz); 2.65 (1H, dd, 3.7 Hz and 14.6 Hz); 5.65 (1H, dd, 3.7 and 8.1 Hz); 7.2-7.6 (5H, m); 7.58 (1H, d, 8.1 Hz); 7.78 (1H, d, 8.1 Hz); 8.31 (1H, s); 8.89 (1H, s) and 10.48 ppm (1H, s).

### Synthesis 187

### Methanesulfonic acid 1-(2-methyl-quinolin-4-yl)ethyl ester (15.12)

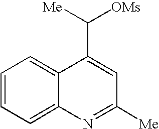

A solution of hydroxymethylquinoline (17) (1.7 g, 10 mmol) in DCM (5 mL) was cooled in an ice bath and to this 0.39 M Dess-Martin periodinane solution in DCM (31 mL, 12 mmol) was added. The resulting mixture was stirred while cooling for 1.5 h and to this saturated aqueous NaHCO₃ was added (15 mL). The mixture was stirred until both organic and aqueous phases become homogeneous. The organic phase was washed with aqueous Na₂S₂O₃ and brine and dried over Na₂SO₄. The extract was filtered and the solvent removed in vacuo. The residue was purified by flash chromatography on silica gel, eluting with a mixture of light petroleum ether and EtOAc (2:1, 1:1) to give crystalline material (0.51 g). This was dissolved in THF (10 mL) and cooled in an ice bath. 1.4 M Solution of MeMgBr in THF (4.3 mL, 6 mmol) was added dropwise while cooling. The mixture was stirred for 30 min while cooling and to this saturated aqueous NH₄Cl (50 mL) and water (50 mL) was added. The mixture was extracted with EtOAc (100+50 mL). Combined organic phase was washed with brine (50 mL) dried over Na₂SO₄, filtered and evaporated. The residue was purified by flash chromatography on silica gel, eluting with a mixture of light petroleum ether and EtOAc (1:1, 1:0) to give crystalline material (0.325 g). This product (300 mg) was dissolved in DCM (5 mL) and the solution cooled in an ice bath. To this triethylamine (0.45 mL, 3.2 mmol) was added in one portion followed by dropwise addition of mesylchloride (0.25 mL, 3.2 mmol). The cooling bath was removed and the resulting mixture was stirred for 30 min at room temperature. The mixture was diluted with DCM (30 mL) and washed with brine (2×30 mL). The organic phase was dried over Na₂SO₄ filtered and evaporated to give (5.12) (0.47 g) as a crude product.

### Synthesis 188

### 4-Chloromethylquinoline hydrochloride (15.13)

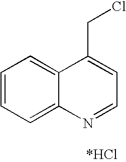

Thionylchloride (1.45 mL, 20 mmol) was added dropwise to a solution of carbinol (18) (1.5 g, 9.4 mmol) in DCM at room temperature. The reaction mixture was stirred at room temperature for 1 h and evaporated to give (15.13) (2.0 g) as a crude product.

### General procedure for the synthesis of chloromethylquinolines (15.14) and (15.15)

Method J: Quinoline carboxylic acid (19.1) or (19.2) (4 mmol) was refluxed in a mixture of MeOH (13 mL) and H₂SO₄ (2.5 mL) for 3 h. The reaction mixture was cooled to room temperature and to this water (25 mL) was added. Saturated aqueous NaHCO₃ was added to adjust pH˜8. The mixture was extracted with EtOAc (2×35 mL), combined organic phase dried over Na₂SO₄ filtered and evaporated to give a crude ester. This was dissolved in MeOH (25 mL) and NaBH₄ (0.76 g, 20 mmol) was added portion wise to maintain gentle reflux. After addition was complete, the reaction mixture was stirred for 1 h at room temperature and water (100 mL) was added. The reaction mixture was extracted with EtOAc (100 mL) washed with brine (50 mL) and dried over Na₂SO₄. The extract was filtered and evaporated to give hydroxymethylquinoline derivative. This was dissolved in DCM (30 mL) and to the solution thionylchloride (0.47 mL, 6.4 mmol) was added dropwise. The reaction mixture was refluxed for 3 h and evaporated to give (15.14) or (15.15) as a crude product.

Following a method analogous to Method J, the following compounds were obtained as a crude product.

| Synthesis | Method | Name | Structure |
|---|---|---|---|
| 189 | J | 4-Chloromethyl-6-fluoro-2- methylquinoline hydrochloride (15.14) | 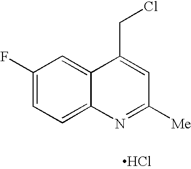 |
| 190 | J | 4-Chloromethyl-6-chloro-2- methylquinoline hydrochloride (15.15) | 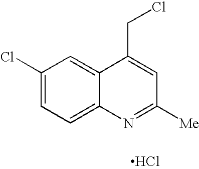 |

### General procedure for the synthesis of chloromethylpyridines (15.16) and (15.17)

Method K: A solution of 4-methylpiridine derivative (20.1) or (20.2) (40 mmol) in dry THF was cooled to −70° C. under inert atmosphere and to this 1.6 M n-BuLi in hexanes (28 mL, 44 mmol) was added dropwise. After addition was complete, the solution was stirred for additional 30 min at −70° C. and DMFA (6.2 mL, 80 mmol) was added. The mixture was stirred for additional 1 h 30 min at −70° C. and quenched with saturated aqueous NH₄Cl (10 mL) and warmed to room temperature. The mixture was partially evaporated, water (100 mL) was added to the residue and extracted with CHCl₃(3×100 mL). Combined organic phase was washed with brine (100 mL), dried over Na₂SO₄ filtered and evaporated. The residue was dissolved in MeOH (30 mL) and added dropwise to the suspension of NaIO₄ (25.7 g, 120 mmol) in MeOH (30 mL) at the rate to maintain gentle reflux. The mixture was passed trough a short celite column and NaBH₄ (4.54 g, 120 mmol) was added to the solution. The mixture was stirred for 30 min and a spoonful of SiO₂ was added. The solvent was removed in vacuo and the residue applied on a silica gel column. Elution with DCM containing 5% MeOH gave hydroxymethylpiridines (20.1) or (20.2). Intermediate (20.1) or (20.2) (7 mmol) was dissolved in DCM (70 mL) and to the solution thionylchloride (1.05 mL, 14.4 mmol) was added dropwise. The reaction mixture was refluxed for 3 h and evaporated to give (15.16) or (15.17) as a crude product.

Following a method analogous to Method J, the following compounds were obtained as a crude product.

| Syn- | | | |
|---|---|---|---|
| thesis | Method | Name | Structure |
| 191 | K | 4-Chloromethyl-2- methylpyridine hydrochloride (15.16) | 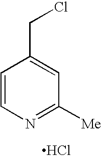 |
| 192 | K | 4-Chloromethyl-2,6- dimethylpyridine hydrochloride (15.17) | 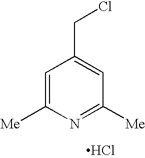 |

### Synthesis 193

### 2-(1,1-Dioxo-2-(3-hydroxymethylphenyl)-2,3-dihydro-1H-benzo[d]isothiazol-3-yl)-N-hydroxyacetamide (24)

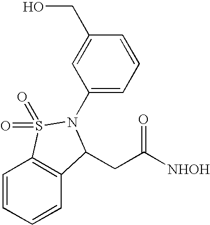

To a solution of sulphonylchloride (1.1) (2 mmol) and 3-hydroxymethylaniline (22) (2 mmol) in dioxane (10 mL) added was 1 M aqueous solution of NaHCO₃ (4 mL). The resulting mixture was stirred at room temperature for 2 hours and diluted with water (50 mL). The precipitate was collected on a filter, washed with water and dried over P₂O₅ in vacuo to give ester (23) (427 mg, 62%). A solution of hydroxylamine hydrochloride (174 mg, 2.5 mmol) and KOH (278 mg, 4 mmol) in methanol (3 mL) was added to a solution of ester (23) (174 mg, 0.5 mmol) in methanol (2 mL). The mixture was stirred at room temperature overnight and evaporated. Water was added to the residue and 20% KHSO₄ was added until neutral pH was reached. The mixture was extracted with ethyl acetate (20 mL). The organic phase was separated and washed with brine (20 mL) and dried over Na₂SO₄. The solution was filtered and evaporated. The residue was treated with acetonitrile, the precipitate collected on a filter and dried to give (24) (15 mg, 9%), melting point, 165-166° C., ¹H-NMR (DMSO-d₆, TMS) δ: 2.29 (1H, dd, 9 Hz and 15 Hz); 2.63 (1H, dd, 4 Hz and 15 Hz); 4.54 (1H, d, 5 Hz); 5.34 (1H, t, 5 Hz); 5.67 (1H, dd, 4 Hz and 9 Hz); 7.2-7.8 (7H, m); 7.98 (1H, d, 8 Hz); 8.9 (1H, br s) and 10.5 ppm (1H, br s).

### Synthesis 194

### N-tert-Butoxycarbonyl-4-(4-nitrophenoxy)-but-2-ynylamine (25)

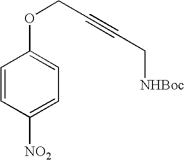

A solution of alcohol (16.7) (278 mg, 1.34 mmol) and triethylamine (0.37 mL, 2.68 mmol) in benzene (11 mL) was cooled in an ice bath under argon atmosphere. To this, mesylchloride (0.21 mL, 2.68 mmol) was added. The mixture was allowed to reach room temperature and stirred for 2 h. It was filtered trough a short column of silica gel eluting with benzene. The solution was washed with saturated aqueous NaHCO₃ (50 mL) and brine (50 mL), dried over Na₂SO₄ and evaporated. The residue (382 mg) was dissolved in DMFA (11 mL) and NaN₃ (231 mg, 3.55 mmol) was added. The reaction mixture was stirred for 4 days at room temperature and diluted with water (30 mL). The mixture was extracted with Et₂O (3×25 mL). Combined organic phase was washed with water (30 mL), dried over Na₂SO₄ and the solvent was removed in vacuo. The residue (311 mg) was dissolved in Et₂O (10 mL) and the mixture cooled in an ice bath. To this triphenylphosphine (352 mg, 1.34 mmol) was added and the mixture was allowed to reach room temperature and stirred for 1 h 30 min. Water (1.25 mL) was added and the mixture stirred at room temperature overnight. The organic phase was removed by decantation, dried over Na₂SO₄ and the solvent removed in vacuo. The residue (276 mg) was dissolved in DCM and Boc₂O was added to the solution. The mixture was stirred at room temperature overnight and the solvent removed in vacuo. The residue was purified by means of flash chromatography on silica gel eluting with a mixture of light petroleum ether and EtOAc (4:1, 2:1) to give the title compound 25 (182 mg).

### Synthesis 195

### N-tert-Butoxycarbonyl-4-(4-aminophenoxy)-but-2-ynylamine (26)

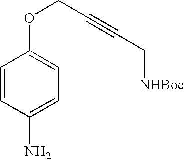

Nitrobenzene derivative (25) (182 mg, 0.6 mmol) was dissolved in methanol (5 mL) and to the solution Na₂S×9H₂O (576 mg, 2.4 mmol) was added and the mixture was set to reflux for 3 h. The solvent was removed in vacuo and the residue partitioned between the water and Et₂O (30 mL). The organic phase was extracted with 1 M aqueous HCl. Acidic aqueous extract was separated and made alkaline with 5 M aqueous NaOH to pH˜10. The mixture was extracted with Et₂O (3×30 mL) and combined organic phase washed with brine (30 mL). The extract was dried over Na₂SO₄, filtered and evaporated to give title compound (26) (40 mg) as a crude product.

### Synthesis 196

### {2-[4-(4-tert-Butoxycarbonylamino-but-2-ynyloxy)-phenyl]-1,1-dioxo-2,3-dihydro-1H-benzo[d]isothiazol-3-yl}-acetic acid (27)

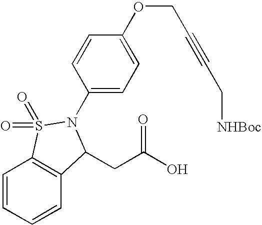

Following a method analogous to Method F (for the preparation of compounds 4), the title compound was obtained from sulphonylchloride (1.1) and aniline (26) as a crude product.

### Synthesis 197

### 2-{2-[4-(4-tert-Butoxycarbonylamino-but-2-ynyloxy)-phenyl]-1,1-dioxo-2,3-dihydro-1H-benzo[d]isothiazol-3-yl}-N-hydroxyacetamide (28)

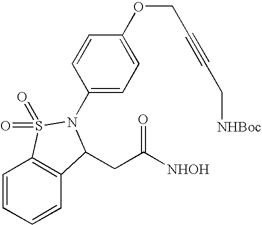

Following a method analogous to Method H (for the preparation of compounds 5) from carboxylic acid (27), the title compound was obtained as a crude product.

Synthesis 198

### 2-{2-[4-(4-amino-but-2-ynyloxy)-phenyl]-1,1-dioxo-2,3-dihydro-1H-benzo[d]isothiazol-3-yl}-N-hydroxyacetamide hydrochloride (29)

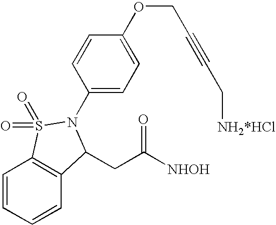

To the solution of N-Boc-protected compound (28) (40 mg, 0.09 mmol) in DCM (2.8 mL) added was trifluoroacetic acid (1.4 mL) dropwise while cooling in an ice bath. After addition was complete, ice bath was removed and the mixture stirred at room temperature for 45 min. Solvent and excess of trifluoroacetic acid was removed in vacuo and the residue was treated with 2 M HCl in Et₂O. The mixture was evaporated and the residue was repeatedly treated with 2 M HCl in Et₂O and again evaporated. The residue was treated with Et₂O and the precipitate collected on a filter to give the title compound (29). Yield 68%, amorphous, ¹H-NMR (DMSO-d₆, TMS) δ: 2.29-2.50 (2H, m, overlapped with DMSO); 3.79-3.83 (2H, m); 4.94 (2H, s); 5.46-5.53 (1H, m); 7.11 (2H, d, 8.1 Hz); 7.44 (2H, d, 8.1 Hz); 7.60-7.80 (3H, m); 7.95 (1H, d, 6.6 Hz); 8.40 (4H, s) and 10.59 ppm (1H, s).

### Synthesis 199

### (E)-3-(2-Chlorosulfonyl-5-hydroxyphenyl)acrylic acid methyl ester (31)

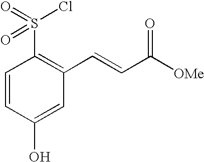

Following a method analogous to Method A (for the synthesis of (1)) from unsaturated ester (30), the title compound was obtained as a crude product.

### Synthesis 200

### (5-Hydroxy-1,1-dioxo-2-phenyl-2,3-dihydro-1H-benzo[d]isothiazol-3-yl)acetic acid methyl (32)

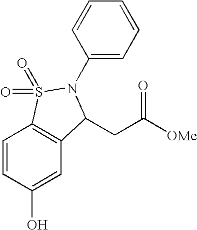

A solution of sulphonylchloride (31) (7.05 g, 25.5 mmol) and aniline (2.1) (4.75 g, 51 mmol) in DCM (200 mL) was stirred for 17 h at room temperature. The solution was washed with 1 M aqueous HCl (200 mL) and brine (3×100 mL) and dried over Na₂SO₄. The solution was filtered and evaporated to give the intermediate product (8.13 g). This was dissolved in DMFA (40 mL) and K₂CO₃ (6.74 g, 48.9 mmol) was added. The resulting mixture and heated at 80° C. for 5 h cooled to room temperature and poured into water (300 mL). The aqueous phase was extracted with EtOAc (2×250 mL). The combined organic phase was washed with brine (3×100 mL), dried over Na₂SO₄ filtered and evaporated. The residue was purified by means of flash chromatography on silica gel eluting with a mixture of light petroleum ether with EtOAc (2:1, 1:1) to give the product (32) (1.52 g).

### Synthesis 201

### (1,1-Dioxo-2-phenyl-5-trifluoromethanesulfonyloxy-2,3-dihydro-1H-benzo[d]isothiazol-3-yl)acetic acid methyl ester (33)

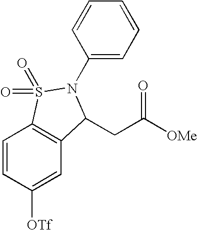

To the solution of (32) (333 mg, 1 mmol) in DCM (5 mL) added was pyridine (0.16 mL, 2 mmol) and the solution cooled in an ice bath. Triflic anhydride (0.2 mL, 1.2 mmol) was added dropwise to the solution and the resulting mixture was allowed to reach room temperature. 1 M Aqueous HCl was added (100 mL) and the mixture extracted with EtOAc (2×150 mL). The combined organic phase was washed with saturated aqueous NaHCO₃ (100 mL) and brine (100 mL) and dried over Na₂SO₄. The extract was filtered and the solvent removed in vacuo to give (33) (460 mg).

### Synthesis 202

### (1,1-Dioxo-2,5-diphenyl-2,3-dihydro-1H-benzo[d]isothiazol-3-yl)acetic acid methyl ester (34)

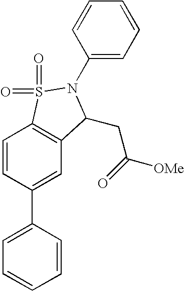

To the solution of compound (33) (460 mg, 1 mmol) in DME (10 mL) added was Pd(PhP)₄ (28 mg, 0.03 mmol), phenylboronic acid (133 mg, 1.1 mmol) and 2 M aqueous Na₂CO₃ (1.3 mL, 2.6 mmol). The resulting mixture was heated at 85° C. for 2 h and cooled to room temperature. This was diluted with water (20 mL) and extracted with EtOAc (2×20 mL). The combined organic phase was dried over Na₂SO₄ filtered and evaporated. The residue was purified by flash chromatography on silica gel eluting with a mixture of light petroleum ether and EtOAc (4:1) to give (34) (340 mg).

### Synthesis 203

### (1,1-Dioxo-2,5-diphenyl-2,3-dihydro-1H-benzo[d]isothiazol-3-yl)acetic acid ester (35)

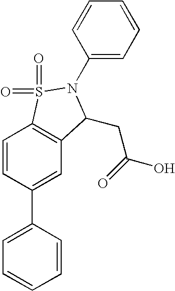

Following a method analogous to Method E (for the synthesis of (4)) from ester (34), the title compound was obtained as a crude product.

### Synthesis 204

### 2-(1,1-Dioxo-2,5-diphenyl-2,3-dihydro-1H-benzo[d]isothiazol-3-yl)-N-hydroxyacetamide (36)

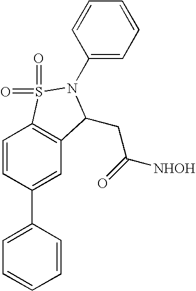

Following a method analogous to Method G (for the synthesis of (5)) from carboxylic acid, the title compound was obtained. Yield 80%, melting point: >115° C. (dec.), ¹H-NMR spectrum (DMSO-d6, TMS) δ: 2.3-2.5 (1H, m, overlapped with DMSO); 2.69 (1H, dd, 3.7 Hz and 14.6 Hz); 5.75 (1H, dd, 4.4 Hz and 8.8 Hz); 7.34 (1H, m); 7.4-7.8 (9H, m); 7.86 (1H, s); 7.9-8.2 (2H, m); 8.97 (1H, s) and 10.55 ppm (1H, s).

### Synthesis 205

### (5-Ethoxy-1,1-dioxo-2-phenyl-2,3-dihydro-1H-benzo[d]isothiazol-3-yl)acetic acid methyl ester (37)

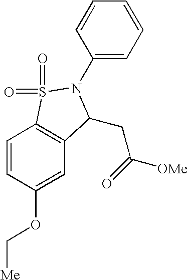

A mixture of compound (32) (167 mg, 0.5 mmol), ethyl iodide (0.08 mL, 1.0 mmol) and K₂CO₃ (415 mg, 1.5 mmol) in DMFA (3 mL) was stirred at room temperature for 2 h 30 min. The mixture was diluted with water (20 mL) and extracted with EtOAc (2×20 mL). The combined organic phase was washed with brine (20 mL) and dried over Na₂SO₄. The extract was filtered and the solvent removed in vacuo to give the title compound (37) (180 mg) as a crude product.

### Synthesis 206

### (5-Ethoxy-1,1-dioxo-2-phenyl-2,3-dihydro-1H-benzo[d]isothiazol-3-yl)acetic acid (38)

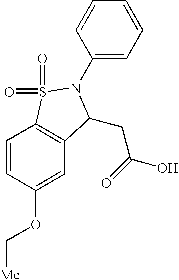

Following a method analogous to Method E (for the synthesis of (4)) from ester (37), the title compound was obtained as a crude product.

### Synthesis 207

### 2-(5-Ethoxy-1,1-dioxo-2-phenyl-2,3-dihydro-1H-benzo[d]isothiazol-3-yl)-N-hydroxyacetamide (39)

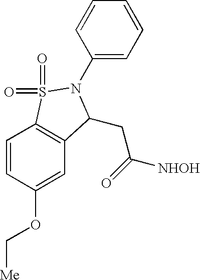

Following a method analogous to Method G (for the synthesis of (5)) from carboxylic acid (38), the title compound was obtained. Yield 35%, amorphous, ¹H-NMR (DMSO-d6, TMS): δ 1.36 (3H, t, 7 Hz); 2.31 (1H, dd, 8 Hz and 15 Hz); 2.60 (1H, dd, 4 Hz and 15 Hz); 4.13 (2H, q, 7 Hz); 5.60 (1H, dd, 7 Hz and 8 Hz); 7.0-7.4 (3H, m); 7.4-7.6 (4H, m); 7.86 (1H, d, 8 Hz) 8.94 (1H, s) and 10.51 ppm (1H, s).

### Synthesis 208

### (E)-3-(4-Bromo-2-phenylsulfamoylphenyl)acrylic acid methyl ester (40)

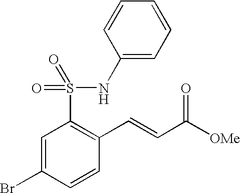

To a solution of aniline (2.1) (1.68 g, 18 mmol) in dioxane (20 mL) added was 1 M aqueous NaHCO₃ (15 mL). To the mixture added was a solution of sulphonylchloride (1.8) (3.3 g, 9.7 mmol) in dioxane (20 mL). The reaction mixture was stirred at room temperature for 1 h and diluted with 5% aqueous KHSO₄ (100 mL) and extracted with EtOAc (100 mL). The organic phase was washed with saturated aqueous NaHCO₃ (100 mL) and brine (100 mL). The extract was dried over Na₂SO₄, filtered and the solvent removed in vacuo. The residue was treated with a mixture of Hex and EtOAc (2:1). The precipitate formed was collected on a filter to give (40) (1.8 g).

### Synthesis 209

### (E)-3-(3-Phenylsulfamoyl-biphenyl-4-yl)acrylic acid methyl ester (41)

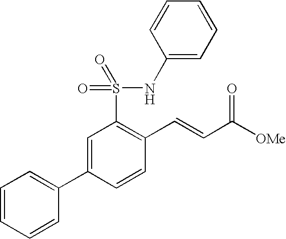

The mixture of arylbromide (40) (0.4 g, 1.0 mmol), phenylboronic acid (0.146 g, 1.2 mmol), Pd(Ph₃P)₄ (35 mg, 0.03 mmol) and Cs₂CO₃ (0.456 g, 1.4 mmol) was heated in dioxane (12 mL) at 90° C. for 7 h. The reaction mixture was cooled to room temperature and diluted with EtOAc (50 mL). The mixture was washed with 5% aqueous KHSO₄ (50 mL), saturated aqueous NaHCO₃ (50 mL) and brine (50 mL). The organic phase was dried over Na₂SO₄, filtered and evaporated. The residue was treated with a mixture of Hex and EtOAc (3:1). The precipitate formed was collected on a filter to give (41) (0.21

### Synthesis 210

### (1,1-Dioxo-2,6-diphenyl-2,3-dihydro-1H-benzo[d]isothiazol-3-yl)acetic acid (42)

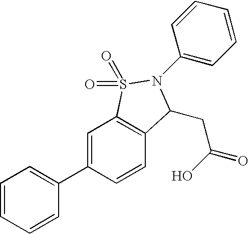

The mixture of compound (41) (0.21 g, 0.54 mmol) in dioxane (4 mL) and 1 M aqueous NaHCO₃ (3 mL) was set to reflux for 8 h. This was cooled to room temperature and diluted with water (40 mL). The product was extracted with EtOAc (50 mL) and the organic phase washed with brine (50 mL). The extract was dried over Na₂SO₄ filtered an evaporated to give the title compound (42) (0.18 g) as a crude product.

### Synthesis 211

### 2-(1,1-Dioxo-2,6-diphenyl-2,3-dihydro-1H-benzo[d]isothiazol-3-yl)-N-hydroxyacetamide (43)

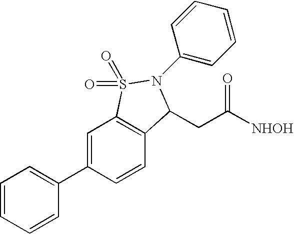

Following a method analogous to Method G (for the preparation of compounds (5)) from carboxylic acid (42), the title compound was obtained. Yield 45%, Melting point: 191-193° C., ¹H-NMR (DMSO-d6, TMS): δ: 2.36 (1H, dd, 9.5 Hz and 14.7 Hz); 2.67 (1H, dd, 3.7 Hz and 14.7 Hz); 5.74 (1H, dd, 3.7 and 8.1 Hz); 7.3-7.6 (8H, m); 7.72 (1H, d, 8.1 Hz); 7.82 (2H, d, 6.6 Hz); 8.12 (1H, d, 8.1 Hz); 8.24 (1H, s); 8.96 (1H, s) and 10.56 ppm (1H, s).

### General procedure for the synthesis of 2-iodo-N-phenylbenzenesulfonamides (45.1) and (45.2)

Method L: A solution of sulphonamide (44) (3.7 mmol) in THF (20 mL) was cooled to 0° C. under argon atmosphere. 1.4 M n-BuLi in hexanes (5.7 mL, 7.9 mmol) was added dropwise and the mixture was allowed to reach room temperature. After stirring at room temperature for 1 h, the temperature of the mixture was set to −78° C. and a solution of I₂ (1.04 g, 4.11 mmol) in THF (12 mL) was added. The mixture was stirred at −78° C. for 1 h and then allowed to reach room temperature. Concentrated aqueous Na₂S₂O₃ was added until the mixture became colorless and the mixture extracted with EtOAc (3×50 mL). The combined organic phase was washed with brine (100 mL) and dried over Na₂SO₄. Evaporation of the solvent gave crude product (45).

Following a method analogous to Method L, the following compounds were obtained as a crude product.

| Syn- | | | |
|---|---|---|---|
| thesis | Method | Name | Structure |
| 212 | L | 2-Iodo-4-methyl-N- phenylbenzenesulfonamide (45.1) | 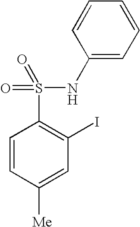 |
| 213 | L | 4-Chloro-2-Iodo- N-phenylbenzenesulfonamide (45.2) | 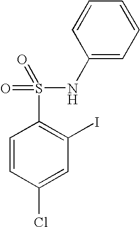 |

### General procedure for the synthesis of 2-(1,1-dioxo-2-phenyl-2,3-dihydro-1H-benzo[d]isothiazol-3-yl)acetic acid methyl esters (46.1) and (46.2)

Method M: A mixture of 2-iodo-N-phenylbenzenesulfonamide (45) (1.3 mmol), Pd(OAc)₂ (28 mg, 0.13 mmol), tri-o-tolylphosphine (77.3 mg, 0.25 mmol), triethylamine (1 mL, 7.2 mmol) and methyl acrylate (2.37 mL, 25.4 mmol) in DMFA (3 mL) was heated at 110° C. for 3 h. After cooling to room temperature, water (50 mL) was added and the mixture extracted with EtOAc (3×30 mL). The combined organic phase was dried over Na₂SO₄, filtered and evaporated. The residue was purified by means of flash chromatography on silica gel eluting with a mixture of light petroleum ether and EtOAc (5:1) to give (46).

Following a method analogous to Method M, the following compounds were obtained as a crude product.

| Synthesis | Method | Name | Structure |
|---|---|---|---|
| 214 | M | 2-(5-Methyl-1,1-dioxo-2-phenyl- 2,3-dihydro-1H- benzo[d]isothiazol-3-yl)acetic acid methyl ester (46.1) | 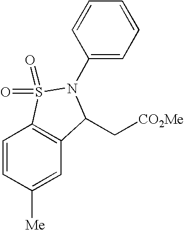 |
| 215 | M | 2-(5-Chloro-1,1-dioxo-2-phenyl- 2,3-dihydro-1H- benzo[d]isothiazol-3-yl)acetic acid methyl ester (46.2) | 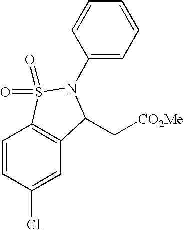 |

### Synthesis 216

### 2-(5-Methyl-1,1-dioxo-2-phenyl-2,3-dihydro-1H-benzo[d]isothiazol-3-yl)acetic acid (47.1)

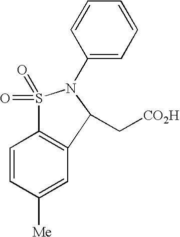

Following a method analogous to Method E (for the synthesis of (4)) from ester (46.1), the title compound was obtained as a crude product.

### Synthesis 217

### 2-(5-Chloro-1,1-dioxo-2-phenyl-2,3-dihydro-1H-benzo[d]isothiazol-3-yl)acetic acid (47.2)

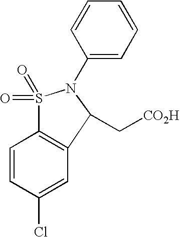

Following a method analogous to Method E (for the synthesis of (4)) from ester (46.2), the title compound was obtained as a crude product.

### Synthesis 218

### 2-(5-Methyl-1,1-dioxo-2-phenyl-2,3-dihydro-1H-benzo[d]isothiazol-3-yl)-N-hydroxyacetamide (481)

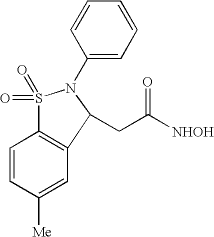

Following a method analogous to Method G (for the synthesis of (5)) from carboxylic (47.1), the title compound was obtained. Yield 45%, melting point: 150-155° C., ¹H-NMR (DMSO-d6, TMS) δ: 2.30 (dd, J=14.7 and 9.0 Hz, 1H); 2.46 (s, overlapped with DMSO, 3H); 2.62 (dd, J=14.73.8 Hz, 1H, overlapped with DMSO); 5.57-5.70 (m, 1H); 7.26-7.38 (m, 1H); 7.40-7.57 (m, 6H); 7.86 (d, J=7.9 Hz, 1H); 8.95 (s, 1H); 10.52 ppm (s, 1H).

### Synthesis 219

### (5-Chloro-1,1-dioxo-2-phenyl-2,3-dihydro-1H-benzo[d]isothiazol-3-yl)-N-hydroxyacetamide (48.2)

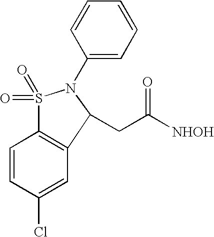

Following a method analogous to Method H (for the synthesis of (5)) from carboxylic (47.1), the title compound was obtained. Yield 80%, melting point: 192-194° C., ¹H-NMR (DMSO-d6, TMS) δ: 2.41 (dd, J=15.0 and 8.3 Hz, 1H); 2.70 (dd, J=15.0 and 4.0 Hz, 1H); 5.70 (dd, J=8.0 and 4.0 Hz, 1H); 7.30-7.41 (m, 1H); 7.44-7.52 (m, 4H); 7.73-7.82 (m, 2H); 8.05 (d, J=8.9 Hz, 1H); 8.94 (s, 1H) and 10.49 ppm (s, 1H).

### General procedure for the preparation of N-phenylbenzenesulfonamides (50.1)-(50.11)

Method N: Aniline (2.1) (0.70 g, 7.5 mmol) was suspended in 1 M aqueous NaHCO₃ (15 mL). A solution of sulphonylchloride (49) (5 mmol) in dioxane (15 mL) was added to the suspension and the mixture was stirred at room temperature for 22 h. This was diluted with 5% aqueous KHSO₄ (40 mL). The precipitate formed was collected on a filter and washed with large amount of water. The material was well dried in vacuo over P₂O₅ to give (50).

Following a method analogous to Method N, the following compounds were obtained as a crude product.

| Syn- | Me- | | |
|---|---|---|---|
| thesis | thod | Name | Structure |
| 220 | N | 3-Methoxy-N- phenylbenzenesulfonamide (50.1) | 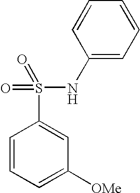 |
| 221 | N | 3-Chloro-N- phenylbenzenesulfonamide (50.2) | 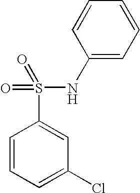 |
| 222 | N | 2-Trifluoromethyl-N- phenylbenzenesulfonamide (50.3) | 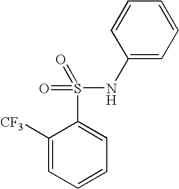 |
| 223 | N | 2-Fluoro-4-methyl-N- phenylbenzenesulfonamide (50.4) | 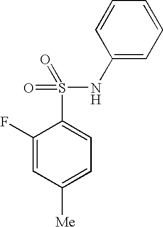 |
| 224 | N | 3-Chloro-4-fluoro-N- phenylbenzenesulfonamide (50.5) | 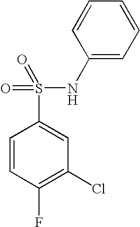 |
| 225 | N | 4-Fluoro-N- phenylbenzenesulfonamide (50.6) | 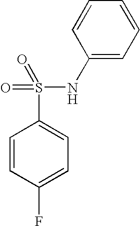 |
| 226 | N | 2-Chloro-N- phenylbenzenesulfonamide (50.7) | 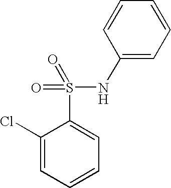 |
| 227 | N | 3,5-Dichloro-N- phenylbenzenesulfonamide (50.8) | 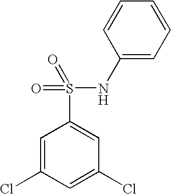 |
| 228 | N | 3-Trifluoromethyl-N- phenylbenzenesulfonamide (50.9) | 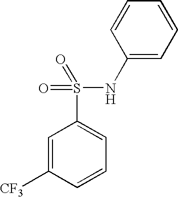 |
| 229 | N | 5-Bromo-2-methoxy-N- phenylbenzenesulfonamide (50.10) | 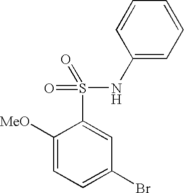 |
| 230 | N | 4-Chloro-2-fluoro-N- phenylbenzenesulfonamide (50.11) | 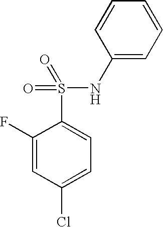 |

### General procedure for the preparation of 1,1-dioxo-2-phenyl-2,3-dihydro-1H-benzo[d]isothiazol-3-ol (51.1)-(51.11)

Method O: A solution of sulphonamide (50) (2.5 mmol) in THF (25 mL) was cooled (to 0° C. for the synthesis of compounds 51.1, 51.3, or to −78° C. for the synthesis of compounds 51.2, 51.4-51.11). 1.6 M n-BuLi in hexanes (3.5 mL, 5.5 mmol) was added dropwise and the mixture kept while cooling for up to 2 h. The temperature of the mixture was set to −78° C. and DMFA (0.39 mL, 5.0 mmol) was added in one portion. The cooling bath was removed and the mixture was allowed to reach room temperature and stirred for 2 h. 5% aqueous KHSO₄ (100 mL) was added and the mixture extracted with EtOAc (200 mL). The organic phase was separated and washed with brine (100 mL). The extract was dried over Na₂SO₄, filtered and evaporated. The residue was purified by flash chromatography on silica gel eluting with a mixture of light petroleum ether and EtOAc.

Following a method analogous to Method O, the following compounds were obtained as a crude product.

| Synthesis | Method | Name | Structure |
|---|---|---|---|
| 231 | O | 4-Methoxy-1,1-dioxo-2-phenyl- 2,3-dihydro-1H- benzo[d]isothiazol-3-ol (51.1) | 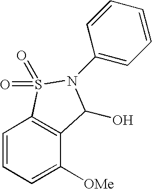 |
| 232 | O | 4-Chloro-1,1-dioxo-2-phenyl- 2,3-dihydro-1H- benzo[d]isothiazol-3-ol (51.2) | 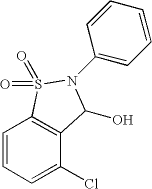 |
| 233 | O | 7-Trifluoromethyl-1,1-dioxo-2- phenyl-2,3-dihydro-1H- benzo[d]isothiazol-3-ol (51.3) | 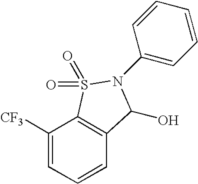 |
| 234 | O | 7-Fluoro-5-methyl-1,1-dioxo-2- phenyl-2,3-dihydro-1H- benzo[d]isothiazol-3-ol (51.4) | 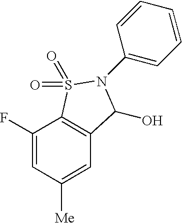 |
| 235 | O | 4-Chloro-5-fluoro-1,1-dioxo-2- phenyl-2,3-dihydro-1H- benzo[d]isothiazol-3-ol (51.5) | 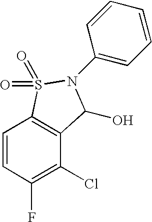 |
| 236 | O | 5-Fluoro-1,1-dioxo-2-phenyl- 2,3-dihydro-1H- benzo[d]isothiazol-3-ol (51.6) | 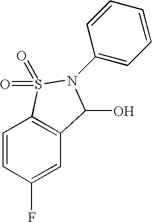 |
| 237 | O | 7-Chloro-1,1-dioxo-2-phenyl- 2,3-dihydro-1H- benzo[d]isothiazol-3-ol (51.7) | 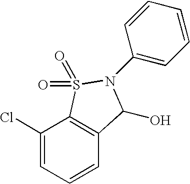 |
| 238 | O | 4,6-Dichloro-1,1-dioxo-2-phenyl- 2,3-dihydro-1H- benzo[d]isothiazol-3-ol (51.8) | 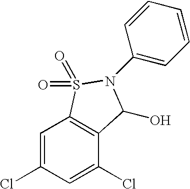 |
| 239 | O | 6-Trifluoromethyl-1,1-dioxo-2- phenyl-2,3-dihydro-1H- benzo[d]isothiazol-3-ol (51.9) | 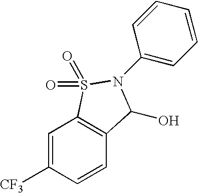 |
| 240 | O | 7-Methoxy-1,1-dioxo-2-phenyl- 2,3-dihydro-1H- benzo[d]isothiazol-3-ol (51.10) | 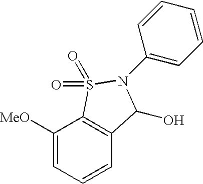 |
| 241 | O | 5-Chloro-7-fluoro-1,1-dioxo-2- phenyl-2,3-dihydro-1H- benzo[d]isothiazol-3-ol (51.11) | 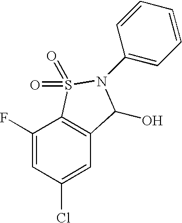 |

### General procedure for the preparation of 2-(1,1-dioxo-2-phenyl-2,3-dihydro-1H-benzo[d]isothiazol-3-yl)acetic acid methyl esters (52.1)-(52.12)

Method P: To a solution of compound (51) (215 mg, 0.75 mmol) and trimethyl phosphonoacetate (0.16 mL, 1.1 mmol) added was 1 M NaOMe in MeOH (1.5 mL, 1.5 mmol) the mixture was stirred at room temperature overnight and diluted with water (50 mL). Typically a white precipitate formed that was collected on a filter, washed with water and dried over P₂O₅ in vacuo to give 52. If no filterable precipitate formed, the product was extracted in EtOAc, and organic phase washed with brine. Drying over Na₂SO₄ filtration and the solvent removal gave the residue that was purified by flash chromatography on silica gel eluting with a mixture of light petroleum ether and EtOAc to give 52.

Following a method analogous to Method P, the following compounds were obtained as a crude product.

| Synthesis | Method | Name | Structure |
|---|---|---|---|
| 242 | P | 2-(4-Methoxy-1,1-dioxo-2- phenyl-2,3-dihydro-1H- benzo[d]isothiazol-3-yl)acetic acid methyl ester (52.1) | 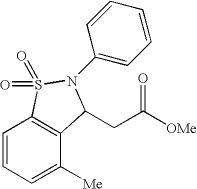 |
| 243 | P | 2-(4-Chloro-1,1-dioxo-2-phenyl- 2,3-dihydro-1H- benzo[d]isothiazol-3-yl)acetic acid methyl ester (52.2) | 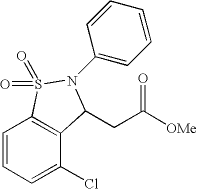 |
| 244 | P | 2-(7-Trifluoromethyl-1,1-dioxo-2- phenyl-2,3-dihydro-1H- benzo[d]isothiazol-3-yl)acetic acid methyl ester (52.3) | 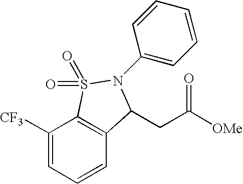 |
| 245 | P | 2-(7-Fluoro-5-methyl-1,1-dioxo- 2-phenyl-2,3-dihydro-1H- benzo[d]isothiazol-3-yl)acetic acid methyl ester (52.4) | 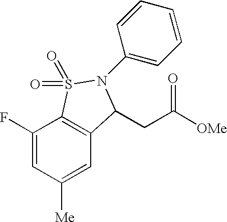 |
| 246 | P | 2-(4-Chloro-5-fluoro-1,1-dioxo- 2-phenyl-2,3-dihydro-1H- benzo[d]isothiazol-3-yl)acetic acid methyl eser (52.5) | 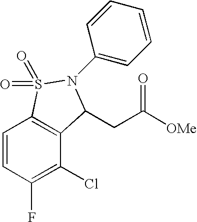 |
| 247 | P | 2-(5-Fluoro-1,1-dioxo-2-phenyl- 2,3-dihydro-1H- benzo[d]isothiazol-3-yl)acetic acid methyl ester (52.6) | 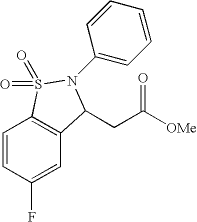 |
| 248 | P | 2-(7-Chloro-1,1-dioxo-2-phenyl- 2,3-dihydro-1H- benzo[d]isothiazol-3-yl)acetic acid methyl ester (52.7) | 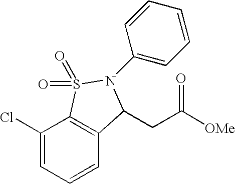 |
| 249 | P | 2-(4,6-Dichloro-1,1-dioxo-2- phenyl-2,3-dihydro-1H- benzo[d]isothiazol-3-yl)acetic acid methyl ester (52.8) | 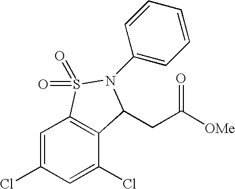 |
| 250 | P | 2-(6-Trifluoromethyl-1,1-dioxo-2- phenyl-2,3-dihydro-1H- benzo[d]isothiazol-3-yl)acetic acid methyl ester (52.9) | 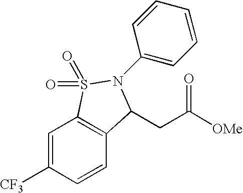 |
| 251 | P | 2-(7-Methoxy-1,1-dioxo-2- phenyl-2,3-dihydro-1H- benzo[d]isothiazol-3-yl)acetic acid methyl ester (52.10) | 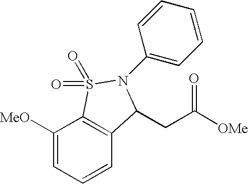 |
| 252 | P | 2-(5-Chloro-7-Methoxy-1,1- dioxo-2-phenyl-2,3-dihydro-1H- benzo[d]isothiazol-3-yl)acetic acid methyl ester (52.11) | 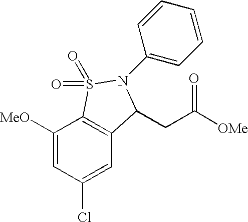 |
| 253 | P | 2-(4-Chloro-5-methoxy-1,1- dioxo-2-phenyl-2,3-dihydro-1H- benzo[d]isothiazol-3-yl)acetic acid methyl ester (52.12) | 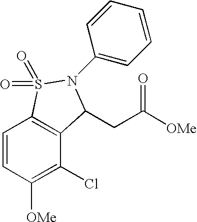 |

Following a method analogous to Method E (for the synthesis of (4)), and using the ester indicated, the following compounds were obtained as crude products.

| | Method + | | |
|---|---|---|---|
| Synthesis | Reagent | Name | Structure |
| 254 | E + (52.1) | 2-(4-Methoxy-1,1-dioxo-2- phenyl-2,3-dihydro-1H- benzo[d]isothiazol-3-yl)acetic acid (53.1) | 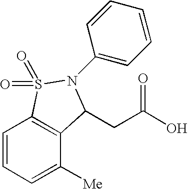 |
| 255 | E + (52.2) | 2-(4-Chloro-1,1-dioxo-2- phenyl-2,3-dihydro-1H- benzo[d]isothiazol-3-yl)acetic acid (53.2) | 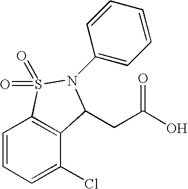 |
| 256 | E + (52.3) | 2-(7-Trifluoromethyl-1,1-dioxo- 2-phenyl-2,3-dihydro-1H- benzo[d]isothiazol-3-yl)acetic acid (53.3) | 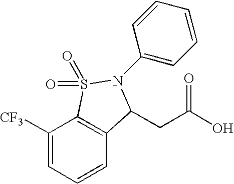 |
| 257 | E + (52.4) | 2-(7-Fluoro-5-methyl-1,1- dioxo-2-phenyl-2,3-dihydro- 1H-benzo[d]isothiazol-3- yl)acetic acid (53.4) | 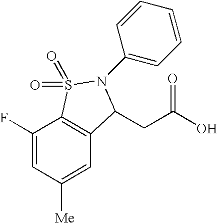 |
| 258 | E + (52.5) | 2-(4-Chloro-5-fluoro-1,1- dioxo-2-phenyl-2,3-dihydro- 1H-benzo[d]isothiazol-3- yl)acetic acid (53.5) | 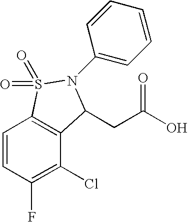 |
| 259 | E + (52.6) | 2-(5-Fluoro-1,1-dioxo-2- phenyl-2,3-dihydro-1H- benzo[d]isothiazol-3-yl)acetic acid (53.6) | 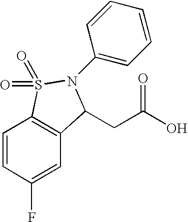 |
| 260 | E + (52.7) | 2-(7-Chloro-1,1-dioxo-2- phenyl-2,3-dihydro-1H- benzo[d]isothiazol-3-yl)acetic acid (53.7) | 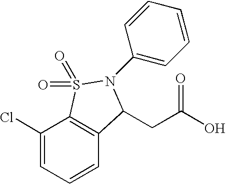 |
| 261 | E + (52.8) | 2-(4,6-Dichloro-1,1-dioxo-2- phenyl-2,3-dihydro-1H- benzo[d]isothiazol-3-yl)acetic acid (53.8) | 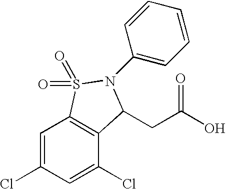 |
| 262 | E + (52.9) | 2-(6-Trifluoromethyl-1,1-dioxo- 2-phenyl-2,3-dihydro-1H- benzo[d]isothiazol-3-yl)acetic acid (53.9) | 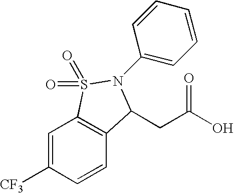 |
| 263 | E + (52.10) | 2-(7-Methoxy-1,1-dioxo-2- phenyl-2,3-dihydro-1H- benzo[d]isothiazol-3-yl)acetic acid (53.10) | 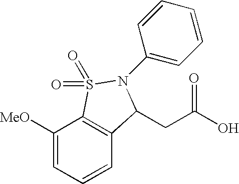 |
| 264 | E + (52.11) | 2-(5-Chloro-7-Methoxy-1,1- dioxo-2-phenyl-2,3-dihydro- 1H-benzo[d]isothiazol-3- yl)acetic acid (53.11) | 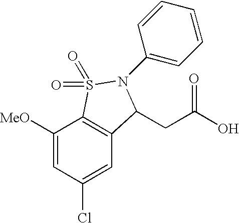 |
| 265 | E + (52.13) | (4-Chloro-5-Methoxy-1,1- dioxo-2-phenyl-2,3-dihydro- 1H-benzo[d]isothiazol-3- yl)acetic acid (53.12) | 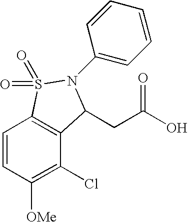 |

Following a method analogous to Method H (for the synthesis of (5)), and using the carboxylic acid indicated, the following compounds were obtained as crude products.

| | Method + | | |
|---|---|---|---|
| Synthesis | Reagent | Name | Structure |
| 266 | H + (53.1) | 2-(4-Methoxy-1,1-dioxo-2- phenyl-2,3-dihydro-1H- benzo[d]isothiazol-3-yl)-N- hydroxyacetamide (54.1) | 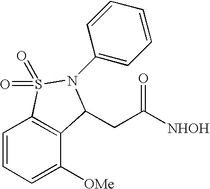 |
| 267 | H + (53.2) | 2-(4-Chloro-1,1-dioxo-2- phenyl-2,3-dihydro-1H- benzo[d]isothiazol-3-yl)-N- hydroxyacetamide (54.2) | 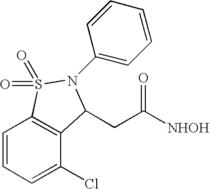 |
| 268 | H + (53.3) | 2-(7-Trifluoromethyl-1,1-dioxo- 2-phenyl-2,3-dihydro-1H- benzo[d]isothiazol-3-yl))-N- hydroxyacetamide (54.3) | 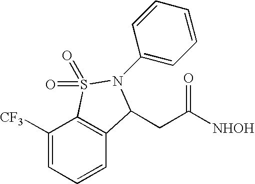 |
| 269 | H + (53.4) | 2-(7-Fluoro-5-methyl-1,1- dioxo-2-phenyl-2,3-dihydro- 1H-benzo[d]isothiazol-3-yl)-N- hydroxyacetamide (54.4) | 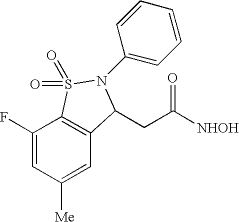 |
| 270 | H + (53.5) | 2-(4-Chloro-5-fluoro-1,1-dioxo- 2-phenyl-2,3-dihydro-1H- benzo[d]isothiazol-3-yl)-N- hydroxyacetamide (54.5) | 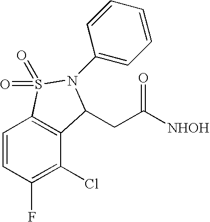 |
| 271 | H + (53.6) | 2-(5-Fluoro-1,1-dioxo-2- phenyl-2,3-dihydro-1H- benzo[d]isothiazol-3-yl)-N- hydroxyacetamide (54.6) | 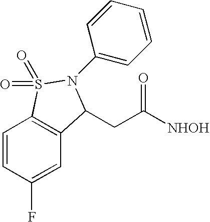 |
| 272 | H + (53.7) | 2-(7-Chloro-1,1-dioxo-2- phenyl-2,3-dihydro-1H- benzo[d]isothiazol-3-yl)-N- hydroxyacetamide (54.7) | 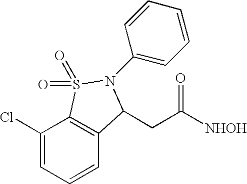 |
| 273 | H + (53.8) | 2-(4,6-Dichloro-1,1-dioxo-2- phenyl-2,3-dihydro-1H- benzo[d]isothiazol-3-yl)-N- hydroxyacetamide (54.8) | 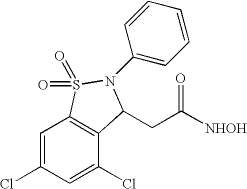 |
| 274 | H + (53.9) | 2-(6-Trifluoromethyl-1,1-dioxo-2- phenyl-2,3-dihydro-1H- benzo[d]isothiazol-3-yl)-N- hydroxyacetamide (54.9) | 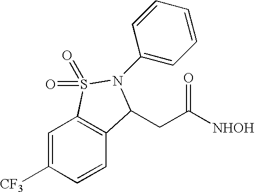 |
| 275 | H + (53.10) | (7-Methoxy-1,1-dioxo-2- phenyl-2,3-dihydro-1H- benzo[d]isothiazol-3-yl)-N- hydroxyacetamide (54.10) | 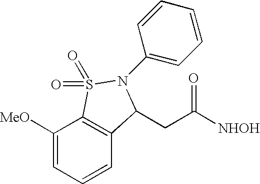 |
| 276 | H + (53.11) | (5-Chloro-7-Methoxy-1,1- dioxo-2-phenyl-2,3-dihydro- 1H-benzo[d]isothiazol-3-yl)-N- hydroxyacetamide (54.11) | 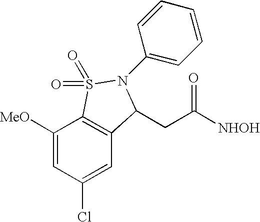 |
| 277 | H + (53.12) | (4-Chloro-5-Methoxy-1,1- dioxo-2-phenyl-2,3-dihydro- 1H-benzo[d]isothiazol-3-yl)-N- hydroxyacetamide (54.12) | 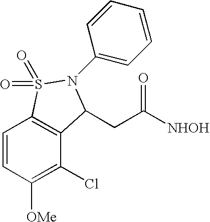 |

Compound (54.1): Yield 64%, melting point 188-190° C., ¹H-NMR (DMSO-d6, TMS) δ: 2.4-2.6 (2H, overlapped with DMSO); 3.90 (3H, s); 5.66 (1H, t, 5.0 Hz); 7.23-7.46 (7H, m); 7.66 (1H, t 8.0 Hz); 8.66 (1H, s) and 10.29 ppm (1H, s).

Compound (54.2): Yield 54%, melting point 204-206° C., ¹H-NMR (DMSO-d6, TMS) δ: 2.63 (1H, dd, 15.4 Hz, 4.4 Hz); 2.75 (1H, dd, 15.4 Hz, 5.9 Hz); 5.81 (1H, t, 4.4 Hz); 7.27-7.51 (5H, m); 7.71 (1H, t, 7.7 Hz); 7.88 (1H, d, 7.3 Hz); 7.95 (1H, d, 7.3 Hz); 8.70 (1H, s) and 10.37 ppm (1H, s).

Compound (54.3): Yield 48%, melting point 187-189° C., ¹H-NMR (DMSO-d6, TMS) δ: 2.3-2.5 (1H, m, overlapped with DMSO); 2.70 (1H, dd, 4.4 and 14.7 Hz); 5.74 (1H, dd, 4.4 Hz and 8.1 Hz); 7.33-7.56 (5H, m); 7.95-8.14 (3H, m); 8.90 (1H, s) and 10.47 ppm (1H, s).

Compound (54.4): Yield 37%, amorphous powder, ¹H-NMR (DMSO-d6, TMS) δ: 2.34 (1H, dd, 8.1 and 14.7 Hz, partly overlapped with DMSO); 2.45 (3H, s, overlapped with DMSO); 2.61 (1H, dd, 4.4 and 8.1 Hz, partly overlapped with DMSO); 5.64 (1H, dd, 3.7 Hz and 8.8 Hz); 7.2-7.6 (7H, m); 8.90 (1H, s) and 10.46 ppm (1H, s). MS: 350.9 (M+).

Compound (54.5): Yield 50%, amorphous powder, ¹H-NMR (DMSO-d6, TMS) δ: 2.6-2.9 (2H, m, partly overlapped with DMSO); 5.82 (1H, t, 4.4 Hz); 7.2-7.5 (5H, m); 7.7-7.9 (1H, m); 8.0-8.2 (1H, m); 8.71 (1H, d, 1.5 Hz) and 10.37 ppm (1H, d, 1.5 Hz). MS: 370.9 (M+).

Compound (54.6): Yield 80%, amorphous powder, ¹H-NMR (DMSO-d6, TMS) δ: 2.3-2.8 (2H, m, partly overlapped with DMSO); 5.64 (1H, dd, 3.7 Hz and 8.1 Hz); 7.2-7.4 (1H, m); 7.4-7.7 (7H, m); 8.08 (1H, dd, 5.1 Hz and 8.8 Hz); 8.90 (1H, s) and 10.47 ppm (1H, s). MS: 337.0 (M+).

Compound (54.7): Yield 99%, melting point: >188° C. (dec.), ¹H-NMR (DMSO-d6, TMS) δ: 2.33 (1H, dd, 8.1 Hz and 14.7 Hz); 2.64 (1H, dd, 3.7 Hz, 14.7 Hz); 5.67 (1H, dd, 4.4 Hz and 8.1 Hz); 7.3-7.9 (8H, m); 8.88 (1H, s) and 10.46 ppm (1H, s). MS: 352.9 (M+).

Compound (54.8): Yield 60%, melting point: >>150° C. (dec.), ¹H-NMR (DMSO-d6, TMS) δ: 2.4-2.7 (1H, m, partly overlapped with DMSO); 2.79 (1H, dd, 16.1 Hz and 5.1 Hz); 5.77 (1H, t, 4.4 Hz); 7.2-7.4 (1H, m); 7.46 (4H, m); 8.08 (1H, d, 2.0 Hz); 8.23 (1H, d, 2.0 Hz); 8.69 (1H, s) and 10.36 ppm (1H, s). MS: 386.8 (M+).

Compound (54.9): Yield 60%, amorphous powder ¹H-NMR (DMSO-d6, TMS) δ: 2.3-2.8 (2H, m, overlapped with DMSO); 5.79 (1H, dd, 3.9 Hz and 7.8 Hz); 7.3-7.6 (5H, m); 7.88 (1H, d, 7.8 Hz); 8.19 (1H, d, 7.8 Hz); 8.52 (1H, s); 8.89 (1H, s) and 10.45 ppm (1H, s). MS: 387.0 (M+).

Compound (54.10): Yield 40%, melting point: >161° C. (dec.), ¹H-NMR (DMSO-d6, TMS) δ: 2.2-2.7 (2H, m, overlapped with DMSO); 3.95 (3H, s); 5.59 (1H, dd, 2.9 Hz and 8.8 Hz); 7.10 (1H, d, 6.7 Hz); 7.2-7.6 (8H, m); 7.72 (1H, t, 8.8 Hz); 8.90 (1H, s) and 10.49 ppm (1H, s). MS: 348.9 (M+).

Compound (54.11): Yield 65%, melting point>170° C. (dec.) ¹H-NMR (DMSO-d6, TMS) δ: 2.2-2.7 (2H, m, partly overlapped with DMSO); 3.98 (3H, s); 5.59 (1H, dd, 3.7 Hz and 8.1 Hz); 7.2-7.6 (7H, m); 8.90 (1H, d, 1.5 Hz) and 10.46 ppm (1H, d, 1.5 Hz). MS: 382.8 (M+).

Compound (54.12): Yield 42%, melting point 184-188° C. (dec.), ¹H-NMR (DMSO-d6, TMS) δ: 2.5-2.7 (2H, m, partly overlapped with DMSO); 3.99 (3H, s); 5.74 (1H, t, 4.4 Hz), 7.2-7.6 (6H, m); 7.7-7.9 (1H, m); 7.94 (1H, d, 8.1 Hz); 8.68 (1H, s) and 10.37 ppm (1H, s). MS: 382.9 (M+).

### Synthesis 278

### 3-(1,1-Dioxo-2-phenyl-2,3-dihydro-1H-benzo[d]isothiazol-3-yl)propionitrile (55)

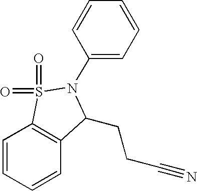

A solution of ester (3.1) (2.9 g 9.14 mmol) in THF (70 mL) was cooled in an ice bath and to this LiAlH₄ (1.04 g, 27.4 mmol) was added in several portions. The mixture was allowed to reach room temperature and stirred for additional 1 h. The residual LiAlH₄ was destroyed by dropwise addition of water until the formation of a gel. Saturated aqueous potassium sodium tartrate was added (100 mL) and the resulting suspension extracted with EtOAc (300 mL). The organic phase was washed with brine (100 mL) and dried over Na₂SO₄. The solution was filtered and evaporated to give intermediate alcohol. The intermediate obtained (1.16 g) was dissolved in DCM (70 mL) and to this PCl₅ (0.92 g, 4.4 mmol) was added. The mixture was stirred at room temperature for 1 h washed with saturated aqueous NaHCO₃ (100 mL). The organic phase was evaporated and the residue purified by flash chromatography on silica gel eluting with a mixture of light petroleum ether and EtOAc (1:1) to give an intermediate chloride (610 mg). This was dissolved in DMFA (15 mL) and KCN (258 mg, 3.96 mmol) was added. The mixture was stirred at 50° C. for 20 h and diluted with water (100 ml). The product was taken in EtOAc (100 mL) and the organic phase was washed with brine (100 mL). The solution was dried over Na₂SO₄, filtered and evaporated. The residue was purified by flash chromatography on silica gel eluting with a mixture of light petroleum ether and EtOAc (1:1) to give the title compound (55) (450 mg).

### Synthesis 279

### 3-(1,1-Dioxo-2-phenyl-2,3-dihydro-1H-benzo[d]isothiazol-3-yl)propionic acid (56)

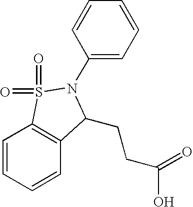

To a solution of nitrile (55) (136 mg, 0.46 mmol) in dioxane (7 mL) added was aqueous concentrated HCl (1.2 mL, 14.4 mmol). The mixture was heated at 115° C. for 72 h and evaporated. The residue was purified by flash chromatography on silica gel eluting with a mixture of light petroleum ether and EtOAc (1:10) to give the title compound (56) (138 mg).

### Synthesis 280

### 3-(1,1-Dioxo-2-phenyl-2,3-dihydro-1H-benzo[d]isothiazol-3-yl)-N-hydroxypropionamide (57)

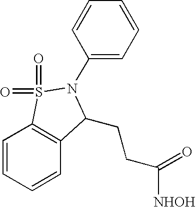

Following a method analogous to Method G (for the synthesis of (5)) from carboxylic acid (56), the title compound was obtained. Yield 63%, viscous oil, ¹H-NMR (DMSO-d6, TMS) δ: 1.38-1.58 (m, 1H); 1.76-1.96 (m, 1H); 2.03-2.35 (m, 2H); 5.68 (t, J=3.1 Hz, 1H); 7.28-7.41 (m, 1H); 7.45-7.57 (m, 4H); 7.65-7.90 (m, 3H); 7.98 (d, J=7.7 Hz, 1H); 8.62 (s, 1H); 10.29 ppm (s, 1H).

### Synthesis 281

### 2-Methyl-N-phenylbenzenesulfonamide (59.1)

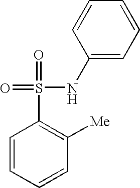

Following a method analogous to Method N (for the synthesis of compounds 50)) from sulphonylchloride (58.1) and aniline (2.1), the title compound was obtained as a crude product.

### Synthesis 282

### 2-Methyl-3-chloro-N-phenylbenzenesulfonamide (59.2)

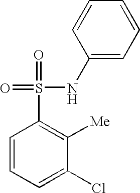

Following a method analogous to Method N (for the synthesis of compounds 50)) from sulphonylchloride (58.2) and aniline (2.1), the title compound was obtained as a crude product

### 1,1-Dioxo-2-phenyl-2,3-dihydro-1H-benzo[d]isothiazole-3-carboxylic acid tert-butyl esters (60.1) and (60. 2)

Method Q: A solution of sulphonamide (58) (2.8 mmol), Boc₂O (1.2 g, 5.54 mmol) and DMAP (338 mg, 2.8 mmol) in THF (35 ml) was stirred at room temperature overnight. The solvent was removed in vacuo and the residue taken into EtOAc (100 mL). The organic phase was washed with 10% aqueous HCl, saturated aqueous NaHCO₃ and brine. The extract was dried over Na₂SO₄, filtered and evaporated. The residue was purified by flash chromatography on silica gel eluting with a mixture of light petroleum ether and EtOAc (4:1). Intermediate compound (2.0 mmol) was dissolved in THF and to this TMEDA (0.66 mL, 4.4 mmol) was added and the mixture was cooled to −78° C. At this temperature, 1.5 M t-BuLi in hexanes (2.9 mL, 4.4 mmol) was added dropwise and the mixture was stirred at −78° C. for additional 30 min. Dimethylaminosulfonylchloride (0.24 mL, 2.2 mmol) was added and the mixture was stirred at −78° C. for 1 h and then allowed to reach room temperature and stirred overnight. The mixture was diluted with EtOAc and washed with brine. The organic phase was dried over Na₂SO₄, filtered and evaporated The residue was purified by flash chromatography on silica gel eluting with a mixture of light petroleum ether and EtOAc (4:1) to give the title compound (60).

Following a method analogous to Method Q, the following compounds were obtained as a crude product.

| Synthesis | Method | Name | Structure |
|---|---|---|---|
| 283 | Q | 1,1-Dioxo-2-phenyl-2,3-dihydro- 1H-benzo[d]isothiazole-3- carboxylic acid tert-butyl ester (60.1) | 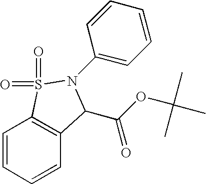 |
| 284 | Q | 4-Chloro-1,1-dioxo-2-phenyl- 2,3-dihydro-1H- benzo[d]isothiazole-3-carboxylic acid tert-butyl ester (60.2) | 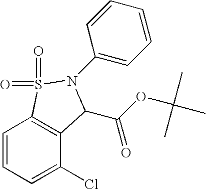 |

Following a method analogous to Method E (for the synthesis of (4)), and using the ester indicated, the following compounds were obtained as crude products.

| Syn- | Method + | | |
|---|---|---|---|
| thesis | Reagent | Name | Structure |
| 285 | E + (60.1) | 1,1-Dioxo-2-phenyl-2,3- dihydro-1H- benzo[d]isothiazole-3- carboxylic acid (61.1) | 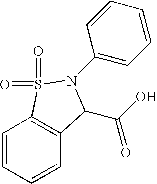 |
| 286 | E + (60.2) | 4-Chloro-1,1-dioxo-2- phenyl- 2,3-dihydro-1H- benzo[d]isothiazole-3- carboxylic acid (61.2) | 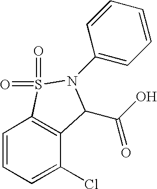 |

Following a method analogous to Method G (for the synthesis of (5)), and using the carboxylic acid indicated, the following compounds were obtained as crude products.

| Syn- | Method + | | |
|---|---|---|---|
| thesis | Reagent | Name | Structure |
| 287 | G + (61.1) | 1,1-Dioxo-2-phenyl- 2,3-dihydro-1H- benzo[d]isothiazole-3- carboxylic acid hydroxyamide (62.1) | 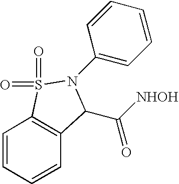 |
| 288 | G + (61.2) | 4-Chloro-1,1-dioxo-2- phenyl- 2,3-dihydro-1H- benzo[d]isothiazole-3- carboxylic acid hydroxyamide (62.2) | 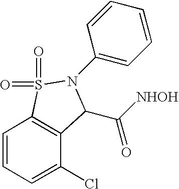 |

Compound (62.1): Yield 95%, melting point 188-191° C., ¹H-NMR (DMSO-d6, TMS) δ: 5.78 (s, 1H); 7.22-7.32 (m, 1H); 7.41-7.51 (m, 4H); 7.62-7.91 (m, 3H); 8.00-8.08 (m, 1H); 9.34 (s, 1H); 11.35 ppm (s, 1H).

Compound (62.2): Yield 43%, melting point 167-170° C., ¹H-NMR (DMSO-d6, TMS) δ: 5.72 (s, 1H); 7.30-7.43 (m, 1H); 7.46-7.55 (m, 4H); 7.78 (dd, J=8.0 and 8.0 Hz, 1H); 7.91 (dd, J=0.9 and 8.0 Hz, 1H); 8.05 (dd, J=0.9 and 8.0 Hz, 1H); 9.35 (d, J=1.0 Hz, 1H); 11.31 ppm (s, 1H).

### Synthesis 289

### (+)- and (−)-1,1-Dioxo-2-phenyl-2,3-dihydro-1H-benzo[d]isothiazol-3-yl)-N-hydroxy-acetamides (+)-(S)-(5A) and (−)-(R)(5.1)

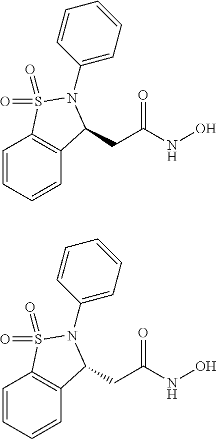

A solution of carboxylic acid (4.1) (606 mg, 2 mmol), (R)-phenylglycinol (274 mg, 2 mmol), HOBt (270 mg, 2 mmol) and EDCl (383 mg, 2 mmol) in DMFA (2 mL) was stirred at room temperature overnight. The solution was partitioned between EtOAc (30 mL) and water (30 mL). Organic phase was separated and washed with brine (20 mL), saturated aqueous NaHCO₃ (20 mL) and brine (20 mL). The solution was dried over Na₂SO₄, filtered and evaporated. Diastereomeric amides were separated by rotating disc chromatography on silica gel, eluting with hexane-ethyl acetate (1:2) to give amide (S,R)-(63.1) as fast eluting diastereomer (structure determined by X-ray spectroscopy) and (R,R)-(63.1) as slow eluting diastereomer.

Each of diastereomeric amides (S,R)-(63.1) and (R,R)-(63.1) was hydrolyzed in 20% aqueous HCl at 80° C. for 7 hours. The product was extracted with CHCl₃ and solution dried over Na₂SO₄. The solution was filtered and evaporated to give carboxylic acids (S)-(4.1) and (R)-(4.1). Carboxylic acids (S)-(4.1) and (R)-(4.1) were converted to hydroxamic acids (+)-(S)-(5.1) ([α]_{D}²⁰=+80° (c=1, acetone)) and (−)-(R)-(5.1) ([α]_{D}²⁰=−92° (c=1, acetone)), respectively by the general procedure described for the synthesis of racemic hydroxamic acid (5.1) and had ¹H-NMR data identical to that of racemic hydroxamic acid (5.1).

### Synthesis 290

### (+)- and (−)-2-[2-(4-But-2-ynyloxyphenyl)-1,1-dioxo-2,3-dihydro-1H-benzo[d]isothiazol-3-yl]-N-hydroxyacetamide (+)-(5.43) and (−)-(5.43)

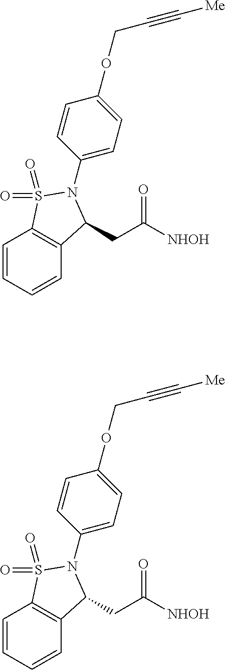

A solution of carboxylic acid (4.43) (1.11 g, 3.0 mmol), (R)-phenylglycinol (0.45 g, 3.3 mmol), HOBt (0.45 g, 3.3 mmol) and EDCl (0.63 g, 3.3 mmol) in DMFA (15 mL) was stirred at room temperature for 24 h. The solution was partitioned between EtOAc (70 mL) and water (100 mL). Organic phase was separated and washed with water (2×100 mL) and brine (100 ml). The solution was dried over Na₂SO₄, filtered and evaporated. Diastereomeric amides were separated by flash chromatography on silica gel, eluting with EtOAc to give amide E1-(63.2) as fast eluting diastereomer (0.67 g) and E2-(63.2) (0.56 g) as slow eluting diastereomer. Each of diastereomeric amides E1-(63.2) (343 mg) and E2-(63.2) (343 mg) was hydrolyzed in a mixture of 1 M aqueous H₂SO₄ (12 mL) and dioxane (12 mL) at reflux temperature for 30 h. Dioxane was removed in vacuo and water (30 ml) was added. The mixture was extracted with EtOAc (50 mL+30 mL) and the combined organic phase was washed with brine (50 mL). The solution was dried over Na₂SO₄, filtered and evaporated to give carboxylic acids E1-(4.43) (242 mg) and E2-(4.43) (269 mg), respectively. Carboxylic acids E144.43) and E2-(4.43) were converted to hydroxamic acids (+)-(5.43) ([α]_{D}²⁰=+71° (c=0.86, acetone)) and (−)-(5.43) ([α]_{D}²⁰=−69° (c=0.84, acetone)), respectively by the general procedure described for the synthesis of racemic hydroxamic acid (5.43) and had ¹H-NMR data identical to that of racemic hydroxamic acid (5.43).

### Synthesis 291

### (+)- and (−)-2-{2-[4-(2-Methylquinolin-4-ylmethoxy)phenyl]-1,1-dioxo-2,3-dihydro-1H-benzo[d]isothiazol-3-yl}-N-hydroxyacetamide (+)-(5.44) and (−)-(5.44)

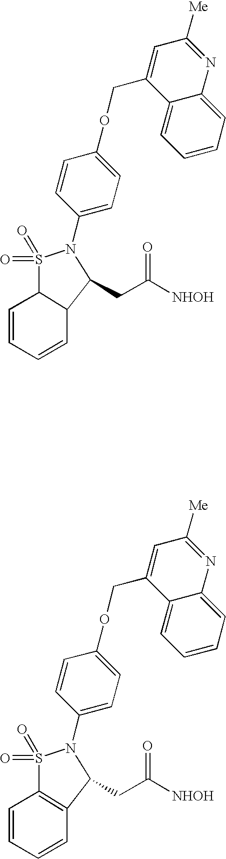

A solution of carboxylic acid (4.44) (1.02 g, 2.0 mmol), (R)-phenylglycinol (0.27 g, 2.0 mmol), HOBt (0.27 g, 2.0 mmol) and EDCl (0.38 g, 2.0 mmol) in DMFA (4 mL) was stirred at room temperature for 15 h. The mixture was diluted with saturated aqueous NaHCO₃ (100 ml) and extracted with EtOAc (2×100 mL). The combined organic phase was separated and washed with brine (100 mL). The solution was dried over Na₂SO₄, filtered and evaporated. Diastereomeric amides were separated by flash chromatography on silica gel, eluting with EtOAc to give amide E1463.3) as fast eluting diastereomer (0.30 g) and E2-(63.3) (0.27 g) as slow eluting diastereomer. Each of diastereomeric amides E1-(63.3) (140 mg) and E2-(63.3) (150 mg) was hydrolyzed in a mixture of 10% aqueous HCl (0.92 mL) and dioxane (0.92 mL) at 110° C. for 2 h. Dioxane was removed in vacuo and water (4 mL) was added. The precipitate was separated by centrifugation and washed with water several times. The residue was dried over P₂O₅ in vacuo to give carboxylic acids E1-(4.44) (0.11 g) and E2-(4.44) (0.10 g), respectively. Carboxylic acid E1-(4.44) (102 mg, 0.2 mmol) was dissolved in dioxane (2 mL) and to this oxalylchloride (0.35 ml, 4 mmol) was added followed by a drop of DMFA. The mixture was heated at 50° C. for 2 h and evaporated. A solution of O-THP hydroxylamine (117 mg, 1 mmol) in DMFA (1 mL) was added to the residue and the resulting mixture stirred at room temperature for 30 min. The mixture was diluted with saturated aqueous NaHCO₃ (10 mL) and extracted with EtOAc (15 mL). The extract was dried over Na₂SO₄, filtered and evaporated. The residue was purified by flash chromatography on silica gel eluting with EtOAc to give O-THP protected hydroxamic acid (64 mg). The intermediate was dissolved in dioxane (1.2 mL) and to this 1 M aqueous HCl (0.6 mL) was added. The mixture was stirred at room temperature overnight and neutralized with aqueous 1 M aqueous NaHCO₃ (10 mL). The precipitate formed was collected on a filter and washed several times with water. The residue was dried over P₂O₅ in vacuo and treated with MeCN (2 mL). The precipitate was collected on a filter and dried over P₂O₅ in vacuo to give hydroxamic acid (+)-5.44 ([α]_{D}²⁰=+62° (c=0.5, DMSO-d₆)) with ¹H-NMR data identical to that of racemic hydroxamic acid (5.44).

Following the procedure described above, carboxylic acid E2-(4.44) (180 mg, 0.35 mmol) was transformed to hydroxamic acid (−)-5.44 ([α]_{D}²⁰=−48° (c=0.5, DMSO-d₆)) with ¹H-NMR data identical to that of racemic hydroxamic acid (5.44).

### Synthesis 292

### O—(N,N-Dimethylthiocarbamoyl)-2-hydroxybenzaldehyde (65)

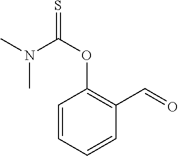

N,N-Dimethylthiocarbamoylchloride (7.42 g, 60 mmol) was added to a solution of salicylaldehyde (64 (4.89 g, 40 mmol) and DABCO (8.96 g, 80 mmol) in DMFA (80 mL). The resulting mixture was stirred at room temperature overnight and poured into water (250 mL). The precipitate was collected on a filter and washed with a large amount of water. After drying over NaOH in vacuo, compound (65) (7.34 g, 87%) was obtained as slightly grey crystals. ¹H-NMR (DMSO-d₆) δ: 3.38 and 3.40 (total 6H, both s); 7.24 (1H, d, 8 Hz); 7.46 (1H, t, 7 Hz); 7.74 (1H, dt, 7 Hz and 2 Hz); 7.86 (1H, dd, 8 Hz and 2 Hz) and 10.00 ppm (1H, s).

### Synthesis 293

### S—(N,N-Dimethylthiocarbamoyl)-2-thiobenzaldehyde (66)

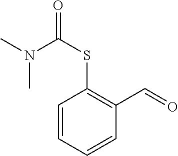

Compound (65) (1.04 g, 5 mmol) was heated in N,N-diethylaniline (1 mL) at 190° C. for 5 h. After cooling, water (10 mL) was added and the mixture acidified with 20% aqueous KHSO₄. The product was taken up in ethyl acetate (20 mL). The organic phase was separated and washed with brine (20 mL). After drying over Na₂SO₄, solvent was removed in vacuo and the residue purified by flash chromatography on silica gel, eluting with a mixture of light petroleum ether and ethyl acetate (2:1) to give compound (66) (495 mg, 48%). ¹H-NMR (DMSO-d₆, TMS) δ: 2.91 (3H, br s) and 3.09 (3H, br s); 7.5-7.8 (3H, m); 7.8-8.0 (2H, m); and 10.14 ppm (1H, s).

### Synthesis 294

### S-Benzyl-2-thiobenzaldehyde (67)

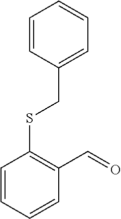

Compound (66) (495 mg, 2.4 mmol) was dissolved in solution of 1M methanolic NaOMe (10 mL). The mixture was stirred overnight at room temperature and to this added was benzylbromide (0.35 mL, 2.9 mmol). Stirring was continued for 2 h and the mixture poured into ice water (50 mL). The product was taken up into CH₂Cl₂ (3×20 mL). The combined organic phase was washed with brine and dried over Na₂SO₄. The solution was filtered and evaporated to give aldehyde dimethylacetal. This was dissolved in a mixture of dioxane (2 mL) and 1 N aqueous HCl (1 mL) and stirred for 2 hours 30 minutes at room temperature Water (20 mL) was added and the mixture extracted with EtOAc (30 mL). Organic phase was separated, washed with brine (20 mL) and dried over Na₂SO₄. The solution was filtered and evaporated to give (67) (417 mg, 76%) as an oil. ¹H-NMR (DMSO-d₆, TMS) δ: 4.26 (2H, s); 7.2-7.5 (6H, m); 7.6-7.7 (2H, m); 7.87 (1H, d, 8 Hz) and 10.10 ppm (1H, s).

### Synthesis 295

### (E)-3-(2-Benzylsulfanylphenyl)acrylic acid methyl ester (68)

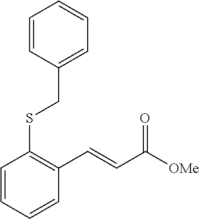

A solution of aldehyde (67) (1.24 g, 5.5 mmol) and methyl (triphenylphoshoranylidene) acetate (1.93 g, 5.8 mmol) in CH₂Cl₂ (30 mL) was stirred at room temperature for 2 hours. Silica gel (3 spoonfuls) was added and the solvent evaporated. The residue was poured onto short silica gel column and the product eluted with a mixture of hexane and EtOAc (10:1) to give ester (68) as colorless crystals (985 mg, 63%). ¹H-NMR (CDCl₃, TMS) δ: 3.81 (3H, s); 4.03 (2H, s); 6.32 (1H, d, 15 Hz); 7.2-7.7 (9H, m) and 8.20 ppm (1H, d, 15 Hz).

### Synthesis 296

### (E)-3-(2-Phenylmethanesulfonylphenyl)acrylic acid methyl ester (69)

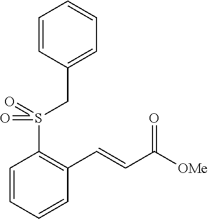

70% MCPBA (1.84 g, 7.5 mmol) was added to a solution of compound (68) (853 mg, 3 mmol) in CH₂Cl₂ (30 mL). The mixture was stirred at room temperature for 1 hour and additional CH₂Cl₂ (30 mL) was added. Organic phase was washed with saturated aqueous Na₂S₂O₃ (20 mL) and saturated aqueous NaHCO₃. The solution was dried over Na₂SO₄ and evaporated. The residue was crystallized from a mixture of hexane and EtOAc (4:1) to give compound (69) (613 mg, 65%) as colorless crystals.

¹H-NMR (DMSO-d₆, TMS) δ: 3.73 (3H, s); 4.61 (2H, s); 6.46 (1H, d, 16 Hz); 7.0-8.0 (9H, m) and 8.17 ppm (1H, d, 16 Hz).

### Synthesis 297

### 2-(1,1-Dioxo-2-phenyl-2,3-dihydro-1H-benzo[b]thiophen-3-yl)acetic acid methyl ester (70)

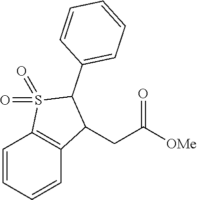

1 M aqueous NaHCO₃ (0.64 mL) was added to a solution of compound (69) (0.32 mmol, 100 mg) in dioxane (1.2 mL). The mixture was refluxed for 45 minutes and evaporated. The residue was partitioned between EtOAc (20 mL) and water (20 mL). The organic phase was separated and washed with brine (20 mL). The solution was dried over Na₂SO₄ filtered and evaporated. The residue was treated with hexane and filtered to give compound (70) (78 mg, 78%) as colorless crystals. ¹H-NMR (DMSO-d₆, TMS) δ: 2.84 (1H, dd, 16 Hz and 6 Hz); 3.02 (1H, dd, 16 Hz and 6 Hz); 3.40 (3H, s); 4.15 (1H, m); 4.88 (1H, d, 9 Hz); 7.45 (5H, s) and 7.5-7.8 ppm (4H, m).

### Synthesis 298

### 2-(1,1-Dioxo-2-phenyl-2,3-dihydro-1H-benzo[b]thiophen-3-yl)acetic acid (71)

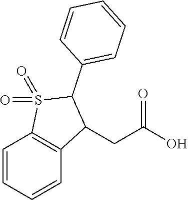

A solution of ester (70) (175 mg, 0.55 mmol) in a mixture of dioxane (3.3 mL) and concentrated aqueous HCl (1.1 mL) was stirred in room temperature for 2 days. Solvents were evaporated and replaced with fresh dioxane (3.3 mL) and concentrated aqueous HCl (3.3 mL). Stirring was continued for additional 2 days, until complete disappearance of starting material. Solvents were removed in vacuo and the residue portioned between EtOAc (30 mL) and saturated aqueous NaHCO₃ (30 mL). Aqueous phase was separated and acidified with concentrated aqueous HCl. The product was taken up into EtOAc (30 mL), organic phase separated and washed with brine (20 mL). The solution was dried over Na₂SO₄, filtered and evaporated to give compound (71) (120 mg, 72%). ¹H-NMR (DMSO-d₆, TMS) δ: 2.79 (2H, m); 4.10 (1H, m); 4.90 (1H, d, 9 Hz); 7.45 (5H, s) and 7.5-7.8 ppm (4H, m).

### Synthesis 299

### 2-(1,1-Dioxo-2-phenyl-2,3-dihydro-1H-benzo[b]thiophen-3-yl)-N-hydroxyacetamide (72)

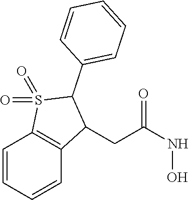

I>

To a solution of carboxylic acid (71) (120 mg, 0.4 mmol) in CH₂Cl₂ (2 mL) added was oxalylchloride (0.17 mL, 2 mmol) and a drop of DMFA. The resulting mixture was stirred at room temperature and evaporated. To the residue, added was a mixture prepared by dissolving hydroxylamine hydrochloride (347 mg, 5 mmol) in a mixture of THF (5 mL) and 1M aqueous NaHCO₃ (5 mL). The resulting suspension was stirred for 1 hour and partitioned between EtOAc (20 mL) and water (20 mL). The organic phase was separated and washed with saturated NaHCO₃ (10 mL) and brine (10 mL). The solution was dried over Na₂SO₄, filtered and evaporated. The residue was crystallized from EtOAc to give hydroxamic acid (72) (22 mg, 17%) as colorless crystals with melting point 113-114° C. ¹H-NMR (DMSO-d₆, TMS) δ: 2.2-2.7 (2H, m, overlapped with DMSO); 4.13 (1H, m); 5.05 (1H, d, 8 Hz); 7.3-7.9 (9H, m); 8.9 (1H, br s) and 10.6 ppm (1H, br s).

### Synthesis 300

### 2-Iodo-pyridine-3-sulfonic acid phenylamide (74)

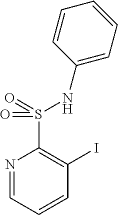

Following a method analogous to Method L (for the synthesis of compounds (45)) from sulphonamide (73), the title compound was obtained as a crude product.

### Synthesis 301

### (1,1-Dioxo-2-phenyl-2,3-dihydro-1H-isothiazolo[4,5-b]pyridin-3-yl)acetic acid methyl ester (75)

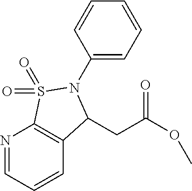

Following a method analogous to Method M (for the synthesis of compounds (46)) from iodide (74), the title compound was obtained as a crude product.

### Synthesis 302

(1,1-Dioxo-2-phenyl-2,3-dihydro-1H-isothiazolo[4,5-b]pyridin-3-yl)acetic acid (76)

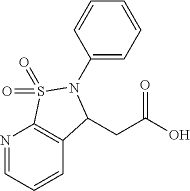

Following a method analogous to Method E (for the synthesis of (4)) from ester (75), the title compound was obtained as a crude product.

### Synthesis 303

### (1,1-Dioxo-2-phenyl-2,3-dihydro-1H-isothiazolo[4,5-b]pyridin-3-yl)acetic acid (77)

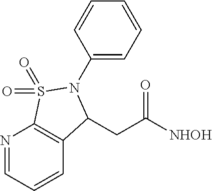

Following a method analogous to Method G (for the synthesis of (5)) from carboxylic acid

(76), the title compound was obtained. Yield 30%, melting point: 123-128° C., ¹H-NMR (DMSO-d6, TMS) δ: 2.37 (1H, dd overlapped with DMSO, J=14.98.4 Hz); 2.71 (1H, dd overlapped with DMSO, J=14.94.0 Hz); 5.87-5.65 (1H, m); 7.45-7.31 (1H, m); 7.67-7.45 (4H, m); 7.95-7.79 (1H, m) 8.17 (1H, d, J=7.7 Hz); 9.02-8.77 (2H, m) and 10.48 ppm (1H, s).

### Biological Methods

### TACE Assay

The activity of the compounds as TACE inhibitors was determined using a commercially available peptide substrate (M-2255, Bachem UK Ltd, St. Helens, UK) and recombinant TACE enzyme (930-ADB, R and D Systems, Abingdon, UK). Human recombinant TACE enzyme (5 ng/30 μL) was incubated for 3.5 hour at 37° C. in assay buffer (25 mM Tris.HCl, 2.5 μM ZnCl₂, 0.005% Brij 35, pH 8.0) with 5 μM substrate in the presence of test compound (TACE inhibitor). The extent of TACE activity was determined by measurement of the fluorescence (excitation 355 nm, emission 460 nm).

Percent activity (% activity) for each test compound was calculated as:

% activity={(S^{C}−B)/(S^{o}−B)}×100

wherein S^{C} denotes signal measured in the presence of enzyme and the compound being tested, S^{o} denotes signal measured in the presence of enzyme but in the absence of the compound being tested, and B denotes the background signal measured in the absence of both enzyme and compound being tested. The IC₅₀ corresponds to the concentration which achieves 50% activity.

### Selectivity Assay: HDAC Activity: Fluorescent Assay

Alternatively, the activity of the compounds as HDAC inhibitors was determined using a commercially available fluorescent assay kit (Fluor de Lys™, BioMol Research Labs, Inc., Plymouth Meeting, USA). HeLa extract was incubated for 1 hour at 37° C. in assay buffer (25 mM HEPES, 137 mM NaCl, 2.7 mM KCl, 1 mM MgCl₂, pH 8.0) with 15 μM acetylated substrate in the presence of test compound (HDAC inhibitor). The extent of deacetylation was determined by the addition of 50 μL of a 1-in-500 dilution of Developer, and subsequent measurement of the fluorescence (excitation 355 nm, emission 460 nm), according to the instructions provided with the kit.

### HeLa Cell Extract

The HeLa cell extract was made from HeLa cells (ATCC Ref. No. CCL-2) by freeze-thawing three times in 60 mM Tris.HCl, pH 8.0, 450 mM NaCl, 30% glycerol. Two cell volumes of extraction buffer were used, and particulate material was centrifuged out (20,800 g, 4° C., 10 minutes). The supernatant extract having deacetylase activity was aliquoted and frozen for storage.

Percent activity (% activity) for each test compound was calculated as:

% activity={(S^{C}−B)/(S^{o}−B)}×100

wherein S^{C} denotes signal measured in the presence of enzyme and the compound being tested, S^{o} denotes signal measured in the presence of enzyme but in the absence of the compound being tested, and B denotes the background signal measured in the absence of both enzyme and compound being tested. The IC₅₀ corresponds to the concentration which achieves 50% activity.

Measurement of cell viability in the presence of increasing concentration of test compound at different time points is used to assess both cytotoxicity and the effect of the compound on cell proliferation.

### Biological Data

IC₅₀ data for several compounds of the present invention, as determined using the TACE assay, as described above, are shown in the following tables.

TACE and HDAC Inhibition Data

| | | Av. TACE Inhibition | Av. HDAC Inhibition |
|---|---|---|---|
| ID No. | Cmpd No. | EC₅₀ (μM) | EC₅₀ (μM) |
| IX-001 | 5.1 | 0.242 | 10%@100 |
| IX-002 | (+)-(S)-5.1 | 4.54 | 43%@100 |
| IX-003 | (−)-(R)-5.1 | 0.27 | 44%@100 |
| IX-004 | 5.2 | 1.1 | 32%@100 |
| IX-005 | 5.3 | 6.71 | |
| IX-006 | 5.4 | 3.39 | |
| IX-007 | 5.5 | 5.52 | |
| IX-008 | 5.6 | 50%@100 | |
| IX-009 | 5.7 | 52%@100 | 24%@100 |
| IX-010 | 5.8 | 0.34 | 17%@100 |
| IX-011 | 5.9 | 86%@100 | |
| IX-012 | 5.1 | 0.51 | 11%@100 |
| IX-013 | 5.11 | 93%@100 | |
| IX-014 | 5.12 | 81%@100 | |
| IX-015 | 5.13 | 54%@100 | |
| IX-016 | 5.14 | 1.33 | |
| IX-017 | 5.15 | 97%@100 | |
| IX-018 | 5.16 | 0.62 | |
| IX-019 | 5.17 | 55%@100 | |
| IX-020 | 5.18 | 0.42 | −2%@100 |
| IX-021 | 5.19 | 6.67 | |
| IX-022 | 5.2 | 2.16 | |
| IX-023 | 5.21 | 1 | |
| IX-024 | 5.22 | 50%@100 | |
| IX-025 | 5.23 | 3.14 | |
| IX-026 | 5.24 | 0.55 | |
| IX-027 | 5.25 | 0.27 | |
| IX-028 | 5.26 | 2.19 | |
| IX-029 | 5.27 | 10.76 | |
| IX-030 | 5.28 | 54%@100 | |
| IX-031 | 5.29 | 70%@100 | |
| IX-032 | 5.3 | 10.08 | |
| IX-033 | 5.31 | 7.42 | |
| IX-034 | 5.32 | 36%@100 | |
| IX-035 | 5.33 | 0.47 | |
| IX-036 | 5.34 | 0.27 | −4%@100 |
| IX-037 | 5.35 | 3.78 | |
| IX-038 | 5.36 | 1.41 | |
| IX-039 | 5.37 | 28%@100 | |
| IX-040 | 5.38 | 28%@100 | |
| IX-041 | 5.39 | 6.01 | |
| IX-042 | 5.4 | 7.22 | |
| IX-043 | 5.41 | 79 | |
| IX-044 | 5.42 | 0@2 | |
| IX-045 | 5.43 | 0.0063 | 38%@100 |
| IX-046 | (+)-5.43 | 0.27 | |
| IX-047 | (−)-5.43 | 0.0031 | |
| IX-048 | 5.44 | 0.00038 | 19%@100 |
| IX-049 | (+)-5.44 | 0.06 | |
| IX-050 | (−)-5.44 | <0.0001 | |
| IX-051 | 5.45 | 81%@100 | |
| IX-052 | 5.46 | 53%@100 | |
| IX-053 | 5.47 | 1.46 | |
| IX-054 | 5.48 | 1.38 | |
| IX-055 | 5.49 | 3.37 | |
| IX-056 | 5.5 | 4.98 | |
| IX-057 | 5.51 | 0.08 | |
| IX-058 | 5.52 | 0.037 | |
| IX-059 | 5.53 | 0.058 | |
| IX-060 | 5.54 | 0.48 | |
| IX-061 | 5.55 | 1.26 | |
| IX-062 | 5.56 | 0.0085 | |
| IX-063 | 5.57 | <0.0001 | |
| IX-064 | 5.58 | <0.0001 | |
| IX-065 | 5.59 | 0.011 | |
| IX-066 | 5.6 | 0.41 | |
| IX-067 | 5.61 | 0.52 | |
| IX-068 | 5.62 | 0.31 | 44%@100 |
| IX-069 | 5.63 | 3.38 | |
| IX-070 | 5.64 | 6.88 | |
| IX-071 | 5.65 | 10%@2 | |
| IX-072 | 5.66 | 15%@2 | |
| IX-073 | 5.67 | 32%@2 | |
| IX-074 | 5.68 | 10%@2 | |
| IX-075 | 24 | 64%@100 | 2%@100 |
| IX-076 | 29 | 0.23 | |
| IX-077 | 36 | 0.12 | |
| IX-078 | 39 | 0.41 | 23.7 |
| IX-079 | 43 | 4%@2 | |
| IX-080 | 48.1 | 0.94 | |
| IX-081 | 48.2 | 0.72 | |
| IX-082 | 54.1 | 35%@2 | |
| IX-083 | 54.2 | 6.39 | |
| IX-084 | 54.3 | 0%@2 | |
| IX-085 | 54.4 | 57%@2 | |
| IX-086 | 54.5 | 20%@2 | |
| IX-087 | 54.6 | 1 | |
| IX-088 | 54.7 | 34@2 | |
| IX-089 | 54.8 | 0@2 | |
| IX-090 | 54.9 | 10@2 | |
| IX-091 | 54.1 | 13@2 | |
| IX-092 | 54.11 | 23@2 | |
| IX-093 | 54.12 | 29@2 | |
| IX-094 | 57 | 26%@100 | |
| IX-095 | 62.1 | 38%@100 | |
| IX-096 | 62.2 | 27%@100 | |
| IX-097 | 72 | 1.07 | 20%@100 |
| IX-098 | 77 | 3.85 | |
| IX-099 | 4.1 | 9%@100 | |
| IX-100 | 4.43 | 50%@100 | |
| IX-101 | 4.44 | 8.59 | |

Several compounds were tested at a single concentration against a panel of matrix metalloproteases including angiotensin converting enzyme (ACE) and the percentage inhibition determined. The data are summarised in the following table.

Inhibition of Matrix Metalloproteases (MMPs)

| | % Inhibition @ 5 μM | | |
|---|---|---|---|
| Enzyme | IX-001 | IX-045 | IX-048 |
| MMP-1 | 0 | −4 | 6 |
| MMP-2 | 19 | 10 | −3 |
| MMP-3 | 1 | 11 | 34 |
| MMP-7 | 0 | −5 | 31 |
| MMP-8 | 30 | 21 | 13 |
| MMP-9 | 19 | −2 | −8 |
| MMP-10 | 0 | 6 | 9 |
| MMP-12 | 56 | 37 | 49 |
| MMP-13 | 24 | −12 | −10 |
| MMP-14 | 2 | 0 | 4 |
| ACE | 6 | −8 | −4 |

These data show that within the class of BCSA compounds described herein, it is possible to achieve exquisite selectivity of TACE inhibitors over other related Zn-metalloproteases such as histone deacetylases or matrix metalloproteases. Thus, it is expected to be possible to use these TACE inhibitors without the side effects arising from HDAC or MMP inhibition.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
Beutler, B., et al., 1988, “Tumor necrosis, cachexia, shock and inflammation: a common mediator”, Ann. Rev. Biochem., Vol. 57, pp. 505-518.
Boutros, A., et al., 2000, “4-Quinolylmethyl and 1-Naphthylmethyl as Benzyl-type Protecting Groups of Carboxylic Acids Removable by Homogeneous Palladium-Catalyzed Hydrogenolysis”, Tetrahedron, Vol. 56, pp. 2239-2246.
Breese, E. J., et al., 1994, “Tumor necrosis factor alpha-producing cells in the intestinal mucosa of children with inflammatory bowel disease”, Gastroenterology, Vol. 106, No. 6, pp. 1455-1466.
Brummond, K. M., et al., 2004, “Rhodium(I)-Catalyzed Ene-Allene Carbocyclization Strategy for the Formation of Azepines and Oxepines”, Org. Lett., Vol. 6, pp. 2161-2164.
Buchman, E. R., et al., 1946, “Potential Antimalarials. (Chloroquinolyl-4)-α-piperidylcarbinols”, J. Am. Chem. Soc., Vol. 68, pp. 2962-2963.
Camussi et al., 1998, “The future role of anti-tumour necrosis factor (TNF) products in the treatment of rheumatoid arthritis”, Drugs, Vol. 55, No. 5, pp. 613-620.
Carrigan, C. N., et al., 2002, “Synthesis and in Vitro Pharmacology of Substituted Quinoline-2,4-Dicarboxylic Acids as Inhibitors of Glutamate Vesicular Transport (VGLUT)”, J. Med. Chem., Vol. 45, pp. 2260-2276.
Duan, J. J.-W. et al., 2002, “Discovery of γ-Lactam Hydroxamic Acids as Selective Inhibitors of Tumor Necrosis Factor “Converting Enzyme: Design, Synthesis, and Structure-Activity Relationships”, J. Med. Chem., Vol. 45, pp. 4954-4957.
Eberbach, W. et al., 1986, “Intramolecular cycloadditions with azomethine ylides for the synthesis of metacyclophanes”, Tetrahedron Letters, Vol. 27, pp. 4003-4006.
El-Batta, A. et al., 2007, “Wittig Reactions in Water Media Employing Stabilized Ylides with Aldehydes. Synthesis of α-β-Unsaturated Esters from Mixing Aldehydes, α-Bromoesters, and Ph₃P in Aqueous NaHCO₃”, J. Org. Chem., Vol. 72, pp. 5244-5259.
Ferrari, R., et al., 1995, “Tumor necrosis factor soluble receptors in patients with various degrees of congestive heart failure”, Circulation, Vol. 92, No. 6, pp. 1479-1486.
Finn, P. W. et al., 2005, “Novel Sulfonamide Derivatives as Inhibitors of Histone Deacetylase”, Helvetica Chimica Acta, Vol. 88, pp. 1630-1657.
Fisher J F and Mobashery S, 2006, “Recent advances in MMP inhibitor design”, Cancer and Metastasis Reviews, Vol 25 No. 1 pp 115-136.
Goulaouic-Dubois, C., et al., 1995, “Protection of Amines by the Pyridine-2-sulfonyl Group and Its Cleavage under Mild Conditions (Sml2 or Electrolysis)”, J. Org. Chem. Vol. 60, pp. 5969-5972.
Grau, G. E., et al., 1987, “Tumor necrosis factor (cachectin) as an essential mediator in murine cerebral malaria”, Science, Vol. 237(4819), pp. 1210-1212
Gu, Q., et al., 2006, “Inhibition of TNF-alpha reduces myocardial injury and proinflammatory pathways following ischemia-reperfusion in the dog”, J. Cardiovasc. Pharmacol., Vol. 48, No. 6, pp. 320-328.
Hotamisligil, G. S., et al., 1993, “Adipose expression of tumor necrosis factor-alpha direct role in obesity-linked insulin resistance”, Science, Vol. 259, pp. 87-91.
Imashiro, R., et al., 2004, “A Catalytic Asymmetric Synthesis of Chiral Glycidic Acid Derivatives through Chiral Dioxirane-Mediated Catalytic Asymmetric Epoxidation of Cinnamic Acid Derivatives”, J. Org. Chem., Vol. 69, pp. 4216-4226.
Isomaki, P., et al., 1997, “Pro- and anti-inflammatory cytokines in rheumatoid arthritis”, Ann. Med., Vol. 29, pp. 499-507.
Jackson, W. R., et al., 1988, “The Stereochemistry of Organometallic Compounds. XXXII. Hydrocyanation of Derivatives of Amino Alkynes”, Aust. J. Chem., Vol. 41, pp. 1201-1208.
Ksontini, R., et al., 1988, “Revisiting the role of tumor necrosis factor alpha and the response to surgical injury and inflammation”, Arch. Surg., Vol. 133, pp. 558-567.
Le et al, 2005, “Inhibitors of TACE and Caspase-1 as anti-inflammatory drugs”, Curr. Med. Chem., Vol. 12, No. 25, pp. 2963-2977.
Levin, J. I., et al., 1999, U.S. Pat. No. 5,929,097.
MacNeil, S. L., et al., 2001, “Selective Ortho and Benzylic Functionalization of Secondary and Tertiary p-Tolylsulfonamides. Ipso-Bromo Desilylation and Suzuki Cross-Coupling Reactions”, J. Org. Chem., Vol. 66., pp. 3662-3670.
Mahajan, R. P., et al., 2005, “Convenient Microwave Assisted Synthesis of Naturally Occurring Methyl (E)-Cinnamates”, Org. Prep. Proced. Int., Vol. 37, pp. 286-290.
Marshall, J. A., et al., 2000, “Synthesis of □-Hydroxy Ketones and Vinylsilanes from Homopropargylic Alcohols by Intramolecular Hydrosilation”, Org. Lett., Vol. 2, pp. 2173-2176.
Mathison et al., 1988, “Participation of tumor necrosis factor in the mediation of gram-negative bacterial lipopolysaccharide-induced injury in rabbits”, J. Clin. Invest., Vol. 81, pp. 1925-1937.
McDonald, 1997, “Characterization of the mucosal cell-mediated immune response in IL-2 knock-out mice before and after the onset of colitis”, Immunology, Vol. 91, No. 1, pp. 73-80.
Miethke et al., 1992, “T cell mediated lethal shock triggered in mice by the superantigen staphylococcal enterotoxin B: critical role of tumor necrosis factor”, J. Exp. Med., Vol. 175, pp. 91-98.

Ohtani, M., et al., 1993, PCT patent publication number WO 93/12075.
Old, L., 1985, “Tumor necrosis factor (TNF)”, Science, Vol. 230, pp. 630-632.
Owen, D., et al., 2000, PCT patent publication number WO 00/56704.
Packer, M., 1995, “Is tumor necrosis factor an important neurohormonal mechanism in chronic heart failure?”, Circulation, Vol. 92, No. 6, pp. 1379-1382.
Palladino, M. A., et al., 2003, “Anti-TNF-alpha Therapies: The next generation”, Nature Reviews Drug Discovery, Vol. 2, pp. 736-746.
Pallares-Trujillo, J., et al., 1995, “TNF and AIDS: two sides of the same coin?”, Med. Res. Rev., Vol. 15, No. 6, pp. 533-546.
Peterson, P. K., et al., 1992, “Human cytomegalovirus-stimulated peripheral blood mononuclear cells induce HIV-1 replication via a tumor necrosis alpha-mediated mechanism”, J. Clin. Invest., Vol. 89, pp. 574-580.
Ragan, J. A., et al., 2002, “Regioselective Hydroxylation of 2,4-Lutidine: A Practical Synthesis of 4-Hydroxymethyl-2-methylpyridine”, Synthesis, pp. 483-486.
Skretas, G., et al., 2007, “Engineered chimeric enzymes as tools for drug discovery: Generating reliable bacterial screens for the detection, discovery, and assessment of estrogen receptor modulators”, J. Am. Chem. Soc., Vol. 129, pp. 8443-8457.
Takahashi, M. et al., 2003, “Cyclization of N-Aryl-N-tert-butoxycarbonyl-o-toluenesulfonamides to 2,3-Dihydro-1,2-benzisothiazole-1,1-dioxides Accompanied by a Rearrangement of a tert-Butoxycarbonyl Group”, Synth. Comm., Vol. 33, pp. 3397-3404.
Thibault, R. J., et al., 2006, “A Versatile New Monomer Family: Functionalized 4-Vinyl-1,2,3-Triazoles via Click Chemistry”, J. Am. Chem. Soc., Vol. 128, pp. 12084-12085.
Westman, J., et al., 2001, “An Efficient Combination of Microwave Dielectric Heating and the Use of Solid-Supported Triphenylphosphine for Wittig Reactions”, Org. Lett., Vol. 3, pp. 3745-3747.
White, W. L., et al., 1982, “Prostaglandins. 3. Synthetic approaches to 11-deoxyprostaglandins”, J. Org. Chem., Vol. 47, pp. 2379-2387.
Yen, V. Q., et al., 1958, “Fluorinated Isatins and Some of Their Heterocyclic Derivatives” J. Org. Chem., Vol. 23, pp. 1858-1861.

1. A compound selected from compounds of the following formula, and pharmaceutically acceptable salts, thereof: 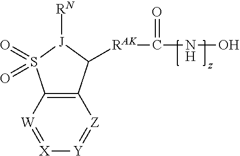
wherein:
W is independently —N═ or —CR^{PW}═;
X is independently —N═ or —CR^{PX}═;
Y is independently —N═ or —CR^{PY}═;
Z is independently —N═ or —CR^{PZ}═;
each of -R^{PW}, -R^{PX}, -R^{PY}, and -R^{PZ}, if present, is independently —H or -R^{RS1};
wherein each -R^{RS1}, if present, is independently a ring substituent;
and wherein:
-J< is independently —N< or —CH<;
and wherein:
—R^{N} is independently —H, -R^{NN}, -R^{NNN}, or -L^{N}-R^{NNN};
wherein:
-L^{N}- is independently saturated aliphatic C₁₋₆alkylene, and is optionally substituted;
—R^{NN} is independently C₁₋₆alkyl, and is optionally substituted; and
—R^{NNN} is independently C₃₋₆cycloalkyl, C₃₋₇heterocyclyl, C₆₋₁₀carboaryl, or
C₅₋₁₀heteroaryl, and is optionally substituted;
and wherein:
-R^{AK}- is independently:
a covalent bond,
-R^{AK2}-, -R^{AK3}-,
R^{AK4}-, -R^{AK1}-R^{AK4}-, -R^{AK4}- R^{AK1}-, -R^{AK1}-R^{AK4}-R^{AK1}-,
-R^{AK5}-, -R^{AK1}-R^{AK5}-, -R^{AK5}-R^{AK1}-, or -R^{AK1}-R^{AK5}-R^{AK1}-;
wherein:
each -R^{AK1}- is independently saturated aliphatic C₁₋₆alkylene, and is optionally substituted;
-R^{AK2}- is independently aliphatic C₂₋₆alkenylene, and is optionally substituted;
-R^{AK3}- is independently aliphatic C₂₋₆alkynylene, and is optionally substituted;
each -R^{AK4}- is independently saturated C₃₋₆cycloalkylene, and is optionally substituted; and
each -R^{AK5}- is independently C₃₋₆cycloalkenylene, and is optionally substituted;
and wherein z is 0 or 1.

2. A compound according to claim 1, wherein:
W is independently —CR^{PW}═,
X is independently —CR^{PK}═,
Y is independently —CR^{PY}═, and
Z is independently —CR^{PZ}═.

3. A compound according to claim 1, wherein:
exactly one or exactly two of W, X, Y, and Z is —N═.

4. A compound according to claim 1, wherein:
exactly one of W, X, Y, and Z is —N═.

5. A compound according to claim 1, wherein:
W is independently —N═,
X is independently —CR^{PX}═,
Y is independently —CR^{PY}═, and
Z is independently —CR^{PZ}═.

6. A compound according to claim 1, wherein:
W is independently —CR^{PW}═,
X is independently —N═,
Y is independently —CR^{PY}═, and
Z is independently —CR^{PZ}═.

7. A compound according to claim 1, wherein:
W is independently —CR^{PW}═,
X is independently —CR^{PX}═,
Y is independently —N═, and
Z is independently —CR^{PZ}═.

8. A compound according to claim 1, wherein:
W is independently —CR^{PW}═,
X is independently —CR^{PX}═,
Y is independently —CR^{PY}═, and
Z is independently —N═.

9. A compound according to claim 1, wherein:
each of -R^{PW}, -R^{PX}, -R^{PY}, and -R^{PZ}, if present, is independently —H.

10. A compound according to any claim 1, wherein z is independently 1.

11. A compound according to claim 1, wherein z is independently 0.

12. A compound according to claim 1, wherein -J< is independently —N<.

13. A compound according to claim 1, wherein -J< is independently —CH<.

14. A compound according to claim 1, wherein -R^{AK}- is independently:
-R^{AK1}-, -R^{AK2}-, -R^{AK3},
-R^{AK4}-, -R^{AK1}-R^{AK4}-, -R^{AK4}-R^{AK1}-, -R^{AK1}-R^{AK4}-R^{AK1}-,
-R^{AK5}-, -R^{AK1}-R^{AK5}-, -R^{AK5}-R^{AK1}-, or -R^{AK1}-R^{AK5}-R^{AK1}—.

15. A compound according to claim 1, wherein -R^{AK}- is independently:
-R^{AK1}, -R^{AK2}-, -R^{AK3}-,
-R^{AK4}, -R^{AK1}-R^{AK4}-, -R^{AK4}-R^{AK1}- , or -R^{AK1}-R^{AK4}-R^{AK1}-.

16. A compound according to claim 1, wherein -R^{AK}- is independently -R^{AK1}, -R^{AK2}-, or -R^{AK3}-.

17. A compound according to claim 1, wherein -R^{AK}- is independently -R^{AK1}- or -R^{AK2}-.

18. A compound according to claim 1, wherein -R^{AK}- is independently -R^{AK1}- .

19. A compound according to claim 1, wherein -R^{AK}- is independently -R^{AK2}-.

20. A compound according to claim 1, wherein -R^{AK}- is independently -R^{AK3}-.

21. A compound according to claim 1, wherein -R^{AK}- is independently -R^{AK1}- or a covalent bond.

22. A compound according to claim 1, wherein -R^{AK}- is independently a covalent bond.

23. A compound according to claim 1, wherein -R^{AK}- is independently: -R^{AK4}-, -R^{AK1}-R^{AK4}-, -R^{AK4}-R^{AK1}l, or -R^{AK1}-R^{AK4}-R^{AK1}-.

24. A compound according to claim 1, wherein -R^{AK}- is independently -R^{AK4}-.

25. A compound according to claim 1, wherein -R^{AK}- is independently -R^{AK1}-R^{AK4}-.

26. A compound according to claim 1, wherein -R^{AK}- is independently -R^{AK4}-R^{AK1}-.

27. A compound according to claim 1, wherein -R^{AK}- is independently -R^{AK1}-R^{AK4}-R^{AK1}-.

28. A compound according to claim 1, wherein each -R^{AK1}-, if present, is independently saturated aliphatic C₁₋₄alkylene; and is optionally substituted.

29. A compound according to claim 1, wherein each -R^{AK1}-, if present, is independently unsubstituted or substituted with one or more substitutents -R^{G1}, wherein each -R^{G1}, if present, is independently —F, —Cl, —Br, —I, —OH, —OR^{A1}, —OCF₃, —C(═O)OH, —C(═O)OR^{A1}, —NH₂, —NHR^{A1}, —NR^{A1}₂, —NR^{A2}R^{A3}, —C(═O)—NH₂, —C(═O)—NHR^{A1}, —C(═O)—NR^{A1}₂, —C(═O)—NR^{A2}R^{A3}, phenyl, or benzyl; wherein each R^{A1} is independently C₁₋₄alkyl, phenyl, or benzyl; and each —NR^{A2}R^{A3} is independently pyrrolidino, piperidino, piperizino, or morpholino, and is independently unsubstituted or substituted with one or more groups selected from C₁₋₃alkyl and —CF₃.

30. A compound according to claim 1, wherein each -R^{AK1}-, if present, is independently unsubstituted or substituted with one or more substitutents —R^{G1}, wherein each —R^{G1}, if present, is independently —F, —Cl, —Br, —I, —OH, —OMe, —OEt, or —OCF₃.

31. A compound according to claim 1, wherein each -R^{AK1}-, if present, is independently unsubstituted.

32. A compound according to claim 1, wherein each -R^{AK1}-, if present, is independently —(CH₂)_{q}—, wherein q is independently 1, 2, 3, 4, 5, or 6.

33. A compound according to claim 1, wherein each -R^{AR1}- , if present, is independently —(CH₂)—, —(CH₂)₂—, —(CH₂)₃—, or —(CH₂)₄—.

34. A compound according to claim 1, wherein each -R^{AK1}-, if present, is independently —(CH₂)—, —(CH₂)₂—, or —(CH₂)₃—.

35. A compound according to claim 1, wherein each -R^{AK1}-, if present, is independently —(CH₂)— or —(CH₂)₂—.

36. A compound according to claim 1, wherein each -R^{AK1}-, if present, is independently —(CH₂)—.

37. A compound according to claim 1, wherein -R^{AK2}-, if present, is independently aliphatic C₂₋₄alkenylene; and is optionally substituted.

38. A compound according to claim 1, wherein -R^{AK2}-, if present, is independently unsubstituted or substituted with one or more substitutents -R^{G1}, wherein each —R^{G1}, if present, is independently —F, —Cl, —Br, —I, —OH, —OR^{A1}, —OCF₃, —C(═O)OH, —C(═O)OR^{A1}, —NH₂, —NHR^{A1}, —NR^{A1}₂, —NR^{A2}R^{A3}, —C(═O)—NH₂, —C(═O)—NHR^{A1}, —C(═O)—NR^{A1}₂, —C(═O)—NR^{A2}R^{A3}, phenyl, or benzyl; wherein each R^{A1} is independently C₁₋₄alkyl, phenyl, or benzyl; and each —NR^{A2}R^{A3} is independently pyrrolidino, piperidino, piperizino, or morpholino, and is independently unsubstituted or substituted with one or more groups selected from C₁₋₃alkyl and —CF₃.

39. A compound according to claim 1, wherein -R^{AK2}-, if present, is independently unsubstituted or substituted with one or more substitutents -R^{G1}, wherein each -R^{G1}, if present, is independently —F, —Cl, —Br, —I, —OH, —OMe, —OEt, or —OCF₃.

40. A compound according to claim 1, wherein -R^{AK2}-, if present, is independently unsubstituted.

41. A compound according to claim 1, wherein -R^{AK2}-, if present, is independently:
—CH═CH—,
—C(CH₃)═CH—, —CH═C(CH₃)—;
—CH═CH—CH₂—,
—C(CH₃)═CH—CH₂—, —CH═C(CH₃)—CH₂—, —CH═CH—CH(CH₃)—,
—CH₂—CH═CH—,
—CH(CH₃)—CH═CH—, —CH₂—C(CH₃)═CH—, —CH₂—CH═C(CH₃)—,
—CH═CH—CH₂—CH₂—, —CH₂—CH═CH—CH₂—, or —CH₂—CH₂—CH═CH—.

42. A compound according to claim 1, wherein -R^{AK3}-, if present, is independently aliphatic C₂₋₄alkynylene; and is optionally substituted.

43. A compound according to claim 1, wherein -R^{AK3}-, if present, is independently unsubstituted or substituted with one or more substituents -R^{G1}, wherein each —R^{G1}, if present, is independently —F, —Cl, —Br, —I, —OH, —OR^{A1}, —OCF₃, —C(═O)OH, —C(═O)OR^{A1}, —NH₂, —NHR^{A1}, —NR^{A1}₂, —NR^{A2}R^{A3}, —C(═O)—NH₂, —C(═O)—NHR^{A1}, —C(═O)—NR^{A1}₂, —C(═O)—NR^{A2}R^{A3}, phenyl, or benzyl; wherein each R^{A1} is independently C₁₋₄alkyl, phenyl, or benzyl; and each —NR^{A2}R^{A3} is independently pyrrolidino, piperidino, piperizino, or morpholino, and is independently unsubstituted or substituted with one or more groups selected from C₁₋₃alkyl and —CF₃.

44. A compound according to claim 1, wherein -R^{AK3}-, if present, is independently unsubstituted or substituted with one or more substituents -R^{G1}, wherein each -R^{G1}, if present, is independently —F, —Cl, —Br, —I, —OH, —OMe, —OEt, or —OCF₃.

45. A compound according to claim 1, wherein -R^{AK3}-, if present, is independently unsubstituted.

46. A compound according to any one of claims 1 to 41, wherein -R^{AK3}-, if present, is independently:
—C≡C—,
—C≡C—CH₂—, —C≡C—CH(CH₃)—,
—CH₂—C≡C—, —CH(CH₃)—C≡C—,
—C≡C—CH₂—CH₂—, —C≡C—CH(CH₃)—CH₂—, —C≡C—CH₂—CH(CH₃)—,
—CH₂—C≡C—CH₂—, —CH(CH₃)—C≡C—CH₂—, —CH₂—C≡C—CH(CH₃)—,
—CH₂—CH₂—C≡C—, —CH(CH₃)—CH₂—C≡C—, —CH₂—CH(CH₃)—C≡C—,
—C≡C—CH═CH—, —C≡C—C(CH₃)═CH—, —C≡C—CH═C(CH₃)—,
—CH═CH—C≡C—, —C(CH₃)═CH—C≡C—, or —CH═C(CH₃)—C≡C—.

47. A compound according to claim 1, wherein each -R^{AK4}-, if present, is independently saturated C₃₋₅cycloalkylene; and is optionally substituted.

48. A compound according to claim 1, wherein each -R^{AK4}-, if present, is independently saturated C₃₋₄cycloalkylene; and is optionally substituted.

49. A compound according to claim 1, wherein each -R^{AK4}-, if present, is independently unsubstituted or substituted with one or more substitutents -R^{G1}, wherein each —R^{G1}, if present, is independently —F, —Cl, —Br, —I, —OH, —OR^{A1}, —OCF₃, —C(═O)OH, —C(═O)OR^{A1}, —NH₂, —NHR^{A1}, —NR^{A1}₂, —NR^{A2}R^{A3}, —C(═O)—NH₂, —C(═O)—NHR^{A1}, —C(═O)—NR^{A1}₂, —C(═O)—NR^{A2}R^{A3}, phenyl, or benzyl; wherein each R^{A1} is independently C₁₋₄alkyl, phenyl, or benzyl; and each —NR^{A2}R^{A3} is independently pyrrolidino, piperidino, piperizino, or morpholino, and is independently unsubstituted or substituted with one or more groups selected from C₁₋₃allyl and —CF₃.

50. A compound according to claim 1, wherein each -R^{AK4}-, if present, is independently unsubstituted or substituted with one or more substitutents —R^{G1}, wherein each —R^{G1}, if present, is independently —F, —Cl, —Br, —I, —OH, —OMe, —OEt, or —OCF₃.

51. A compound according to claim 1, wherein each -R^{AK4}-, if present, is independently unsubstituted.

52. A compound according to claim 1, wherein each -R^{AK4}-, if present, is independently: cyclopropyl-di-yl, cyclobutyl-di-yl, cyclopentyl-di-yl, or cyclohexyl-di-yl.

53. A compound according to claim 1, wherein each -R^{AK4}-, if present, is independently cyclopropyl-di-yl.

54. A compound according to claim 1, wherein each -R^{AK4}-, if present, is independently cyclopropyl-1,1-di-yl.

55. A compound according to claim 1, wherein each -R^{AK1}-R^{AK4}-, if present, is independently: methylene-cyclopropyl-di-yl, methylene-cyclobutyl-di-yl, methylene-cyclopentyl-di-yl, or methylene-cyclohexyl-di-yl.

56. A compound according to claim 1, wherein each -R^{AK4}-R^{AK1}-, if present, is independently: cyclopropyl-di-yl-methylene, cyclobutyl-di-yl-methylene, cyclopentyl-di-yl-methylene, or cyclohexyl-di-yl-methylene.

57. A compound according to claim 1, wherein -R^{AK1}-R^{AK4}-R^{AK1}, if present, is independently:
methylene-cyclopropyl-di-yl-methylene, methylene-cyclobutyl-di-yl-methylene, methylene-cyclopentyl-di-yl-methylene, or methylene-cyclohexyl-di-yl-methylene.

58. A compound according to claim 1, wherein each -R^{AK5}-, if present, is independently C₃₋₅cycloalkenylene; and is optionally substituted.

59. A compound according to claim 1, wherein each -R^{AK5}-, if present, is independently C₃₋₄cycloalkenylene; and is optionally substituted.

60. A compound according to claim 1, wherein each -R^{AK5}-, if present, is independently unsubstituted or substituted with one or more substitutents -R^{G1}, wherein each —R^{G1}, if present, is independently —F, —Cl, —Br, —I, —OH, —OR^{A1}, —OCF₃, —C(═O)OH, —C(═O)OR^{A1}, —NH₂, —NBR^{A1}, —NR^{A1}₂, —NR^{A2}R^{A3}, —C(═O)—NH₂, —C(═O)—NHR^{A1}, —C(═O)—NR^{A1}₂, —C(═O)—NR^{A2}R^{A3}, phenyl, or benzyl; wherein each R^{A1} is independently C₁₋₄alkyl, phenyl, or benzyl; and each —NR^{A2}R^{A3} is independently pyrrolidino, piperidino, piperizino, or morpholino, and is independently unsubstituted or substituted with one or more groups selected from C₁₋₃alkyl and —CF₃.

61. A compound according to claim 1, wherein each -R^{AK5}-, if present, is independently unsubstituted or substituted with one or more substitutents -R^{G1}, wherein each —R^{G1}, if present, is independently —F, —Cl, —Br, —I, —OH, —OMe, —OEt, or —OCF₃.

62. A compound according to claim 1, wherein each -R^{AK5}-, if present, is independently unsubstituted.

63. A compound according to claim 1, wherein each -R^{AK5}-, if present, is independently: cyclopropenyl-di-yl, cyclobutenyl-di-yl, cyclopentenyl-di-yl, or cyclohexenyl-di-yl.

64. A compound according to claim 1, wherein each -R^{AK1}-R^{AK5}-, if present, is independently:
methylene-cyclopropenyl-di-yl, methylene-cyclobutenyl-di-yl, methylene-cyclopentenyl-di-yl, or methylene-cyclohexenyl-di-yl.

65. A compound according to claim 1, wherein each -R^{AK5}-R^{AK1}-, if present, is independently:
cyclopropenyl-di-yl-methylene, cyclobutenyl-di-yl-methylene, cyclopentenyl-di-yl-methylene, or cyclohexenyl-di-yl-methylene.

66. A compound according to claim 1, wherein -R^{AK1}-R^{AK5}-R^{AK1}-, if present, is independently:
methylene-cyclopropenyl-di-yl-methylene, methylene-cyclobutenyl-di-yl-methylene, methylene-cyclopentenyl-di-yl-methylene, or
methylene-cyclohexenyl-di-yl-methylene.

67. A compound according to claim 1, wherein -R^{N} is independently —H, -R^{NNN}, or -L^{N}-R^{NNN}.

68. A compound according to claim 1, wherein -R^{N} is independently —H or -R^{NN}.

69. A compound according to claim 1, wherein -R^{N} is independently -R^{NNN} or -L^{N}-R^{NNN}.

70. A compound according to claim 1, wherein -R^{N} is independently —H.

71. A compound according to claim 14, wherein -R^{N} is independently -R^{NN}.

72. A compound according to claim 1, wherein -R^{N} is independently -R^{NNN}.

73. A compound according to claim 1, wherein -R^{N} is independently -L^{N}-R^{NNN}.

74. A compound according to claim 1, wherein:
W is independently —CR^{PW}═;
X is independently —CR^{PX}═;
Y is independently —CR^{PY}═;
Z is independently —CR^{PZ}═;
each of -R^{PW}, -R^{PX}, -R^{PY}, and -R^{PZ}, if present, is independently —H or -R^{RS1};
z is 1;
-J< is independently —N<;
-R^{AK}- is independently -R^{AK1}-;
-R^{AK1}- is independently —CH₂—; and
-R^{N} is independently -R^{NNN}.

75. A compound according to claim 1, wherein -L^{N}-, if present, is independently C₁₋₃alkylene, and is optionally substituted.

76. A compound according to claim 1, wherein -L^{N}-, if present, is independently unsubstituted or substituted with one or more substitutents -R^{G2}, wherein each -R^{G2}, if present, is independently —F, —Cl, —Br, —I, —OH, —OR^{A1}, —OCF₃, —C(═O)OH, —C(═O)OR^{A1}, —NH₂, —NHR^{A1}, —NR^{A1}₂, —NR^{A2}R^{A3}, —C(═O)—NH₂, —C(═O)—NHR^{A1}; —C(═O)—NR^{A1}₂, —C(═O)—NR^{A2}R^{A3}, phenyl, or benzyl; wherein each R^{A1} is independently C₁₋₄alkyl, phenyl, or benzyl; and each —NR^{A2}R^{A3} is independently pyrrolidino, piperidino, piperizino, or morpholino, and is independently unsubstituted or substituted with one or more groups selected from C₁₋₃alkyl and —CF₃.

77. A compound according to claim 1, wherein -L^{N}-, if present, is independently unsubstituted or substituted with one or more substitutents —R^{G2}, wherein each —R^{G2}, if present, is independently —F, —Cl, —Br, —I, —OH, —OMe, —OEt, or —OCF₃.

78. A compound according to claim 1, wherein -L^{N}-, if present, is independently unsubstituted.

79. A compound according to claim 1, wherein -L^{N}-, if present, is independently —CH₂—, —CH₂CH₂—, or —CH₂CH₂CH₂—.

80. A compound according to claim 1, wherein -L^{N}-, if present, is independently —CH₂— or —CH₂CH₂—.

81. A compound according to claim 1, wherein -L^{N}-, if present, is independently —CH₂—.

82. A compound according to claim 1, wherein -R^{NN}, if present, is independently C₁₋₄alkyl, and is optionally substituted.

83. A compound according to claim 1, wherein -R^{NN}, if present, is independently unsubstituted or substituted with one or more substitutents -R^{G3} wherein each —R^{G3} if present, is independently —F, —Cl, —Br, —I, —OH, —OR^{A1}, —OCF₃, —C(═O)OH, —C(═O)OR^{A1}, —NH₂, —NHR^{A1}, —NR^{A1}₂, —NR^{A2}R^{A3}, —C(═O)—NH₂, —C(═O)—NHR^{A1}, —C(═O)—NR^{A1}₂, —C(═O)—NR^{A2}R^{A3}; wherein each R^{A1} is independently C₁₋₄alkyl, phenyl, or benzyl; and each —NR^{A2}R^{A3} is independently pyrrolidino, piperidino, piperizino, or morpholino, and is independently unsubstituted or substituted with one or more groups selected from C₁₋₃allyl and —CF₃.

84. A compound according to claim 1, wherein -R^{NN}, if present, is independently unsubstituted or substituted with one or more substitutents -R^{G3} wherein each —R^{G3} if present, is independently —F, —Br, —I, —OH, —OMe, —OEt, or —OCF₃.

85. A compound according to claim 1, wherein -R^{NN}, if present, is independently unsubstituted.

86. A compound according to claim 1, wherein -R^{NN}, if present, is independently -Me, -Et, -nPr, or -iPr.

87. A compound according to claim 1, wherein -R^{NNN}, if present, is independently cyclopropyl, cyclobutyl, cyclopentyl, cyclohexyl, pyrrolidinyl, imidazolidinyl, pyrazolidinyl, piperidinyl, piperizinyl, morpholinyl, thiomorpholinyl, azepinyl, diazepinyl, phenyl, naphthyl, furanyl, thienyl, pyrrolyl, imidazolyl, pyrazolyl, triazolyl, oxazolyl, isoxazolyl, thiazolyl, isothiazolyl, pyridyl, pyrazinyl, pyrimidinyl, pyridazinyl, benzofuranyl, isobenzofuranyl, indazolyl, purinyl, quinolinyl, isoquinolinyl, naphthyridinyl, quinoxalinyl, quinazolinyl, cinnolinyl, indolyl, isoindolyl, carbazolyl, carbolinyl, acridinyl, phenoxazinyl, or phenothiazinyl; and is optionally substituted.

88. A compound according to claim 86, wherein -R^{NNN}, if present, is independently C₆₋₁₀carboaryl or C₅₋₁₀heteroaryl, and is optionally substituted.

89. A compound according to claim 1, wherein -R^{NNN}, if present, is independently phenyl, naphthyl, furanyl, thienyl, pyrrolyl, imidazolyl, pyrazolyl, triazolyl, oxazolyl, isoxazolyl, thiazolyl, isothiazolyl, pyridyl, pyrazinyl, pyrimidinyl, pyridazinyl, benzofuranyl, isobenzofuranyl, indazolyl, purinyl, quinolinyl, isoquinolinyl, naphthyridinyl, quinoxalinyl, quinazolinyl, cinnolinyl, indolyl, isoindolyl, carbazolyl, carbolinyl, acridinyl, phenoxazinyl, or phenothiazinyl; and is optionally substituted.

90. A compound according to claim 1, wherein -R^{NNN}, if present, is independently phenyl, naphthyl, furanyl, thienyl, pyrrolyl, imidazolyl, pyrazolyl, triazolyl, oxazolyl, isoxazolyl, thiazolyl, isothiazolyl, pyridyl, pyrazinyl, pyrimidinyl, or pyridazinyl; and is optionally substituted.

91. A compound according to claim 1, wherein -R^{NNN}, if present, is independently phenyl, naphthyl, pyridyl, pyrazinyl, pyrimidinyl, pyridazinyl, or pyrazolyl; and is optionally substituted.

92. A compound according to claim 1, wherein -R^{NNN}, if present, is independently phenyl, naphthyl, pyridyl, or pyrazolyl; and is optionally substituted.

93. A compound according to claim 1, wherein -R^{NNN}, if present, is independently phenyl or naphthyl; and is optionally substituted.

94. A compound according to claim 1, wherein -R^{NNN}, if present, is independently phenyl; and is optionally substituted.

95. A compound according to claim 1, wherein -R^{NNN}, if present, is independently phenyl; and is optionally substituted at the para position; and is unsubstituted at all other positions.

96. A compound according to claim 1, wherein -R^{NNN}, if present, is independently unsubstituted.

97. A compound according to claim 1, wherein each —R^{RS1}, if present, is independently —F, —Cl, —Br, —I, —R^{A1}, —CF₃, —OH, —OR^{A1}, —OCF₃, —C(═O)OH, —C(═O)OR^{A1}, —NH₂, —NHR^{A1}, —NR^{A1}₂, —NR^{A2}R^{A3}, —C(═O)—NH₂, —(═O)—NHR^{A1}, —C(═O)—NR^{A1}₂, —C(═O)—NR^{A2}R^{A3}, phenyl, or benzyl; wherein each R^{A1} is independently C₁₋₄alkyl, phenyl, or benzyl; and each —NR^{A2}R^{A3} is independently pyrrolidino, piperidino, piperizino, or morpholino, and is independently unsubstituted or substituted with one or more groups selected from C₁₋₃alkyl and —CF₃; and additionally, two adjacent groups -R^{RS1}, if present, may form —OCH₂O—, —OCH₂CH₂O—, or —OCH₂CH₂CH₂O—.

98. A compound according to claim 1, wherein each —R^{RS1}, if present, is independently —F, —Cl, —Br, —I, -Me, -Et, —CF₃, —OH, —OMe, —OEt, —OCF₃, or phenyl; and additionally, two adjacent groups —R^{RS1}, if present, may form —OCH₂CH₂O—.

99. A compound according to claim 1, wherein each —R^{RS1}, if present, is independently —F, —Cl, —Br, -Me, —CF₃, —OMe, —OEt, or phenyl; and additionally, two adjacent groups —R^{RS1}, if present, may form —OCH₂CH₂O—.

100. A compound according to claim 1, wherein each substituent on -R^{NNN}, if present, is independently -R^{S}, and wherein each -R^{S}, if present, is independently:
—F, —Cl, —Br, —I,
—R^{D1},
—CF₃, —CH₂CF₃, —CF₂CF₂H,
—OH,
-L¹-OH,
—O-L¹-OH,
—OR^{D1},
-L¹-OR^{D1},
—O-L¹-OR^{D1}
—OCF₃, —OCH₂CF₃, —OCF₂CF₂H,
—SH,
—SR^{D1}, —SCF₃,
—CN,
—NO₂,
—NH₂, —NHR^{D1}, —NR^{D1}₂, —NR^{N1}R^{N2},
-L¹-NH₂, -L¹-NHR^{D1}, -L¹-NR^{D1}₂, -L¹-NR^{N1}R^{N2},
—O-L¹-NH₂, —O-L¹-NHR^{D1}, —O-L¹-NR^{D1}₂, —O-L¹-NR^{N1}R^{N2},
—NH-L¹-NH₂, —NH-L¹-NHR^{D1}, —NH-L¹-NR^{D1}₂, —NH-L¹-NR^{N1}R^{N2},
—NR^{D1}-L¹-NH₂, —NR^{D1}-L¹-NHR^{D1}, —NR^{D1}-L¹-NR^{D1}₂, —NR^{D1}-L¹-NR^{N1}R^{N2},
—C(═O)OH,
—C(═O)OR^{D1},
—C(═O)NH₂, —C(═O)NHR^{D1}, —C(═O)NR^{D1}₂, —C(═O)NR^{N1}R^{N2},
—NHC(═O)R^{D1}, —NR^{D1}C(═O)R^{D1},
—NHC(═O)OR^{D1}, —NR^{D1}C(═O)OR^{D1},
—OC(═O)NH₂, —OC(═O)NHR^{D1}, —OC(═O)NR^{D1}₂, —OC(═O)NR^{N1}R^{N2},
—OC(═O)R^{D1},
—C(═O)R^{D1},
—NHC(═O)NH₂, —NHC(═O)NHR^{D1}, —NHC(═O)NR^{D1}₂, —NHC(═O)NR^{N1}R^{N2},
—NR^{D1}C(═O)NH₂, —NR^{D1}C(═O)NHR^{D1}, —NR^{D1}C(═O)NR^{D1}₂, —NR^{D1}C(═O)NR^{N1}R^{N2},
—NHS(═O)₂R^{D1}, —NR^{D1}S(═O)₂R^{D1},
—S(═O)₂NH₂, —S(═O)₂NHR^{D1}, —S(═O)₂NR^{D1}₂, —S(═O)₂NR^{N1}R^{N2},
—S(═O)R^{D1},
—S(═O)₂R^{D1},
—OS(═O)₂R^{D1},
—S(═O)₂OR^{D1},
═O,
═NR^{D1},
═NOH, or
═NOR^{G1};
and additionally, two ring adjacent groups -R^{S}, if present, may together form a group —O-L²-O—;
wherein:
each -L¹- is independently saturated aliphatic C₁₋₅alkylene, aliphatic C₂₋₅alkenylene, or aliphatic C₂₋₅alkynylene;
each -L²- is independently saturated aliphatic C₁₋₃alkylene;
in each group —NR^{N1}R^{N2}, -R^{N1} and -R^{N2}, taken together with the nitrogen atom to which they are attached, form a 5-, 6-, or 7-membered non-aromatic ring having exactly 1 ring heteroatom or exactly 2 ring heteroatoms, wherein one of said exactly 2 ring heteroatoms is N, and the other of said exactly 2 ring heteroatoms is independently N, O, or S;
each -R^{D1} is independently:
-R^{E1}, -R^{E2}, -R^{E3}, -R^{E4}, -R^{E5}, -R^{E6}, -R^{E7}, -R^{E8},
L³-R^{E4}, -L³-R^{E5}, -L³-R^{E6}, -L³-R^{E7}, or -L³-R^{E8};
wherein:
each -R^{E1} is independently saturated aliphatic C₁₋₆alkyl;
each -R^{E2} is independently aliphatic C₂₋₆alkenyl;
each -R^{E3} is independently aliphatic C₂₋₆alkynyl;
each -R^{E4} is independently saturated C₃₋₆cycloalkyl;
each -R^{E5} is independently C₃₋₆cycloalkenyl;
each -R^{E6} is independently non-aromatic C₃₋₇heterocyclyl;
each -R^{E7} is independently C₆₋₁₄carboaryl;
each -R^{E8} is independently C₅₋₁₄heteroaryl;
each -L³- is independently saturated aliphatic C₁₋₃alkylene;
and wherein:
each C₁₋₆alkyl, C₂₋₆alkenyl, C₂₋₄alkynyl, C₃₋₆cycloalkyl, C₃₋₆cycloalkenyl, non-aromatic C₃₋₇heterocyclyl, C₆₋₁₄carboaryl, C₅₋₁₄heteroaryl, and C₁₋₃alkylene is optionally substituted, for example, with one or more substituents -R^{G4}, wherein each -R^{G4} is independently:
—F, —Cl, —Br, —I,
-R^{F1},
—CF₃, —CH₂CF₃, —CF₂CF₂H,
—OH,
-L⁴-OH,
—O-L⁴-OH,
—OR^{F1},
-L⁴-OR^{F1},
—O-L⁴-OR^{F1},
—OCF₃, —OCH₂CF₃, —OCF₂CF₂H,
—SH,
—SR^{F1}, —SCF₃,
—CN,
—NO₂,
—NH₂, —NHR^{F1}, —NR^{F1}₂, —NR^{N3}R^{N4},
-L⁴-NH₂, -L⁴-NHR^{F1}, -L⁴-NR^{F1}₂, or -L⁴-NR^{N3}R^{N4},
—O-L⁴-NH₂, —O-L⁴-NHR^{F1}, —O-L⁴-NR^{F1}₂, —O-L⁴NR^{N3}R^{N4},
—NH-L⁴-NH₂, —NH-L⁴-NHR^{F1}, —NH-L⁴-NR^{F1}₂, —NH-L⁴-NR^{N3}R^{N4},
—NR^{F1}-L⁴-NH₂, —NR^{F1}-L⁴-NHR^{F1}, —NR^{F1}-L⁴-NR^{F1}₂, —NR^{F1}-L⁴-NR^{N3}R^{N4},
—C(═O)OH,
—C(═O)OR^{F1},
—C(═O)NH₂, —C(═O)NHR^{F1}, —C(═O)NR^{F1}₂, or —C(═O)NR^{N3}R^{N4};
wherein:
each -R^{F1} is independently saturated aliphatic C₁₋₄alkyl, phenyl, or benzyl;
each -L⁴- is independently saturated aliphatic C₁₋₅alkylene; and
in each group —NR^{N3}R^{N4}, —R^{N3} and -R^{N4}, taken together with the nitrogen atom to which they are attached, form a 5-, 6-, or 7-membered non-aromatic ring having exactly 1 ring heteroatom or exactly 2 ring heteroatoms, wherein one of said exactly 2 ring heteroatoms is N, and the other of said exactly 2 ring heteroatoms is independently N, O, or S.

101. A compound according to claim 1, wherein each substituent on -R^{NNN}, if present, is independently -R^{S}, and wherein each -R^{S}, if present, is independently:
—F, —Cl, —Br, —I,
—R^{D1},
—CF₃, —CH₂CF₃, —CF₂CF₂H,
—OH,
-L¹-OH,
—O-L¹- OH,
—OR^{D1},
-L¹-OR^{D1},
—O-L¹-OR^{D1},
—OCF₃, —OCH₂CF₃, —OCF₂CF₂H,
—SH,
—SR^{D1}, —SCF₃,
—CN,
—NO₂,
—NH₂, —NHR^{D1}, —NR^{D1}₂, —NR^{N1}R^{N2},
-L¹-NH₂, -L¹-NHR^{D1}, -L¹-NR^{D1}₂, -L¹-NR^{N1}R^{N2},
—O-L¹-NH₂, —O-L¹-NHR^{D1}, —O-L¹-NR^{D1}₂, —O-L¹-NR^{N1}R^{N2},
—NH-L¹-NH₂, —NH-L¹-NHR^{D1}, —NH-L¹-NR^{D1}₂, —NH-L¹-NR^{N1}R^{N2},
—NR^{D1}-L¹-NH₂, —NR^{D1}-L¹-NHR^{D1}, —NR^{D1}-L¹-NR^{D1}₂, —NR^{D1}-L¹-NR^{N1}R^{N2},
—C(═O)OH,
—C(═O)OR^{D1},
—C(═O)NH₂, —C(═O)NHR^{D1}, —C(═O)NR^{D1}₂, —C(═O)NR^{N1}R^{N2},
—NHC(═O)R^{D1}, —NR^{D1}C(═O)R^{D1},
—OC(═O)R^{D1},
—C(═O)R^{D1},
—NHS(═O)₂R^{D1}, —NR^{D1}S(═O)₂R^{D1},
—S(═O)₂NH₂, —S(═O)₂NHR^{D1}, —S(═O)₂NR^{D1}₂, or —S(═O)₂NR^{N1}R^{N2};
and additionally, two ring adjacent groups —R^{S}, if present, may together form a group —O-L²-O—.

102. A compound according to claim 1, wherein each substituent on -R^{NNN}, if present, is independently -R^{S}, and wherein each -R^{S}, if present, is independently —OR^{D1}.

103. A compound according to claim 100, wherein each group —NR^{N1}R^{N2}, if present, is independently pyrrolidino, imidazolidino, pyrazolidino, piperidino, piperizino, morpholino, thiomorpholino, azepino, or diazepino, and is independently unsubstituted or substituted with one or more groups selected from C₁₋₃alkyl and —CF₃.

104. A compound according to claim 100, wherein each group —NR^{N1}R^{N2}, if present, is independently pyrrolidino, piperidino, piperizino, or morpholino, and is independently unsubstituted or substituted with one or more groups selected from C₁₋₃alkyl and —CF₃.

105. A compound according to claim 100, wherein each -R^{D1}, if present, is independently:
-R^{E1}, -R^{E3}, -R^{E4}, -R^{E7}, -R^{E8},
-L³-R^{E4}, -L³-R^{E7}, or -L³-R^{E8}.

106. A compound according to claim 100, wherein each -R^{D1}, if present, is independently -R^{E1}, -R^{E3}, -R^{E7}, -R^{E8}, -L³-R^{E7}, or -L³-R^{E8}.

107. A compound according to claim 100, wherein each -R^{D1}, if present, is independently -L³-R^{E7} or -L³-R^{E8}.

108. A compound according to claim 100, wherein each -R^{D1}, if present, is independently -R^{E3}.

109. A compound according to claim 100, wherein each -R^{E1}, if present, is independently methyl, ethyl, n-propyl, i-propyl, n-butyl, i-butyl, s-butyl, or t-butyl, and is optionally substituted.

110. A compound according to claim 100, wherein each -R^{E2}, if present, is independently aliphatic C₂₋₄alkenyl, and is optionally substituted.

111. A compound according to claim 100, wherein each -R^{E2}, if present, is independently —CH₂—CH═CH₂, and is optionally substituted.

112. A compound according to claim 100, wherein each -R^{E3}, if present, is independently aliphatic C₃₋₅alkynyl, and is optionally substituted.

113. A compound according to claim 100, wherein each -R^{E3}, if present, is independently —CH₂—C≡CH, —CH(CH₃)—C≡CH, —CH₂—C≡C—CH₃, —CH(CH₃)—C≡C—CH₃, —CH₂—C≡C—CH₂—CH₃, or —CH₂—CH₂—C≡CH, and is optionally substituted.

114. A compound according to claim 100, wherein each -R^{E4}, if present, is independently cyclopropyl, cyclobutyl, cyclopentyl, or cyclohexyl, and is optionally substituted.

115. A compound according to claim 100, wherein each -R^{E6}, if present, is independently azetidinyl, pyrrolidinyl, imidazolidinyl, pyrazolidinyl, piperidinyl, piperazinyl, morpholinyl, azepinyl, diazepinyl, tetrahydrofuranyl, tetrahydropyranyl, dioxanyl, and is optionally substituted.

116. A compound according to claim 100, wherein each -R^{E6}, if present, is independently pyrrolidinyl, piperidinyl, piperazinyl, morpholinyl, tetrahydrofuranyl, or tetrahydropyranyl, and is optionally substituted.

117. A compound according to claim 100, wherein each -R^{E7}, if present, is independently phenyl or naphthyl; and is optionally substituted.

118. A compound according to claim 100, wherein each -R^{E7}, if present, is independently phenyl; and is optionally substituted.

119. A compound according to claim 100, wherein each -R^{E8}, if present, is independently furanyl, thienyl, pyrrolyl, imidazolyl, pyrazolyl, triazolyl, oxazolyl, isoxazolyl, thiazolyl, isothiazolyl, pyridyl, pyrazinyl, pyrimidinyl, pyridazinyl, benzofuranyl, isobenzofuranyl, indazolyl, purinyl, quinolinyl, isoquinolinyl, naphthyridinyl, quinoxalinyl, quinazolinyl, cinnolinyl, indolyl, isoindolyl, carbazolyl, carbolinyl, acridinyl, phenoxazinyl, or phenothiazinyl; and is optionally substituted.

120. A compound according to claim 100, wherein each -R^{E8}, if present, is independently furanyl, thienyl, pyrrolyl, imidazolyl, pyrazolyl, triazolyl, oxazolyl, isoxazolyl, thiazolyl, isothiazolyl, pyridyl, pyrazinyl, pyrimidinyl, pyridazinyl, quinolinyl, or isoquinolinyl; and is optionally substituted.

121. A compound according to claim 100, wherein each -R^{E8}, if present, is independently furanyl, pyrrolyl, pyrazolyl, triazolyl, oxazolyl, isoxazolyl, thiazolyl, isothiazolyl, pyridyl, quinolinyl, or isoquinolinyl; and is optionally substituted.

122. A compound according to claim 100, wherein each -L¹-, if present, is independently saturated aliphatic C₁₋₅alkylene or aliphatic C₂₋₅alkynylene.

123. A compound according to claim 100, wherein each -L¹-, if present, is independently saturated aliphatic C₁₋₅alkylene.

124. A compound according to claim 100, wherein each -L¹-, if present, is independently saturated aliphatic C₂₋₅alkylene.

125. A compound according to claim 100, wherein each -L²-, if present, is independently —CH₂— or —CH₂CH₂—.

126. A compound according to claim 100, wherein each -L²-, if present, is independently —CH₂CH₂—.

127. A compound according to claim 100, wherein each -L³-, if present, is independently —CH₂L .

128. A compound according to claim 100, wherein each -R^{G4}, if present, is independently selected from:
—F, —Cl, —Br, —I,
—R^{F1},
—CF₃, —CH₂CF₃, —CF₂CF₂H,
—OH,
-L⁴-OH,
—O-L⁴-OH,
—OR^{F1},
-L⁴-OR^{F1},
—O-L⁴-OR^{F1},
—OCF₃, —OCH₂CF₃, —OCF₂CF₂H,
—SR^{F1},
—NH₂, —NHR^{F1}, —NR^{F1}₂, —NR^{N3}R^{N4},
-L⁴-NH₂, -L⁴-NHR^{F1}, -L⁴-NR^{F1}₂, or -L⁴-NR^{N3}R^{N4},
—O-L⁴-NH₂, —O-L⁴-NHR^{F1}, —O-L⁴-NR^{F1}₂, —O-L⁴-NR^{N3}R^{N4},
—NH-L⁴-NH₂, —NH-L⁴-NHR^{F1}, —NH-L⁴-NR^{F1}₂, —NH-L⁴-NR^{N3}R^{N4},
—NR^{F1}-L⁴-NH₂, —NR^{F1}-L⁴-NHR^{F1}, -NR^{F1}-L⁴-NR^{F1}₂, or —NR^{F1}-L⁴-NR^{N3}R^{N4}.

129. A compound according to claim 100, wherein each -R^{G4}, if present, is independently selected from:
—F, —Cl, —Br, —I,
—R^{F1},
—OH,
—OR^{F1},
—NH₂, —NHR^{F1}, —NR^{F1}₂, and —NR^{N3}R^{N4}.

130. A compound according to claim 100, wherein each group —NR^{N3}R^{N4}, if present, is independently pyrrolidino, imidazolidino, pyrazolidino, piperidino, piperizino, morpholino, thiomorpholino, azepino, or diazepino, and is independently unsubstituted or substituted, for example, with one or more groups selected from C₁₋₃allyl and —CF₃.

131. A compound according to claim 100, wherein each group —NR^{N3}R^{N4}, if present, is independently pyrrolidino, piperidino, piperizino, or morpholino, and is independently unsubstituted or substituted, for example, with one or more groups selected from C₁₋₃alkyl and —CF₃.

132. A compound according to claim 100, wherein each -R^{F1}, if present, is independently saturated aliphatic C₁₋₄alkyl.

133. A compound according to claim 100, wherein each -L⁴-, if present, is independently saturated aliphatic C₂₋₅alkylene.

134. A compound according to claim 1, the ring carbon atom adjacent to the group J is in the (R) configuration.

135. A compound according to claim 1, the ring carbon atom adjacent to the group J is in the (S) configuration.

136. A compound according to claim 1, selected from the following compounds and pharmaceutically acceptable salts thereof: IX-001 through IX-096.

137. A compound according to claim 1, selected from the following compound and pharmaceutically acceptable salts thereof: IX-097.

138. A compound according to claim 1, selected from the following compound and pharmaceutically acceptable salts thereof: IX-098.

139. A compound according to claim 1, selected from the following compounds and pharmaceutically acceptable salts thereof: IX-099 through IX-101.

140. A pharmaceutical composition comprising a compound according to claim 1, and a pharmaceutically acceptable carrier, diluent, or excipient.

141. A method of preparing a pharmaceutical composition comprising admixing a compound according to claim 1 and a pharmaceutically acceptable carrier, diluent, or excipient.

142.-158. (canceled)

159. A method of treatment of a disease or disorder that is mediated by TACE comprising administering to a patient in need of treatment a therapeutically effective amount of a compound according to claim 1.

160. A method of treatment of a disease or disorder that is ameliorated by the inhibition of TACE comprising administering to a patient in need of treatment a therapeutically effective amount of a compound according to claim 1.

161. A method of treatment of a disease or disorder that is treated by a TACE inhibitor comprising administering to a patient in need of treatment a therapeutically effective amount of a compound according to claim 1.

162. A method of treatment of rheumatoid arthritis; inflammation; psoriasis; septic shock; graft rejection; cachexia; anorexia; congestive heart failure; post-ischaemic reperfusion injury; inflammatory disease of the central nervous system; inflammatory bowel disease; insulin resistance; HIV infection; cancer; chronic obstructive pulmonary disease (COPD); or asthma comprising administering to a patient in need of treatment a therapeutically effective amount of a compound according to claim 1.

163. A method of treatment of osteoarthritis, ulcerative colitis, Crohn's disease, multiple sclerosis, or degenerative cartilage loss comprising administering to a patient in need of treatment a therapeutically effective amount of a compound according to claim 1.

164. A method of treatment of inflammation comprising administering to a patient in need of treatment a therapeutically effective amount of a compound according to claim 1.

165. A method of treatment of rheumatoid arthritis comprising administering to a patient in need of treatment a therapeutically effective amount of a compound according to claim 1.

166. A method of treatment of psoriasis comprising administering to a patient in need of treatment a therapeutically effective amount of a compound according to claim 1.

167. A method of inhibiting TACE in a cell, in vitro or in vivo, comprising contacting said cell with an effective amount of a compound according to claim 1.

168. A method of regulating (e.g., inhibiting) cytokine release (e.g., TNF-α release) in a cell, in vitro or in vivo, comprising contacting said cell with an effective amount of a compound according to claim 1.

169. A kit comprising (a) a compound according to claim 1, preferably provided as a pharmaceutical composition and in a suitable container and/or with suitable packaging; and (b) instructions for use, for example, written instructions on how to administer the compound/composition.
